(19) United States
(12) Patent Application Publication
(10) Pub. No.: US 2022/0054656 A1
(43) Pub. Date: Feb. 24, 2022

(54) THERAPEUTIC ADENO-ASSOCIATED VIRUS FOR TREATING POMPE DISEASE

(71) Applicant: ASKLEPIOS BIOPHARMACEUTICAL, INC., Research Triangle Park, NC (US)

(72) Inventors: Michael W. O'Callaghan, Research Triangle Park, NC (US); Achille Francois, Research Triangle Park, NC (US)

(73) Assignee: ASKLEPIOS BIOPHARMACEUTICAL, INC., Research Triangle Park, NC (US)

(21) Appl. No.: 17/294,074

(22) PCT Filed: Nov. 15, 2019
(86) PCT No.: PCT/US2019/061653

§ 371 Date: May 14, 2021

Related U.S. Application Data
(60) Provisional application No. 62/768,449, filed on Nov. 16, 2018.
(60) Provisional application No. 62/769,702, filed on Nov. 20, 2018.

(51) Int. Cl. A61K 48/00; C12N 15/86; A61P 3/08

(52) U.S. Cl. CPC A61K 48/0058; C12N 15/86; A61P 3/08; C12N 2830/002; C12N 2750/14171; C12N 2830/008; C12N 2750/14143

(57) ABSTRACT

Recombinant AAV (rAAV) vectors comprising a rAVV genome comprising a heterologous nucleic acid encoding a signal peptide and optionally a IGF-2 sequence, fused to an acid alpha-glucosidase (GAA) polypeptide, enabling the GAA polypeptide to be secreted from the liver and targeted to the lysosomes. Particular embodiments relate to a recombinant AAV (rAAV) vector encoding an alpha-glucosidase (GAA) polypeptide, having a liver secretory signal peptide and a targeting IGF2 sequence that binds human cation-independent mannose-6-phosphate receptor (CI-MPR) or to the IGF2 receptor, permitting proper subcellular localization of the GAA polypeptide to lysosomes. Also encompassed are cells, and methods to treat a glycogen storage disease type II (GSD II) disease and/or Pompe Disease with the rAAV vector.

### CROSS-REFERENCE TO RELATED APPLICATIONS

This invention claims benefit under 35 U.S.C. § 119(e) of U.S. Provisional Application 62/768,449 filed on Nov. 16, 2018 and U.S. Provisional Application 62/769,702 filed on Nov. 20, 2018, the contents of each are incorporated herein in their entirety by reference.

### SEQUENCE LISTING

The instant application contains a Sequence Listing which has been submitted electronically in ASCII format, and is hereby incorporated by reference in its entirety. Said ASCII copy, created on Nov. 15, 2019 is named 046192-093900WOPT_SL.txt and is 189,408 bytes in size.

### FIELD OF THE INVENTION

The present invention relates to adeno-associated virus (AAV) particles, virions and vectors for targeted translocation of an alpha-glucidase (GAA) polypeptide, and method of use for the treatment of Pompe disease.

### BACKGROUND

Acid alpha-glucosidase (GAA) is a lysosomal enzyme that hydrolyzes the alpha 1-4 linkage in maltose and other linear oligosaccharides, including the outer branches of glycogen, thereby breaking down excess glycogen in the lysosome (Hirschhorn et al. (2001) in The Metabolic and Molecular Basis of Inherited Disease, Scriver, et al., eds. (2001), McGraw-Hill: New York, p. 3389-3420). Like other mammalian lysosomal enzymes, GAA is synthesized in the cytosol and traverses the ER where it is glycosylated with N-linked, high mannose type carbohydrate. In the golgi, the high mannose carbohydrate is modified on lysosomal proteins by the addition of mannose-6-phosphate (M6P) which targets these proteins to the lysosome. The M6P-modified proteins are delivered to the lysosome via interaction with either of two M6P receptors. The most favorable form of modification is when two M6Ps are added to a high mannose carbohydrate.

Insufficient GAA activity in the lysosome results in Pompe disease, a disease also known as acid maltase deficiency (AMD), glycogen storage disease type II (GSDII), glycogenosis type II, or GAA deficiency. The diminished enzymatic activity occurs due to a variety of missense and nonsense mutations in the gene encoding GAA. Consequently, glycogen accumulates in the lysosomes of all cells in patients with Pompe disease. In particular, glycogen accumulation is most pronounced in lysosomes of cardiac and skeletal muscle, liver, and other tissues. Accumulated glycogen ultimately impairs muscle function. In the most severe form of Pompe disease, death occurs before two years of age due to cardio-respiratory failure.

There is a need for an effective treatment of Pompe disease. Enzyme replacement therapeutics for Pompe require a recombinant GAA protein to be administered and taken up by muscle and liver cells in the subject where it is subsequently transported to the lysosomes in those cells in a M6P-dependent fashion. That is, a recombinant GAA protein with the exposed M6P binds to M6P receptors in the trans-Golgi and is transported to the endosome and then to the lysosome. However, two of the main sources of recombinant GAA protein used for enzyme replacement therapy; recombinant GAA produced in engineered CHO cells or in the milk of transgenic rabbits, contains extremely little M6P which is required for targeting the protein to lysosomes (Van Hove et al. (1996) Proc Natl Acad Sci USA, 93(1):65-70; and U.S. Pat. No. 6,537,785). Therefore, M6P-dependent delivery of a recombinant GAA protein to lysosomes is not efficient and requires both high dosages and frequent infusions.

Therefore, while enzyme therapy has demonstrated reasonable efficacy for severe infantile GSD II, the benefit of GAA enzyme therapy is limited by the need for frequent infusions as well as the subject developing inhibitor or neutralizing antibodies against recombinant hGAA protein (Amalfitano, A., et al. (2001) Genet. In Med. 3:132-138).

Gene therapy has the potential to not only cure genetic disorders, but to also facilitate the long-term non-invasive treatment of acquired and degenerative disease using a virus. One gene therapy vector is adeno-associated virus (AAV). AAV itself is a non-pathogenic-dependent parvovirus that needs helper viruses for efficient replication. AAV has been utilized as a virus vector for gene therapy because of its safety and simplicity. AAV has a broad host and cell type tropism capable of transducing both dividing and non-dividing cells. To date, 12 AAV serotypes and more than 100 variants have been identified. It has been shown that the different AAV serotypes can have differing abilities to infect cells of different tissues, either in vivo or in vitro and that these differences in infectivity are likely tied to the particular receptors and co-receptors located on the cell surface of each AAV serotype or may be tied to the intracellular trafficking pathway itself.

Accordingly, as an alternative to, or adjunct to enzyme therapy, the feasibility of gene therapy approaches to treat GSD-II have been investigated (Amalfitano, A., et al., (1999) Proc. Natl. Acad. Sci. USA 96:8861-8866, Ding, E., et al. (2002) Mol. Ther. 5:436-446, Fraites, T. J., et al., (2002) Mol. Ther. 5:571-578, Tsujino, S., et al. (1998) Hum. Gene Ther. 9:1609-1616).

However, AAV delivery of the GAA polypeptide has some challenges with respect to achieving sufficient expression in the liver and/or delivery to lysosomes with patients reporting to experience glycaemia. It has been reported that in in vivo studies using an adenovirus (Ad) vector encoding hGAA that was targeted to mouse liver in the GAA-KO mouse model, reversed the glycogen accumulation in skeletal and cardiac muscle within 12 days through secretion of hGAA from the liver and uptake in other tissues (Amalfitano, A., et al., (1999) Proc. Natl. Acad. Sci. USA 96:8861-8866). Introduction of adeno-associate virus 2 (AAV2) vectors encoding GAA normalized the GAA activity in the injected skeletal muscle and the injected cardiac muscle, and glycogen content was normalized in muscle when an AAV1-pseudotyped vector was administered with improved muscle transduction (Fraites, T. J., et al. (2002) Mol. Ther. 5:571-578). Muscle-targeted Ad vector gene therapy was attempted in the Japanese quail model, although only localized reversal of glycogen accumulation at the site of vector injection was achieved (Tsujino, S., et al. (1998) Hum. Gene Ther. 9:1609-1616).

However, in human subjects, the administration of rAAV vectors encoding GAA polypeptide have resulted in a number of patients experiencing hypoglycemia or becoming hyperglycemic due to non-specific update in cells (see, e.g., Byrne et al., A study on the safety and efficacy of Reveglucosidease alfa in patients with late-onset Pompe disease; Orphanet J. of Rare diseases; 2017; 12: 144).

Accordingly, there is a need in the art for improved methods of producing lysosomal polypeptides such as GAA in vitro and in vivo, for example, to treat lysosomal polypeptide deficiencies. Moreover, there is a need for improved secretion from the liver as well as improved targeting of GAA to the lysosomes to help reduce any side effects from overexpression of the GAA polypeptide, and reducing the risk of hypoglycemia. Further, there is a need for methods that result in systemic delivery of GAA and other lysosomal polypeptides to affected tissues and organs. In particular, there remains a need for more efficient methods for administering GAA protein to subjects and targeting GAA protein to patient lysosomes, while reducing any potential side effects.

### SUMMARY OF THE INVENTION

The technology described herein relates generally to gene therapy constructs, methods and composition, for the treatment of Pompe Disease. More particularly, the technology relates to adeno-associated (AAV) virions configured for delivering a GAA polypeptide to a subject. Accordingly, described herein are rAAV vectors that comprises a nucleotide sequence containing inverted terminal repeats (ITRs), a promoter, a heterologous gene, a poly-A tail and potentially other regulator elements for use to treat Pompe Disease, wherein the heterologous gene encodes the acid alpha-glucosidase (GAA) protein and wherein the rAAV expressing GAA protein can be administered to a patient in a therapeutically effective dose that is delivered to the appropriate tissue and/or organ for expression of the heterologous gene encoding the GAA protein for the treatment of a subject with Pompe disease.

Accordingly, the technology described herein relates in general to a means of expressing GAA protein in the liver using a rAAV vector and effectively targeting the expressed GAA protein to the lysosomes of mammalian cells, for example, human cardiac and skeletal muscle cells. Described herein are rAAV vectors, rAAV genomes and isolated nucleic acid compositions that encode lysosomal polypeptides (e.g., GAA) that are fused to a signal peptide, where the signal peptide enhances targeting of the GAA polypeptide to the secretory pathway, and where the GAA polypeptide is also optionally fused to targeting sequences to aid update into lysosomes. As such, the methods and compositions enable secretion of GAA polypeptides from the cell, e.g., liver cell, and lysosomal-targeting of GAA protein to the lysosomes of muscles. Lysosomal-targeting of GAA protein provides a number of advantages. For example, administration of rAAV vectors encoding GAA polypeptide have resulted in a number of patients experiencing hypoglycemia or becoming hyperglycemic due to non-specific update in cells (see, e.g., Byrne et al., A study on the safety and efficacy of Reveglucosidease alfa in patients with late-onset Pompe disease; Orphanet J. of Rare diseases; 2017; 12: 144). Optimal or improved secretion from the liver as well as improved targeting of GAA to the lysosomes will enable GAA to be expressed at lower levels and help reduce any side effects from overexpression of the GAA polypeptide, including reduce risk of hypoglycemia.

Accordingly, herein the inventors describe a rAAV vector, comprising in its genome a heterologous nucleic acid encoding a chimeric gene that encodes a secretory signal peptide (SS) operatively linked to a IGF2 sequence (e.g., a targeting peptide or TP) fused to the N-terminus of a GAA polypeptide at the native signal peptide cleavage site or at appropriate downstream sites. Expression of such a chimeric gene will direct the production of a recombinant GAA fusion protein that is secreted at high levels and that contains a high affinity ligand for the M6P/IGF2 receptor.

In some embodiments of the compositions and methods described herein, the rAAV vector disclosed herein comprises, in its genome: 5′ and 3′ AAV inverted terminal repeats (ITR) sequences, and located between the 5′ and 3′ ITRs, a heterologous nucleic acid sequence encoding a fusion polypeptide comprising (i) a secretory signal peptide, (ii) an IGF2 sequence; and (iii) an alpha-glucosidase (GAA) polypeptide, wherein the heterologous nucleic acid is operatively linked to a promoter, for example, but not limited to, a liver-specific promoter. In some embodiments, the rAAV vector disclosed herein comprises, in its genome: 5′ and 3′ AAV inverted terminal repeats (ITR) sequences, and located between the 5′ and 3′ ITRs, a heterologous nucleic acid sequence encoding a fusion polypeptide comprising (i) a secretory signal peptide, and (ii) an alpha-glucosidase (GAA) polypeptide, wherein the heterologous nucleic acid is operatively linked to a promoter, a liver-specific promoter.

In some embodiments of the compositions and methods described herein, the secretory signal peptide is selected from any of: AAT signal peptide, a fibronectin signal peptide (FN1), a GAA signal peptide, or an active fragment thereof having secretory signal activity.

In some embodiments of the compositions and methods described herein, the alpha-glucosidase (GAA) polypeptide is linked to the IGF2 sequence at the N-terminal end of the GAA polypeptide. In some embodiments, the IGF2 sequence is linked to the N-terminal at amino acid 70 of human acid alpha-glucosidase (GAA) polypeptide (SEQ ID NO: 10) (i.e., linked to the N-terminal of residues 70-952 of human acid alpha-glucosidase (GAA) polypeptide). In alternative embodiments, the IGF2 sequence is linked to the N-terminal at amino acid 40 of human acid alpha-glucosidase (GAA) polypeptide (SEQ ID NO: 10) (i.e., linked to the N-terminal of residues 40-952 of human acid alpha-glucosidase (GAA) polypeptide). In some embodiments of the compositions and methods described herein, the GAA polypeptide is encoded by the wild-type GAA nucleic acid sequence (e.g., SEQ ID NO: 11 or SEQ ID NO: 72), or can be a codon optimized GAA nucleic acid sequence, e.g., for any one of increasing expression in vivo, reducing CpG islands and/or reducing innate immune response in a subject. Exemplary codon optimized GAA nucleic acid sequences include, but are not limited to SEQ ID NO; 73, SEQ ID NO: 74, SEQ ID NO: 75 and SEQ ID NO: 76.

In some embodiments of the compositions and methods described herein, the IGF2 sequence is a nucleic acid sequence that encodes any of: residue 1 followed by residues 8-67 of wild-type mature human insulin-like growth factor II (IGF2) of SEQ ID NO: 5 (i.e., IGF2-delta 2-7 or IGF2Δ2-7; which corresponds to SEQ ID NO: 6); residues 8-67 of wild-type mature human insulin-like growth factor II (IGF2) of SEQ ID NO: 5 (i.e., IGF2-delta 1-7 or IGF2Δ1-7, which corresponds to SEQ ID NO: 7) or residues 43-67 of wild-type mature human insulin-like growth factor II (IGF2) of SEQ ID NO: 5 (i.e., IGF2 delta 1-42 or IGF2Δ1-42, which corresponds to SEQ ID NO: 8). In some embodiments of the compositions and methods described herein, the IGF2 sequence is a nucleic acid sequence that has a modification of amino acid residue 43, for example residue 43 is modified to a start codon, for example IGF2-V43M (corresponding to SEQ ID NO: 9).

In some embodiments of the compositions and methods described herein, the IGF2 sequence is a nucleic acid sequence comprising any of: SEQ ID NO: 2 (i.e., IGF2-delta 2-7); SEQ ID NO: 3 (i.e., IGF2-delta 1-7) or SEQ ID NO: 4 (i.e., IGF2-V43M).

In some embodiments of the compositions and methods described herein, the fusion protein comprising the GAA polypeptide and a IGF2 sequence comprises amino acid residues 40-952 or residues 70-952 of human acid alpha-glucosidase (GAA) polypeptide (SEQ ID NO: 10) that is attached to an IGF2 sequence that comprises residue 1 followed by residues 8-67 of wild-type mature human insulin-like growth factor II (IGF2) (SEQ ID NO: 5), (that is—residues 2-7 of mature human IGF2 (SEQ ID NO:5) are not present), wherein the IGF2 sequence is linked to amino acid residue 70 of human GAA (SEQ ID NO: 10).

In some embodiments of the compositions and methods described herein, the fusion protein comprising the GAA polypeptide and a IGF2 sequence comprises amino acid residues 40-952 or residues 70-952 of human acid alpha-glucosidase (GAA) polypeptide (SEQ ID NO: 10) that is attached to an IGF2 sequence that comprises residues 8-67 of wild-type mature human insulin-like growth factor II (IGF2) (SEQ ID NO: 5), (that is—residues 1-7 of mature human IGF2 (i.e., Y R P S E T; SEQ ID NO: 63) are not present), wherein the IGF2 sequence is linked to amino acid residue 70 of human GAA (SEQ ID NO: 10).

In some embodiments of the compositions and methods described herein, the fusion protein comprising the GAA polypeptide and a IGF2 sequence comprises amino acid residues 40-952 or residues 70-952 of human acid alpha-glucosidase (GAA) (SEQ ID NO: 10) that is attached to a modified IGF2 sequence that comprises residues 43-67 of wild-type mature human insulin-like growth factor II (IGF2) (SEQ ID NO: 5), (where residues 1-42 of mature human IGF2 (SEQ ID NO: 5) are not present), and where the IGF2 sequence is linked to amino acid residue 70 of human GAA (SEQ ID NO: 10).

In some embodiments of the compositions and methods described herein, the IGF2 sequence (i.e., the delta 1-7, delta 2-7 or delta 1-42 as disclosed herein) binds the cation-independent mannose-6-phosphate receptor (CI-MPR). In one embodiment, the IGF2 sequence is fused directly to the N- or C-terminus of the GAA polypeptide. In another embodiment, a IGF2 sequence is fused to the N- or C-terminus of the GAA polypeptide by a spacer. In one specific embodiment, a IGF2 sequence is fused to the GAA polypeptide by a spacer of 10-25 amino acids. In another specific embodiment, a IGF2 sequence is fused to the GAA polypeptide by a spacer including glycine residues. In another specific embodiment, a IGF2 sequence is fused to the GAA polypeptide by a spacer including a helical structure. In another specific embodiment, a IGF2 sequence is fused to the GAA polypeptide by a spacer at least 50% identical to the sequence GGGTVGDDDDK (SEQ ID NO: 35).

In some embodiments of the compositions and methods described herein, the secretory signal serves a general purpose of assisting the secretion of the fusion polypeptide, e.g., the IGF2 sequence-GAA fusion polypeptide from the liver cells into the blood, where it can travel and be targeted to the lysosomes of mammalian cells, for example, human cardiac and skeletal muscle cells, as described herein. In some embodiments, the secretory signal is selected from any of: a AAT signal peptide, a fibronectin signal peptide (FN1), a GAA signal peptide, or an active fragment of AAT, FN1 or GAA signal peptide having secretory signal activity.

All aspects of the compositions and methods of the technology disclosed herein are discussed below.

In some embodiments, the technology relates to a recombinant adenovirus associated (AAV) vector composition and its methods of use, the rAAV vector comprising in its genome: (a) 5′ and 3′ AAV inverted terminal repeats (ITR) sequences, and (b) located between the 5′ and 3′ ITRs, a heterologous nucleic acid sequence encoding a fusion polypeptide comprising a secretory signal peptide and an alpha-glucosidase (GAA) polypeptide, wherein the heterologous nucleic acid is operatively linked to a promoter. In some embodiments of the methods and compositions disclosed herein, the rAAV composition comprises a heterologous nucleic acid sequence encoding a fusion polypeptide further comprises a IGF-2 sequence located between the secretory signal peptide and the alpha-glucosidase (GAA) polypeptide. In some embodiments of the methods and compositions disclosed herein, the rAAV composition comprises a AAV genome, which comprises, in the 5′ to 3′ direction: (a) 5′ ITR, (b) a promoter sequence, (c) an intron sequence, (d) a nucleic acid encoding a secretory signal peptide, (e) a nucleic acid encoding an IGF-2 sequence, a nucleic acid encoding an alpha-glucosidase (GAA) polypeptide, (f) a poly A sequence, and (g) a 3′ ITR.

In some embodiments, the technology relates to a recombinant adenovirus associated (AAV) vector composition and its method of use, where the recombinant AAV (rAAV) vector comprises in its genome: (a) 5′ and 3′ AAV inverted terminal repeats (ITR) sequences, and (b) located between the 5′ and 3′ ITRs, a heterologous nucleic acid sequence encoding a fusion polypeptide comprising an alpha-glucosidase (GAA) polypeptide, where the heterologous nucleic acid is operatively linked to a liver specific promoter, and where the recombinant AAV vector comprises a capsid protein of the AAV3b serotype. In such embodiments, the fusion polypeptide further comprises a secretory signal peptide located at the N-terminal of the GAA polypeptide. In some embodiments of the methods and compositions disclosed herein, such a recombinant AAV vector comprises a heterologous nucleic acid sequence that encodes a fusion polypeptide further comprises a IGF-2 sequence located between the secretory signal peptide and the an alpha-glucosidase (GAA) polypeptide.

In some embodiments of the methods and compositions disclosed herein, a recombinant AAV vector comprises in its genome, in the 5′ to 3′ direction: (a) a 5′ ITR, (b) a liver specific promoter sequence, (c) an intron sequence, (d) a nucleic acid encoding a secretory signal peptide, (e) a nucleic acid encoding an IGF-2 sequence, (f) a nucleic acid encoding an alpha-glucosidase (GAA) polypeptide, (g) a poly A sequence, and (h) a 3′ ITR.

In some embodiments of the methods and compositions disclosed herein, the rAAV vector composition comprises the nucleic acid encoding a secretory signal peptide, e.g., encoding a secretory signal peptide selected from an AAT signal peptide (e.g., SEQ ID NO: 17), a fibronectin signal peptide (FN) (e.g., SEQ ID NO: 18-21), a GAA signal peptide, an hIGF2 signal peptide (e.g., SEQ ID NO: 22) or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NOs: 17-22. In some embodiments of the methods and compositions disclosed herein, the recombinant AAV vector comprises a heterologous nucleic acid sequence that encodes a IGF-2 leader sequence which binds human cation-independent mannose-6-phosphate receptor (CI-MPR) or the IGF-2 receptor, for example, the heterologous nucleic acid sequence encodes a IGF-2 sequence having the amino acid sequence of SEQ ID NO: 5 or comprises at least one amino modification in SEQ ID NO: 5 that binds to the IGF-2 receptor. In some embodiments, the recombinant AAV vector comprises a heterologous nucleic acid sequence that encodes a IGF-2 leader sequence that has at least one amino modification in SEQ ID NO: 5 is a V43M amino acid modification (SEQ ID NO: 8 or SEQ ID NO: 9) or 42-7 (SEQ ID NO: 6) or 41-7 (SEQ ID NO: 7), or is a IGF2 peptide having at least about 75%, or 80%, or 85%, or 90%, or 95%, or 98%, or 99% sequence identity to SEQ ID NOs: 5-9.

In some embodiments of the methods and compositions disclosed herein, the recombinant AAV vector comprises a promoter that is constitutive, cell specific or inducible. In some embodiments of the methods and compositions disclosed herein, the recombinant AAV vector comprises a liver-specific promoter, for example but not limited to, a liver specific promoter is selected from any of: transthyretin promoter (TTR), LSP promoter (LSP), a synthetic liver specific promoter.

In some embodiments of the methods and compositions disclosed herein, a recombinant AAV vector comprises a heterologous nucleic acid sequence that encodes a wild-type GAA polypeptide (wtGAA) or a modified GAA polypeptide. In some embodiments of the methods and compositions disclosed herein, a recombinant AAV vector comprises a heterologous nucleic acid sequence encoding the GAA polypeptide that is the human GAA gene or a human codon optimized GAA gene (coGAA) or a modified GAA nucleic acid sequence. In all aspects of the methods and compositions as disclosed herein, a nucleic acid sequence encoding the GAA polypeptide is codon optimized for any one or more of: enhanced expression in vivo, to reduce CpG islands, or to reduce the innate immune response. In all aspects of the methods and compositions as disclosed herein, a nucleic acid sequence encoding the GAA polypeptide is codon optimized to reduce CpG islands and to reduce the innate immune response.

In some embodiments of the methods and compositions disclosed herein, a recombinant AAV vector comprises a heterologous nucleic acid sequence where the encoded fusion polypeptide further comprises a spacer comprising a nucleotide sequence for at least 1 amino acids located amino-terminal to the GAA polypeptide, and C-terminal to the IGF-2 sequence. In some embodiments of the methods and compositions disclosed herein, a recombinant AAV vector comprises a heterologous nucleic acid sequence that comprises a nucleic acid encoding a spacer of at least 1 amino acids located between the nucleic acid encoding the IGF-2 sequence and the nucleic acid encoding the GAA polypeptide.

In some embodiments of the methods and compositions disclosed herein, a recombinant AAV vector comprises at least one polyA sequence located 3′ of the nucleic acid encoding the GAA gene and 5′ of the 3′ ITR sequence.

In some embodiments of the methods and compositions disclosed herein, a recombinant AAV vector comprises a heterologous nucleic acid sequence that further comprises at collagen stability (CS) sequence located 3′ of the nucleic acid encoding the GAA polypeptide and 5′ of the 3′ ITR sequence. In some embodiments of the methods and compositions disclosed herein, a recombinant AAV vector further comprising a nucleic acid encoding a collagen stability (CS) sequence located between the nucleic acid encoding the GAA polypeptide and the poly A sequence.

In some embodiments of the methods and compositions disclosed herein, a recombinant AAV vector comprises a heterologous nucleic acid sequence that further comprises an intron sequence located 5′ of the sequence encoding the secretory signal peptide, and 3′ of the promoter. In some embodiments, the intron sequence comprises a MVM sequence or a HBB2 sequence, wherein the MVN sequence comprises the nucleic acid sequence of SEQ ID NO: 13, or a nucleic acid sequence at least about 75%, or 80%, or 85%, or 90%, or 95%, or 98%, or 99% sequence identity to SEQ ID NO: 13, and the HBB2 sequence comprises the nucleic acid sequence of SEQ ID NO: 14, or a nucleic acid sequence at least about 75%, or 80%, or 85%, or 90%, or 95%, or 98%, or 99% sequence identity to SEQ ID NO: 14.

In some embodiments of the methods and compositions disclosed herein, a recombinant AAV vector comprises an ITR sequence that comprises an insertion, deletion or substitution. In some embodiments of the methods and compositions disclosed herein, a recombinant AAV vector comprises at least one ITR sequence where one or more CpG islands in the ITR are removed.

In some embodiments of the methods and compositions disclosed herein, a recombinant AAV vector comprises a heterologous nucleic acid sequence that encodes a secretory signal peptide which is a fibronectin signal peptide (FN1) or an active fragment thereof having secretory signal activity (e.g., a FN1 signal peptide has the sequence of any of SEQ ID NO: 18-21, or an amino acid sequence at having at least about 75%, or 80%, or 85%, or 90%, or 95%, or 98%, or 99% sequence identity to any of SEQ ID NOs: 18-21), and the heterologous nucleic acid sequence encodes a IGF-2 sequence selected from any of: SEQ ID NO: 5, SEQ ID NO: 6, SEQ ID NO: 7, SEQ ID NO: 8 or SEQ ID NO: 9, or a IGF2 peptide having at least about 75%, or 80%, or 85%, or 90%, or 95%, or 98%, or 99% sequence identity to SEQ ID NOs: 5-9. In some embodiments of the methods and compositions disclosed herein, a recombinant AAV vector comprises a heterologous nucleic acid sequence that encodes a secretory signal peptide is AAT signal peptide or an active fragment thereof having secretory signal activity, (e.g., a AAT signal peptide has the sequence of SEQ ID NO: 17, or an amino acid sequence at having at least about 75%, or 80%, or 85%, or 90%, or 95%, or 98%, or 99% sequence identity to SEQ ID NO: 17), and the heterologous nucleic acid sequence encodes a IGF-2 sequence selected from any of: SEQ ID NO: 5, SEQ ID NO: 6, SEQ ID NO: 7, SEQ ID NO: 8 or SEQ ID NO: 9, or a IGF2 peptide having at least about 75%, or 80%, or 85%, or 90%, or 95%, or 98%, or 99% sequence identity to SEQ ID NOs: 5-9.

In some embodiments of the methods and compositions disclosed herein, a recombinant AAV vector comprises a heterologous nucleic acid sequence that encodes an IGF2 peptide, where the IGF2 peptide sequence is SEQ ID NO: 8 or SEQ ID NO: 9, or a IGF2 peptide having at least about 75%, or 80%, or 85%, or 90%, or 95%, or 98%, or 99% sequence identity to SEQ ID NO: 8 or 9.

In some embodiments of the methods and compositions disclosed herein, a recombinant AAV vector is a chimeric AAV vector, haploid AAV vector, a hybrid AAV vector or polyploid AAV vector, for example, but not limited to, where the recombinant AAV vector comprises a capsid protein selected from any AAV serotype in the group consisting of those listed in Table 1 and any combination thereof. In some embodiments of the methods and compositions disclosed herein, a recombinant AAV vector is serotype AAV3b. In some embodiments of the methods and compositions disclosed herein, a recombinant AAV vector is a AAV3b serotype which comprises one or mutations in a capsid protein selected from any of: 265D, 549A, Q263Y. In some embodiments of the methods and compositions disclosed herein, a recombinant AAV vector is an AAV3b serotype selected from any of: AAV3b265D, AAV3b265D549A, AAV3b549A or AAV3bQ263Y, or AAV3bSASTG.

Another aspect of the technology herein relates to a pharmaceutical composition comprising any of the recombinant AAV vector compositions disclosed herein, and a pharmaceutically acceptable carrier.

Another aspect of the technology herein relates to a composition comprising a nucleic acid sequence comprising: a liver specific promoter operatively linked to a nucleic acid sequence comprising, in the following order: (a) a nucleic acid encoding a secretory signal peptide, (b) a nucleic acid encoding a IGF-2 sequence, and (c) a nucleic acid encoding a GAA polypeptide.

Another aspect of the technology herein relates to a composition comprising a nucleic acid sequence for a recombinant adenovirus associated (rAAV) vector genome, the nucleic acid sequence comprising: (a) a 5′ and a 3′ AAV inverted terminal repeats (ITR) nucleic acid sequences, and (b) located between the 5′ and 3′ ITR sequence, a heterologous nucleic acid sequence encoding a fusion polypeptide comprising a secretory signal peptide and an alpha-glucosidase (GAA) polypeptide, wherein the heterologous nucleic acid is operatively linked to a promoter.

In some embodiments of the methods and compositions disclosed herein, the nucleic acid sequence comprises a heterologous nucleic acid sequence encoding a fusion polypeptide further comprises a IGF-2 sequence located between the secretory signal peptide and the an alpha-glucosidase (GAA) polypeptide. In some embodiments of the methods and compositions as disclosed herein, the nucleic acid sequence comprises a nucleic acid encoding the secretory signal is selected from any of SEQ ID NO: 17, 22-26, or a nucleic acid sequence at least about 75%, or 80%, or 85%, or 90%, or 95%, or 98%, or 99% sequence identity to any of SEQ ID NOs: 17 or 22-26.

In some embodiments of the methods and compositions disclosed herein, the nucleic acid sequence comprises a heterologous nucleic acid sequence encoding a IGF-2 sequence is selected from any of SEQ ID NO: 2 (IGF2-Δ2-7), SEQ ID NO: 3 (IGF2-Δ1-7), or SEQ ID NO: 4 (IGF2 V43M), or a nucleic acid sequence at least about 75%, or 80%, or 85%, or 90%, or 95%, or 98%, or 99% sequence identity to any of SEQ ID NOs: 2, 3 or 4.

In some embodiments of the methods and compositions disclosed herein, the nucleic acid sequence comprises a heterologous nucleic acid sequence encoding a GAA polypeptide, where the nucleic acid sequence is a human GAA gene or a human codon optimized GAA gene (coGAA) or a modified GAA nucleic acid sequence. In some embodiments of the methods and compositions disclosed herein, the nucleic acid sequence comprises a heterologous nucleic acid sequence that is a codon optimized (coGAA) GAA gene, for any one or more of enhanced expression in vivo, to reduce CpG islands or to reduce the innate immune response. In some embodiments of the methods and compositions disclosed herein, the nucleic acid sequence comprises a heterologous nucleic acid sequence that is a codon optimized (coGAA) GAA gene to reduce CpG islands and to reduce the innate immune response.

In some embodiments of the methods and compositions disclosed herein, the nucleic acid sequence comprises a heterologous nucleic acid sequence encoding a GAA polypeptide selected from any of SEQ ID NO: 11 (full length hGAA), SEQ ID NO: 55 (Dwight cDNA), SEQ ID NO: 56 (hGAA Δ1-66) or a nucleic acid sequence at least about 75%, or 80%, or 85%, or 90%, or 95%, or 98%, or 99% sequence identity to any of SEQ ID NOs: 11, 55 or 56.

In some embodiments of the methods and compositions disclosed herein, the nucleic acid sequence comprises a heterologous nucleic acid sequence encoding the GAA polypeptide, where the nucleic acid encoding the GAA polypeptide is selected from any of SEQ ID NO: 74 (codon optimized 1), SEQ ID NO: 75 (codon optimized 2), and SEQ ID NO: 76 (codon optimized 3), or a nucleic acid sequence at least about 75%, or 80%, or 85%, or 90%, or 95%, or 98%, or 99% sequence identity to any of SEQ ID NOs: 74, 75 or 76.

In some embodiments of the methods and compositions disclosed herein, the nucleic acid sequence is selected from any of: SEQ ID NO: 57 (AAT-V43M-wtGAA (delta1-69aa)); SEQ ID NO: 58 (ratFN1-IGF2V43M-wtGAA (delta1-69aa)); SEQ ID NO: 59 (hFN1-IGF2V43M-wtGAA (delta1-69aa)); SEQ ID NO: 60 (ATT-IGF2Δ2-7-wtGAA (delta 1-69)); SEQ ID NO: 61 (FN1rat-IGFΔ2-7-wtGAA (delta 1-69)); SEQ ID NO: 62 (hFN1-IGFΔ2-7-wtGAA (delta 1-69)), SEQ ID NO: 79 (AAT_hIGF2-V43M_wtGAA_del1-69_Stuffer.V02); SEQ ID NO: 80 (FIBrat_hIGF2-V43M_wtGAA_del1-69_Stuffer.V02); SEQ ID NO: 81 (FIBhum_hIGF2-V43M_wtGAA_del1-69_Stuffer.V02); SEQ ID NO: 82 (AAT_GILT_wtGAA_del1-69 Stuffer.V02); SEQ ID NO: 83 (FIBrat_GILT_wtGAA_del1-69_Stuffer.V02); SEQ ID NO: 84 (FIBhum_GILT_wtGAA_del1-69_Stuffer.V02) or a nucleic acid sequence having at least 80%, 85%, 90%, 95% or 98% identity to SEQ ID Nos: 57, 58, 59, 60, 61, 62, 79, 80, 81, 82, 83 or 84.

In some embodiments of the methods and compositions disclosed herein, the rAAV vector comprises a nucleic acid sequence is selected from any of: SEQ ID NO: 57 (AAT-V43M-wtGAA (delta1-69aa)); SEQ ID NO: 58 (ratFN1-IGF2V43M-wtGAA (delta1-69aa)); SEQ ID NO: 59 (hFN1-IGF2V43M-wtGAA (delta1-69aa)); SEQ ID NO: 60 (ATT-IGF2Δ2-7-wtGAA (delta 1-69)); SEQ ID NO: 61 (FN1rat-IGFΔ2-7-wtGAA (delta 1-69)); SEQ ID NO: 62 (hFN1-IGFΔ2-7-wtGAA (delta 1-69)), SEQ ID NO: 79 (AAT_hIGF2-V43M_wtGAA_del1-69_Stuffer.V02); SEQ ID NO: 80 (FIBrat_hIGF2-V43M_wtGAA_del1-69_Stuffer.V02); SEQ ID NO: 81 (FIBhum_hIGF2-V43M_wtGAA_del1-69_Stuffer.V02); SEQ ID NO: 82 (AAT_GILT_wtGAA_del1-Stuffer.V02); SEQ ID NO: 83 (FIBrat_GILT_wtGAA_del1-69_Stuffer.V02); SEQ ID NO: 84 (FIBhum_GILT_wtGAA_del1-69_Stuffer.V02) or a nucleic acid sequence having at least 80%, 85%, 90%, 95% or 98% identity to SEQ ID Nos: 57, 58, 59, 60, 61, 62, 79, 80, 81, 82, 83 or 84.

Another aspect of the technology herein relates to use of the rAAV and nucleic acid compositions disclosed herein in a method to treat a disease. In particular, one aspect of the technology herein relates to use of the rAAV vector compositions and nucleic acid compositions disclosed herein, in a method to treat a subject with a glycogen storage disease type II (GSD II, Pompe Disease, Acid Maltase Deficiency) or having a deficiency in alpha-glucosidase (GAA) polypeptide, the method comprising administering any of the recombinant AAV vector, or the rAAV genome or the nucleic acid sequence disclosed herein to the subject. In some embodiments of the methods disclosed herein, the expressed GAA polypeptide is secreted from the subject's liver and there is uptake of the secreted GAA by skeletal muscle tissue, cardiac muscle tissue, diaphragm muscle tissue or a combination thereof, wherein uptake of the secreted GAA results in a reduction in lysosomal glycogen stores in the tissue(s). In some embodiments in the disclosed methods, the recombinant AAV vector, or the rAAV genome or the nucleic acid sequence is administered to the subject by any suitable administration method, for example, but not limited to, an administration method selected from any of: intramuscular, sub-cutaneous, intraspinal, intracisternal, intrathecal, intravenous administration. In some embodiments, the pharmaceutical composition disclosed herein can be used in the methods disclosed herein.

Another aspect of the technology herein relates to a cell comprising any one or more of a rAAV composition, a rAAV genome composition, or a nucleic acid composition as disclosed herein. In some embodiments, the cell is a human cell, or a non-human cell mammalian cell, or an insect cell.

Another aspect of the technology herein relates to host animal comprising any one or more of a rAAV composition, a rAAV genome composition, or a nucleic acid composition as disclosed herein. In some embodiments, the host animal is a mammal, a non-human mammal or a human.

Another aspect of the technology herein relates to host animal comprising at least one cell that comprises any one or more of a rAAV composition, a rAAV genome composition, or a nucleic acid composition as disclosed herein. In some embodiments, the host animal comprising such a modified cell is a mammal, a non-human mammal or a human.

Aspects of the present invention teach certain benefits in construction and use which give rise to the exemplary advantages described below.

In some embodiments, disclosed herein is a pharmaceutical formulation comprising an rAAV vectors, nucleic acid encoding a rAAV genome as disclosed herein, and a pharmaceutically acceptable carrier.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

This application file contains at least one drawing executed in color. Copies of this patent application publication with color drawings will be provided by the Office upon request and payment of the necessary fee. The accompanying drawings illustrate aspects of the present invention. In such drawings:

FIG. 1 is a graph illustrating a y-axis of vector genomes per diploid genome and an x-axis of different AAV serotypes AAV3b, AAV3ST, AAV8, and AAV9, as measured in whole blood, in accordance with at least one embodiment.

FIG. 2 is a graph illustrating a y-axis of vector genomes per diploid genome and an x-axis of different AAV serotypes AAV3b, AAV3ST, AAV8, and AAV9, as measured in left, median and right liver lobes, in accordance with at least one embodiment.

FIG. 3 is an illustration of a plasmid map of an adeno-associated virus vector plasmid, in accordance with at least one embodiment.

FIG. 4 is an illustration of a plasmid map of pAAV-LSPhGAA plasmid, in accordance with at least one embodiment.

FIGS. 5A-5G are illustrations of exemplary nucleic acid constructs for a rAAV genome as disclosed herein. FIG. 5A shows a nucleic acid construct for a rAAV genome comprising a 5′ ITR, a promoter, operatively linked to a nucleic acid encoding a secretory signal peptide (SS), a targeting peptide and a human GAA polypeptide, and a 3′ ITR. FIG. 5A a nucleic acid construct for a rAAV genome, comprising a 5′ ITR, a promoter, operatively linked to a heterologous nucleic acid encoding a secretory signal peptide (SS), a targeting peptide (TP) and a human GAA (hGAA) polypeptide and a 3′ ITR. FIG. 5B shows an exemplary nucleic acid construct for a rAAV genome as disclosed herein, comprising the same elements as FIG. 5A, and additionally comprising at least one polyA signal 3′ of the hGAA polypeptide and 5′ of the 3′-ITR. FIG. 5C shows an exemplary nucleic acid construct for a rAAV genome as disclosed herein, comprising the same elements as FIG. 5B, except comprising with an intron sequence 3′ of the promoter. FIG. 5D shows an exemplary nucleic acid construct for a rAAV genome as disclosed herein, comprising the same elements as FIG. 5C, except comprising a collagen stability (CS) sequence located 3′ of the hGAA polypeptide nucleic acid sequence and before the poly A sequence. FIG. 5E shows an exemplary nucleic acid construct for a rAAV genome as disclosed herein, comprising the same elements as FIG. 5D, except also comprising a nucleic acid encoding a spacer of at least 1 amino acid that is located between the nucleic acid encoding the hGAA polypeptide and the nucleic acid encoding the targeting peptide (TP), e.g., IGF2 sequence. FIG. 5F shows an exemplary nucleic acid construct for a rAAV genome as disclosed herein, comprising the same elements as FIG. 5E, wherein the promoter is a liver promoter, the intron sequence is selected from a MVM or HBB2 intron sequence, the secretory signal peptide is selected from any of FN1 signal peptide (e.g., hFN1, ratFN1), a AAT signal peptide or a hGAA signal peptide; the targeting peptide is a IGF2 sequence as disclosed herein, and the at least polyA sequence is selected from hGHpA or a synPA poly A sequence. FIG. 5G shows an exemplary nucleic acid construct for a rAAV genome as disclosed herein, comprising the same elements as FIG. 5F, except where the IGF2 sequence is a nucleic acid sequence selected from SEQ ID NO: 2 (IGF2Δ2-7), SEQ ID NO: 3 (IGF2Δ1-7), or SEQ ID NO: 4 (IGF2 V43M).

FIG. 6 shows an exemplary nucleic acid construct for a rAAV genome comprising a 5′ ITR, a liver specific promoter, operatively linked to an intron sequence (e.g., MVM or HBB2 intron sequence), a nucleic acid encoding a signal secretory peptide selected from any of FN1, ATT or GAA signal peptide, a nucleic acid encoding a human GAA polypeptide, a collagen stability (CS) sequence, at least one polyA sequence (e.g., hGHpA and/or synPA polyA sequence) and a 3′ ITR.

FIG. 7 shows an illustration of the Gibson cloning technique to generate rAAV genomes as disclosed herein. In particular, a triple ligation is performed to ligate 3 blocks of nucleic acid sequence together, which can then be cloned into a vector with the promoter, e.g., liver specific promoter, and 5′ and 3′ ITRs to generate the rAAV genome. The Gibson cloning methodology was used to generate the following rAAV genomes: SEQ ID NO: 57 (AAT-V43M-wtGAA (delta1-69aa)); SEQ ID NO: 58 (ratFN1-IGF2V43M-wtGAA (delta1-69aa)); SEQ ID NO: 59 (hFN1-IGF2V43M-wtGAA (delta′-69aa)); SEQ ID NO: 60 (ATT-IGF2Δ2-7-wtGAA (delta 1-69)); SEQ ID NO: 61 (FN1rat-IGFΔ2-7-wtGAA (delta 1-69)); SEQ ID NO: 62 (hFN1-IGFΔ2-7-wtGAA (delta 1-69)).

FIG. 8 shows the generation of an exemplary rAAV genome of SEQ ID NO: 57 comprising AAT-V43M-wtGAA (delta1-69aa)) using Gibson cloning of nucleic acid sequence blocks (1, 2 and 3). Also shown in the AAT-V43M-wtGAA (delta1-69aa)) vector is the location a 3 amino acid (3aa) spacer nucleic acid sequence (showing the exemplary 3aa sequence “G-A-P” as SEQ ID NO: 31) which is located 3′ of the nucleic acid sequence encoding the IGF(V42M) targeting peptide and 5′ of the nucleic acid encoding wtGAA(41-69) enzyme, and a stuffer nucleic acid sequence (referred to in FIG. 8. as a “spacer” sequence) which is located 3′ of the polyA sequence and 5′ of the 3′ITR sequence.

FIG. 9 shows the generation of an exemplary rAAV genome of SEQ ID NO: 58 comprising ratFN1-IGF2V43M-wtGAA (delta1-69aa), using Gibson cloning of nucleic acid sequence blocks (4, 2 and 3). Also shown in the ratFN1-IGF2V43M-wtGAA (delta1-69aa)vector is the location a 3 amino acid (3aa) spacer nucleic acid sequence (showing the exemplary 3aa sequence “G-A-P” as SEQ ID NO: 31) which is located 3′ of the nucleic acid sequence encoding the IGF(V42M) targeting peptide and 5′ of the nucleic acid encoding wtGAA(41-69) enzyme, and a stuffer nucleic acid sequence (referred to in FIG. 9. as a “spacer” sequence) which is located 3′ of the polyA sequence and 5′ of the 3′ITR sequence.

FIG. 10 shows the generation of an exemplary rAAV genome of SEQ ID NO: 59 comprising hFN1-IGF2V43M-wtGAA (delta1-69aa), using Gibson cloning of nucleic acid sequence blocks (5, 2 and 3). Also shown in the hFN1-IGF2V43M-wtGAA (delta1-69aa) vector is the location a 3 amino acid (3aa) spacer nucleic acid sequence (showing the exemplary 3aa sequence “G-A-P” as SEQ ID NO: 31) which is located 3′ of the nucleic acid sequence encoding the IGF(V42M) targeting peptide and 5′ of the nucleic acid encoding wtGAA(41-69) enzyme, and a stuffer nucleic acid sequence (referred to in FIG. 10. as a “spacer” sequence) which is located 3′ of the polyA sequence and 5′ of the 3′ITR sequence.

FIG. 11 shows the generation of an exemplary rAAV genome of SEQ ID NO: 60 comprising ATT-IGF2Δ2-7-wtGAA (delta 1-69); using Gibson cloning of nucleic acid sequence blocks (6, 2 and 3). Also shown in the ATT-IGF2Δ2-7-wtGAA (delta 1-69)vector is the location a 3 amino acid (3aa) spacer nucleic acid sequence (showing the exemplary 3aa sequence “G-A-P” as SEQ ID NO: 31) which is located 3′ of the nucleic acid sequence encoding the IGF2Δ2-7 targeting peptide and 5′ of the nucleic acid encoding wtGAA(41-69) enzyme, and a stuffer nucleic acid sequence (referred to in FIG. 11. as a “spacer” sequence) which is located 3′ of the polyA sequence and 5′ of the 3′ITR sequence.

FIG. 12 shows the generation of a rAAV genome of SEQ ID NO: 61 comprising FN1rat-IGFΔ2-7-wtGAA (delta 1-69), using Gibson cloning of nucleic acid sequence blocks (7, 2 and 3). Also shown in the FN1rat-IGFΔ2-7-wtGAA (delta 1-69) vector is the location a 3 amino acid (3aa) spacer nucleic acid sequence (showing the exemplary 3aa sequence “G-A-P” as SEQ ID NO: 31) which is located 3′ of the nucleic acid sequence encoding the IGFΔ2-7 targeting peptide and 5′ of the nucleic acid encoding wtGAA(41-69) enzyme, and a stuffer nucleic acid sequence (referred to in FIG. 12. as a “spacer” sequence) which is located 3′ of the polyA sequence and 5′ of the 3′ITR sequence.

FIG. 13 shows the generation of a rAAV genome of SEQ ID NO: 62 comprising hFN1-IGFΔ2-7-wtGAA (delta 1-69), using Gibson cloning of nucleic acid sequence blocks (8, 2 and 3). Also shown in the hFN1-IGFΔ2-7-wtGAA (delta 1-69) vector is the location a 3 amino acid (3aa) spacer nucleic acid sequence (showing the exemplary 3aa sequence “G-A-P” as SEQ ID NO: 31) which is located 3′ of the nucleic acid sequence encoding the IGFΔ2-7 targeting peptide and 5′ of the nucleic acid encoding wtGAA(41-69) enzyme, and a stuffer nucleic acid sequence (referred to in FIG. 13. as a “spacer” sequence) which is located 3′ of the polyA sequence and 5′ of the 3′ITR sequence.

FIGS. 14A-14F shows schematics of exemplary constructs of rAAV genomes expressing wild-type GAA. FIG. 14A shows a schematic of exemplary rAAV genome construct of Candidate 1_AAT_hIGF2-V43M_wtGAA_del1-69_Stuffer.V02 (SEQ ID NO: 79). FIG. 14B shows a schematic of exemplary rAAV genome construct of Candidate 2_FIBrat_hIGF2-V43M_wtGAA_del1-69_Stuffer.V02 (SEQ ID NO: 80). FIG. 14C shows a schematic of exemplary rAAV genome construct of Candidate 3_FIBhum_hIGF2-V43M_wtGAA_del1-69_Stuffer.V02 (SEQ ID NO: 81) FIG. 14D shows a schematic of exemplary rAAV genome construct of Candidate 4AAT_GILT_wtGAA_del1-69_Stuffer.V02 (SEQ ID NO: 82). FIG. 14E shows a schematic of exemplary rAAV genome construct of Candidate 5_FIBrat_GILT_wtGAA_del1-69_Stuffer.V02 (SEQ ID NO: 83). FIG. 14F shows a schematic of exemplary rAAV genome construct of Candidate 6 FIBhum_GILT_wtGAA_del1-69_Stuffer.V02 (SEQ ID NO: 84)

The above described figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

The disclosure described herein generally relates to recombinant AAV (rAAV) vectors and constructs for rAAV genomes for gene therapy for delivering a GAA polypeptide to a subject. In particular, the technology described herein relates in general to a rAAV vector, or a rAAV genome for producing a GAA polypeptide that is expressed in the liver and effectively targeted to the lysosomes of mammalian cells, for example, human cardiac and skeletal muscle cells. For example, the technology relates to a rAAV vector for transducing liver cells, where the transduced liver cells secrete the GAA polypeptide, and the secreted GAA polypeptide is targeted to lysosomes in skeletal muscle tissue, cardiac muscle tissue, diaphragm muscle tissue or a combination thereof.

Accordingly, one aspect of the technology described herein provides rAAV vector comprising a rAAV genome that can be used to produce GAA that is more effectively secreted from cells, e.g., liver cells, and then targeted to the lysosomes of mammalian cells, for example, human cardiac and skeletal muscle cells.

In particular, in some embodiments, the GAA polypeptide is expressed as a fusion protein comprising at least a signal peptide that promotes secretion of the GAA polypeptide from the liver. In some embodiments, the GAA polypeptide is expressed as a fusion protein comprising at least a signal peptide that promotes secretion of the GAA polypeptide from the liver, and also a targeting sequence, that allows effective targeting to lysosomes in mammalian cells, e.g., muscle cells, for example, human cardiac and skeletal muscle cells. In some embodiments, the targeting peptide is a IGF2 sequence a described herein.

One aspect of the technology described herein relates to a rAAV vector that comprises a nucleotide sequence containing inverted terminal repeats (ITRs), a promoter, a heterologous gene, a poly-A tail and potentially other regulator elements for use to treat a disease, such as Pompe Disease, and further, for the treatment of Pompe Disease, wherein the heterologous gene is a GAA and wherein the rAAV GAA can be administered to a patient in a therapeutically effective dose that is delivered to the appropriate tissue and/or organ for expression of the heterologous gene and treatment of the disease.

One aspect of the technology described herein relates to a rAAV vector that comprises in its genome the following in a 5′ to 3′ direction: 5′- and 3′-AAV inverted terminal repeats (ITR) sequences, and located between the 5′ and 3′ ITRs, a heterologous nucleic acid sequence encoding a fusion polypeptide comprising (i) a secretory signal peptide (SS), (ii) an IGF2 sequence; and (iii) an alpha-glucosidase (GAA) polypeptide, wherein the heterologous nucleic acid is operatively linked to a promoter. In some embodiments of the methods and compositions as disclosed herein, the secretory signal peptide is selected from any of: AAT signal peptide, a fibronectin signal peptide (FN1), a GAA signal peptide, or an active fragment thereof having secretory signal activity.

In some embodiments, the a rAAV vector described herein is from any serotype. In some embodiments, the rAAV vector is a AAV3b serotype, including, but not limited to, an AAV3b265D virion, an AAV3b265D549A virion, an AAV3b549A virion, an AAV3bQ263Y virion, or an AAV3bSASTG virion (i.e., a virion comprising a AAV3b capsid comprising Q263A/T265 mutations).

Aspects of the technology relate to use of the rAAV vector described herein in methods of treating a deficiency of a GAA polypeptide in a subject, comprising administering to the subject a rAAV vector as disclosed herein, in a pharmaceutically acceptable carrier in a therapeutically effective amount. In some embodiments, the rAAV vector is for use in the treatment or prophylaxis of Pompe Disease (also known as Glycogen storage disease type 2 or GSD II). In some embodiments, the subject is a mammal and wherein the mammal is a human, a primate, a canine, a horse, a cow, a feline.

In some embodiments, a rAAV vector that comprises nucleic acids encoding for a GAA polypeptide comprising at least a N-terminal secretory signal peptide, where the liver cells transduced with the rAAV vector express the GAA polypeptide and N-terminal secretory peptide, and secrete the GAA polypeptide. Furthermore, the secreted GAA polypeptide can also optionally comprise a targeting sequence, e.g., a IGF2 sequence attached to the N-terminal or C-terminal of the GAA polypeptide, to enhance uptake and targeting the GAA polypeptide to lysosomes in skeletal muscle tissue, cardiac muscle tissue, diaphragm muscle tissue, nerve cells that trigger muscle tissue or a combination thereof. Further, in an embodiment, the uptake of the secreted GAA polypeptide in muscle cells results in a reduction in lysosomal glycogen stores in the tissue(s) and a reduction or elimination of the symptoms associated with Pompe Disease.

In an embodiment, the rAAV vector comprises a capsid and within the capsid is a nucleotide sequence, herein referred to as the “rAAV vector genome”. The rAAV vector genome includes multiple elements, including, but not limited to two inverted terminal repeats (ITRs, e.g., the 5′-ITR and the 3′-ITR), and located between the ITRs are additional elements, including a promoter, a heterologous gene and a poly-A tail. In a further embodiment, there can be additional elements between the ITRs including seed region sequences for the binding of miRNA or an shRNA sequence.

### I. Definitions

The following terms are used in the description herein and the appended claims:

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Furthermore, the term “about,” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461,463 (CCPA 1976) (emphasis in the original); see also MPEP § 2111.03. Thus, the term “consisting essentially of” when used in a claim of this invention is not intended to be interpreted to be equivalent to “comprising.” Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate further, if, for example, the specification indicates that a particular amino acid can be selected from A, G, I, Land/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed (e.g., by negative proviso). For example, in particular embodiments the amino acid is not A, G or I; is not A; is not G or V; etc. as if each such possible disclaimer is expressly set forth herein.

The term “parvovirus” as used herein encompasses the family Parvoviridae, including autonomously replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, Muscovy duck parvovirus, B19 virus, and any other autonomous parvovirus now known or later discovered. Other autonomous parvoviruses are known to those skilled in the art. See, e.g., BERNARD N. FIELDS et al., VIROLOGY, volume 2, chapter 69 (4th ed., Lippincott-Raven Publishers).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). A number of relatively new AAV serotypes and clades have been identified (see, e.g., Gao et al., (2004) J. Virology 78:6381-6388; Moris et al., (2004) Virology 33-375-383; and Table 1).

The genomic sequences of various serotypes of AAV and the autonomous parvoviruses, as well as the sequences of the native inverted terminal repeats (ITRs), Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, NC_001358, NC_001540, AF513851, AF513852, AY530579; the disclosures of which are incorporated by reference herein for teaching parvovirus and AAV nucleic acid and amino acid sequences. See also, e.g., Srivistava et al., (1983) J Virology 45:555; Chiarini et al., (1998) J. Virology 71:6823; Chiarini et al., (1999) J. Virology 73:1309; Bantel-Schaal et al., (1999) J. Virology 73:939; Xiao et al., (1999) J. Virology 73:3994; Muramatsu et al., (1996) Virology 221:208; Shade et al., (1986) J. Viral. 58:921; Gao et al., (2002) Proc. Nat. Acad. Sci. USA 99:11854; Morris et al., (2004) Virology 33-: 375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1 and 5 disclosed herein.

The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99:10405-10), AAV4 (Padron et al., (2005) J. Viral. 79: 5047-58), AAV5 (Walters et al., (2004) J. Viral. 78: 3361-71) and CPV (Xie et al., (1996) J. Mal. Biol. 6:497-520 and Tsao et al., (1991) Science 251: 1456-64).

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest.

As used here, “systemic tropism” and “systemic transduction” (and equivalent terms) indicate that the virus capsid or virus vector of the invention exhibits tropism for and/or transduces tissues throughout the body (e.g., brain, lung, skeletal muscle, heart, liver, kidney and/or pancreas). In embodiments of the invention, systemic transduction of the central nervous system (e.g., brain, neuronal cells, etc.) is observed. In other embodiments, systemic transduction of cardiac muscle tissues is achieved.

As used herein, “selective tropism” or “specific tropism” means delivery of virus vectors to and/or specific transduction of certain target cells and/or certain tissues.

In some embodiments of this invention, an AAV particle comprising a capsid of this invention can demonstrate multiple phenotypes of efficient transduction of 30 certain tissues/cells and very low levels of transduction (e.g., reduced transduction) for certain tissues/cells, the transduction of which is not desirable.

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotides), but in representative embodiments are either single or double stranded DNA sequences.

A “chimeric nucleic acid” comprises two or more nucleic acid sequences covalently linked together to encode a fusion polypeptide. The nucleic acids may be DNA, RNA, or a hybrid thereof.

The term “fusion polypeptide” comprises two or more polypeptides covalently linked together, typically by peptide bonding.

As used herein, an “isolated” polynucleotide (e.g., an “isolated DNA” or an “isolated RNA”) means a polynucleotide at least partially separated from at least some of the other components of the naturally occurring organism or virus, for example; the cell or viral structural components or other polypeptides or nucleic acids commonly found associated with the polynucleotide. In representative embodiments an “isolated” nucleotide is enriched by at least about 10-fold, 100′-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

As used herein, by “isolate” or “purify” (or grammatical equivalents) a virus vector or virus particle or population of virus particles, it is meant that the virus vector or virus particle or population of virus particles is at least partially separated from at least some of the other components in the starting material. In representative embodiments an “isolated” or “purified” virus vector or virus particle or population of virus particles is enriched by at least about 10-fold, 100-fold, 1000-fold, 10,000-fold or more as compared with the starting material.

Unless indicated otherwise, “efficient transduction” or “efficient tropism,” or similar terms, can be determined by reference to a suitable control (e.g., at least about 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 500% or more of the transduction or tropism, respectively, of the control). In particular embodiments, the virus vector efficiently transduces or has efficient tropism for neuronal cells and cardiomyocytes. Suitable controls will depend on a variety of factors including the desired tropism and/or transduction profile.

A “therapeutic polypeptide” is a polypeptide that can alleviate, reduce, prevent, delay and/or stabilize symptoms that result from an absence or defect in a protein in a cell or subject and/or is a polypeptide that otherwise confers a benefit to a subject, e.g., enzyme replacement to reduce or eliminate symptoms of a disease, or improvement in transplant survivability or induction of an immune response.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is substantially less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

The terms “heterologous nucleotide sequence” and “heterologous nucleic acid molecule” are used interchangeably herein and refer to a nucleic acid sequence that is not naturally occurring in the virus. Generally, the heterologous nucleic acid molecule or heterologous nucleotide sequence comprises an open reading frame that encodes a polypeptide and/or nontranslated RNA of interest (e.g., for delivery to a cell and/or subject), for example the GAA polypeptide.

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone.

An “rAAV vector genome” or “rAAV genome” is an AAV genome (i.e., vDNA) that comprises one or more heterologous nucleic acid sequences. rAAV vectors generally require only the inverted terminal repeat(s) (TR(s)) in cis to generate virus. All other viral sequences are dispensable and may be supplied in trans (Muzyczka, (1992) Curr. Topics Microbial. Immunol. 158:97). Typically, the rAAV vector genome will only retain the one or more TR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one ITR sequence (e.g., AAV TR sequence), optionally two ITRs (e.g., two AAV TRs), which typically will be at the 5′ and 3′ ends of the vector genome and flank the heterologous nucleic acid, but need not be contiguous thereto. The TRs can be the same or different from each other.

The term “terminal repeat” or “TR” includes any viral terminal repeat or synthetic sequence that forms a hairpin structure and functions as an inverted terminal repeat (i.e., an ITR that mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV terminal repeat” or “AAV TR,” including an “AAV inverted terminal repeat” or “AAV ITR” may be from any AAV, including but not limited to serotypes 1, 2, 3, 4, 5, 6, 7, 8, 9, 10, 11 or 12 or any other AAV now known or later discovered (see, e.g., Table 3). An AAV terminal repeat need not have the native terminal repeat sequence (e.g., a native AAV TR or AAV ITR sequence may be altered by insertion, deletion, truncation and/or missense mutations), as long as the terminal repeat mediates the desired functions, e.g., replication, virus packaging, integration, and/or provirus rescue, and the like.

AAV proteins VP1, VP2 and VP3 are capsid proteins that interact together to form an AAV capsid of an icosahedral symmetry. VP1.5 is an AAV capsid protein described in US Publication No. 2014/0037585.

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention.

Further, the viral capsid or genomic elements can contain other modifications, including insertions, deletions and/or substitutions.

A “chimeric” capsid protein as used herein means an AAV capsid protein that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a significant number of chimeric capsid proteins are described in the literature as well as herein that can be included in the capsid of this invention.

As used herein, the term “haploid AAV” shall mean that AAV as described in PCT/US18/22725, which is incorporated herein.

The term a “hybrid” AAV vector or parvovirus refers to a rAAV vector where the viral TRs or ITRs and viral capsid are from different parvoviruses. Hybrid vectors are described in international patent publication WO 00/28004 and Chao et al., (2000) Molecular Therapy 2:619. For example, a hybrid AAV vector typically comprises the adenovirus 5′ and 3′ cis ITR sequences sufficient for adenovirus replication and packaging (i.e., the adenovirus terminal repeats and PAC sequence).

The term “polyploid AAV” refers to a AAV vector which is composed of capsids from two or more AAV serotypes, e.g., and can take advantages from individual serotypes for higher transduction but not in certain embodiments eliminate the tropism from the parents.

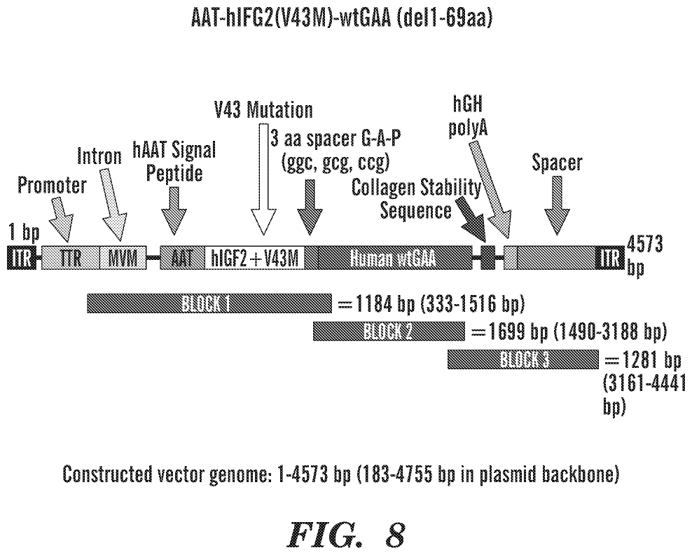

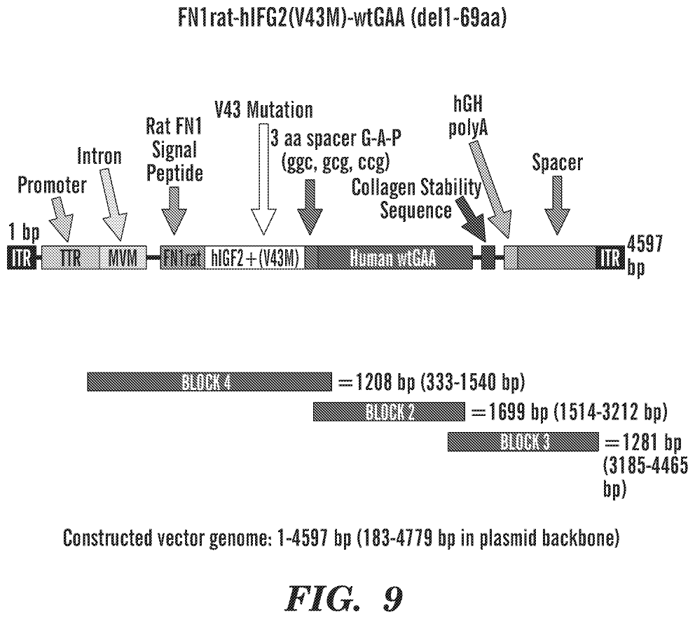

The term “GAA” or “GAA polypeptide,” as used herein, encompasses mature (^{˜}76 or ^{˜}67 kDa) and precursor (e.g., ^{˜}110 kDa) GAA as well as modified (e.g., truncated or mutated by insertion(s), deletion(s) and/or substitution(s)) GAA proteins or fragments thereof that retain biological function (i.e., have at least one biological activity of the native GAA protein, e.g., can hydrolyze glycogen, as defined above) and GAA variants (e.g., GAA II as described by Kunita et al., (1997) Biochemica et Biophysica Acta 1362:269; GAA polymorphisms and SNPs are described by Hirschhorn, R. and Reuser, A. J. (2001) in The Metabolic and Molecular Basis for Inherited Disease (Scriver, C. R., Beaudet. A. L., Sly, W. S. & Valle, D. Eds.), pp. 3389-3419, McGraw-Hill, New York, see pages 3403-3405; each incorporated herein by reference in its entirety). Any GAA coding sequence known in the art may be used, for example, see the coding sequences of FIGS. 8 and 9; GenBank Accession number NM_00152 and Hoefsloot et al., (1988) EMBO J. 7:1697 and Van Hove et al., (1996) Proc. Natl. Acad. Sci. USA 93:65 (human), GenBank Accession number NM_008064 (mouse), and Kunita et al., (1997) Biochemica et Biophysics Acta 1362:269 (quail); the disclosures of which are incorporated herein by reference for their teachings of GAA coding and noncoding sequences.

The term “targeting peptide” is also referred to as a “targeting sequence” as used herein is intended to refer to a peptide that targets a particular subcellular compartment, for example, a mammalian lysosome. A targeting peptide encompassed for use herein is a lysosome targeting peptide that is mannose-6-phosphate-independent.

The term “IGF2 sequence” or “IGF-2 sequence” is used in conjunction with “IGF2 leader sequence” and “IGF-2 leader sequence” are used interchangeably herein and refer to a sequence of the IGF2 polypeptide that binds to the CI-MBR on the surface of the cell. In particular, the IGF2 sequence is a peptide that comprises a part of the IGF-2 uptake sequence of SEQ ID NO: 5, or comprises a modification in amino acid of SEQ ID NO:5. An IGF2 sequence refers to a peptide sequence that binds to a receptor domain consisting essentially of repeats 11-12, repeat 11 or amino acids 1508-1566 of the human cation-independent mannose-6-phosphate receptor (CI-MPR or CA-M6P receptor).

The terms “secretory signal sequence” or “signal sequence” variations thereof are used herein interchangeably, and intended to refer to amino acid sequences that function to enhance (as defined above) secretion of an operably linked polypeptide, e.g., GAA or GAA fusion protein from the cell as compared with the level of secretion seen with the native polypeptide. As defined above, by “enhanced” secretion, it is meant that the relative proportion of lysosomal polypeptide synthesized by the cell that is secreted from the cell is increased; it is not necessary that the absolute amount of secreted protein is also increased. In particular embodiments of the invention, essentially all (i.e., at least 95%, 97%, 98%, 99% or more) of the GAA-polypeptide is secreted. It is not necessary, however, that essentially all or even most of the GAA polypeptide is secreted, as long as the level of secretion is enhanced as compared with the native GAA polypeptide.

As used herein, the term “amino acid” encompasses any naturally occurring amino acid, modified forms thereof, and synthetic amino acids.

Additional patents incorporated for reference herein that are related to, disclose or describe an AAV or an aspect of an AAV, including the DNA vector that includes the gene of interest to be expressed are: U.S. Pat. Nos. 6,491,907; 7,229,823; 7,790,154; 7,201898; 7,071,172; 7,892,809; 7,867,484; 8,889,641; 9,169,494; 9,169,492; 9,441,206; 9,409,953; and, 9,447,433; 9,592,247; and, 9,737,618.

### II. rAAV Genome Elements

As disclosed herein, one aspect of the technology relates to a rAAV vector comprising a capsid, and within its capsid, a nucleotide sequence referred to as the “rAAV vector genome”. The rAAV vector genome (also referred to as “rAAV genome) includes multiple elements, including, but not limited to two inverted terminal repeats (ITRs, e.g., the 5′-ITR and the 3′-ITR), and located between the ITRs are additional elements, including a promoter, a heterologous gene and a poly-A tail.

In some embodiments, the rAAV genome disclosed herein comprises a 5′ ITR and 3′ ITR sequence, and located between the 5′ITR and the 3′ ITR, a promoter, e.g., a liver specific promoter sequence, which operatively linked to a heterologous nucleic acid encoding a nucleic acid encoding an alpha-glucosidase (GAA) polypeptide, where the heterologous nucleic acid sequence can further comprise one or more of the following elements: an intron sequence, a nucleic acid encoding a secretory signal peptide, a nucleic acid encoding an IGF2 sequence, and a poly A sequence.

In some embodiments, the rAAV genome disclosed herein comprises a 5′ ITR and 3′ ITR sequence, and located between the 5′ITR and the 3′ ITR, a promoter operatively linked to a heterologous nucleic acid encoding a secretory peptide and nucleic acid encoding an alpha-glucosidase (GAA) polypeptide (i.e., the heterologous nucleic acid encodes a GAA fusion polypeptide comprising a signal peptide-GAA polypeptide), where the rAAV genome optionally further comprises one or more of: an intron sequence, a collagen stability (CS) sequence, a polyA tail and a nucleic acid encoding a spacer of at least 1 amino acid. In some embodiments, the rAAV genome disclosed herein comprises a 5′ ITR and 3′ ITR sequence, and located between the 5′ITR and the 3′ ITR, a liver promoter operatively linked to a heterologous nucleic acid encoding a secretory peptide (e.g., FIV, ATT or GAA signal peptides) and nucleic acid encoding an alpha-glucosidase (GAA) polypeptide, where the rAAV genome optionally further comprises one or more of: an intron sequence (e.g., MVM or HBB2 intron sequence), a collagen stability (CS) sequence, a polyA tail and a nucleic acid encoding a spacer of at least 1 amino acid.

In some embodiments, the rAAV genome disclosed herein comprises a 5′ ITR and 3′ ITR sequence, and located between the 5′ITR and the 3′ ITR, a promoter operatively linked to a heterologous nucleic acid encoding a secretory peptide, a targeting peptide and a GAA polypeptide (i.e., the heterologous nucleic acid encodes a GAA fusion polypeptide comprising a signal peptide-targeting sequence-GAA polypeptide), where targeting peptide is a IGF2 sequence as described herein, and where the rAAV genome can optionally further comprise one or more of: an intron sequence, a collagen stability (CS) sequence, a polyA tail and a nucleic acid encoding a spacer of at least 1 amino acid.

Each of the elements in the rAAV genome are discussed herein.

### A. Alpha-Glucosidase (GAA) Polypeptide

Alpha-glucosidase (GAA) polypeptide is a member of family 31 of glycoside hydrolyases. Human GAA is synthesized as a 110 kDal precursor (Wisselaar et al. (1993) J. Biol. Chem. 268(3): 2223-31). The mature form of the enzyme is a mixture of monomers of 70 and 76 kDal (Wisselaar et al. (1993) J. Biol. Chem. 268(3): 2223-31). The precursor enzyme has seven potential glycosylation sites and four of these are retained in the mature enzyme (Wisselaar et al. (1993) J. Biol. Chem. 268(3): 2223-31). The proteolytic cleavage events which produce the mature enzyme occur in late endosomes or in the lysosome (Wisselaar et al. (1993) J. Biol. Chem. 268(3): 2223-31).

The rAAV vector genome can encode a GAA polypeptide can include, for example, amino acid residues 40-952 or 70-952 of human GAA, or a smaller portion, such as amino acid residues 40-790 or 70-790.

In one embodiment, a IGF2 sequence is fused to amino acid 40, or amino acid 70, or to an amino acid within one or two positions of amino acid 40 or 70. In some embodiments, the IGF2 sequence is a ligand for an extracellular receptor, for example, the IGF2 sequence binds to human cation-independent mannose-6-phosphate receptor (CI-MPR) or the IGF2 receptor.

The first 27 amino acids of the human GAA polypeptide are typical of signal peptides of lysosomal and secretory proteins. GAA may be targeted to lysosomes via the phosphomannosyl receptor and/or by sequences associated with the delayed cleavage of the signal peptide (Hirschhorn, R. and Reuser, A. J. (2001), in The Metabolic and Molecular Basis for Inherited Disease, (eds, Scriver, C. R. et al.) pages 3389-3419 (McGraw-Hill, New York). A membrane-bound precursor form of the enzyme (i.e., anchored by the uncleaved signal peptide) has been identified in the lumen of the endoplasmic reticulum (see, e.g., Wisselaar et al., (1993) J. Biol. Chem. 268:2223-31).

The C-terminal 160 amino acids are absent from the mature 70 and 76 kDal GAA polypeptide species. However, certain Pompe alleles resulting in the complete loss of GAA activity map to this region, for example Val949Asp (Becker et al. (1998) J. Hum. Genet. 62:991). The phenotype of this mutant indicates that the C-terminal portion of the protein, although not part of the 70 or 76 kDal species, plays an important role in the function of the protein. It has also been reported that the C-terminal portion of the protein, although cleaved from the rest of the protein during processing, remains associated with the major species (Moreland et al. (Nov. 1, 2004) J. Biol. Chem., Manuscript 404008200). Accordingly, the C-terminal residues could play a direct role in the catalytic activity of the protein, and/or may be involved in promoting proper folding of the N-terminal portions of the protein.

The native GAA gene encodes a precursor polypeptide which possesses a signal sequence and an adjacent putative trans-membrane domain, a trefoil domain (PFAM PF00088) which is a cysteine-rich domain of about 45 amino acids containing 3 disulfide linkages (Thim (1989) FEBS Lett. 250:85), the domain defined by the mature 70/76 kDal polypeptide, and the C-terminal domain. It has been reported that both the trefoil domain and the C-terminal domain are required for the production of functional GAA, and that it is possible that the C-terminal domain interacts with the trefoil domain during protein folding perhaps facilitating appropriate disulfide bond formation in the trefoil domain.

The GAA polypeptide is described in U.S. Pat. Nos. 5,962,313 and 6,537,785, which are incorporated herein in their entireties by reference. One of ordinary skill in the art can appreciate particular positions of GAA to which a secretory signal peptide (SS) or alternatively, the targeting peptide (e.g., IGF2 sequence) can be fused. Accordingly, in one aspect the invention relates to a GAA fusion protein, where the SP or IGF2 sequence is fused to amino acid 40, 68, 69, 70, 71, 72, 779, 787, 789, 790, 791, 792, 793, or 796 of human GAA or a portion thereof.

In some embodiments of the methods and compositions as disclosed herein, the human GAA protein expressed by the AAV comprises amino acids of SEQ ID NO: 10, or fragments thereof, for example a human GAA protein beginning at residue 40, 68, 69, 70, 71, 72, 779, 787, 789, 790, 791, 792, 793, or 796 of SEQ ID NO: 10. In some embodiments of the methods and compositions as disclosed herein, the human GAA protein expressed by the AAV comprises amino acids of SEQ ID NO: 10, or a protein at least 60%, or 70%, or 80%, 85% or 90% or 95%, or 98%, or 99% identical to SEQ ID NO: 10. In some embodiments of the methods and compositions as disclosed herein, the human GAA protein expressed by the AAV comprises amino acids is a human GAA protein beginning at residue 40, 68, 69, 70, 71, 72, 779, 787, 789, 790, 791, 792, 793, or 796 of SEQ ID NO: 10, or a protein at least 60%, or 70%, or 80%, 85% or 90% or 95%, or 98%, or 99% identical thereto.

In some embodiments, one of ordinary skill in the art can appreciate particular positions of GAA to which a secretory signal peptide (SS) or alternatively, the targeting peptide (e.g., IGF2 sequence) can be fused. For example, International Patent application WO2018046774A1, which is incorporated herein in its entirety, discloses truncated GAA polypeptides to which the secretory signal peptide (SS) or alternatively, the targeting peptide (e.g., IGF2 sequence) can be attached, discloses truncated GAA polypeptide variants as follows: Δ1, Δ2, Δ3, Δ4, Δ5, Δ6, Δ7, Δ8, Δ9, Δ10, Δ11, Δ12, Δ13, Δ14, Δ15, Δ16, Δ17, Δ18, Δ19, Δ20, Δ21, Δ22, Δ23, Δ24, Δ25, Δ26, Δ27, Δ28, Δ29, Δ30, Δ31, Δ32, Δ33, Δ34, Δ35, Δ36, Δ37, Δ38, Δ39, Δ40, Δ41, Δ42, Δ43, Δ44, Δ45, Δ46, Δ47, Δ48, Δ49, Δ50, Δ51, Δ52, Δ53, Δ54, Δ55, Δ56, Δ57, Δ58, Δ59, Δ60, Δ61, Δ62, Δ63, Δ64, Δ65, Δ66, Δ67, Δ68, Δ69, Δ70, Δ71, Δ72, Δ73, Δ74 or Δ75 GAA truncated form.

In some embodiments, the GAA-fusion polypeptides encoded by the rAAV genome as described herein can include, for example, amino acid residues 40-952 or residues 70-952 of human GAA, or a smaller portion, such as amino acid residues 40-790 or 70-790. In one embodiment, a secretory signal peptide (SS) or targeting peptide, e.g., IGF2 sequence is fused to amino acid 40, or to amino acid 70, or to an amino acid within one or two positions of amino acid 40 or 70.

In some embodiments, the fusion protein comprising the secretory signal peptide (SS) and GAA polypeptide and optionally an IGF2 sequence (i.e., a SS-GAA fusion polypeptide, or a SS-IGF2-GAA fusion protein) comprises amino acid residues 40-952 or residues 70-952 of human acid alpha-glucosidase (GAA) (SEQ ID NO: 10). In some embodiments, the N-terminal of the GAA polypeptide is attached to the C-terminus of the SS and in some embodiments, the N-terminal of the GAA polypeptide is attached to the C-terminus of the IGF2 sequence, and the N-terminus of the IGF2 sequence is attached to the C-terminus of the secretory signal peptide.

In one embodiment, the rAAV genome comprises a heterologous nucleic acid sequence encoding a secretory signal peptide or IGF2 sequence fused in frame to the 3′ terminus of a GAA nucleic acid sequence that encodes the entire GAA polypeptide (e.g., the N-terminal/catalytic and the C-terminal domain). For example, heterologous nucleic acid sequence encoding a secretory signal peptide, or IGF2 sequence is fused in frame to the 3′ terminus of a GAA nucleic acid sequence that encodes the 70 kDa and 76 kDa GAA polypeptides, such both polypeptides are expressed from the rAAV genome when the rAAV vector transduces a mammalian cell. In some embodiments, expression of the GAA nucleic acid can be driven by two promoters in the rAAV genome or by one promoter driving expression of a bicistronic construct.

In some embodiments of the methods and compositions as disclosed herein, the rAAV vector comprises a nucleic acid sequence encoding a GAA protein is a wild type GAA nucleic acid sequence, e.g., SEQ ID NO: 11 or SEQ ID NO: 72. In some embodiments of the methods and compositions as disclosed herein, the rAAV vector comprises a nucleic acid sequence encoding a GAA protein which is a codon optimized GAA nucleic acid sequence, for enhanced expression in vivo and/or to reduce CpG islands and/or to reduce the innate immune response. Exemplary codon optimized GAA nucleic sequences encompassed for use in the methods and rAAV compositions as disclosed herein can be selected from any of: SEQ ID NO: 73, SEQ ID NO: 74, SEQ ID NO: 75 and SEQ ID NO: 76, or a nucleic acid sequence having at least 60%, or 70%, or 80%, 85% or 90% or 95%, or 98%, or 99% sequence identity to SEQ ID NO: 73, SEQ ID NO: 74, SEQ ID NO: 75 and SEQ ID NO: 76.

The C-terminal domain of GAA functions in trans in conjunction with the 70/76 kDal species to generate active GAA. The boundary between the catalytic domain and the C-terminal domain appears to be at about amino acid residue 791, based on its presence in a short region of less than 18 amino acids that is absent from most members of the family 31 hydrolyases and which contains 4 consecutive proline residues in GAA. It has been reported that the C-terminal domain associated with the mature species begins at amino acid residue 792 (Moreland et al. (Nov. 1, 2004) J. Biol. Chem., Manuscript 404008200). Accordingly, in some embodiments, the GAA nucleic acid sequence that encodes the entire GAA polypeptide, with the exception of the C-terminal domain. Thus, in such an embodiment, the rAAV vector can be used to transduce a mammalian cell that expresses the C-terminal domain of GAA as a separate polypeptide.

Furthermore, patients having Pompe or GSDs may benefit from the administration of an optimized form of GAA. For example, it has been shown (Sun et al. (2013) Mol Genet Metab 108(2): 145; WO2010/005565) that administration of GAA reduces glycogen in primary myoblasts from glycogen storage disease type III (GSD III) patients.

### B. Secretory Signal Peptide

Native GAA signal peptide is not cleaved in the ER thereby causing native GAA polypeptide to be membrane bound in the ER (Tsuji et al. (1987) Biochem. Int. 15(5):945-952). In some cell types, GAA polypeptide can be found bound to the plasma membrane with retention of the membrane topology of the ER presumably due to the failure to cleave the signal peptide (Hirschhorn et al., in The Metabolic and Molecular Basis of Inherited Disease, Valle, ed., 2001, McGraw-Hill: New York, pp. 3389-3420).

Disruption of the membrane association of GAA can be accomplished by replacing the endogenous GAA signal peptide (and optionally adjacent sequences) with an alternate signal peptide for GAA.

Accordingly, the rAAV genome disclosed herein comprises a heterologous nucleic acid sequence that encodes a secretory signal peptide. In representative embodiments, the rAAV vector and rAAV genome as disclosed herein further comprises a heterologous nucleic acid encoding a GAA polypeptide to be transferred to a target cell. The heterologous nucleic acid is operatively associated with the segment encoding the secretory signal peptide, such that upon transcription and translation a fusion polypeptide is produced containing the secretory signal sequence operably associated with (e.g., directing the secretion of) the GAA polypeptide.

In some embodiments, the secretory signal peptide is heterologous to (i.e., foreign or exogenous to) the polypeptide of interest. For example, if the secretory signal peptide is a fibronectin secretory signal peptide, the polypeptide of interest is not fibronectin. In some embodiments, the secretory signal peptide is selected from any of: AAT signal peptide, a fibronectin signal peptide (FN1), or an active fragment of AAT, FN1 or GAA signal peptide having secretory signal activity. In alternative embodiments, the secretory signal peptide is not heterologous to GAA, i.e., the signal peptide is the GAA signal peptide (i.e., residues 1-27 of the native GAA polypeptide).

In general, the secretory signal peptide will be at the amino-terminus (N-terminus) of the fusion polypeptide (i.e., the nucleic acid segment encoding the secretory signal peptide is 5′ to the heterologous nucleic acid encoding the GAA peptide or GAA-fusion peptide in the rAAV vector or rAAV genome as disclosed herein). Alternatively, the secretory signal may be at the carboxy-terminus or embedded within the GAA polypeptide or GAA fusion polypeptide (e.g., IGF2-GAA fusion polypeptide), as long as the secretory signal is operatively associated therewith and directs secretion of the GAA polypeptide or GAA fusion polypeptide of interest (either with or without cleavage of the signal peptide from the GAA polypeptide) from the cell.

The secretory signal is operatively associated with the polypeptide of interest so that the GAA polypeptide or GAA fusion polypeptide is targeted to the secretory pathway. Alternatively stated, the secretory signal is operatively associated with the GAA polypeptide such that the GAA-polypeptide or GAA fusion polypeptide is secreted from the cell at a higher level (i.e., a greater quantity) than in the absence of the secretory signal peptide. The degree to which the secretory signal peptide directs the secretion of the GAA-polypeptide or GAA fusion polypeptide is not critical, as long as it provides a desired level of secretion and/or regulation of expression of the GAA polypeptide. Those skilled in the art will appreciate that when secretory proteins are over-expressed they often saturate the cellular secretion mechanisms and are retained within the cell. In general, typically at least about 20%, 30%, 40%, 50%, 70%, 80%, 85%, 90%, 95% or more of the GAA-polypeptide or IGF2-GAA fusion polypeptide (alone and/or fused with the signal peptide) is secreted from the cell. In other embodiments, essentially all of the detectable polypeptide (alone and/or in the form of the fusion polypeptide) is secreted from the cell.

By the phrase “secreted from the cell”, the polypeptide may be secreted into any compartment (e.g., fluid or space) outside of the cell including but not limited to: the interstitial space, blood, lymph, cerebrospinal fluid, kidney tubules, airway passages (e.g., alveoli, bronchioles, bronchia, nasal passages, etc.), the gastrointestinal tract (e.g., esophagus, stomach, small intestine, colon, etc.), vitreous fluid in the eye, and the cochlear endolymph, and the like.

In one embodiment, the rAAV genome comprises a heterologous nucleic acid that encodes a secretory signal peptide (SP) fused to the GAA polypeptide. In alternative embodiments, the rAAV genome comprises a heterologous nucleic acid that encodes a secretory signal peptide (SP) fused to the GAA-fusion polypeptide, where the GAA-fusion polypeptide is comprises a targeting peptide (e.g., IGF2 sequence) fused to a GAA polypeptide. Accordingly, the signal peptide disclosed herein increases the efficacy of secretion of the GAA polypeptide or IGF2-GAA fusion polypeptide from the cell transduced with the rAAV vector or comprising the rAAV genome as described herein

Accordingly, in some embodiments, the rAAV genome disclosed herein comprises a 5′ ITR and 3′ ITR sequence, and located between the 5′ ITR and the 3′ ITR, a promoter operatively linked to a heterologous nucleic acid encoding a secretory peptide and nucleic acid encoding an alpha-glucosidase (GAA) polypeptide (i.e., the heterologous nucleic acid encodes a GAA fusion polypeptide comprising a signal peptide-GAA polypeptide).

In alternative embodiments, the rAAV genome disclosed herein comprises a 5′ ITR and 3′ ITR sequence, and located between the 5′ITR and the 3′ ITR, a promoter operatively linked to a heterologous nucleic acid encoding a secretory peptide and nucleic acid encoding an alpha-glucosidase (GAA) fusion polypeptide, where the fusion protein comprises IGF2 sequence and a GAA polypeptide (i.e., the heterologous nucleic acid encodes a GAA fusion polypeptide comprising a signal peptide-IGF2-GAA polypeptide).

In some embodiments, the secretory signal peptide (also referred to as a signal peptide) results in at least about 50%, 60%, 75%, 85%, 90%, 95%, 98% or more of the GAA-polypeptide or GAA-fusion polypeptide secreted from the cell. The relative proportion of GAA-polypeptide (e.g., fusion polypeptide comprising signal peptide-GAA (SP-GAA), or a fusion protein comprising a signal peptide-targeting peptide-GAA, e.g., SP-IGF2-GAA fusion polypeptide) expressed from the rAAV genome that is secreted from the cell can be routinely determined by methods known in the art and as described in the Examples, e.g., by measuring GAA activity in the supernatant. Secreted proteins can be detected by directly measuring the protein itself (e.g., by Western blot) or by protein activity assays (e.g., enzyme assays) in cell culture medium, serum, milk, etc.

Generally, secretory signal peptides are cleaved within the endoplasmic reticulum and, in some embodiments, the secretory signal peptide is cleaved from the GAA polypeptide prior to secretion. It is not necessary, however, that the secretory signal peptide is cleaved as long as secretion of the GAA polypeptide or IGF2-GAA fusion polypeptide from the cell is enhanced and the GAA polypeptide is functional. Thus, in some embodiments, the secretory signal peptide is partially or entirely retained.

In some embodiments, the rAAV genome, or an isolated nucleic acid as disclosed herein comprises a nucleic acid encoding a chimeric polypeptide comprising a GAA polypeptide operably linked to a secretory signal peptide, and the chimeric polypeptide is expressed and produced from a cell transduced with the rAAV vector and the GAA polypeptide is secreted from the cell. The GAA polypeptide or GAA fusion polypeptide (e.g., IGF2-GAA fusion polypeptide) can be secreted after cleavage of all or part of the secretory signal peptide. Alternatively, the GAA polypeptide or GAA fusion polypeptide (e.g., IGF2-GAA fusion polypeptide) can retain the secretory signal peptide (i.e., the secretory signal is not cleaved). Thus, in this context, the “GAA polypeptide or GAA fusion polypeptide” can be a chimeric polypeptide comprising the secretory peptide.

Those skilled in the art will further understand that the chimeric polypeptide can contain additional amino acids, e.g., as a result of manipulations of the nucleic acid construct such as the addition of a restriction site, as long as these additional amino acids do not render the secretory signal sequence or the GAA polypeptide or GAA fusion polypeptide (e.g., IGF2-GAA fusion polypeptide) non-functional. The additional amino acids can be cleaved or can be retained by the mature GAA polypeptide as long as retention does not result in a nonfunctional GAA polypeptide.

In representative embodiments, the secretory signal peptide replaces most, essentially all or all of the sequence found in the native GAA polypeptide. In particular embodiments, most or all of the native sequence of GAA is retained, as long as secretion of the GAA polypeptide or GAA fusion polypeptide (e.g., IGF2-GAA fusion polypeptide) is enhanced and the mature GAA polypeptide is functional.

Without wishing to limited to theory, it is generally believed that secretory signal sequences direct the insertion of the nascent polypeptide into the endoplasmic reticulum from whence it is transported to the golgi, which then fuses with the cellular membrane to secrete the polypeptide from the cell. Typically, the secretory signal is cleaved from the polypeptide during processing, which is believed to occur in the endoplasmic reticulum. In the case of the fusion polypeptides of the present invention, it is not necessary that the secretory signal peptide be cleaved from the chimeric GAA polypeptide or chimeric IGF2-GAA fusion polypeptide completely or at all. In some embodiments, the secretory signal peptide may be essentially completely cleaved; alternatively, in some cells, there may be incomplete cleavage or essentially no cleavage. While not wishing to be limited to any particular theory, in some embodiments it appears that retention (i.e., non-cleavage) of some or all of the secretory signal peptide stabilizes the resulting chimeric GAA polypeptide or chimeric IGF2-GAA fusion polypeptide.

In some embodiments, the secretory signal peptide is only partially removed from polypeptide, i.e., at least about one, two, three, four, five, six, seven, eight, nine, ten, twelve or even fifteen or more of the amino acid residues are retained by the secreted polypeptide. For illustrative purposes only using Fibronectin signal peptide as an exemplary signal peptide, amino acids 22 (Val) to 32 (Arg), 23 (Arg) to 32 (Arg), 24 (Cys) to 32 (Arg), 25 (Thr) to 32 (Arg) or 26 (Glu) to 32 (Arg) of SEQ ID NO: 18 may be retained by the secreted polypeptide.

A secretory signal peptide encompassed for use in the rAAV genome as disclosed herein can be derived in whole or in part from the secretory signal of a secreted polypeptide (i.e., from the precursor) and/or can be in whole or in part synthetic. As one skilled in the art will appreciate, secretory signal sequences are generally operative across species. Accordingly, a secretory signal peptide can be from any species of origin, including animals (e.g., avians and mammals such as humans, simians and other non-human primates, bovines, ovines, caprines, equines, porcines, canines, felines, rats, mice, lagomorphs), plants, yeast, bacteria, protozoa or fungi. The length of the secretory signal sequence is not critical; generally, known secretory signal sequences are from about 10-15 to 50-60 amino acids in length. Further, known secretory signals from secreted polypeptides can be altered or modified (e.g., by substitution, deletion, truncation or insertion of amino acids) as long as the resulting secretory signal sequence functions to enhance secretion of an operably linked GAA polypeptide or GAA fusion polypeptide (e.g., IGF2-GAA fusion polypeptide).

The secretory signal sequences of the invention are not limited to any particular length as long as they direct the polypeptide of interest to the secretory pathway. In representative embodiments, the signal peptide is at least about 6, 8, 10 12, 15, 20, 25, 30 or 35 amino acids in length up to a length of about 40, 50, 60, 75, or 100 amino acids or longer.

Secretory signal peptide encoded by the rAAV genome and in the rAAV vector as disclosed herein can comprise, consist essentially of or consist of a naturally occurring secretory signal sequence or a modification thereof. Numerous secreted proteins and sequences that direct secretion from the cell are known in the art. Exemplary secreted proteins (and their secretory signals) include but are not limited to: erythropoietin, coagulation Factor IX, cystatin, lactotransferrin, plasma protease C1 inhibitor, apolipoproteins (e.g., APO A, C, E), MCP-1, α-2-HS-glycoprotein, α-1-microgolubilin, complement (e.g., C1Q, C3), vitronectin, lymphotoxin-α, azurocidin, VIP, metalloproteinase inhibitor 2, glypican-1, pancreatic hormone, clusterin, hepatocyte growth factor, insulin, α-1-antichymotrypsin, growth hormone, type IV collagenase, guanylin, properdin, proenkephalin A, inhibin β (e.g., A chain), prealbumin, angiocenin, lutropin (e.g., β chain), insulin-like growth factor binding protein 1 and 2, proactivator polypeptide, fibrinogen (e.g., 13 chain), gastric triacylglycerol lipase, midkine, neutrophil defensins 1, 2, and 3, α-1-antitrypsin, matrix gla-protein, α-tryptase, bile-salt-activated lipase, chymotrypsinogen B, elastin, IG lambda chain V region, platelet factor 4 variant, chromogranin A, WNT-1 proto-oncogene protein, oncostatin M, β-neoendorphin-dynorphin, von Willebrand factor, plasma serine protease inhibitor, serum amyloid A protein, nidogen, fibronectin, rennin, osteonectin, histatin 3, phospholipase A2, cartilage matrix Protein, GM-CSF, matrilysin, neuroendocrine protein 7B2, placental protein 11, gelsolin, M-CSF, transcobalamin I, lactase-phlorizin hydrolase, elastase 2B, pepsinogen A, MIP 1-β, prolactin, trypsinogen II, gastrin-releasing peptide II, atrial natriuretic factor, secreted alkaline phosphatase, pancreatic α-amylase, secretogranin I, β-casein, serotransferrin, tissue factor pathway inhibitor, follitropin β-chain, coagulation factor XII, growth hormone-releasing factor, prostate seminal plasma protein, interleukins (e.g., 2, 3, 4, 5, 9, 11), inhibin (e.g., alpha chain), angiotensinogen, thyroglobulin, IG heavy or light chains, plasminogen activator inhibitor-1, lysozyme C, plasminogen activator, antileukoproteinase 1, statherin, fibulin-1, isoform B, uromodulin, thyroxine-binding globulin, axonin-1, endometrial α-2 globulin, interferon (e.g., alpha, beta, gamma), β-2-microglobulin, procholecystokinin, progastricsin, prostatic acid phosphatase, bone sialoprotein II, colipase, Alzheimer's amyloid A4 protein, PDGF (e.g., A or B chain), coagulation factor V, triacylglycerol lipase, haptoglobuin-2, corticosteroid-binding globulin, triacylglycerol lipase, prorelaxin H2, follistatin 1 and 2, platelet glycoprotein IX, GCSF, VEGF, heparin cofactor II, antithrombin-III, leukemia inhibitory factor, interstitial collagenase, pleiotrophin, small inducible cytokine A1, melanin-concentrating hormone, angiotensin-converting enzyme, pancreatic trypsin inhibitor, coagulation factor VIII, α-fetoprotein, α-lactalbumin, senogelin II, kappa casein, glucagon, thyrotropin beta chain, transcobalamin II, thrombospondin 1, parathyroid hormone, vasopressin copeptin, tissue factor, motilin, MPIF-1, kininogen, neuroendocrine convertase 2, stem cell factor procollagen al chain, plasma kallikrein keratinocyte growth factor, as well as any other secreted hormone, growth factor, cytokine, enzyme, coagulation factor, milk protein, immunoglobulin chain, and the like.

In some embodiments, other secretory signal peptides encoded by the rAAV genome and in the rAAV vector as disclosed herein can be selected from, but are not limited to, the secretory signal sequences from prepro-cathepsin L (e.g., GenBank Accession Nos. KHRTL, NP_037288; NP_034114, AAB81616, AAA39984, P07154, CAA68691; the disclosures of which are incorporated by reference in their entireties herein) and prepro-alpha 2 type collagen (e.g., GenBank Accession Nos. CAA98969, CAA26320, CGHU2S, NP_000080, BAA25383, P08123; the disclosures of which are incorporated by reference in their entireties herein) as well as allelic variations, modifications and functional fragments thereof (as discussed above with respect to the fibronectin secretory signal sequence). Exemplary secretory signal sequences include for preprocathepsin L (Rattus norvegicus, MTPLLLLAVLCLGTALA [SEQ ID NO: 27]; Accession No. CAA68691) and for prepro-alpha 2 type collagen (Homo sapiens, MLSFVDTRTLLLLAVTLCLATC [SEQ ID NO: 28]; Accession No. CAA98969). Also encompassed are longer amino acid sequences comprising the full-length secretory signal sequence from preprocathepsin L and prepro-alpha 2 type collagen or functional fragments thereof (as discussed above with respect to the fibronectin secretory signal sequence

In some embodiments, the secretory signal peptide is derived in part or in whole from a secreted polypeptide that is produced by liver cells. In some embodiments, a secretory signal peptide can further be in whole or in part synthetic or artificial. Synthetic or artificial secretory signal peptides are known in the art, see e.g., Barash et al., “Human secretory signal peptide description by hidden Markov model and generation of a strong artificial signal peptide for secreted protein expression,” Biochem. Biophys. Res. Comm. 294:835-42 (2002); the disclosure of which is incorporated herein in its entirety. In particular embodiments, the secretory signal peptide comprises, consists essentially of, or consists of the artificial secretory signal: MWWRLWWLLLLLLLLWPMVWA (SEQ ID NO: 29) or variations thereof having 1, 2, 3, 4, or 5 amino acid substitutions (optionally, conservative amino acid substitutions, conservative amino acid substitutions are known in the art).

Fibronectin Secretory Signal Peptide:

In some embodiments, the secretory signal peptide is a fibronectin secretory signal peptide, which term includes modifications of naturally occurring sequences (as described in more detail below).

In some embodiments, the secretory signal peptide is a fibronectin signal peptide, e.g., a signal sequence of human fibronectin or a signal sequence from rat fibronectin. Fibronectin (FN1) signal sequences and modified FN1 signal peptides encompassed for use in the rAAV genome and rAAV vectors described herein are disclosed in U.S. Pat. No. 7,071,172, which is incorporated herein in its entirety by reference.

Accordingly, the fibronectin secretory signal sequence of the invention may be derived from any species including, but not limited to, avians (e.g., chicken, duck, turkey, quail, etc.), mammals (e.g., human, simian, mouse, rat, bovine, ovine, caprine, equine, porcine, lagamorph, feline, canine, etc.), and other animals including Caenorhabditis elegans, Xenopus laevis, and Danio rerio. Examples of exemplary fibronectin secretory signal sequences include, but are not limited to those listed in Table 1 of U.S. Pat. No. 7,071,172, which is incorporated herein in its entirety by reference.

TABLE 3
Exemplary Fibronectin (FN1) secretory signal peptides

| Species | Secretory Signal sequence | Nucleic acid sequence |
|---|---|---|
| H. Sapiens | MLRGPGPGLLLLAVQCLGTAV | ATG CTT AGG GGT CCG GGG CCC GGG CTG |
| | PSTGA (SEQ ID NO: 20) | CTG CTG CTG GCC GTC CAG TGC CTG GGG |
| | | ACA GCG GTG CCC TCC ACG GGA GCC |
| | | (SEQ ID NO: 25) |
| R. | MLRGPGPGRLLLLAVLCLGTSV | 5′- |
| Norvegicus | RCTETGKSKR (SEQ ID NO: 18) | ATGCTCAGGGGTCCGGGACCCGGGCGG |
| | | CTGCTGCTGCTAGCAGTCCTGTGCCTGG |
| | | GGACATCGGTGCGCTGCACCGAAACCG |
| | | GGAAGAGCAAGAGG-3 (SEQ ID NO: 23) |
| | | (nucleotides 208-303) |
| R. | MLRGPGPGRLLLLAVLCLGTSV | 5'-ATG CTC AGG GGT CCG GGA CCC GGG |
| Norvegicus | RCTETGKSKR ↑ LALQIV | CGG CTG CTG CTG CTA GCA GTC CTG TGC |
| | (SEQ ID NO: 19) | CTG GGG ACA TCG GIG CGC TGC |
| | | ACC GAA ACC GGG AAG AGC AAG AGG ↑ |
| | | CAG GCT CAG CAA ATC GIG-3′. (SEQ ID |
| | | NO: 24) |
| | | (↑ denotes the cleavage site) |
| X. laevis | MRRGALTGLLLVLCLSVVLRA | ATG CGC CGG GGG GCC CTG ACC GGG CTG |
| | APSATSKKRR (SEQ ID NO: 21) | CTC CTG GTC CTG TGC CTG AGT GTT GTG |
| | | CTA CGT GCA GCC CCC TCT GCA ACA AGC |
| | | AAG AAG CGC AGG (SEQ ID NO: 26) |

An exemplary nucleotide sequence encoding the fibronectin secretory signal sequence of Rattus norvegicus is found at GenBank accession number X15906 (the disclosure of which is incorporated herein by reference). As yet another illustrative sequence, the nucleotide sequence encoding the secretory signal peptide of human fibronectin 1, transcript variant 1 (Accession No. NM_002026, nucleotides 268-345; the disclosure of Accession No. NM_002026 is incorporated herein by reference in its entirety). Another exemplary secretory signal sequence is encoded by the nucleotide sequence encoding the secretory signal peptide of the Xenopus laevis fibronectin protein (Accession No. M77820, nucleotides 98-190; the disclosure of Accession No. M77820 incorporated herein by reference in its entirety).

In another embodiment, the fibronectin signal sequence (FN1, nucleotides 208-303, 5′-ATG CTC AGG GGT CCG GGA CCC GGG CGG CTG CTG CTG CTA GCA GTC CTG TGC CTG GGG ACA TCG GTG CGC TGC ACC GAA ACC GGG AAG AGC AAG AGG-3′, SEQ ID NO: 23) was derived from the rat fibronectin mRNA sequence (Genbank accession #X15906) and codes for the following peptide signal sequence: Met Leu Arg Gly Pro Gly Pro Gly Arg Leu Leu Leu Leu Ala Val Leu Cys Leu Gly Thr Ser Val Arg Cys Thr Glu Thr Gly Lys Ser Lys Arg (SEQ ID NO: 18).

In some embodiments, a nucleic acid sequence encoding the rat fibronectin signal peptide does not include the nucleotide sequences that are 3′ to the cleavage site (i.e., encode the amino acids C-terminal to the cleavage site). As those skilled in the art will appreciate, the fibronectin secretory signal peptide is typically cleaved from the fibronectin precursor by the cleavage action of an intracellular peptidase.

Those skilled in the art will appreciate that the secretory signal sequence may encode one, two, three, four, five or all six or more of the amino acids at the C-terminal side of the peptidase cleavage site (identified by an ↑) (see e.g., SEQ ID NO: 19 and 24 in Table 3). Those skilled in the art will appreciate that additional amino acids (e.g., 1, 2, 3, 4, 5, 6 or more amino acids) on the carboxy-terminal side of the cleavage site may be included in the secretory signal sequence.

In some embodiments, the rAAV genome can encode a fibronectin secretory signal peptide from species other than those disclosed specifically herein as well as allelic variations and modifications thereof that retain secretory signal activity (e.g., confers a greater level [i.e., quantity] of secretion of the associated polypeptide than is observed in the absence of the secretory signal peptide, alternatively stated, has at least 50%, 70%, 80% or 90% or more of the secretory signal activity of the secretory signal peptides specifically disclosed herein or even has a greater level of secretory signal activity). For illustrative purposes only, a fibronectin secretory signal peptide encoded in the rAAV genome as disclosed herein can also include functional portions or fragments of the full-length secretory signal peptide (e.g., functional fragments of the amino acid sequences shown in Table 3 (Fibronectin signal sequences)). The length of the fragment is not critical as long as it has secretory signal activity (e.g., confers a greater level [i.e., quantity] of secretion of the associated polypeptide than is observed in the absence of the secretory signal peptide). Illustrative fragments comprise at least 10, 12, 15, 18, 20, 25 or 27 contiguous amino acids of the full-length secretory signal peptide (e.g., fragments of the amino acid sequences shown in Table 3, i.e., FN1 signal peptides of SEQ ID NO: 18, 19, 20, 22, encoded by nucleic acids of SEQ ID NO: 23, 24, 25 and 26 respectively).

). In embodiments of the invention, the functional fragment has at least about 50%, 70%, 80%, 90% or more secretory signal activity as compared with the sequences specifically disclosed herein or even has a greater level of secretory signal activity.

Likewise, those skilled in the art will appreciate that longer amino acid sequences (and nucleotide sequences encoding the same) that comprise the full-length fibronectin secretory signal (or fragment thereof with secretory signal activity) are encompassed by the term “fibronectin signal sequence” according to the present invention. Additional amino acids (e.g., 1, 2, 4, 6, 8, 10, 15 or even more amino acids) may be added to the fibronectin secretory signal sequence without unduly affecting secretory signal activity thereof (e.g., confers a greater level [i.e., quantity] of secretion of the associated polypeptide than is observed in the absence of the secretory signal peptide, alternatively stated, has at least about 50%, 70%, 80%, 90% or more secretory signal activity as compared with the sequences specifically disclosed herein or even has a greater level of secretory signal activity). For example, those skilled in the art will appreciate that peptide cleavage sites (described above) or restriction enzyme sites may be added, typically at either end of the secretory signal sequence. Additional sequences having other functions may also be fused to the fibronectin secretory signal sequence (e.g., sequences encoding FLAG sequences or poly-His tails that facilitate purification of the polypeptide or spacer sequences). Additionally, sequences that encode polypeptides that enhance the stability of the polypeptide of interest may be added, e.g., sequences encoding maltose binding protein (MBP) or glutathione-S-transferase.

A secretory signal sequence can further be from any species as described above with respect to fibronectin secretory signal sequences. A comparison of the fibronectin secretory signal sequence with the secretory signal sequences from the cathepsin L and alpha 2 type collagen precursors has resulted in identification of a core or canonical amino acid sequence: LLLLAVLCLGT (SEQ ID NO: 64). Accordingly, in some embodiments, a rAAV genome comprises a chimeric nucleic acid sequences comprising the canonical amino acid sequence of LLLLAVLCLGT (SEQ ID NO: 64).

Likewise, those skilled in the art will appreciate that the secretory signal sequences specifically disclosed herein will typically tolerate substitutions in the amino acid sequence and retain secretory signal activity (e.g., at least 50%, 70%, 80%, 90%, 95% or higher of the secretory signal activity the secretory signal peptides specifically disclosed herein). To identify secretory signal peptides of the invention other than those specifically disclosed herein, amino acid substitutions may be based on any characteristic known in the art, including the relative similarity or differences of the amino acid side-chain substituents, for example, their hydrophobicity, hydrophilicity, charge, size, and the like.

Peptidase Cleavage Sites

In some embodiments, one or more exogenous peptidase cleavage site may be inserted into the secretory signal peptide-GAA fusion polypeptide, e.g., between the secretory signal peptide and the GAA polypeptide. In particular embodiments, an autoprotease (e.g., the foot and mouth disease virus 2A autoprotease) is inserted between the secretory signal peptide and the GAA polypeptide or IGF2-GAA fusion polypeptide. In other embodiments, a protease recognition site that can be controlled by addition of exogenous protease is employed (e.g., Lys-Arg recognition site for trypsin, the Lys-Arg recognition site of the Aspergillus KEX2-like protease, the recognition site for a metalloprotease, the recognition site for a serine protease, and the like).

In some embodiments, the signal peptide is flanked by peptidase cleavage sites, so the signal peptide can be removed. Accordingly, in some embodiments, the rAAV genome comprises a nucleic acid encoding a signal peptide that has a N-terminal or C-terminal cleavage site, or N-terminal and C-terminal cleavage sites. In some embodiments, the N-terminal cleavage site is cleaved by the same enzyme as the C-terminal cleavage site and in some embodiments, the N-terminal cleavage site and the C-terminal cleavage site are cleaved by different enzymes.

While not necessary, in particular embodiments of the invention, the heterologous nucleic acid encoding the GAA polypeptide of the rAAV genome encodes a mature form of the GAA polypeptide (e.g., excluding any precursor sequences that are normally removed during processing of the polypeptide). Likewise, the GAA polypeptide sequence may be modified to delete or inactivate native targeting or processing signals (for example, if they interfere with the desired level of secretion of the polypeptide according to the present invention).

### C. IGF2 Sequence

In one embodiment, the rAAV genome comprises a heterologous nucleic acid that encodes a targeting peptide fused to the GAA polypeptide. In some embodiments, the targeting peptide is a ligand for an extracellular receptor. In some embodiments, a targeting peptide is a targeting domain that binds an extracellular domain of a receptor on the surface of a target cell and, upon internalization of the receptor, permits localization of the polypeptide in a human lysosome. In one embodiment, the targeting peptide includes a urokinase-type plasminogen receptor moiety capable of binding the cation-independent mannose-6-phosphate receptor. In some embodiments, the targeting peptide incorporates one or more amino acid sequences of a IGF2 sequence.

IGF2 is also known by alias; chromosome 11 open reading frame 43, insulin-like growth factor 2, IGF-II, FLJ44734; IGF2, somatomedin A and preptin. The mRNA of wild-type human IGF2 sequence is corresponds to: GCTTACCGCCCCAGTGAGACCCTGTGCGGCGGGGAGCTGGTGGACACCCTCCAGTTCGTC TGTGGGGACCGCGGCTTCTACTTCAGCAGGCCCGCAAGCCGTGTGAGCCGTCGCAGCCGT GGCATCGTTGAGGAGTGCTGTTTCCGCAGCTGTGACCTGGCCCTCCTGGAGACGTACTGT GCTACCCCCGCCAAGTCCGAG (SEQ ID NO: 1). The full length IGF2 protein (including the IGF2 leader sequence) is encoded by the nucleic acid sequence of NM_000612.6, and encodes the full length IGF2 protein NP_000603.1.

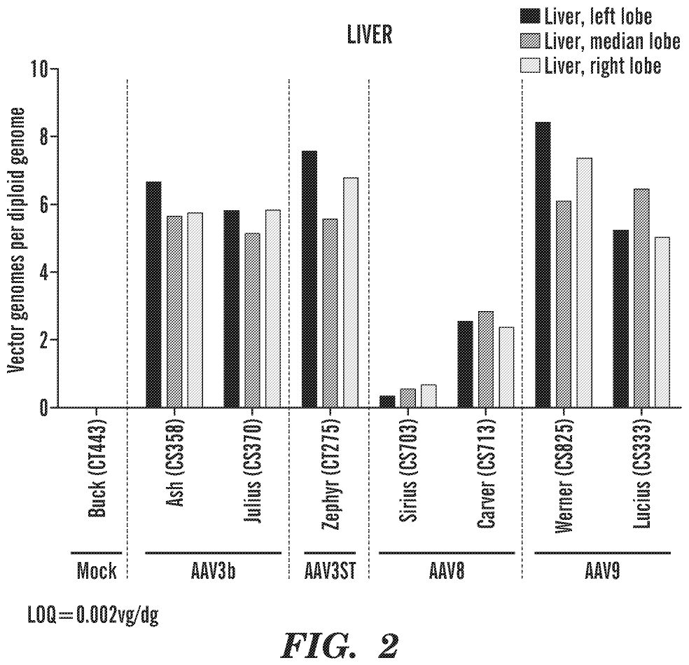

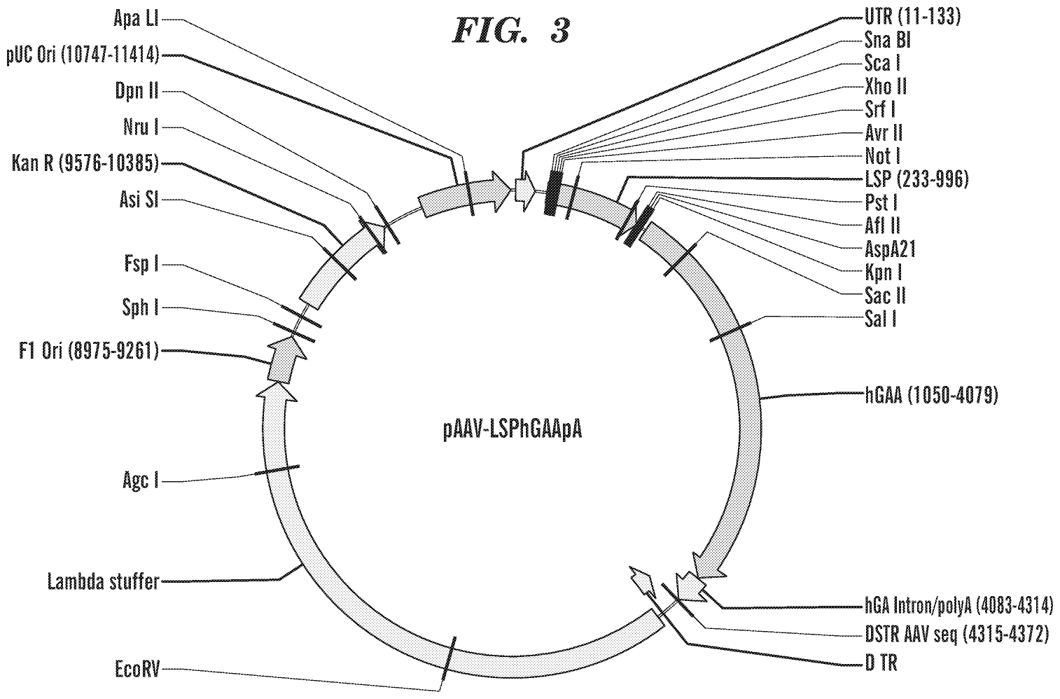

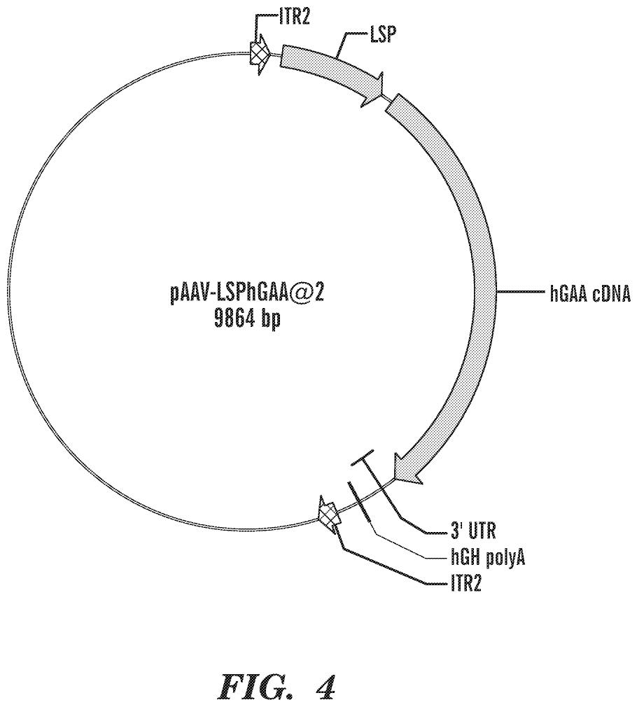

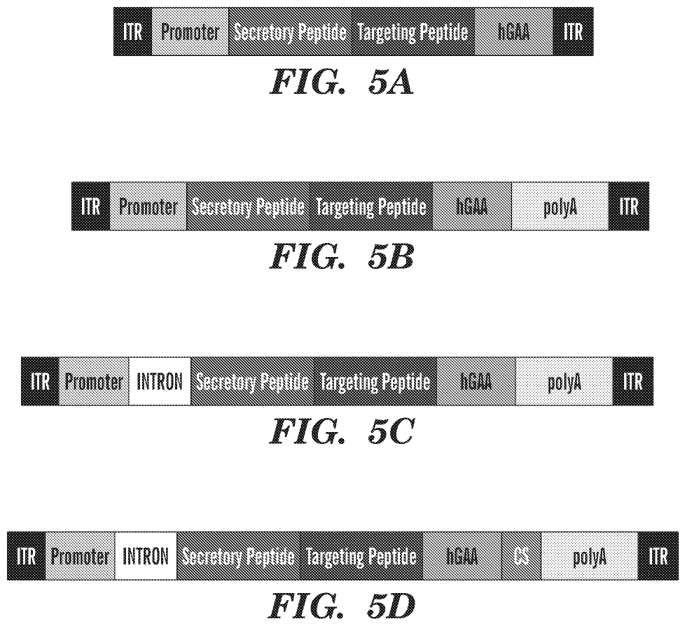

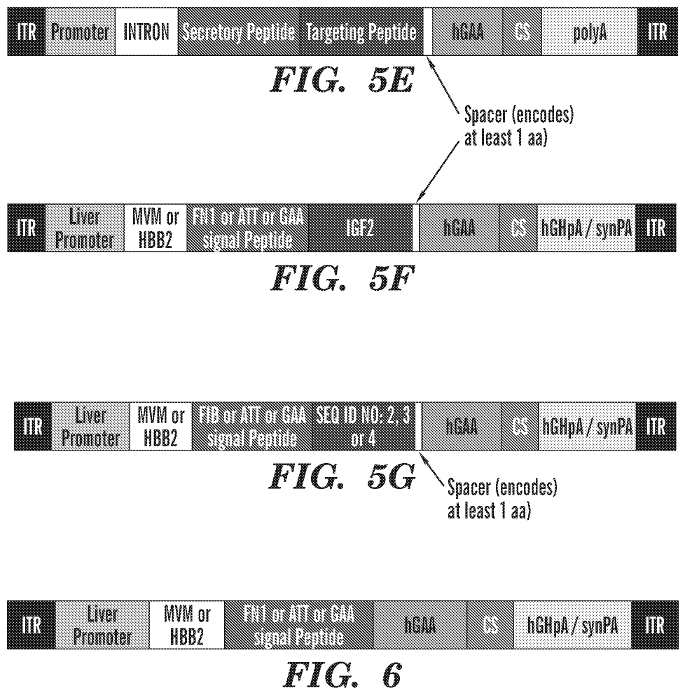

The coding sequence of human IGF2 is disclosed in U.S. Pat. No. 8,492,388 (see e.g., FIG. 2) which is incorporated herein in its entirety by reference. IGF2 protein is synthesized as a pre-pro-protein with a 24 amino acid signal peptide at the amino terminus and an 89 amino acid carboxy terminal region both of which are removed post-translationally, reviewed in O'Dell et al. (1998) Int. J. Biochem Cell Biol. 30(7):767-71. The mature protein is 67 amino acids. A Leishmania codon optimized version of the mature IGF2 is disclosed in U.S. Pat. No. 8,492,388 (see, e.g., FIG. 3 of 8,492,388) (Langford et al. (1992) Exp. Parasitol. 74(3):360-1). Additional cassettes containing a deletion of amino acids 1-7 of the mature polypeptide (Δ1-7), alteration of residue 27 from tyrosine to leucine (Y27L) or both mutations (Δ1-7,Y27L) were made to produce IGF-II cassettes with specificity for only the desired receptor as described below. wildtype, Y27L, Δ1-7, and Y27LΔ1-7 IGF2 variants are encompassed for use herein.

The mature human IGF2 sequence is shown below:

(SEQ ID NO: 5)
A Y R P S E T L C G G E L V D T L Q F V C G D R G
F Y F S R P A S R V S R R S R G I V E E C C F R S
C D L A L L E T Y C A T P A K S E

In some embodiments, the rAAV genome comprises a nucleic acid encoding a fusion protein, where the nucleic acid encoding the mature IGF2 polypeptide (SEQ ID NO: 5) or a IGF2 sequence variant (e.g., SEQ ID NO: 6 (IGF2-Δ2-7); SEQ ID NO: 7 (IGF2-Δ1-7); SEQ ID NO: 8 (IGF2-Δ1-42), SEQ ID NO: 9 (IGF2-V43M)) or sequences having at least 85%, or 90% or 95% sequence identity to SEQ ID NO: 5-9, is fused to the 5′ end of nucleic acid encoding the GAA protein, fusion proteins (e.g., IGF2-GAA fusion polypeptides) are created that can be taken up by a variety of cell types and transported to the lysosome. Alternatively, a nucleic acid encoding a precursor IGF2 polypeptide can be fused to the 3′ end of a GAA gene; the precursor includes a carboxy-terminal portion that is cleaved in mammalian cells to yield the mature IGF2 polypeptide, but the IGF2 signal peptide is preferably omitted (or moved to the 5′ end of the GAA gene). This method has numerous advantages over methods involving glycosylation including simplicity and cost effectiveness, because once the protein is isolated, no further modifications need be made.

The rAAV genome can encode a targeting peptide derived from IGF2 to target the CI-MPR. Alternatively, in some embodiments, a targeting peptide preferentially bind to receptors on the surface of myotubes can be employed. Such peptides have been described (Samoylova et al. (1999) Muscle and Nerve 22:460; U.S. Pat. No. 6,329,501). Other cell surface receptors, such as the Fc receptor, the LDL receptor, or the transferrin receptor are also appropriate targets and can promote targeting of GAA.

In some embodiments, the IGF2 sequence encompassed for use herein is described U.S. Pat. Nos. 7,785,856 and 9,873,868 which are each incorporated herein in their entirety by reference.

Deletion Mutants of IGF2:

In some embodiments, a IGF-sequence comprises a minimal regions of IGF2 that can bind with high affinity to the M6P/IGF2 receptor. The residues that have been implicated in IGF2 binding to the M6P/IGF2 receptor mostly cluster on one face of IGF2 (Terasawa et al. (1994) EMBO J. 13(23):5590-7). Although IGF2 tertiary structure is normally maintained by three intramolecular disulfide bonds, a peptide incorporating the amino acid sequence on the M6P/IGF2 receptor binding surface of IGF2 can be designed to fold properly and have binding activity. Such a minimal binding peptide is a highly preferred targeting portion. Designed peptides based on the region around amino acids 48-55 can be tested for binding to the M6P/IGF2 receptor. Alternatively, a random library of peptides can be screened for the ability to bind the M6P/IGF2 receptor either via a yeast two hybrid assay, or via a phage display type assay.

In some embodiments, a IGF2 sequence is a minimal region or regions of IGF2 that can bind with high affinity to the M6P/IGF2 receptor. The residues that have been implicated in IGF2 binding to the M6P/IGF2 receptor mostly cluster on one face of IGF2 (Terasawa et al. (1994) EMBO J. 13(23):5590-7). Although IGF2 tertiary structure is normally maintained by three intramolecular disulfide bonds, a peptide incorporating the amino acid sequence on the M6P/IGF2 receptor binding surface of IGF2 can be designed to fold properly and have binding activity. Such a minimal binding peptide is a highly preferred IGF2 sequence herein. Designed peptides based on the region around amino acids 43-55 or 48-55 can be tested for binding to the M6P/IGF2 receptor.

In particular embodiments, the IGF2 sequence comprises a modification at valine 43, where valine is modified to a met (V43M), such that translation initiation starts at amino acid 43. A IGF2 sequence with a modification of V43M encompassed for use herein as a targeting peptide or IGF2 sequence binds the cation-independent mannose-6-phosphate receptor. In alterative embodiments, the IGF2 sequence is delta 1-42 of IGF2 with V43 changed to an Met (i.e., IGF2-Δ1-42 (SEQ ID NO: 8) or IGF2-V43M (SEQ ID NO:9).

The binding surfaces for the IGF-I and cation-independent M6P receptors are on separate faces of IGF2, and functional cation-independent M6P binding domains can be constructed that are substantially smaller than human IGF2. For example, the amino terminal amino acids 2-7 or 1-7 and/or the carboxy terminal residues 62-67 of the human IGF2 protein can be deleted or replaced. Additionally, amino acids 29-40 can optionally be eliminated or replaced without altering the folding of the remainder of the polypeptide or binding to the cation-independent M6P receptor. Thus, in some embodiments, a IGF2 sequence for fusion to a GAA-polypeptide can comprise amino acids 8-28 and 41-61 of IGF2. In some embodiments, these stretches of amino acids can be joined directly or separated by a linker. Alternatively, amino acids 8-28 and 41-61 can be provided on separate polypeptide chains. In some embodiments, amino acids 8-28 of IGF2, or a conservative substitution variant thereof, could be fused to GAA polypeptide to express a IGF2-GAA fusion protein from the rAVV vector, and a separate rAAV vector could express IGF2 amino acids 41-61, or a conservative substitution variant thereof.

In order to facilitate proper presentation and folding of the IGF2 sequence, longer portions of IGF2 proteins can be used. For example, an IGF2 tag including amino acid residues 1-67, 1-87, or the entire precursor form can be used.

In some embodiments, the IGF2 sequence is a nucleic acid sequence that encodes an IGF2 targeting peptide of any of the following: residue 1 followed by residues 8-67 of wild-type mature human insulin-like growth factor II (IGF2) of SEQ ID NO: 5 (i.e., SEQ ID NO: 6; i.e., IGF2-delta 2-7); residues 8-67 of wild-type mature human insulin-like growth factor II (IGF2) of SEQ ID NO: 5 (i.e., SEQ ID NO: 7; IGF2-delta 1-7) or residues 43-67 of wild-type mature human insulin-like growth factor II (IGF2) of SEQ ID NO: 5 (i.e., IGF2-V43M (SEQ ID NO: 9) or IGF-delta 1-42 (SEQ ID NO: 8).

In some embodiments of the methods and compositions as disclosed herein, the IGF2 sequence is a nucleic acid sequence selected from any nucleic acid sequence comprising any of: SEQ ID NO: 2 (i.e., IGF2-delta 2-7); SEQ ID NO: 3 (i.e., IGF2-delta 1-7) or SEQ ID NO: 4 (i.e., IGF2-V43M) or a sequence at least sequence at least 85%, 90%, 95%, 96%, 97%, 98% or 99% sequence identity thereto.

SEQ ID NO: 2 (i.e., IGF2-delta 2-7) is as follows:

(SEQ ID NO: 2)
GCT|CTGTGCGGCGGGGAGCTGGTGGACACCCTCCAGTTCGTCTGTGGGG
ACCGCGGCTTCTACTTCAGCAGGCCCGCAAGCCGTGTGAGCCGTCGCAGC
CGTGGCATCGTTGAGGAGTGCTGTTTCCGCAGCTGTGACCTGGCCCTCCT
GGAGACGTACTGTGCTACCCCCGCCAAGTCCGAG)

SEQ ID NO: 3 (i.e., IGF2-delta 1-7) is as follows:

(SEQ ID NO: 3)
CTGTGCGGCGGGGAGCTGGTGGACACCCTCCAGTTCGTCTGTGGGGACCG
CGGCTTCTACTTCAGCAGGCCCGCAAGCCGTGTGAGCCGTCGCAGCCGTG
GCATCGTTGAGGAGTGCTGTTTCCGCAGCTGTGACCTGGCCCTCCTGGAG
ACGTACTGTGCTACCCCCGCCAAGTCCGAG

SEQ ID NO: 4 (i.e., IGF2-V43M) is as follows:

(SEQ ID NO: 4)
GCTTACCGCCCCAGTGAGACCCTGTGCGGCGGGGAGCTGGTGGACACCCT
CCAGTTCGTCTGTGGGGACCGCGGCTTCTACTTCAGCAGGCCCGCAAGCC
GTGTGAGCCGTCGCAGCCGTGGCATCATGGAGGAGTGCTGTTTCCGCAGC
TGTGACCTGGCCCTCCTGGAGACGTACTGTGCTACCCCCGCCAAGTCCGA
G

In some embodiments, in order to facilitate proper presentation and folding of the IGF2 sequence, longer portions of IGF2 proteins can be used. For example, an IGF2 sequence including amino acid residues 1-67, 1-87, or the entire precursor form can be used.

Modified IGF2 Sequences and IGF2 Homologues

In some embodiments, the nucleic acid encoding IGF2 can be modified to diminish their affinity for IGFBPs, and/or decreasing affinity for binding to IGF-I receptor, thereby increasing targeting to the lysosomes and increasing the bioavailability of the fused GAA-polypeptide.

IGF2 sequence is preferably targeted specifically to the M6P receptor. Particularly useful are IGF2 sequences which have mutations in the IGF2 polypeptide that result in a protein that binds the CI-MPR/M6P receptor with high affinity while no longer binding the other two receptors with appreciable affinity.

The IGF2 sequence can also be modified to minimize binding to serum IGF-binding proteins (IGFBPs) (Baxter (2000) Am. J. Physiol Endocrinol Metab. 278(6):967-76) and to IGF-I receptor, in order to avoid sequestration of IGF2 constructs. A number of studies have localized residues in IGF-1 and IGF2 necessary for binding to IGF-binding proteins. Constructs with mutations at these residues can be screened for retention of high affinity binding to the M6P/IGF2 receptor and for reduced affinity for IGF-binding proteins. For example, replacing Phe 26 of IGF2 with Ser is reported to reduce affinity of IGF2 for IGFBP-1 and -6 with no effect on binding to the M6P/IGF2 receptor (Bach et al. (1993) J. Biol. Chem. 268(13):9246-54). Other substitutions, such as Ser for Phe 19 and Lys for Glu 9, can also be advantageous. The analogous mutations, separately or in combination, in a region of IGF-I that is highly conserved with IGF2 result in large decreases in IGF-BP binding (Magee et al. (1999) Biochemistry 38(48): 15863-70).

IGF2 binds to the IGF2/M6P and IGF-I receptors with relatively high affinity and binds with lower affinity to the insulin receptor. Substitution of IGF2 residues 48-50 (Phe Arg Ser) with the corresponding residues from insulin, (Thr Ser Ile), or substitution of residues 54-55 (Ala Leu) with the corresponding residues from IGF-I (Arg Arg) result in diminished binding to the IGF2/M6P receptor but retention of binding to the IGF-I and insulin receptors (Sakano et al. (1991) J. Biol. Chem. 266(31):20626-35).

IGF2 binds to repeat 11 of the cation-independent M6P receptor. Indeed, a minireceptor in which only repeat 11 is fused to the transmembrane and cytoplasmic domains of the cation-independent M6P receptor is capable of binding IGF2 (with an affinity approximately one tenth the affinity of the full length receptor) and mediating internalization of IGF2 and its delivery to lysosomes (Grimme et al. (2000) J. Biol. Chem. 275(43):33697-33703). The structure of domain 11 of the M6P receptor is known (Protein Data Base entries 1GP0 and 1GP3; Brown et al. (2002) EMBO J. 21(5):1054-1062). The putative IGF2 binding site is a hydrophobic pocket believed to interact with hydrophobic amino acids of IGF2; candidate amino acids of IGF2 include leucine 8, phenylalanine 48, alanine 54, and leucine 55. Although repeat 11 is sufficient for IGF2 binding, constructs including larger portions of the cation-independent M6P receptor (e.g. repeats 10-13, or 1-15) generally bind IGF2 with greater affinity and with increased pH dependence (see, for example, Linnell et al. (2001) J. Biol. Chem. 276(26):23986-23991).

Substitution of IGF2 residues Tyr 27 with Leu, or Ser 26 with Phe diminishes the affinity of IGF2 for the IGF-I receptor by 94-, 56-, and 4-fold respectively (Torres et al. (1995) J. Mol. Biol. 248(2):385-401). Deletion of residues 1-7 of human IGF2 resulted in a 30-fold decrease in affinity for the human IGF-I receptor and a concomitant 12 fold increase in affinity for the rat IGF2 receptor (Hashimoto et al. (1995) J. Biol. Chem. 270(30):18013-8). Truncation of the C-terminus of IGF2 (residues 62-67) also appear to lower the affinity of IGF2 for the IGF-I receptor by 5 fold (Roth et al. (1991) Biochem. Biophys. Res. Commun. 181(2):907-14).

Substitution of IGF2 residue phenylalanine 26 with serine reduces binding to IGFBPs 1-5 by 5-75 fold (Bach et al. (1993) J. Biol. Chem. 268(13):9246-54). Replacement of IGF2 residues 48-50 with threonine-serine-isoleucine reduces binding by more than 100 fold to most of the IGFBPs (Bach et al. (1993) J. Biol. Chem. 268(13):9246-54); these residues are, however, also important for binding to the cation-independent mannose-6-phosphate receptor. The Y27L substitution that disrupts binding to the IGF-I receptor interferes with formation of the ternary complex with IGFBP3 and acid labile subunit (Hashimoto et al. (1997) J. Biol. Chem. 272(44):27936-42); this ternary complex accounts for most of the IGF2 in the circulation (Yu et al. (1999) J. Clin. Lab Anal. 13(4):166-72). Deletion of the first six residues of IGF2 also interferes with IGFBP binding (Luthi et al. (1992) Eur. J. Biochem. 205(2):483-90).

Studies on IGF-I interaction with IGFBPs revealed additionally that substitution of serine for phenylalanine 16 did not affect secondary structure but decreased IGFBP binding by between 40 and 300 fold (Magee et al. (1999) Biochemistry 38(48):15863-70). Changing glutamate 9 to lysine also resulted in a significant decrease in IGFBP binding. Furthermore, the double mutant lysine 9/serine 16 exhibited the lowest affinity for IGFBPs. The conservation of sequence between this region of IGF-I and IGF2 suggests that a similar effect will be observed when the analogous mutations are made in IGF2 (glutamate 12 lysine/phenylalanine 19 serine).

In some embodiments, the IGF2 sequence comprises at least amino acids 48-55; at least amino acids 8-28 and 41-61; or at least amino acids 8-87, or a sequence variant thereof (e.g. R68A) or truncated form thereof (e.g. C-terminally truncated from position 62) that binds the cation-independent mannose-6-phosphate receptor.

Decrease Binding of the IGF2 sequence to the IGF-I Receptor: Substitution of IGF2 residues Tyr 27 with Leu, or Ser 26 with Phe diminishes the affinity of IGF2 for the IGF-I receptor by 94-, 56-, and 4-fold respectively (Tones et al. (1995) J. Mol. Biol. 248(2):385-401). Deletion of residues 1-7 of human IGF2 resulted in a 30-fold decrease in affinity for the human IGF-I receptor and a concomitant 12 fold increase in affinity for the rat IGF2 receptor (Hashimoto et al. (1995) J. Biol. Chem. 270(30):18013-8). The NMR structure of IGF2 shows that Thr 7 is located near residues 48 Phe and 50 Ser as well as near the 9 Cys-47 Cys disulfide bridge. It is thought that interaction of Thr 7 with these residues can stabilize the flexible N-terminal hexapeptide required for IGF-I receptor binding (Terasawa et al. (1994) EMBO J. 13(23)5590-7). At the same time this interaction can modulate binding to the IGF2 receptor. Truncation of the C-terminus of IGF2 (residues 62-67) also appear to lower the affinity of IGF2 for the IGF-I receptor by 5 fold (Roth et al. (1991) Biochem. Biophys. Res. Commun. 181(2):907-14).

In some embodiments, a targeting peptide (e.g., a IGF2 sequence) encompassed for use herein binds to CI-MPR with a submicromolar dissociation constant. Generally speaking, lower dissociation constants (e.g. less than 10-7 M, less than 10-8 M, or less than 10-9 M) are preferred. Determination of dissociation constants can be determined by one of ordinary skill in the art, e.g., by surface plasmon resonance as described in Linnell et al. (2001) J. Biol. Chem. 276(26):23986-23991. In some embodiments, assessing the ability of a targeting peptide (e.g., a IGF2 sequence) to bind to CI-MPR can be determined using an assay comprising a soluble form of the extracellular domain of the CI-MPR (e.g. repeats 1-15 of the cation-independent M6P receptor) which is immobilized to a chip through an avidin-biotin interaction. The targeting peptide (e.g., a IGF2 sequence) is passed over the chip, and kinetic and equilibrium constants are detected and calculated by measuring changes in mass associated with the chip surface.

In another embodiment of the invention, the rAAV genome encoding the targeting peptide (e.g., IGF2 sequence) is inserted into the native GAA coding sequence at the junction of the mature 70/76 kDal polypeptide and the C-terminal domain, for example at position 791. This creates a single chimeric polypeptide. Because the targeting peptide (e.g., IGF2 sequence) may be unable to bind to its cognate receptor in this configuration, a protease cleavage site may be inserted just downstream of the targeting peptide (e.g., IGF2 sequence). Once the protein is produced in correct folded form, the C-terminal domain can be cleaved by protease treatment.

It may be desirable to employ a protease cleavage site that is acted upon by a protease normally found in human serum. In this way, the targeting peptide (e.g., IGF2 sequence) tagged GAA can be introduced into the blood stream in a prodrug form and become activated for uptake by the serum resident protease. This might improve the distribution of the enzyme. As before, the peptide tag could be the IGF2 sequence-tag or a muscle-specific tag.

In another embodiment of the invention, the targeting peptide (e.g., IGF2 sequence) is fused at the N-terminus of GAA in such a way as to retain enzymatic activity. In the case of N-terminal fusions, it is possible to affect the level of secretion of the enzyme by substituting a heterologous signal peptide for the native GAA signal peptide.

In some embodiments, the rAAV genome encoding the targeting peptide (e.g., IGF2 sequence) is inserted into the native GAA coding sequence at the junction of the mature 70/76 kDal polypeptide and the C-terminal domain, for example at position 791. This creates a single fusion (or chimeric) polypeptide. Because the targeting peptide (e.g., IGF2 sequence) may be unable to bind to its cognate receptor in this configuration, a protease cleavage site may be inserted just downstream of the targeting peptide (e.g., IGF2 sequence). Once the GAA polypeptide is produced in correct folded form, the C-terminal domain can be cleaved by protease treatment.

Accordingly, in some embodiments it may be desirable to employ a protease cleavage site that is acted upon by a protease normally found in human serum. In this way, the targeting peptide (e.g., IGF2 sequence) fused to the GAA polypeptide can be introduced into the blood stream in a prodrug form and become activated for uptake by the serum resident protease. This might improve the distribution of the GAA polypeptide. As before, the targeting peptide is a IGF2 sequence as disclosed herein) or a muscle-specific sequence.

In another embodiment of the invention, the targeting peptide (e.g., IGF2 sequence) is fused at the N-terminus of GAA in such a way as to retain enzymatic activity (e.g., see the Examples which describes an assay to measure GAA activity). In the case of N-terminal fusions, it is possible to increase the level of secretion of the GAA by substituting a heterologous signal peptide as described herein for the native GAA signal peptide.

In one embodiment, a targeting peptide, e.g., IGF2 sequence as defined herein, is fused directly to the N- or C-terminus of the GAA polypeptide. In another embodiment, a IGF2 sequence is fused to the N- or C-terminus of the GAA polypeptide by a spacer. In one specific embodiment, a IGF2 sequence is fused to the GAA polypeptide by a spacer of 10-25 amino acids. In another embodiment, a IGF2 sequence is fused to the GAA polypeptide by a spacer including glycine residues.

In some embodiments, a IGF2 sequence is fused to the GAA polypeptide by a spacer of at least 1, 2, or 3 amino acids. In some embodiments, the spacer comprises amino acids GAP or Gly-Ala-Pro (SEQ ID NO: 31), or an amino acid sequence at least 50% identical thereto. In some embodiments, the spacer is GGG or GA or AP, or GP or variants thereof. In some embodiments, the spacer is encoded by nucleic acids ggc gcg ccg (SEQ ID NO: 30).

In some embodiments, a IGF2 sequence is fused to the GAA polypeptide by a spacer including a helical structure. In another specific embodiment, a IGF2 sequence is fused to the GAA polypeptide by a spacer at least 50% identical to the sequence GGGTVGDDDDK (SEQ ID NO: 35). In some embodiments of the methods and compositions as disclosed herein, the spacer is SEQ ID NO: 31 (encoded by nucleic acids of SEQ ID NO: 30). In some embodiments of the methods and compositions as disclosed herein, the spacer is selected from any of: SEQ ID NO: 31, SEQ ID NO: 32, SEQ ID NO: 33, SEQ ID NO: 34 or SEQ ID NO: 35, or a sequence at least sequence at least 85%, 90%, 95%, 96%, 97%, 98% or 99% sequence identity thereto.

Cation-Independent M6P Receptor

In some embodiments, the targeting peptide is a lysosomal targeting peptide or protein, or other moiety that binds to the cation independent M6P/IGF2 receptor (CI-MPR) in a mannose-6-phosphate-independent manner. Advantageously, this embodiment mimics the normal biological mechanism for uptake of LSD proteins, yet does so in a manner independent of mannose-6-phosphate.

The cation-independent M6P receptor is a 275 kDa single chain transmembrane glycoprotein expressed ubiquitously in mammalian tissues. It is one of two mammalian receptors that bind M6P: the second is referred to as the cation-dependent M6P receptor. The cation-dependent M6P receptor requires divalent cations for M6P binding; the cation-independent M6P receptor does not. These receptors play an important role in the trafficking of lysosomal enzymes through recognition of the M6P moiety on high mannose carbohydrate on lysosomal enzymes. The extracellular domain of the cation-independent M6P receptor contains 15 homologous domains (“repeats”) that bind a diverse group of ligands at discrete locations on the receptor.

The cation-independent M6P receptor (CI-MPR) contains two binding sites for M6P: one located in repeats 1-3 and the other located in repeats 7-9. The receptor binds monovalent M6P ligands with a dissociation constant in the μM range while binding divalent M6P ligands with a dissociation constant in the nM range, probably due to receptor oligomerization. Uptake of IGF2 by CI-MPR is enhanced by concomitant binding of multivalent M6P ligands such as lysosomal enzymes to the receptor.

The CI-MPR also contains binding sites for at least three distinct ligands that can be used as targeting peptides. As disclosed herein, IGF2 ligand binds to CI-MPR with a dissociation constant of about 14 nM at or about pH 7.4, primarily through interactions with repeat 11. Consistent with its function in targeting IGF2 to the lysosome, the dissociation constant is increased approximately 100-fold at or about pH 5.5 promoting dissociation of IGF2 in acidic late endosomes. The CI-MPR is capable of binding high molecular weight O-glycosylated IGF2 forms. Accordingly, in some embodiments, the IGF2 sequence comprises O-glycosylation.

In an alternative embodiment, the targeting peptide that binds to CI-MPR is retinoic acid. Retinoic acid binds to the receptor with a dissociation constant of 2.5 nM. Affinity photolabeling of the cation-independent M6P receptor with retinoic acid does not interfere with IGF2 or M6P binding to the receptor, indicating that retinoic acid binds to a distinct site on the receptor. Binding of retinoic acid to the receptor alters the intracellular distribution of the receptor with a greater accumulation of the receptor in cytoplasmic vesicles and also enhances uptake of M6P modified β-glucuronidase. Retinoic acid has a photoactivatable moiety that can be used to link it to a therapeutic agent without interfering with its ability to bind to the cation-independent M6P receptor.

The urokinase-type plasminogen receptor (uPAR) also binds CI-MPR with a dissociation constant of 9 μM. uPAR is a GPI-anchored receptor on the surface of most cell types where it functions as an adhesion molecule and in the proteolytic activation of plasminogen and TGF-β. Binding of uPAR to the CI-M6P receptor targets it to the lysosome, thereby modulating its activity. Thus, fusing the extracellular domain of uPAR, or a portion thereof competent to bind the cation-independent M6P receptor, to a therapeutic agent permits targeting of the agent to a lysosome.

In some embodiments, a IGF2 sequence is modified to be furin resistant, i.e., resistant to degradation by furin protease, which recognizes Arg-X-X-Arg cleavage sites. Such IGF2 sequences are disclosed in US application 22012/0213762 which is incorporated herein in its entirety by reference. In some embodiments, a furin resistant IGF2 sequence for use in a rAAV genome as described herein contains a mutation within a region corresponding to amino acids 30-40 (e.g., 31-40, 32-40, 33-40, 34-40, 30-39, 31-39, 32-39, 34-37, 32-39, 33-39, 34-39, 35-39, 36-39, 37-40, 34-40) of SEQ ID NO: 5 (wt IGF2 sequence) can be substituted with any other amino acid or deleted. For example, substitutions at position 34 may affect furin recognition of the first cleavage site. Insertion of one or more additional amino acids within each recognition site may abolish one or both furin cleavage sites. Deletion of one or more of the residues in the degenerate positions may also abolish both furin cleavage sites.

In some embodiments, a furin-resistant IGF2 sequence contains amino acid substitutions at positions corresponding to Arg37 (R37) or Arg40 (R40) of SEQ ID NO:5. In some embodiments, a furin-resistant IGF2 sequence contains a Lys (K) or Ala (A) substitution at positions Arg37 or Arg40 of SEQ ID NO: 5. Other substitutions are possible, including combinations of Lys and/or Ala mutations at both positions 37 and 40, or substitutions of amino acids other than Lys (K) or Ala (A). In some embodiments, the IGF2 sequence encompassed for use in the rAVV genome as disclosed herein is IGFΔ2-7-K37, or IGFΔ2-7-K40 or IGFΔ1-7-K37 or IGFΔ1-7-K40, indicating that the IGF2 sequences has a deletion of aa 2-7 or 1-7 and a modification of a Arg (R) residue at position 37 to a lysine (i.e., R37K modification) or R40K respectively. In some embodiments, the IGF2 sequence encompassed for use in the rAVV genome as disclosed herein is IGFΔ2-7-K37-K40, or IGFΔ1-7-R37K-R40K indicating that the IGF2 sequences has a deletion of residues 2-7 or residues 1-7 and a modification of a R residue at position 37 and position 40 to lysinines (R37K and R40K). In some embodiments, the IGF2 sequence encompassed for use in the rAVV genome as disclosed herein is selected from any of: IGFΔ2-7-R37A, or IGFΔ2-7-R40A or IGFΔ1-7-R37A or IGFΔ1-7-R40A, IGFΔ2-7-R37A-R40A, or IGFΔ1-7-R37A-R40A. Exemplary constructs for the IGF2 sequence encompassed for use in the rAVV genome as disclosed herein are disclosed in US application 2012/0213762, which is incorporated herein in its entirety by reference.

In some embodiments, the furin-resistant IGF-2 sequence suitable for the invention may contain additional mutations. For example, up to 30% or more of the residues of SEQ ID NO: 5 may be changed (e.g., up to 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or more residues may be changed). Thus, a furin-resistant IGF2 mutein suitable for the invention may have an amino acid sequence at least 70%, including at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, identical to SEQ ID NO: 5.

Moreover, use of a IGF2 sequence as disclosed herein is also referred to in the art as Glycosylation Independent Lysosomal Targeting (GILT) because the IGF2 sequence replaces M6P as the moiety targeting the lysosomes. Details of the GILT technology are described in U.S. Application Publication Nos. 2003/0082176, 2004/0006008, 2004/0005309, 2003/0072761, 2005/0281805, 2005/0244400, and international publications WO 03/032913, WO 03/032727, WO 02/087510, WO 03/102583, WO 2005/078077, the disclosures of all of which are hereby incorporated by reference.

### D. Spacer and Fusion Junction of the GAA Polypeptide

Where GAA is expressed as a fusion protein with a secretory signal peptide (e.g., SS-GAA polypeptide) or with a targeting peptide (i.e., SS-IGF2-GAA polypeptide double fusion polypeptide), the signal peptide or IGF2 sequence can be fused directly to the GAA polypeptide or can be separated from the GAA polypeptide by a linker. An amino acid linker (also referred to herein as a “spacer”) incorporates one or more amino acids other than that appearing at that position in the natural protein. Spacers can be generally designed to be flexible or to interpose a structure, such as an a-helix, between the two protein moieties.

In some embodiments, a spacer or linker can be relatively short, e.g., at least 1, 2, 3, 4 or 5 amino acids, or such as the sequence Gly-Ala-Pro (SEQ ID NO: 31) or Gly-Gly-Gly-Gly-Gly-Pro (SEQ ID NO: 32), or can be longer, such as, for example, 5-10 amino acids in length or 10-25 amino acids in length. For example, flexible repeating linkers of 3-4 copies of the sequence (GGGGS (SEQ ID NO:33)) and a-helical repeating linkers of 2-5 copies of the sequence (EAAAK (SEQ ID NO:34)) have been described (Arai et al. (2004) Proteins: Structure, Function and Bioinformatics 57:829-838).

The use of another linker, GGGTVGDDDDK (SEQ ID NO: 35), in the context of an IGF2 fusion protein has also been reported (DiFalco et al. (1997) Biochem. J. 326:407-413) and is encompassed for use. Linkers incorporating an a-helical portion of a human serum protein can be used to minimize immunogenicity of the linker region.

In some embodiments, the spacer is encoded by nucleic acids ggc gcg ccg (SEQ ID NO: 30) which encodes the amino acid spacer comprising amino acids GAP or Gly-Ala-Pro (SEQ ID NO: 31).

The site of a fusion junction in the GAA polypeptide to fuse with either the signal peptide (to generate a SS-GAA fusion protein) or with the targeting peptide (e.g., to generate a SP-IGF2-GAA double fusion polypeptide) should be selected with care to promote proper folding and activity of each polypeptide in the fusion protein and to prevent premature separation of a signal peptide from a GAA polypeptide.

In some embodiments, a IGF2 sequence is fused to the GAA polypeptide by a spacer including a helical structure. In another specific embodiment, a IGF2 sequence is fused to the GAA polypeptide by a spacer at least 50% identical to the sequence GGGTVGDDDDK (SEQ ID NO: 35). In some embodiments of the methods and compositions as disclosed herein, the spacer is SEQ ID NO: 31 (encoded by nucleic acids of SEQ ID NO: 30). In some embodiments of the methods and compositions as disclosed herein, the spacer is selected from any of: SEQ ID NO: 31, SEQ ID NO: 32, SEQ ID NO: 33, SEQ ID NO: 34 or SEQ ID NO: 35.

Four exemplary strategies for creating a IGF2-GAA fusion protein can be generated, which are as follows:

1. Fusion of the IGF2 sequence at the amino terminus of GAA.

2. Insertion of the IGF2 sequence between the trefoil domain and the mature region of GAA.

3. Insertion of the IGF2 sequence between the mature region of GAA and the C-terminal domain of GAA.

4. Fusion of the IGF2 sequence to the C-terminus of a truncated GAA polypeptide and co-expressing the C-terminal domain.

For example, a targeting peptide (e.g., a IGF2 sequence) can be fused, directly or by a spacer, to amino acid 40 or amino acid 70 of GAA, a position permitting expression of the protein, catalytic activity of the GAA protein, and proper targeting by the IGF2 sequence as described herein in the Examples. Alternatively, a targeting peptide (e.g., a IGF2 sequence) can be fused at or near the cleavage site separating the C-terminal domain of GAA from the mature polypeptide. This permits synthesis of a GAA protein with an internal targeting peptide (e.g., a IGF2 sequence), which optionally can be cleaved to liberate the mature polypeptide or the C-terminal domain from the targeting domain, depending on placement of cleavage sites. Alternatively, the mature polypeptide can be synthesized as a fusion protein at about position 791 without incorporating C-terminal sequences in the open reading frame of the expression construct.

In order to facilitate folding of the IGF2 sequence, GAA amino acid residues adjacent to the fusion junction can be modified. For example, since it is possible that GAA cystine residues may interfere with proper folding of the targeting peptide (e.g., a IGF2 sequence), the terminal GAA cystine 952 can be deleted or substituted with serine to accommodate a C-terminal targeting peptide (e.g., a IGF2 sequence). The targeting peptide (e.g., a IGF2 sequence) can also be fused immediately preceding the final Cys952. The penultimate cys938 can be changed to proline in conjunction with a mutation of the final Cys952 to serine.

### E. CS Sequence

In some embodiments, the rAAV genome disclosed herein comprises a heterologous nucleic acid sequence that can optionally comprise a Collagen stability sequence (CS or CSS), which is positioned 3′ of the GAA gene and 5′ of a polyA signal. Exemplary collagen stability sequences include CCCAGCCCACTTTTCCCCAA (SEQ ID NO: 65) or a sequence at least 85%, 90%, 95%, 96%, 97%, 98% or 99% sequence identity thereto. An exemplary collagen stability sequence can have an amino acid sequence of P S P L F P (SEQ ID NO: 66) or an amino acid sequence having at least 85%, 90%, 95%, 96%, 97%, 98% or 99% sequence identity thereto. CS sequences are disclosed in Holick and Liebhaber, Proc. Nat. Acad. Sci. 94: 2410-2414, 1997 (See, e.g. FIG. 3, p. 5205), which is incorporated herein its entirety by reference.

In some embodiments, the rAAV genome disclosed herein comprises a heterologous nucleic acid sequence that can optionally comprise an alternative stability sequence in place of the Collagen stability sequence (CS). Other stability sequences are known to one of ordinary skill in the art and are encompassed for use in the rAAV genome in place of, or in addition to, the collagen stability sequence disclosed herein.

### F. Promoters

In some embodiments, to achieve appropriate levels of GAA expression, the rAAV genotype comprises a promoter. A suitable promoter can be selected from any of a number of promoters known to one of ordinary skill in the art. In some embodiments, a promoter is a cell-type specific promotor. In a further embodiment, a promoter is an inducible promotor. In an embodiment, a promotor is located upstream 5′ and is operatively linked to the heterologous nucleic acid sequence. In some embodiments, the promotor is a liver cell-type specific promotor, a heart muscle cell-type specific promoter, a neuron cell-type specific promoter, a nerve cell-type specific promoter, a muscle cell-type specific promoter or another cell-type specific promoter.

In some embodiments, a constitutive promoter can be selected from a group of constitutive promoters of different strengths and tissue specificity. Some examples of these promoters are set forth in Table 4. An rAAV vector genome can include one or more constitutive promoters, such as viral promoters or promoters from mammalian genes that are generally active in promoting transcription. Examples of constitutive viral promoters are: Herpes Simplex virus (HSV) promoter, thymidine kinase (TK) promoter. Rous Sarcoma Virus (RSV) promoter, Simian Virus 40 (SV40) promoter, Mouse Mammary Tumor Virus (MMTV) promoter, Ad EIA promoter and cytomegalovirus (CMV) promoters. Examples of constitutive mammalian promoters include various housekeeping gene promoters, as exemplified by the β-actin promoter and the chicken beta-actin (CB) promoter, wherein the CB promoter has proven to be a particularly useful constitutive promoter for expressing GAA.

In an embodiment, the promoter is a tissue-specific promoter. Examples of tissue specific promoters that may be used with the rAAV vector genomes of the invention include the creatine kinase promoter, the myogenin promoter, the alpha myosin heavy chain promoter, the myocyte specific enhancer factor 2 (MEF2) promoter, the myoD enhancer element, albumin, alpha-1-antitrypsin promoter and hepatitis B virus core protein promoters, wherein the hepatitis B virus core protein promoters are specific for liver cells.

In an embodiment, a promoter is an inducible promoter. Examples of suitable inducible promoters include those from genes such as cytochrome P450 genes, heat shock protein genes, metallothionein genes, and hormone-inducible genes, including the estrogen gene promoter. Another example of an inducible promoter is the tetVP16 promoter that is responsive to tetracycline.

Promoters in a rAAV genome according to the disclosure herein include, but are not limited to neuron-specific promoters, such as synapsin 1 (SYN) promoter; muscle creatine kinase (MCK) promoters; and desmin (DES) promoters. In one embodiment, the AAV-mediated expression of heterologous nucleic acids (such as human GAA) can be achieved in neurons via a Synapsin promoter or in skeletal muscles via an MCK promoter. Other promoters that can be used include, EF, B19p6, CAG, neurone specific enolase gene promoter; chicken beta-actin/CMV hybrid promoter; platelet derived growth factor gene promoter; bGH, EF1a, CamKIIa, GFAP, RPE, ALB, TBG, MBP, MCK, TNT, aMHC, GFP, RFP, mCherry, CFP and YFP promoters.

TABLE 4
Exemplary promoters:

| Promoter | Description/Loci | | Target cell | | |
|---|---|---|---|---|---|
| name | (plasmid names) | Size | type | notes | references |
| CMV | Cytomegalovirus | ~600 bps | most cell types | Can undergo | Zolotukhin |
| | immediate early | | | silencing in- | et al. 1996; |
| | promoter(pTR-UF5) | | | vivo | Zolotukhin |
| | | | | | et a. 1999 |
| CBAaka: CB, | Hybrid CMV/Chicken | 1720 bps | most cell types | Contains | Acland et |
| CAG | beta actin | | | 381 bps | al. 2001; |
| | promoter(pTR- UF11, | | | version of | Cideciyan |
| | pTR-UF-SB) | | | CMV i.e. | et al. 2008 |
| | | | | enhancer | |
| smCBAaka: | Truncated CBA | 953 bps | most cell types | Chimeric | Pang et al. |
| small CBA | promoter | | | Intron | 2008; |
| | | | | collapsed. Used | |
| | | | | for ScAAV | |
| MOPS aka: | Proximal murine | ~500 bps | Photoreceptors, | | Flannery et |
| mOP, mRHO, | rhodopsin promoter | | primarily rods | | al. 1997; |
| MOPS500 | | | | | |
| GRK1aka: | Human rhodopsin | 292 bps | Photoreceptor, | Does not | Khani et |
| hGRK, hRK, | kinase 1 promoter | | rods and cones | transduce | al. 2007; |
| RK1 | | | (mouse and | cones in dog | Boye et al. |
| | | | primate) | | 2010; |
| | | | | | Boye et al. |
| | | | | | 2012 |
| IRBPaka: | Human inter- | 241 bps | Photoreceptors, | | Beltran et |
| hIRBP241 | photoreceptor retinoid | | rods and cones | | al. 2012 |
| | binding | | (mouse and | | |
| | protein/Retinol- | | dog) | | |
| | binding protein 3 | | | | |
| PR2.1aka: | Human red opsin | ~2100 bps | L and M cones | | Alexander |
| CHOPS2053 | promoter | | | | et al. 2007; |
| | | | | | Mancuso |
| | | | | | et al. 2009; |
| | | | | | Komaromy |
| | | | | | et al. 2010 |
| IRBP/GNAT2 | hIRBP enhancer fused | 524 bps | L/M and S | Efficiently | |
| | to cone transducin | | cones | transduces all | |
| | alpha promoter | | | classes of | |
| | | | | cones | |
| VMD2Aka: | Human vitelliform | 625 bps | RPE | Highly | Deng et al. |
| BEST1 | macular | | | selective for | 2012 |
| | dystrophy/Bestrophin | | | RPE | |
| | 1 promoter | | | | |
| VEcadaka: | VE-cadherin/Cadherin | 2530 bps | Vascular | | Cai et al. |
| VEcadherin | 5 (CDH5)/CD144 | | endothelial | | 2011; Qi et |
| | promoter | | cells | | al. 2012 |

Liver-Specific Promoters

In some embodiments of the methods and compositions as disclosed herein, the promoter is a liver specific promoter, and can be selected from any liver specific promoters including, but not limited to, a transthyretin promoter (TTR), a Liver specific promoter (LSP), for example, as disclosed in U.S. Pat. No. 5,863,541 (TTR promoter), or LSP promoter (PNAS; 96: 3906-3910, 1999. See e.g. p. 3906, Materials and Methods, rAAV construction), a synthetic liver promoter, the references which are incorporated herein in their entireties by reference. Other liver promoters can be used, for example, synthetic liver promoters.

In some embodiments, the TTR promoter is a truncated TTR promoter, e.g., comprising SEQ ID NO: 12 or a variant having at least sequence at least 85%, 90%, 95%, 96%, 97%, 98% or 99% nucleotide sequence identity thereto.

Other liver specific promoters include, but are not limited to promoters for the LDL receptor, Factor VIII, Factor IX, phenylalanine hydroxylase (PAH), ornithine transcarbamylase (OTC), and α1-antitrypsin (hAAT), and HCB promoter. In Other liver specific promoters include the AFP (alpha fetal protein) gene promoter and the albumin gene promoter, as disclosed in EP Patent Publication 0 415 731, the α-1 antitrypsin gene promoter, as disclosed in Rettenger, Proc. Natl. Acad. Sci. 91 (1994) 1460-1464, the fibrinogen gene promoter, the APO-A1 (Apolipoprotein A1) gene promoter, and the promoter genes for liver transference enzymes such as, for example, SGOT, SGPT and γ-glutamyle transferase. See also 2001/0051611 and PCT Patent Publications WO 90/07936 and WO 91/02805, which are incorporated herein in their entirety by reference. In some embodiments, the liver specific promoter is a recombinant liver specific promoter, e.g., as disclosed in US20170326256A1, which is incorporated herein in its entirety by reference.

In some embodiments, a liver specific promoter is the hepatitis B X-gene promoter and the hepatitis B core protein promoter. In some embodiments, liver specific promoters can be used with their respective enhancers. The enhancer element can be linked at either the 5′ or the 3′ end of the nucleic acid encoding the GAA polypeptide. The hepatitis B X gene promoter and its enhancer can be obtained from the viral genome as a 332 base pair EcoRV-NcoI DNA fragment employing the methods described in Twu, J Virol. 61 (1987) 3448-3453. The hepatitis B core protein promoter can be obtained from the viral genome as a 584 base pair BamHI-BgIII DNA fragment employing the methods described in Gerlach, Virol 189 (1992) 59-66. It may be necessary to remove the negative regulatory sequence in the BamHI-BgIII fragment prior to inserting it.

### G. Intron Sequence

In some embodiments, the rAAV genotype comprises an intron sequence located 3′ of the promoter sequence and 5′ of the secretory signal peptide. Intron sequences serve to increase one or more of: mRNA stability, mRNA transport out of nucleus and/or expression and/or regulation of the expressed GAA fusion polypeptide (e.g., SS-GAA fusion polypeptide or SS-IGF2-GAA polypeptide).

In some embodiments, the intron sequence is a MVM intron sequence, for example, but not limited to and intron sequence of SEQ ID NO: 13 or nucleic acid sequence having at least 80%, 85%, 90%, 95%, 96%, 97%, 98% or 99% nucleotide sequence identity thereto.

In some embodiments, the intron sequence is a HBB2 intron sequence, for example, but not limited to and intron sequence of SEQ ID NO: 14 or nucleic acid sequence having at least 80%, 85%, 90%, 95%, 96%, 97%, 98% or 99% nucleotide sequence identity thereto.

In some embodiments, the rAAV genotype comprises an intron sequence selected in the group consisting of a human beta globin b2 (or HBB2) intron, a FIX intron, a chicken beta-globin intron, and a SV40 intron. In some embodiments, the intron is optionally a modified intron such as a modified HBB2 intron (see, e.g., SEQ ID NO: 17 in of WO2018046774A1): a modified FIX intron (see, e.g., SEQ ID NO: 19 in WO2018046774A1), or a modified chicken beta-globin intron (e.g., see SEQ ID NO: 21 in WO2018046774A1), or modified HBB2 or FIX introns disclosed in WO2015/162302, which are incorporated herein in their entirety by reference.

### H. Poly-A

In some embodiments, an rAAV vector genome includes at least one poly-A tail that is located 3′ and downstream from the heterologous nucleic acid gene encoding the in one embodiment, a GAA fusion polypeptide (e.g., SS-GAA fusion polypeptide or SS-IGF2-GAA polypeptide). In some embodiments, the polyA signal is 3′ of a stability sequence or CS sequence as defined herein. Any polyA sequence can be used, including but not limited to hGH poly A, synpA polyA and the like. In some embodiments, the polyA is a synthetic polyA sequence. In some embodiments, the rAAV vector genome comprises two poly-A tails, e.g., a hGH poly A sequence and another polyA sequence, where a spacer nucleic acid sequence is located between the two poly A sequences. In some embodiments, the rAAV genome comprises 3′ of the nucleic acid encoding the GAA fusion polypeptide (e.g., SS-GAA fusion polypeptide or SS-IGF2-GAA polypeptide), or alternatively, 3′ of the CS sequence the following elements; a first polyA sequence, a spacer nucleic acid sequence (of between 100-400 bp, or about 250 bp), a second poly A sequence, a spacer nucleic acid sequence, and the 3′ ITR. In some embodiments, the first and second poly A sequence is a hGH poly A sequence, and in some embodiments, the first and second poly A sequences are a synthetic poly A sequence. In some embodiments, the first poly A sequence is a hGH poly A sequence and the second poly A sequence is a synthetic sequence, or vice versa—that is, in alternative embodiments, the first poly A sequence is a synthetic poly A sequence and the second poly A sequence is a hGH polyA sequence. An exemplary poly A sequence is, for example, SEQ ID NO: 15 (hGH poly A sequence), or a poly A nucleic acid sequence having at least 80%, 85%, 90%, 95%, 96%, 97%, 98% or 99% nucleotide sequence identity to SEQ ID NO: 15. In some embodiments, the hGHpoly sequence encompassed for use is described in Anderson et al. J. Biol. Chem 264(14); 8222-8229, 1989 (See, e.g. p. 8223, 2nd column, first paragraph) which is incorporated herein in its entirety by reference.

In some embodiments, a poly-A tail can be engineered to stabilize the RNA transcript that is transcribed from an rAAV vector genome, including a transcript for a heterologous gene, which in one embodiment is a GAA, and in alternative embodiments, the poly-A tail can be engineered to include elements that are destabilizing.

In an embodiment, a poly-A tail can be engineered to become a destabilizing element by altering the length of the poly-A tail. In an embodiment, the poly-A tail can be lengthened or shortened. In a further embodiment, the 3′ untranslated region that lies between the heterologous gene, in one embodiment a GAA, and the poly-A tail can be lengthened or shortened to alter the expression levels of the heterologous gene or alter the final polypeptide that is produced. In some embodiments, the 3′ untranslated region comprises GAA 3′ UTR (SEQ ID NO: 85).

In another embodiment, a destabilizing element is a microRNA (miRNA) that has the ability to silence (repress translation and promote degradation) the RNA transcripts the miRNA bind to that encode a heterologous gene. Modulation of the expression of a heterologous gene, in one embodiment, a GAA, can be undertaken by modifying, adding or deleting seed regions within the poly-A tail to which the miRNA bind. In an embodiment, addition or deletion of seed regions within the poly-A tail can increase or decrease expression of a protein, in one embodiment, a GAA, encoded by a heterologous gene in an rAAV vector genome. In a further embodiment, such increase or decrease in expression resultant from the addition or deletion of seed regions is dependent on the cell type transduced by the AAV containing an rAAV vector genome. For instance, seed regions specific for miRNA expressed in muscle and cardiac cells, but not found in liver cells, can be used to allow for production of the polypeptide encoded by a heterologous gene, in one embodiment, a GAA, in liver cells, but not muscle cells or cardiac cells.

In another embodiment, seed regions can also be engineered into the 3′ untranslated regions located between the heterologous gene and the poly-A tail. In a further embodiment, the destabilizing agent can be an siRNA. The coding region of the siRNA can be included in an rAAV vector genome and is generally located downstream, 3′ of the poly-A tail. In an embodiment, the expression of a heterologous gene, in one embodiment, GAA, can be undertaken by inclusion of the coding region for an siRNA in the rAAV cassette, for instance, downstream, 3′ of the poly-A tail. In a further embodiment, the promoter to induce expression of the siRNA can be tissue specific, such that the siRNA is silenced in tissues where expression of a heterologous gene, in one embodiment, a GAA, is not desired and siRNA expression does not occur in tissues where expression of a heterologous gene, in one embodiment, a GAA, is desired.

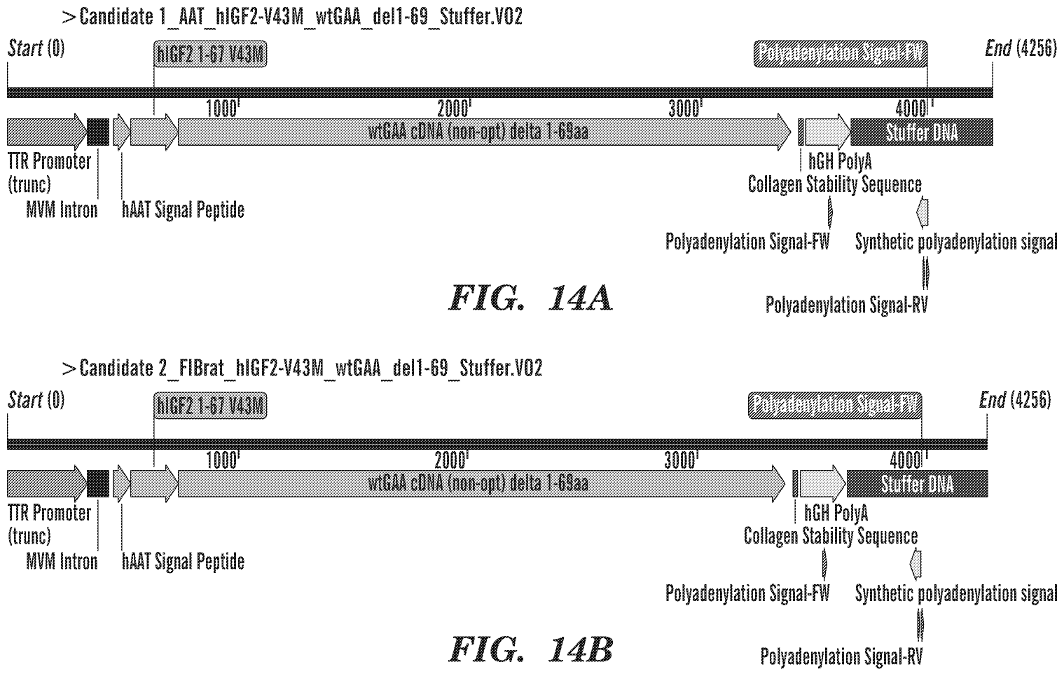

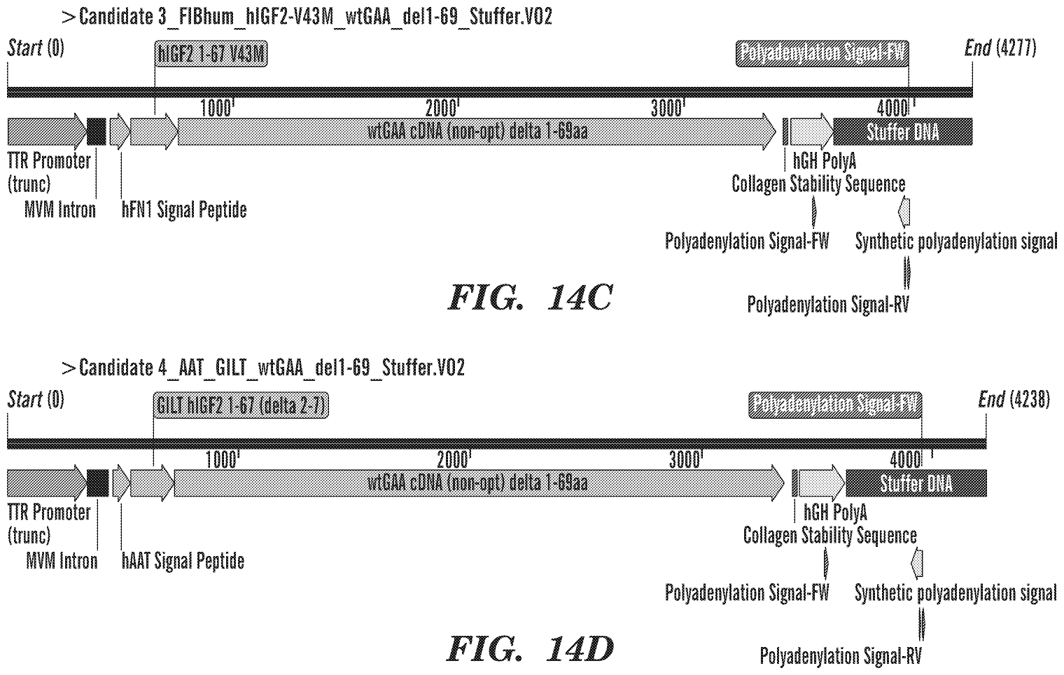

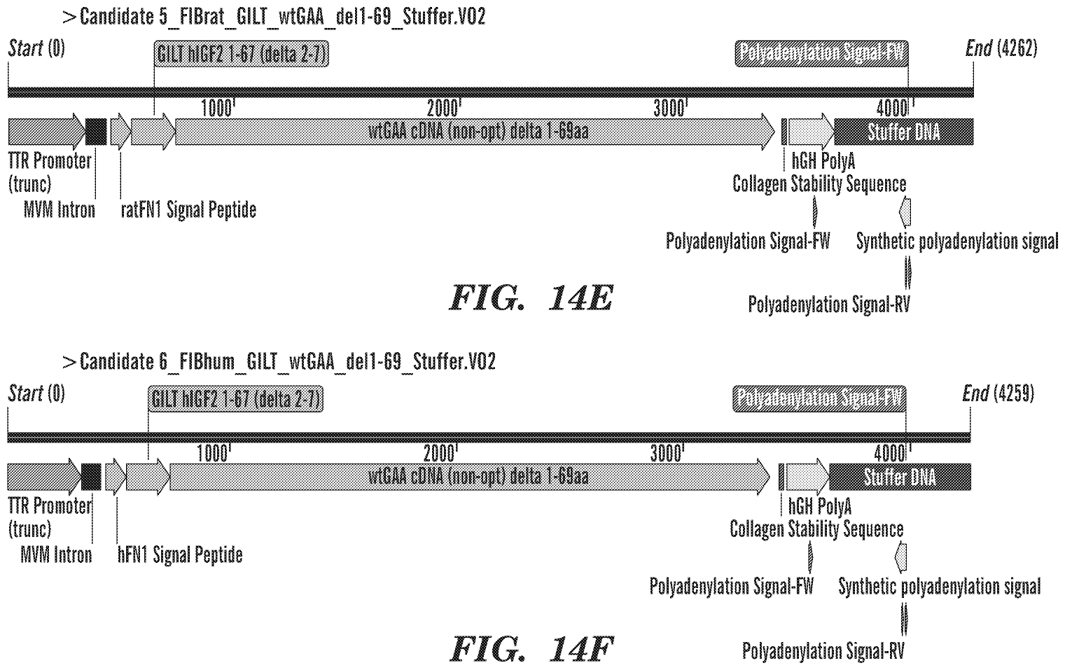

In all aspects of the methods and compositions as disclosed herein, the rAAV genome may also comprise a Stuffer DNA nucleic sequence. An exemplary stuffer DNA sequence is SEQ ID NO: 71, or a nucleic acid sequence having at least 80%, 85%, 90%, 95%, 96%, 97%, 98% or 99% nucleotide sequence identity thereto. As shown in FIGS. 8-13 and FIGS. 14A-14E, the stuffer sequence is located 3 of the poly A tail, for example, and is located 5′ of the ′3 ITR sequence. In some embodiments, the stuffer DNA sequence comprises a synthetic polyadenylation signal in the reverse orientation.

In some embodiments, a stuffer nucleic acid sequence (also referred to as a “spacer” nucleic acid fragment, see FIGS. 8-14) can be located between the poly A sequence and the 3′ ITR (i.e., a stuffer nucleic acid sequence is located 3′ of the polyA sequence and 5′ of the 3′ ITR) (see, e.g., FIG. 8-10). Such a stuffer nucleic acid sequence can be about 30 bp, 50pb, 75 bp, 100 bp, 150 bp, 200 bp, 250 bp, 300 bp or longer than 300 bp. In some embodiments of the methods and compositions as disclosed herein, a stuffer nucleic acid fragment is between 20-50 bp, 50-100 bp, 100-200 bp, 200-300 bp, 300-500 bp, or any integer between 20-500 bp. Exemplary stuffer (or spacer) nucleic acid sequence comprise SEQ ID NO: 16, SEQ ID NO: 71 or SEQ ID NO: 78, or a nucleic acid sequence at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, identical to SEQ ID NO: 16 or SEQ ID NO: 71 or SEQ ID NO: 78.

### I. AAV ITRs

The rAAV genome as disclosed here comprises AAV ITRs that have desirable characteristics and can be designed to modulate the activities of, and cellular responses to vectors that incorporate the ITRs. In another embodiment, the AAV ITRs are synthetic AAV ITRs that has desirable characteristics and can be designed to manipulate the activities of and cellular responses to vectors comprising one or two synthetic ITRs, including, as set forth in U.S. Pat. No. 9,447,433, which is incorporated herein by reference.

The AAV ITRs for use in the rAAV genome as disclosed herein may be of any serotype suitable for a particular application. In some embodiments, the AAV vector genome is flanked by AAV ITRs. In some embodiments, the rAAV vector genome is flanked by AAV ITRs, wherein an ITR comprises a full length ITR sequence, an ITR with sequences comprising CPG islands removed, an ITR with sequences comprising CPG sequences added, a truncated ITR sequence, an ITR sequence with one or more deletions within an ITR, an ITR sequence with one or more additions within an ITR, or a combination of comprising any portion of the aforementioned ITRs linked together to form a hybrid ITR.

In order to facilitate long term expression, in an embodiment, the polynucleotide encoding GAA is interposed between AAV inverted terminal repeats (ITRs) (e.g., the first or 5′ and second 3′ AAV ITRs). AAV ITRs are found at both ends of a WT rAAV vector genome, and serve as the origin and primer of DNA replication. ITRs are required in cis for AAV DNA replication as well as for rescue, or excision, from prokaryotic plasmids. In an embodiment, the AAV ITR sequences that are contained within the nucleic acid of the rAAV genome can be derived from any AAV serotype (e.g. 1, 2, 3, 3b, 4, 5, 6, 7, 8, 9, and 10) or can be derived from more than one serotype, including combining portions of two or more AAV serotypes to construct an ITR. In an embodiment, for use in the rAAV vector, including an rAAV vector genome, the first and second ITRs should include at least the minimum portions of a WT or engineered ITR that are necessary for packaging and replication. In some embodiments, an rAAV vector genome is flanked by AAV ITRs.

In some embodiments, the rAAV vector genome comprises at least one AAV ITR, wherein said ITR comprises, consists essentially of, or consists of; (a) an AAV rep binding element; (b) an AAV terminal resolution sequence; and (c) an AAV RBE (Rep binding element); wherein said ITR does not comprise any other AAV ITR sequences. In another embodiment, elements (a), (b), and (c) are from an AAV2 ITR and the ITR does not comprise any other AAV2 ITR sequences. In a further embodiment, elements (a), (b) and (c) are from any AAV ITR, including but not limited to AAV2, AAV8 and AAV9. In some embodiments, the polynucleotide comprises two synthetic ITRs, which may be the same or different.

In some embodiments, the polynucleotide in the rAAV vector, including an rAAV vector genome comprises two ITRs, which may be the same or different. The three elements in the ITR have been determined to be sufficient for ITR function. This minimal functional ITR can be used in all aspects of AAV vector production and transduction. Additional deletions may define an even smaller minimal functional ITR. The shorter length advantageously permits the packaging and transduction of larger transgenic cassettes.

In another embodiment, each of the elements that are present in a synthetic ITR can be the exact sequence as exists in a naturally occurring AAV ITR (the WT sequence) or can differ slightly (e.g., differ by addition, deletion, and/or substitution of 1, 2, 3, 4, 5 or more nucleotides) so long as the functioning of the elements of the AAV ITR continue to function at a level sufficient to are not substantially different from the functioning of these same elements as they exist in a naturally occurring AAV ITR.

In a further embodiment, rAAV vector, including an rAAV vector genome can comprise, between the ITRs, one or more additional non-AAV cis elements, e.g., elements that initiate transcription, mediate enhancer function, allow replication and symmetric distribution upon mitosis, or alter the persistence and processing of transduced genomes. Such elements are well known in the art and include, without limitation, promoters, enhancers, chromatin attachment sequences, telomeric sequences, cis-acting microRNAs (miRNAs), and combinations thereof.

In another embodiment, an ITR exhibits modified transcription activity relative to a naturally occurring ITR, e.g., ITR2 from AAV2. It is known that the ITR2 sequence inherently has promoter activity. It also inherently has termination activity, similar to a poly(A) sequence. The minimal functional ITR of the present invention exhibits transcription activity as shown in the examples, although at a diminished level relative to ITR2. Thus, in some embodiments, the ITR is functional for transcription. In other embodiments, the ITR is defective for transcription. In certain embodiments, the ITR can act as a transcription insulator, e.g., preventing transcription of a transgenic cassette present in the vector when the vector is integrated into a host chromosome.

One aspect of the invention relates to an rAAV vector genome comprising at least one synthetic AAV ITR, wherein the nucleotide sequence of one or more transcription factor binding sites in the ITR is deleted and/or substituted, relative to the sequence of a naturally occurring AAV ITR such as ITR2. In some embodiments, it is the minimal functional ITR in which one or more transcription factor binding sites are deleted and/or substituted. In some embodiments at least 1 transcription factor binding site is deleted and/or substituted, e.g., at least 5 or more or 10 or more transcription factor binding sites, e.g., at least 1, 2, 3, 4, 5, 6, 7, 8, 9, 10, 11, 12, 13, 14, 15, 16, 17, 18, 19, 20, or 21 transcription factor binding sites.

Another embodiment, a rAAV vector, including an rAAV vector genome as described herein comprises a polynucleotide comprising at least one synthetic AAV ITR, wherein one or more CpG islands (a cytosine base followed immediately by a guanine base (a CpG) in which the cytosines in such arrangement tend to be methylated) that typically occur at, or near the transcription start site in an ITR are deleted and/or substituted. In an embodiment, deletion or reduction in the number of CpG islands can reduce the immunogenicity of the rAAV vector. This results from a reduction or complete inhibition in TLR-9 binding to the rAAV vector DNA sequence, which occurs at CpG islands. It is also well known that methylation of CpG motifs results in transcriptional silencing. Removal of CpG motifs in the ITR is expected to result in decreased TLR-9 recognition and/or decreased methylation and therefore decreased transgene silencing. In some embodiments, it is the minimal functional ITR in which one or more CpG islands are deleted and/or substituted. In an embodiment, AAV ITR2 is known to contain 16 CpG islands of which one or more, or all 16 can be deleted.

In some embodiments, at least 1 CpG motif is deleted and/or substituted, e.g., at least 4 or more or 8 or more CpG motifs, e.g., at least 1, 2, 3, 4, 5, 6, 7, 8, 9, 10, 11, 12, 13, 14, 15, or 16 CpG motifs. The phrase “deleted and/or substituted” as used herein means that one or both nucleotides in the CpG motif is deleted, substituted with a different nucleotide, or any combination of deletions and substitutions.

In another embodiment, the synthetic ITR comprises, consists essentially of, or consists of one of the nucleotide sequences listed below. In other embodiments, the synthetic ITR comprises, consist essentially of, or consist of a nucleotide sequence that is at least 80% identical, e.g., at least 85%, 90%, 95%, 96%, 97%, 98%, or 99% identical to one of the nucleotide sequences listed below.

MH-257
(SEQ ID NO: 36)
AGGAACCCCTAGTGATGGAGTTGGCCACTCCCTCTCTGCGCGCTCGCTCG
CTCACTGAGGCAATTTGATAAAAATCGTCAAATTATAAACAGGCTTTGCC
TGTTTAGCCTCAGTGAGCGAGCGAGCGCGCAGAGAGGGAGTGGCCAACTC
CATCACTAGGGGTTCCT
MH-258
(SEQ ID NO: 37)
AGGAACCCCTAGTGATGGAGTTGGCCACTCCCTCTCTGCGCGCTCGCTCG
CTCACTGAGGGATAAAAATCCAGGCTTTGCCTGCCTCAGTGAGCGAGCGA
GCGCGCAGAGAGGGAGTGGCCAACTCCATCACTAGGGGTTCCT
MH Delta 258
(SEQ ID NO: 38)
AGGAACCCCTAGTGATGGAGTTGGCCACTCCCTCTCTGCGCGCTCGCTCG
CTCACTGAGGGATAAAAATCCAGGCTTTGCCTGCCTCAGTGAGCGAGCGA
GCGCGCAGAGAGGGAGTGGCCAACTCCATCACTAGGGGTTCCT
MH Telomere-1 ITR
(SEQ ID NO: 39)
AGGAACCCCTAGTGATGGAGTTGGCCACTCCCTCTCTGGGATTGGGATTG
CGCGCTCGCTCGCGGGATTGGGATTGGGATTGGGATTGGGATTGGGATTG
ATAAAAATCAATCCCAATCCCAATCCCAATCCCAATCCCAATCCCGCGAG
CGAGCGCGCAATCCCAATCCCAGAGAGGGAGTGGCCAACTCCATCACTAG
GGGTTCCT
MH Telomere-2 ITR
(SEQ ID NO: 40)
AGGAACCCCTAGTGATGGAGTTGGCCACTCCCTCTCTGCGCGCTCGCTCG
CTCGGGATTGGGATTGGGATTGGGATTGGGATTGGGATTGATAAAAATCA
ATCCCAATCCCAATCCCAATCCCAATCCCAATCCCGCGAGCGAGCGCGCA
GGAGAGGGAGTGGCCAACTCCATCACTAGGGGTTCCTAAGCTTATTATA
MH PolII 258 ITR
(SEQ ID NO: 41)
AGGAACCCCTAGTGATGGAGTTGGCCACTCCCTCTCTGCGCGCTCGCTCG
CTCACTGAGGGCGCCTATAAAGATAAAAATCCAGGCTTTGCCTGCCTCAG
TTAGCGAGCGAGCGCGCAGAGAGGGAGTGGCCAACTCCATCACTAGGGGT
TCCT
MH 258 Delta D conservative
(SEQ ID NO: 42)
CTAGTGATGGAGTTGGCCACTCCCTCTCTGCGCGCTCGCTCGCTCACTGA
GGGATAAAAATCCAGGCTTTGCCTGCCTCAGTGAGCGAGCGAGCGCGCAG
AGAGGGAGTGGCCAACTCCATCACTAG

In certain embodiments, a rAAV vector genome as described herein comprises a synthetic ITR that is capable of producing AAV virus particles that can transduce host cells. Such ITRs can be used, for example, for viral delivery of heterologous nucleic acids. Examples of such ITRs include MH-257, MH-258, and MH Delta 258 listed above.

In other embodiments, a rAAV vector genome as described herein containing a synthetic ITR is not capable of producing AAV virus particles. Such ITRs can be used, for example, for non-viral transfer of heterologous nucleic acids. Examples of such ITRs include MH Telomere-1, MH Telomere-2, and MH Pol II 258 listed above.

In a further embodiment, an rAAV vector genome as described herein comprising the synthetic ITR of the invention further comprises a second ITR which may be the same as or different from the first ITR. In one embodiment, an rAAV vector genome further comprises a heterologous nucleic acid, e.g., a sequence encoding a protein or a functional RNA. In an additional embodiment, a second ITR cannot be resolved by the Rep protein, i.e., resulting in a double stranded viral DNA.

In an embodiment, an rAAV vector genome comprises a polynucleotide comprising a synthetic ITR of the invention. In a further embodiment, the viral vector can be a parvovirus vector, e.g., an AAV vector. In another embodiment, a recombinant parvovirus particle (e.g., a recombinant AAV particle) comprises a synthetic ITR.

Another embodiment of the invention relates to a method of increasing the transgenic DNA packaging capacity of an AAV capsid, comprising generating an rAAV vector genome comprising at least one synthetic AAV ITR, wherein said ITR comprises: (a) an AAV rep binding element; (b) an AAV terminal resolution sequence; and (c) an AAV RBE element; wherein said ITR does not comprise any other AAV ITR sequences.

A further embodiment of the invention relates to a method of altering the cellular response to infection by an rAAV vector genome, comprising generating an rAAV vector genome comprising at least one synthetic ITR, wherein the nucleotide sequence of one or more transcription factor binding sites in said ITR is deleted and/or substituted, and further wherein an rAAV vector genome comprises at least one synthetic ITR that produces an altered cellular response to infection.

An additional embodiment of the invention relates to a method of altering the cellular response to infection by an rAAV vector genome, comprising generating an rAAV vector genome comprising at least one synthetic ITR, wherein one or more CpG motifs in said ITR are deleted and/or substituted, wherein the vector comprising at least one synthetic ITR produces an altered cellular response to infection.

### III. Vectors and Virions

In one embodiment, the rAAV vector (also referred to as a rAAV virion) as disclosed herein comprises a capsid protein, and a rAAV genome in the capsid protein. A rAAV capsid of the rAAV virion used to treat Pompe Disease is any of those listed in Table 1, or any combination thereof.

TABLE 1
TABLE 1: AAV Serotypes and exemplary Published corresponding capsid sequence

| Serotype and where capsid sequence is | Serotype and where capsid sequence is |
|---|---|
| published | published |
| AAV3.3b (See SEQ ID NO: 72 in US20030138772) | AAV3-3 (See SEQ ID NO: 200 US20150315612) |
| AAV3-3 (See SEQ ID NO: 217 US20150315612) | AAV3a ((See SEQ ID NO: 5 in US6156303) |
| AAV3a (See SEQ ID NO: 9 in US6156303) | AAV3b (See SEQ ID NO: 6 in US6156303) |
| AAV3b (See SEQ ID NO: 10 in US6156303) | AAV3b (See SEQ ID NO: 1 in US6156303) |
| AAV4 (See SEQ ID NO: 17 US20140348794) | AAV4 ((See SEQ ID NO: 5 in US20140348794) |
| AAV4 (See SEQ ID NO: 3 in US20140348794) | AAV4 (See SEQ ID NO: 14 in US20140348794) |
| AAV4 (See SEQ ID NO: 15 in US20140348794) | AAV4 (See SEQ ID NO: 19 in US20140348794) |
| AAV4 (See SEQ ID NO: 12 in US20140348794) | AAV4 (See SEQ ID NO: 13 in US20140348794) |
| AAV4 (See SEQ ID NO: 7 in US20140348794) | AAV4 (See SEQ ID NO: 8 in US20140348794) |
| AAV4 (See SEQ ID NO: 9 in US20140348794) | AAV4 (See SEQ ID NO: 2 in US20140348794) |
| AAV4 (See SEQ ID NO: 10 in US20140348794) | AAV4 (See SEQ ID NO: 11 in US20140348794) |
| AAV4 (See SEQ ID NO: 18 in US20140348794) | AAV4 (See SEQ ID NO: 63 in |
| | US20030138772) and US20160017295 SEQ |
| ID NO: (See SEQ ID NO: 4 in US20140348794) | AAV4 (See SEQ ID NO: 16 in US20140348794) |
| AAV4 (See SEQ ID NO: 20 in US20140348794) | AAV4 (See SEQ ID NO: 6 in US20140348794) |
| AAV4 (See SEQ ID NO: 1 in US20140348794) | AAV42.2 (See SEQ ID NO: 9 in US20030138772) |
| AAV42.2 (See SEQ ID NO: 102 in US20030138772) | AAV42.3b (See SEQ ID NO: 36 in US20030138772) |
| AAV42.3B (See SEQ ID NO: 107 in US20030138772) | AAV42.4 (See SEQ ID NO: 33 in US20030138772) |
| AAV42.4 (See SEQ ID NO: 88 in US20030138772) | AAV42.8 (See SEQ ID NO: 27 in US20030138772) |
| AAV42.8 (See SEQ ID NO: 85 in US20030138772) | AAV43.1 (See SEQ ID NO: 39 in US20030138772) |
| AAV43.1 (See SEQ ID NO: 92 in US20030138772) | AAV43.12 (See SEQ ID NO: 41 in US20030138772) |
| AAV43.12 (See SEQ ID NO: 93 in US20030138772) | AAV8 (See SEQ ID NO: 15 in US20150159173) |
| AAV8 (See SEQ ID NO: 7 in US20150376240) | AAV8 (See SEQ ID NO: 4 in US20030138772; |
| | US20150315612 SEQ |
| ID NO: 182 | AAV8 (See SEQ ID NO: 95 in |
| | US20030138772), US20140359799 SEQ |
| AAV8 (See SEQ ID NO: 31 in US20150159173) | AAV8 (See, e.g., SEQ ID NO: 8 in |
| | US20160017295, or SEQ ID NO: 7 in |
| | US7198951, or SEQ ID NO: 223 in |
| | US20150315612) |
| AAV8 (See SEQ ID NO: 8 in US20150376240) | AAV8 (See SEQ ID NO: 214 in US20150315612) |
| AAV-8b (See SEQ ID NO: 5 in US20150376240) | AAV-8b (See SEQ ID NO: 3 in US20150376240) |
| AAV-8h (See SEQ ID NO: 6 in US20150376240) | AAV-8h (See SEQ ID NO: 4 in US20150376240) |
| AAV9 (See SEQ ID NO: 5 in US20030138772) | AAV9 (See SEQ ID NO: 1 in US7198951) |
| AAV9 (See SEQ ID NO: 9 in US20160017295) | AAV9 (See SEQ ID NO: 100 in |
| | US20030138772), US7198951 SEQ ID NO: 2 |
| | AAV9 (See SEQ ID NO: 3 in US7198951) |
| AAV9 (AAVhu.14) (See SEQ ID NO: 3 in | AAV9 (AAVhu.14) (See SEQ ID NO: 123 in |
| US20150315612) | US20150315612) |
| AAVA3.1 (See SEQ ID NO: 120 in | AAVA3.3 (See SEQ ID NO: 57 in |
| US20030138772) | US20030138772) |
| AAVA3.3 (See SEQ ID NO: 66 in | AAVA3.4 (See SEQ ID NO: 54 in |
| US20030138772) | US20030138772) |
| AAVA3.4 (See SEQ ID NO: 68 in | AAVA3.5 (See SEQ ID NO: 55 in |
| US20030138772) | US20030138772) |
| AAVA3.5 (See SEQ ID NO: 69 in | AAVA3.7 (See SEQ ID NO: 56 in |
| US20030138772) | US20030138772) |
| AAVA3.7 (See SEQ ID NO: 67 in | AAV29. (See SEQ ID NO: 11 in (AAVbb. l) |
| US20030138772) | 161 US20030138772) |
| AAVC2 (See SEQ ID NO: 61 in US20030138772) | AAVCh.5 (See SEQ ID NO: 46 in |
| | US20150159173); US20150315612 SEQ |
| ID NO: 234 | AAVcy.2 (AAV13.3) (See SEQ ID NO: 15 in |
| | US20030138772) |
| AAV24.1 (See SEQ ID NO: 101 in | AAVcy.3 (AAV24.1) (See SEQ ID NO: 16 in |
| US20030138772) | US20030138772) |
| AAV27.3 (See SEQ ID NO: 104 in | AAVcy.4 (AAV27.3) (See SEQ ID NO: 17 in |
| US20030138772) | US20030138772) |
| AAVcy.5 (See SEQ ID NO: 227 in | AAV7.2 (See SEQ ID NO: 103 in |
| US20150315612) | US20030138772) |
| AAVcy.5 (AAV7.2) (See SEQ ID NO: 18 in | AAV16.3 (See SEQ ID NO: 105 in |
| US20030138772) | US20030138772) |
| AAVcy.6 (AAV16.3) (See SEQ ID NO: 10 in | AAVcy.5 (See SEQ ID NO: 8 in |
| US20030138772) | US20150159173) |
| AAVcy.5 (See SEQ ID NO: 24 in | AAVCy.5Rl (See SEQ ID NO: in |
| US20150159173) | US20150159173 |
| AAVCy.5R2 (See SEQ ID NO: in | AAVCy.5R3 (See SEQ ID NO: in |
| US20150159173) | US20150159173 |
| AAVCy.5R4 (See SEQ ID NO: in | AAVDJ (See SEQ ID NO: 3 in |
| US20150159173) | US20140359799) and SEQ ID NO: 2 in |
| | US7588772) |
| | AAVDJ (See SEQ ID NO: 2 in |
| | US20140359799; and SEQ ID NO: 1 in |
| | US7588772) |
| | AAVDJ-8 (See SEQ ID NO: in US7588772; |
| | Grimm et al. 2008 |
| AAVDJ-8 (See SEQ ID NO: in US7588772; | AAVF5 (See SEQ ID NO: 110 in |
| Grimm et al. 2008 | US20030138772) |
| AAVH2 (See SEQ ID NO: 26 in US20030138772) | AAVH6 (See SEQ ID NO: 25 in |
| | US20030138772) |
| AAVhEl.l (See SEQ ID NO: 44 in US9233131) | AAVhErl.14 (See SEQ ID NO: 46 in |
| | US9233131) |
| AAVhErl.16 (See SEQ ID NO: 48 in US9233131) | AAVhErl.18 (See SEQ ID NO: 49 in |
| | US9233131) |
| AAVhErl.23 (AAVhEr2.29) (See SEQ ID NO: 53 | AAVhErl.35 (See SEQ ID NO: 50 in |
| in US9233131) | US9233131) |
| AAVhErl.36 (See SEQ ID NO: 52 in US9233131) | AAVhErl.5 (See SEQ ID NO: 45 in |
| | US9233131) |
| AAVhErl.7 (See SEQ ID NO: 51 in US9233131) | AAVhErl.8 (See SEQ ID NO: 47 in |
| | US9233131) |
| AAVhEr2.16 (See SEQ ID NO: 55 in US9233131) | AAVhEr2.30 (See SEQ ID NO: 56 in |
| | US9233131) |
| AAVhEr2.31 (See SEQ ID NO: 58 in US9233131) | AAVhEr2.36 (See SEQ ID NO: 57 in |
| | US9233131) |
| AAVhEr2.4 (See SEQ ID NO: 54 in US9233131) | AAVhEr3.1 (See SEQ ID NO: 59 in |
| | US9233131) |
| AAVhu.l (See SEQ ID NO: 46 in US20150315612) | AAVhu.l (See SEQ ID NO: 144 in |
| | US20150315612) |
| AAVhu.lO (AAV16.8) (See SEQ ID NO: 56 in | AAVhu.lO (AAV16.8) (See SEQ ID NO: 156 |
| US20150315612) | in US20150315612) |
| AAVhu.ll (AAV16.12) (See SEQ ID NO: 57 in | AAVhu.ll (AAV16.12) (See SEQ ID NO: 153 |
| US20150315612) | in US20150315612) |
| AAVhu.12 (See SEQ ID NO: 59 in | AAVhu.12 (See SEQ ID NO: 154 in |
| US20150315612) | US20150315612) |
| AAVhu.13 (See SEQ ID NO: 16 in | |
| US2015015917 and ID NO: 71 in US20150315612) | |
| AAVhu.13 (See SEQ ID NO: 32 in | |
| US20150159173 and ID NO: 129 US20150315612) | |
| AAVhu.136.1 (See SEQ ID NO: 165 in | AAVhu.140.1 (See SEQ ID NO: 166 in |
| US20150315612) | US20150315612) |
| AAVhu.140.2 (See SEQ ID NO: 167 in | AAVhu.145.6 (See SEQ ID NO: 178 in |
| US20150315612) | US20150315612) |
| AAVhu.15 (See SEQ ID NO: 147 in | AAVhu.15 (AAV33.4) (See SEQ ID NO: 50 in |
| US20150315612) | US20150315612) |
| AAVhu.156.1 (See SEQ ID NO: 179 in | AAVhu.16 (See SEQ ID NO: 148 in |
| US20150315612) | US20150315612) |
| AAVhu.l6 (AAV33.8) (See SEQ ID NO: 51 in | AAVhu.17 (See SEQ ID NO: 83 in |
| US20150315612) | US20150315612) |
| AAVhu.l7 (AAV33.12) (See SEQ ID NO: 4 in | AAVhu.172.1 (See SEQ ID NO: 171 in |
| US20150315612) | US20150315612) |
| AAVhu.172.2 (See SEQ ID NO: 172 in | AAVhu.173.4 (See SEQ ID NO: 173 in |
| US20150315612) | US20150315612) |
| AAVhu.173.8 (See SEQ ID NO: 175 in | AAVhu.18 (See SEQ ID NO: 52 in |
| US20150315612) | US20150315612) |
| AAVhu.18 (See SEQ ID NO: 149 in | AAVhu.19 (See SEQ ID NO: 62 in |
| US20150315612) | US20150315612) |
| AAVhu.19 (See SEQ ID NO: 133 in | AAVhu.2 (See SEQ ID NO: 48 in |
| US20150315612) | US20150315612) |
| AAVhu.2 (See SEQ ID NO: 143 in | AAVhu.20 (See SEQ ID NO: 63 in |
| US20150315612) | US20150315612) |
| AAVhu.20 (See SEQ ID NO: 134 in | AAVhu.21 (See SEQ ID NO: 65 in |
| US20150315612) | US20150315612) |
| AAVhu.21 (See SEQ ID NO: 135 in | AAVhu.22 (See SEQ ID NO: 67 in |
| US20150315612) | US20150315612) |
| AAVhu.22 239 (See SEQ ID NO: 138 in | AAVhu.23 (See SEQ ID NO: 60 in |
| US20150315612) | US20150315612) |
| AAVhu.23.2 (See SEQ ID NO: 137 in | AAVhu.24 (See SEQ ID NO: 66 in |
| US20150315612) | US20150315612) |
| AAVhu.24 (See SEQ ID NO: 136 in | AAVhu.25 (See SEQ ID NO: 49 in |
| US20150315612) | US20150315612) |
| AAVhu.25 (See SEQ ID NO: 146 in | AAVhu.26 (See SEQ ID NO: 17 in |
| US20150315612) | US20150159173 and SEQ ID NO: 61 in |
| | US20150315612) |
| | AAVhu.26 (See SEQ ID NO: 33 in |
| | US20150159173), US20150315612 SEQ |
| | AAVhu.27 (See SEQ ID NO: 64 in |
| | US20150315612) |
| AAVhu.27 (See SEQ ID NO: 140 in | AAVhu.28 (See SEQ ID NO: 68 in |
| US20150315612) | US20150315612) |
| AAVhu.28 (See SEQ ID NO: 130 in | AAVhu.29 (See SEQ ID NO: 69 in |
| US20150315612) | US20150315612) |
| AAVhu.29 (See SEQ ID NO: 42 in | |
| US20150159173 and SEQ ID NO: 132 in | |
| US20150315612) | |
| AAVhu.29 (See SEQ ID NO: 225 in | AAVhu.29R (See SEQ ID NO: in |
| US20150315612) | US20150159173 |
| AAVhu.3 (See SEQ ID NO: 44 in | AAVhu.3 (See SEQ ID NO: 145 in |
| US20150315612) | US20150315612) |
| AAVhu.30 (See SEQ ID NO: 70 in | AAVhu.30 (See SEQ ID NO: 131 in |
| US20150315612) | US20150315612) |
| AAVhu.31 (See SEQ ID NO: 1 in | AAVhu.31 (See SEQ ID NO: 121 in |
| US20150315612) | US20150315612) |
| AAVhu.32 (See SEQ ID NO: 2 in | AAVhu.32 (See SEQ ID NO: 122 in |
| US20150315612) | US20150315612) |
| AAVhu.33 (See SEQ ID NO: 75 in | AAVhu.33 (See SEQ ID NO: 124 in |
| US20150315612) | US20150315612) |
| AAVhu.34 (See SEQ ID NO: 72 in | AAVhu.34 (See SEQ ID NO: 125 in |
| US20150315612) | US20150315612) |
| AAVhu.35 (See SEQ ID NO: 73 in | AAVhu.35 (See SEQ ID NO: 164 in |
| US20150315612) | US20150315612) |
| AAVhu.36 (See SEQ ID NO: 74 in | AAVhu.36 (See SEQ ID NO: 126 in |
| US20150315612) | US20150315612) |
| AAVhu.37 (See SEQ ID NO: 34 in | |
| US20150159173 and SEQ ID NO: 88 in | |
| US20150315612) | |
| AAVhu.37 (AAV106.1) (See SEQ ID NO: 10 in | |
| US20150315612 and SEQ ID NO: 18 in | |
| US20150159173) | |
| AAVhu.38 (See SEQ ID NO: 161 in | AAVhu.39 (See SEQ ID NO: 102 in |
| US20150315612) | US20150315612) |
| AAVhu.39 (AAVLG-9) (See SEQ ID NO: 24 in | AAVhu.4 (See SEQ ID NO: 47 in |
| US20150315612) | US20150315612) |
| AAVhu.4 (See SEQ ID NO: 141 in | AAVhu.40 (See SEQ ID NO: 87 in |
| US20150315612) | US20150315612) |
| AAVhu.40 (AAV114.3) (See SEQ ID NO: 11 in | AAVhu.41 (See SEQ ID NO: 91 in |
| US20150315612) | US20150315612) |
| AAVhu.41 (AAV127.2) (See SEQ ID NO: 6 in | AAVhu.42 (See SEQ ID NO: 85 in |
| US20150315612) | US20150315612) |
| AAVhu.42 (AAV127.5) (See SEQ ID NO:8 in | AAVhu.43 (See SEQ ID NO: 160 in |
| US20150315612) | US20150315612) |
| AAVhu.43 (See SEQ ID NO: 236 in | AAVhu.43 (AAV128.1) (See SEQ ID NO: 80 |
| US20150315612) | in US20150315612) |
| AAVhu.44 (See SEQ ID NO: 45 in | |
| US20150159173 and SEQ ID NO: 158 in | |
| US20150315612) | |
| AAVhu.44 (AAV128.3) (See SEQ ID NO: 81 in | AAVhu.44Rl (See SEQ ID NO: in |
| US20150315612) | US20150159173 |
| AAVhu.44R2 (See SEQ ID NO: in | AAVhu.44R3 (See SEQ ID NO: in |
| US20150159173 | US20150159173 |
| AAVhu.45 (See SEQ ID NO: 76 in | AAVhu.45 (See SEQ ID NO: 127 in |
| US20150315612) | US20150315612) |
| AAVhu.46 (See SEQ ID NO: 82 in | AAVhu.46 (See SEQ ID NO: 159 in |
| US20150315612) | US20150315612) |
| AAVhu.46 (See SEQ ID NO: 224 in | AAVhu.47 (See SEQ ID NO: 77 in |
| US20150315612) | US20150315612) |
| AAVhu.47 (See SEQ ID NO: 128 in | AAVhu.48 (See SEQ ID NO: 38 in |
| US20150315612) | US20150159173) |
| AAVhu.48 (See SEQ ID NO: 157 in | AAVhu.48 (AAV130.4) (See SEQ ID NO: 78 |
| US20150315612) | in US20150315612) |
| AAVhu.48Rl (See SEQ ID NO: in | AAVhu.48R2 (See SEQ ID NO: in |
| US20150159173 | US20150159173 |
| AAVhu.48R3 (See SEQ ID NO: in | AAVhu.49 (See SEQ ID NO: 209 in |
| US20150159173 | US20150315612) |
| AAVhu.49 (See SEQ ID NO: 189 in | AAVhu.5 (See SEQ ID NO: 45 in |
| US20150315612) | US20150315612) |
| AAVhu.5 (See SEQ ID NO: 142 in | AAVhu.51 (See SEQ ID NO: 208 in |
| US20150315612) | US20150315612) |
| AAVhu.51 (See SEQ ID NO: 190 in | AAVhu.52 (See SEQ ID NO: 210 in |
| US20150315612) | US20150315612) |
| AAVhu.52 (See SEQ ID NO: 191 in | AAVhu.53 (See SEQ ID NO: 19 in |
| US20150315612) | US20150159173) |
| AAVhu.53 (See SEQ ID NO: 35 in | AAVhu.53 (AAV145.1) (See SEQ ID NO: 176 |
| US20150159173) | in US20150315612) |
| AAVhu.54 (See SEQ ID NO: 188 in | AAVhu.54 (AAV145.5) (See SEQ ID NO: 177 |
| US20150315612) | in US20150315612) |
| AAVhu.55 (See SEQ ID NO: 187 in | AAVhu.56 (See SEQ ID NO: 205 in |
| US20150315612) | US20150315612) |
| AAVhu.56 (AAV145.6) (See SEQ ID NO: 168 in | AAVhu.56 (AAV145.6) (See SEQ ID NO: 192 |
| US20150315612) | in US20150315612) |
| AAVhu.57 (See SEQ ID NO: 206 in | AAVhu.57 (See SEQ ID NO: 169 in |
| US20150315612) | US20150315612) |
| AAVhu.57 (See SEQ ID NO: 193 in | AAVhu.58 (See SEQ ID NO: 207 in |
| US20150315612) | US20150315612) |
| AAVhu.58 (See SEQ ID NO: 194 in | AAVhu.6 (AAV3.1) (See SEQ ID NO: 5 in |
| US20150315612) | US20150315612) |
| AAVhu.6 (AAV3.1) (See SEQ ID NO: 84 in | AAVhu.60 (See SEQ ID NO: 184 in |
| US20150315612) | US20150315612) |
| AAVhu.60 (AAV161.10) (See SEQ ID NO: 170 in | AAVhu.6l (See SEQ ID NO: 185 in |
| US20150315612) | US20150315612) |
| AAVhu.61 (AAV161.6) (See SEQ ID NO: 174 in | AAVhu.63 (See SEQ ID NO: 204 in |
| US20150315612) | US20150315612) |
| AAVhu.63 (See SEQ ID NO: 195 in | AAVhu.64 (See SEQ ID NO: 212 in |
| US20150315612) | US20150315612) |
| AAVhu.64 (See SEQ ID NO: 196 in | AAVhu.66 (See SEQ ID NO: 197 in |
| US20150315612) | US20150315612) |
| AAVhu.67 (See SEQ ID NO: 215 in | AAVhu.67 (See SEQ ID NO: 198 in |
| US20150315612) | US20150315612) |
| AAVhu.7 (See SEQ ID NO: 226 in | AAVhu.7 (See SEQ ID NO: 150 in |
| US20150315612) | US20150315612) |
| AAVhu.7 (AAV7.3) (See SEQ ID NO: 55 in | AAVhu.71 (See SEQ ID NO: 79 in |
| US20150315612) | US20150315612) |
| AAVhu.8 (See SEQ ID NO: 53 in | AAVhu.8 (See SEQ ID NO: 12 in |
| US20150315612) | US20150315612) |
| AAVhu.8 (See SEQ ID NO: 151 in | AAVhu.9 (AAV3.1) (See SEQ ID NO: 58 in |
| US20150315612) | US20150315612) |
| AAVhu.9 (AAV3.1) (See SEQ ID NO: 155 in | AAV-LK01 (See SEQ ID NO: 2 in |
| US20150315612) | US20150376607) |
| AAV-LK01 (See SEQ ID NO: 29 in | AAV-LK02 (See SEQ ID NO: 3 in |
| US20150376607) | US20150376607) |
| AAV-LK02 (See SEQ ID NO: 30 in | AAV-LK03 (See SEQ ID NO: 4 in |
| US20150376607) | US20150376607) |
| AAV-LK03 (See SEQ ID NO: 12 in | |
| WO2015121501 and SEQ ID NO: 31 in | |
| US20150376607) | |
| AAV-LK04 (See SEQ ID NO: 5 in | AAV-LK04 (See SEQ ID NO: 32 in |
| US20150376607) | US20150376607) |
| AAV-LK05 (See SEQ ID NO: 6 in | AAV-LK05 (See SEQ ID NO: 33 in |
| US20150376607) | US20150376607) |
| AAV-LK06 (See SEQ ID NO: 7 in | AAV-LK06 (See SEQ ID NO: 34 in |
| US20150376607) | US20150376607) |
| AAV-LK07 (See SEQ ID NO: 8 in | AAV-LK07 (See SEQ ID NO: 35 in |
| US20150376607) | US20150376607) |
| AAV-LK08 (See SEQ ID NO: 9 in | AAV-LK08 (See SEQ ID NO: 36 in |
| US20150376607) | US20150376607) |
| AAV-LK09 (See SEQ ID NO: 10 in | AAV-LK09 (See SEQ ID NO: 37 in |
| US20150376607) | US20150376607) |
| AAV-LK10 (See SEQ ID NO: 11 in | AAV-LK10 (See SEQ ID NO: 38 in |
| US20150376607) | US20150376607) |
| AAV-LK11 (See SEQ ID NO: 12 in | AAV-LK11 (See SEQ ID NO: 39 in |
| US20150376607) | US20150376607) |
| AAV-LK12 (See SEQ ID NO: 13 in | AAV-LK12 (See SEQ ID NO: 40 in |
| US20150376607) | US20150376607) |
| AAV-LK13 (See SEQ ID NO: 14 in | AAV-LK13 (See SEQ ID NO: 41 in |
| US20150376607) | US20150376607) |
| AAV-LK14 (See SEQ ID NO: 15 in | AAV-LK14 (See SEQ ID NO: 42 in |
| US20150376607) | US20150376607) |
| AAV-LK15 (See SEQ ID NO: 16 in | AAV-LK15 (See SEQ ID NO: 43 in |
| US20150376607) | US20150376607) |
| AAV-LK16 (See SEQ ID NO: 17 in | AAV-LK16 (See SEQ ID NO: 44 in |
| US20150376607) | US20150376607) |
| AAV-LK17 (See SEQ ID NO: 18 in | AAV-LK17 (See SEQ ID NO: 45 in |
| US20150376607) | US20150376607) |
| AAV-LK18 (See SEQ ID NO: 19 in | AAV-LK18 (See SEQ ID NO: 46 in |
| US20150376607) | US20150376607) |
| AAV-LK19 (See SEQ ID NO: 20 in | AAV-LK19 (See SEQ ID NO: 47 in |
| US20150376607) | US20150376607) |
| AAV-PAEC (See SEQ ID NO: 1 in | AAV-PAEC (See SEQ ID NO: 48 in |
| US20150376607) | US20150376607) |
| AAV-PAEC11 (See SEQ ID NO: 26 in | AAV-PAEC11 (See SEQ ID NO: 54 in |
| US20150376607) | US20150376607) |
| AAV-PAEC 12 (See SEQ ID NO: 27 in | AAV-PAEC 12 (See SEQ ID NO: 51 in |
| US20150376607) | US20150376607) |
| AAV-PAEC 13 (See SEQ ID NO: 28 in | AAV-PAEC 13 (See SEQ ID NO: 49 in |
| US20150376607) | US20150376607) |
| AAV-PAEC2 (See SEQ ID NO: 21 in | AAV-PAEC2 (See SEQ ID NO: 56 in |
| US20150376607) | US20150376607) |
| AAV-PAEC4 (See SEQ ID NO: 22 in | AAV-PAEC4 (See SEQ ID NO: 55 in |
| US20150376607) | US20150376607) |
| AAV-PAEC6 (See SEQ ID NO: 23 in | AAV-PAEC6 (See SEQ ID NO: 52 in |
| US20150376607) | US20150376607) |
| AAV-PAEC7 (See SEQ ID NO: 24 in | AAV-PAEC7 (See SEQ ID NO: 53 in |
| US20150376607) | US20150376607) |
| AAV-PAEC8 (See SEQ ID NO: 25 in | AAV-PAEC8 (See SEQ ID NO: 50 in |
| US20150376607) | US20150376607) |
| AAVpi.l (See SEQ ID NO: 28 in US20150315612) | AAVpi.l (See SEQ ID NO: 93 in |
| | US20150315612; AAVpi.2 408, see SEQ ID |
| | NO: 30 in US20150315612) |
| AAVpi.2 (See SEQ ID NO: 95 in | AAVpi.3 (See SEQ ID NO: 29 in |
| US20150315612) | US20150315612) |
| AAVpi.3 (See SEQ ID NO: 94 in | AAVrh.10 (See SEQ ID NO: 9 in |
| US20150315612) | US20150159173) |
| AAVrh.10 (See SEQ ID NO: 25 in | AAV44.2 (See SEQ ID NO: 59 in |
| US20150159173) | US20030138772) |
| AAVrh.10 (AAV44.2) (See SEQ ID NO: 81 in | AAV42.1B (See SEQ ID NO: 90 in |
| US20030138772) | US20030138772) |
| AAVrh.l2 (AAV42.1b) (See SEQ ID NO: 30 in | AAVrh.13 (See SEQ ID NO: 10 in |
| US20030138772) | US20150159173) |
| AAVrh.13 (See SEQ ID NO: 26 in | AAVrh.13 (See SEQ ID NO: 228 in |
| US20150159173) | US20150315612) |
| AAVrh.l3R (See SEQ ID NO: in US20150159173 | AAV42.3A (See SEQ ID NO: 87 in |
| | US20030138772) |
| AAVrh.l4 (AAV42.3a) (See SEQ ID NO: 32 in | AAV42.5A (See SEQ ID NO: 89 in |
| US20030138772) | US20030138772) |
| AAVrh.l7 (AAV42.5a) (See SEQ ID NO: 34 in | AAV42.5B (See SEQ ID NO: 91 in |
| US20030138772) | US20030138772) |
| AAVrh.l8 (AAV42.5b) (See SEQ ID NO: 29 in | AAV42.6B (See SEQ ID NO: 112 in |
| US20030138772) | US20030138772) |
| AAVrh.l9 (AAV42.6b) (See SEQ ID NO: 38 in | AAVrh.2 (See SEQ ID NO: 39 in |
| US20030138772) | US20150159173) |
| AAVrh.2 (See SEQ ID NO: 231 in | AAVrh.20 (See SEQ ID NO: 1 in |
| US20150315612) | US20150159173) |
| AAV42.10 (See SEQ ID NO: 106 in | AAVrh.21 (AAV42.10) (See SEQ ID NO: 35 |
| US20030138772) | in US20030138772) |
| AAV42.11 (See SEQ ID NO: 108 in | AAVrh.22 (AAV42.11) (See SEQ ID NO: 37 |
| US20030138772) | in US20030138772) |
| AAV42.12 (See SEQ ID NO: 113 in | AAVrh.23 (AAV42.12) (See SEQ ID NO: 58 |
| US20030138772) | in US20030138772) |
| AAV42.13 (See SEQ ID NO: 86 in | AAVrh.24 (AAV42.13) (See SEQ ID NO: 31 |
| US20030138772) | in US20030138772) |
| AAV42.15 (See SEQ ID NO: 84 in | AAVrh.25 (AAV42.15) (See SEQ ID NO: 28 |
| US20030138772) | in US20030138772) |
| AAVrh.2R (See SEQ ID NO: in US20150159173 | AAVrh.31 (AAV223.1) (See SEQ ID NO: 48 |
| | in US20030138772) |
| AAVC1 (See SEQ ID NO: 60 in US20030138772) | AAVrh.32 (AAVC1) (See SEQ ID NO: 19 in |
| | 446 US20030138772) |
| AAVrh.32/33 (See SEQ ID NO: 2 in | AAVrh.51 (AAV2-5) (See SEQ ID NO: 104 in |
| US20150159173) | US20150315612) |
| AAVrh.52 (AAV3-9) (See SEQ ID NO: 18 in | AAVrh.52 (AAV3-9) (See SEQ ID NO: 96 in |
| US20150315612) | US20150315612) |
| AAVrh.53 (See SEQ ID NO: in US20150315612) | AAVrh.53 (AAV3-11) (See SEQ ID NO: 17 in |
| | US20150315612) |
| AAVrh.53 (AAV3-11) (See SEQ ID NO: 186 in | AAVrh.54 (See SEQ ID NO: 40 in |
| US20150315612) | US20150315612) |
| AAVrh.54 (See SEQ ID NO: 49 in | |
| US20150159173 and SEQ ID NO: 116 in | |
| US20150315612) | |
| AAVrh.55 (See SEQ ID NO: 37 in | AAVrh.55 (AAV4-19) (See SEQ ID NO: 117 |
| US20150315612) | in US20150315612) |
| AAVrh.56 (See SEQ ID NO: 54 in | AAVrh.56 (See SEQ ID NO: 152 in |
| US20150315612) | US20150315612) |
| AAVrh.57 (See SEQ ID NO: in 497 | AAVrh.57 (See SEQ ID NO: 105 in |
| US20150315612 SEQ ID NO: 26 | US20150315612) |
| AAVrh.58 (See SEQ ID NO: 27 in | AAVrh.58 (See SEQ ID NO: 48 in |
| US20150315612) | US20150159173 and SEQ ID NO: 106 in |
| | US20150315612) |
| | AAVrh.58 (See SEQ ID NO: 232 in |
| | US20150315612) |
| AAVrh.59 (See SEQ ID NO: 42 in | AAVrh.59 (See SEQ ID NO: 110 in |
| US20150315612) | US20150315612) |
| AAVrh.60 (See SEQ ID NO: 31 in | AAVrh.60 (See SEQ ID NO: 120 in |
| US20150315612) | US20150315612) |
| AAVrh.61 (See SEQ ID NO: 107 in | AAVrh.61 (AAV2-3) (See SEQ ID NO: 21 in |
| US20150315612) | US20150315612) |
| AAVrh.62 (AAV2-15) (See SEQ ID NO: 33 in | AAVrh.62 (AAV2-15) (See SEQ ID NO: 114 |
| US20150315612) | in US20150315612) |
| AAVrh.64 (See SEQ ID NO: 15 in | AAVrh.64 (See SEQ ID NO: 43 in |
| US20150315612) | US20150159173 and SEQ ID NO: 99 in |
| | US20150315612) |
| | AAVrh.64 (See SEQ ID NO: 233 in |
| | US20150315612) |
| AAVRh.64Rl (See SEQ ID NO: in | AAVRh.64R2 (See SEQ ID NO: in |
| US20150159173 | US20150159173 |
| AAVrh.65 (See SEQ ID NO: 35 in | AAVrh.65 (See SEQ ID NO: 112 in |
| US20150315612) | US20150315612) |
| AAVrh.67 (See SEQ ID NO: 36 in | AAVrh.67 (See SEQ ID NO: 230 in |
| US20150315612) | US20150315612) |
| AAVrh.67 (See SEQ ID NO: 47 in | |
| US20150159173 and SEQ ID NO: 47 in | |
| US20150315612) | |
| AAVrh.68 (See SEQ ID NO: 16 in | AAVrh.68 (See SEQ ID NO: 100 in |
| US20150315612) | US20150315612) |
| AAVrh.69 (See SEQ ID NO: 39 in | AAVrh.69 (See SEQ ID NO: 119 in |
| US20150315612) | US20150315612) |
| AAVrh.70 (See SEQ ID NO: 20 in | AAVrh.70 (See SEQ ID NO: 98 in |
| US20150315612) | US20150315612) |
| AAVrh.71 (See SEQ ID NO: 162 in | AAVrh.72 (See SEQ ID NO: 9 in |
| US20150315612) | US20150315612) |
| AAVrh.73 (See SEQ ID NO: 5 in | AAVrh.74 (See SEQ ID NO: 6 in |
| US20150159173) | US20150159173) |
| AAVrh.8 (See SEQ ID NO: 41 in | AAVrh.8 (See SEQ ID NO: 235 in |
| US20150159173) | US20150315612) |
| AAVrh.8R (See SEQ ID NO: 9 in | AAVrh.8R A586R mutant (See SEQ ID NO: 10 |
| US20150159173, WO2015168666) | in WO2015168666) |
| AAVrh.8R R533A mutant (See SEQ ID NO: 11 in | BAAV (bovine AAV) (See SEQ ID NO: 8 in |
| WO2015168666) | US9193769) |
| BAAV (bovine AAV) (See SEQ ID NO: 10 in | BAAV (bovine AAV) (See SEQ ID NO: 4 in |
| US9193769) | US9193769) |
| BAAV (bovine AAV) (See SEQ ID NO: 2 in | BAAV (bovine AAV) (See SEQ ID NO: 6 in |
| US9193769) | US9193769) |
| BAAV (bovine AAV) (See SEQ ID NO: 1 in | BAAV (bovine AAV) (See SEQ ID NO: 5 in |
| US9193769) | US9193769) |
| BAAV (bovine AAV) (See SEQ ID NO: 3 in | BAAV (bovine AAV) (See SEQ ID NO: 11 in |
| US9193769) | US9193769) |
| BAAV (bovine AAV) (See SEQ ID NO: 5 in | BAAV (bovine AAV) (See SEQ ID NO: 6 in |
| US7427396) | US7427396) |
| BAAV (bovine AAV) (See SEQ ID NO: 7 in | BAAV (bovine AAV) (See SEQ ID NO: 9 in |
| US9193769) | US9193769) |
| BNP61 AAV (See SEQ ID NO: 1 in | BNP61 AAV (See SEQ ID NO: 2 in |
| US20150238550) | US20150238550) |
| BNP62 AAV (See SEQ ID NO: 3 in | BNP63 AAV (See SEQ ID NO: 4 in |
| US20150238550) | US20150238550) |
| caprine AAV (See SEQ ID NO: 3 in US7427396) | caprine AAV (See SEQ ID NO: 4 in |
| | US7427396) |
| true type AAV (ttAAV) (See SEQ ID NO: 2 in | AAAV (Avian AAV) (See SEQ ID NO: 12 in |
| WO2015121501) | US9238800) |
| AAAV (Avian AAV) (See SEQ ID NO: 2 in | AAAV (Avian AAV) (See SEQ ID NO: 6 in |
| US9238800) | US9238800) |
| AAAV (Avian AAV) (See SEQ ID NO: 4 in | AAAV (Avian AAV) (See SEQ ID NO: 8 in |
| US9238800) | US9238800) |
| AAAV (Avian AAV) (See SEQ ID NO: 14 in | AAAV (Avian AAV) (See SEQ ID NO: 10 in |
| US9238800) | US9238800) |
| AAAV (Avian AAV) (See SEQ ID NO: 15 in | AAAV (Avian AAV) (See SEQ ID NO: 5 in |
| US9238800) | US9238800) |
| AAAV (Avian AAV) (See SEQ ID NO: 9 in | AAAV (Avian AAV) (See SEQ ID NO: 3 in |
| US9238800) | US9238800) |
| AAAV (Avian AAV) (See SEQ ID NO: 7 in | AAAV (Avian AAV) (See SEQ ID NO: 11 in |
| US9238800) | US9238800) |
| AAAV (Avian AAV) (See SEQ ID NO: in | AAAV (Avian AAV) (See SEQ ID NO: 1 in |
| US9238800) | US9238800) |
| AAV Shuffle 100-1 (See SEQ ID NO: 23 in | AAV Shuffle 100-1 (See SEQ ID NO: 11 in |
| US20160017295) | US20160017295) |
| AAV Shuffle 100-2 (See SEQ ID NO: 37 in | AAV Shuffle 100-2 (See SEQ ID NO: 29 in |
| US20160017295) | US20160017295) |
| AAV Shuffle 100-3 (See SEQ ID NO: 24 in | AAV Shuffle 100-3 (See SEQ ID NO: 12 in |
| US20160017295) | US20160017295) |
| AAV Shuffle 100-7 (See SEQ ID NO: 25 in | AAV Shuffle 100-7 (See SEQ ID NO: 13 in |
| US20160017295) | US20160017295) |
| AAV Shuffle 10-2 (See SEQ ID NO: 34 in | AAV Shuffle 10-2 (See SEQ ID NO: 26 in |
| US20160017295) | US20160017295) |
| AAV Shuffle 10-6 (See SEQ ID NO: 35 in | AAV Shuffle 10-6 (See SEQ ID NO: 27 in |
| US20160017295) | US20160017295) |
| AAV Shuffle 10-8 (See SEQ ID NO: 36 in | AAV Shuffle 10-8 (See SEQ ID NO: 28 in |
| US20160017295) | US20160017295) |
| AAV SM 100-10 (See SEQ ID NO: 41 in | AAV SM 100-10 (See SEQ ID NO: 33 in |
| US20160017295) | US20160017295) |
| AAV SM 100-3 (See SEQ ID NO: 40 in | AAV SM 100-3 (See SEQ ID NO: 32 in |
| US20160017295) | US20160017295) |
| AAV SM 10-1 (See SEQ ID NO: 38 in | AAV SM 10-1 (See SEQ ID NO: 30 in |
| US20160017295) | US20160017295) |
| AAV SM 10-2 (See SEQ ID NO: 10 in | AAV SM 10-2 (See SEQ ID NO: 22 in |
| US20160017295) | US20160017295) |
| AAV SM 10-8 (See SEQ ID NO: 39 in | AAV SM 10-8 (See SEQ ID NO: 31 in |
| US20160017295) | US20160017295) |
| AAV CBr-7.1 (See SEQ ID NO: 4 in | AAV CBr-7.1 (See SEQ ID NO: 54 in |
| WO2016065001) | WO2016065001) |
| AAV CBr-7.10 (See SEQ ID NO: 11 in | AAV CBr-7.10 (See SEQ ID NO: 61 in |
| WO2016065001) | WO2016065001) |
| AAV CBr-7.2 (See SEQ ID NO: 5 in | AAV CBr-7.2 (See SEQ ID NO: 55 in |
| WO2016065001) | WO2016065001) |
| AAV CBr-7.3 (See SEQ ID NO: 6 in | AAV CBr-7.3 (See SEQ ID NO: 56 in |
| WO2016065001) | WO2016065001) |
| AAV CBr-7.4 (See SEQ ID NO: 7 in | AAV CBr-7.4 (See SEQ ID NO: 57 in |
| WO2016065001) | WO2016065001) |
| AAV CBr-7.5 (See SEQ ID NO: 8 in | AAV CHt-6.6 (See SEQ ID NO: 35 in |
| WO2016065001) | WO2016065001) |
| AAV CHt-6.6 (See SEQ ID NO: 85 in | AAV CHt-6.7 (See SEQ ID NO: 36 in |
| WO2016065001) | WO2016065001) |
| AAV CHt-6.7 (See SEQ ID NO: 86 in | AAV CHt-6.8 (See SEQ ID NO: 37 in |
| WO2016065001) | WO2016065001) |
| AAV CHt-6.8 (See SEQ ID NO: 87 in | AAV CHt-Pl (See SEQ ID NO: 29 in |
| WO2016065001) | WO2016065001) |
| AAV CHt-Pl (See SEQ ID NO: 79 in | AAV CHt-P2 (See SEQ ID NO: 1 in |
| WO2016065001) | WO2016065001) |
| AAV CHt-P2 (See SEQ ID NO: 51 in | AAV CHt-P5 (See SEQ ID NO: 2 in |
| WO2016065001) | WO2016065001) |
| AAV CHt-P5 (See SEQ ID NO: 52 in | AAV CHt-P6 (See SEQ ID NO: 30 in |
| WO2016065001) | WO2016065001) |
| AAV CHt-P6 (See SEQ ID NO: 80 in | AAV CHt-P8 (See SEQ ID NO: 31 in |
| WO2016065001) | WO2016065001) |
| AAV CHt-P8 (See SEQ ID NO: 81 in | AAV CHt-P9 (See SEQ ID NO: 3 in |
| WO2016065001) | WO2016065001) |
| AAV CHt-P9 (See SEQ ID NO: 53 in | AAV CKd-1 (See SEQ ID NO: 57 in |
| WO2016065001) | US8734809) |
| AAV CKd-1 (See SEQ ID NO: 131 in | AAV CKd-10 (See SEQ ID NO: 58 in |
| US8734809) | US8734809) |
| AAV CKd-10 (See SEQ ID NO: 132 in | AAV CKd-2 (See SEQ ID NO: 59 in |
| US8734809) | US8734809) |
| AAV CKd-2 (See SEQ ID NO: 133 in | AAV CKd-3 (See SEQ ID NO: 60 in |
| US8734809) | US8734809) |
| AAV CKd-3 (See SEQ ID NO: 134 in | AAV CKd-4 (See SEQ ID NO: 61 in |
| US8734809) | US8734809) |
| AAV CKd-4 (See SEQ ID NO: 135 in | AAV CKd-6 (See SEQ ID NO: 62 in |
| US8734809) | US8734809) |
| AAV CKd-6 (See SEQ ID NO: 136 in | AAV CKd-7 (See SEQ ID NO: 63 in |
| US8734809) | US8734809) |
| AAV CKd-7 (See SEQ ID NO: 137 in | AAV CKd-8 (See SEQ ID NO: 64 in |
| US8734809) | US8734809) |
| AAV CKd-8 (See SEQ ID NO: 138 in | AAV CKd-B 1 (See SEQ ID NO: 73 in |
| US8734809) | US8734809) |
| AAV CKd-B 1 (See SEQ ID NO: 147 in | AAV CKd-B2 (See SEQ ID NO: 74 in |
| US8734809) | US8734809) |
| AAV CKd-B2 (See SEQ ID NO: 148 in | AAV CKd-B3 (See SEQ ID NO: 75 in |
| US8734809) | US8734809) |
| AAV CKd-B3 (See SEQ ID NO: in US8734809 | AAV CKd-B3 (See SEQ ID NO: 149 in |
| | US8734809) |
| AAV CLv-1 (See SEQ ID NO: 65 in US8734809) | AAV CLv-1 (See SEQ ID NO: 139 in |
| | US8734809) |
| AAV CLvl-1 (See SEQ ID NO: 171 in | AAV Civ 1-10 (See SEQ ID NO: 178 in |
| US8734809) | US8734809) |
| AAV CLvl-2 (See SEQ ID NO: 172 in | AAV CLv-12 (See SEQ ID NO: 66 in |
| US8734809) | US8734809) |
| AAV CLv-12 (See SEQ ID NO: 140 in | AAV CLvl-3 (See SEQ ID NO: 173 in |
| US8734809) | US8734809) |
| AAV CLv-13 (See SEQ ID NO: 67 in | AAV CLv-13 (See SEQ ID NO: 141 in |
| US8734809) | US8734809) |
| AAV CLvl-4 (See SEQ ID NO: 174 in | AAV Civ 1-7 (See SEQ ID NO: 175 in |
| US8734809) | US8734809) |
| AAV Civ 1-8 (See SEQ ID NO: 176 in | AAV Civ 1-9 (See SEQ ID NO: 177 in |
| US8734809) | US8734809) |
| AAV CLv-2 (See SEQ ID NO: 68 in US8734809) | AAV CLv-2 (See SEQ ID NO: 142 in |
| | US8734809) |
| AAV CLv-3 (See SEQ ID NO: 69 in US8734809) | AAV CLv-3 (See SEQ ID NO: 143 in |
| | US8734809) |
| AAV CLv-4 (See SEQ ID NO: 70 in US8734809) | AAV CLv-4 (See SEQ ID NO: 144 in |
| | US8734809) |
| AAV CLv-6 (See SEQ ID NO: 71 in US8734809) | AAV CLv-6 (See SEQ ID NO: 145 in |
| | US8734809) |
| AAV CLv-8 (See SEQ ID NO: 72 in US8734809) | AAV CLv-8 (See SEQ ID NO: 146 in |
| | US8734809) |
| AAV CLv-Dl (See SEQ ID NO: 22 in | AAV CLv-Dl (See SEQ ID NO: 96 in |
| US8734809) | US8734809) |
| AAV CLv-D2 (See SEQ ID NO: 23 in | AAV CLv-D2 (See SEQ ID NO: 97 in |
| US8734809) | US8734809) |
| AAV CLv-D3 (See SEQ ID NO: 24 in | AAV CLv-D3 (See SEQ ID NO: 98 in |
| US8734809) | US8734809) |
| AAV CLv-D4 (See SEQ ID NO: 25 in | AAV CLv-D4 (See SEQ ID NO: 99 in |
| US8734809) | US8734809) |
| AAV CLv-D5 (See SEQ ID NO: 26 in | AAV CLv-D5 (See SEQ ID NO: 100 in |
| US8734809) | US8734809) |
| AAV CLv-D6 (See SEQ ID NO: 27 in | AAV CLv-D6 (See SEQ ID NO: 101 in |
| US8734809) | US8734809) |
| AAV CLv-D7 (See SEQ ID NO: 28 in | AAV CLv-D7 (See SEQ ID NO: 102 in |
| US8734809) | US8734809) |
| AAV CLv-D8 (See SEQ ID NO: 29 in | AAV CLv-D8 (See SEQ ID NO: 103 in |
| US8734809) | US8734809); AAV CLv-Kl 762, see SEQ ID |
| | NO: 18 in WO2016065001) |
| AAV CLv-Kl (See SEQ ID NO: 68 in | AAV CLv-K3 (See SEQ ID NO: 19 in |
| WO2016065001) | WO2016065001) |
| AAV CLv-K3 (See SEQ ID NO: 69 in | AAV CLv-K6 (See SEQ ID NO: 20 in |
| WO2016065001) | WO2016065001) |
| AAV CLv-K6 (See SEQ ID NO: 70 in | AAV CLv-L4 (See SEQ ID NO: 15 in |
| WO2016065001) | WO2016065001) |
| AAV CLv-L4 (See SEQ ID NO: 65 in | AAV CLv-L5 (See SEQ ID NO: 16 in |
| WO2016065001) | WO2016065001) |
| AAV CLv-L5 (See SEQ ID NO: 66 in | AAV CLv-L6 (See SEQ ID NO: 17 in |
| WO2016065001) | WO2016065001) |
| AAV CLv-L6 (See SEQ ID NO: 67 in | AAV CLv-Ml (See SEQ ID NO: 21 in |
| WO2016065001) | WO2016065001) |
| AAV CLv-Ml (See SEQ ID NO: 71 in | AAV CLv-Mll (See SEQ ID NO: 22 in |
| WO2016065001) | WO2016065001) |
| AAV CLv-Ml 1 (See SEQ ID NO: 72 in | AAV CLv-M2 (See SEQ ID NO: 23 in |
| WO2016065001) | WO2016065001) |
| AAV CLv-M2 (See SEQ ID NO: 73 in | AAV CLv-M5 (See SEQ ID NO: 24 in |
| WO2016065001) | WO2016065001) |
| AAV CLv-M5 (See SEQ ID NO: 74 in | AAV CLv-M6 (See SEQ ID NO: 25 in |
| WO2016065001) | WO2016065001) |
| AAV CLv-M6 (See SEQ ID NO: 75 in | AAV CLv-M7 (See SEQ ID NO: 26 in |
| WO2016065001) | WO2016065001) |
| AAV CLv-M7 (See SEQ ID NO: 76 in | AAV CLv-M8 (See SEQ ID NO: 27 in |
| WO2016065001) | WO2016065001) |
| AAV CLv-M8 (See SEQ ID NO: 77 in | AAV CLv-M9 (See SEQ ID NO: 28 in |
| WO2016065001) | WO2016065001) |
| AAV CLv-M9 (See SEQ ID NO: 78 in | AAV CLv-Rl (See SEQ ID NO: 30 in |
| WO2016065001) | US8734809) |
| AAV CLv-Rl (See SEQ ID NO: 104 in | AAV CLv-R2 (See SEQ ID NO: 31 in |
| US8734809) | US8734809) |
| AAV CLv-R2 (See SEQ ID NO: 105 in | AAV CLv-R3 (See SEQ ID NO: 32 in |
| US8734809) | US8734809) |
| AAV CLv-R3 (See SEQ ID NO: 106 in | AAV CLv-R4 (See SEQ ID NO: 33 in |
| US8734809) | US8734809) |
| AAV CLv-R4 (See SEQ ID NO: 107 in | AAV CLv-R5 (See SEQ ID NO: 34 in |
| US8734809) | US8734809) |
| AAV CLv-R5 (See SEQ ID NO: 108 in | AAV CLv-R6 (See SEQ ID NO: 35 in |
| US8734809) | US8734809) |
| AAV CLv-R6 (See SEQ ID NO: 109 in | AAV CLv-R7 (See SEQ ID NO: 110 in |
| US8734809); AAV CLv-R7 802 (see SEQ ID NO: | US8734809) |
| 36 in US8734809) | |
| AAV CLv-R8 (See SEQ ID NO: 37 in | AAV CLv-R8 (See SEQ ID NO: 111 in |
| US8734809) | US8734809) |
| AAV CLv-R9 (See SEQ ID NO: 38 in | AAV CLv-R9 (See SEQ ID NO: 112 in |
| US8734809) | US8734809) |
| AAV CSp-1 (See SEQ ID NO: 45 in US8734809) | AAV CSp-1 (See SEQ ID NO: 119 in |
| | US8734809) |
| AAV CSp-10 (See SEQ ID NO: 46 in US8734809) | AAV CSp-10 (See SEQ ID NO: 120 in |
| | US8734809) |
| AAV CSp-11 (See SEQ ID NO: 47 in US8734809) | AAV CSp-11 (See SEQ ID NO: 121 in |
| | US8734809) |
| AAV CSp-2 (See SEQ ID NO: 48 in US8734809) | AAV CSp-2 (See SEQ ID NO: 122 in |
| AAV CSp-3 (See SEQ ID NO: 49 in US8734809) | US8734809) |
| | AAV CSp-3 (See SEQ ID NO: 123 in |
| | US8734809) |
| AAV CSp-4 (See SEQ ID NO: 50 in US8734809) | AAV CSp-4 (See SEQ ID NO: 124 in |
| | US8734809) |
| AAV CSp-6 (See SEQ ID NO: 51 in US8734809) | AAV CSp-6 (See SEQ ID NO: 125 in |
| | US8734809) |
| AAV CSp-7 (See SEQ ID NO: 52 in US8734809) | AAV CSp-7 (See SEQ ID NO: 126 in |
| | US8734809) |
| AAV CSp-8 (See SEQ ID NO: 53 in US8734809) | AAV CSp-8 (See SEQ ID NO: 127 in |
| | US8734809) |
| AAV CSp-8.10 (See SEQ ID NO: 38 in | AAV CSp-8.10 (See SEQ ID NO: 88 in |
| WO2016065001) | WO2016065001) |
| AAV CSp-8.2 (See SEQ ID NO: 39 in | AAV CSp-8.2 (See SEQ ID NO: 89 in |
| WO2016065001) | WO2016065001) |
| AAV CSp-8.4 (See SEQ ID NO: 40 in | AAV CSp-8.4 (See SEQ ID NO: 90 in |
| WO2016065001) | WO2016065001) |
| AAV CSp-8.5 (See SEQ ID NO: 41 in | AAV CSp-8.5 (See SEQ ID NO: 91 in |
| WO2016065001) | WO2016065001) |
| AAV CSp-8.6 (See SEQ ID NO: 42 in | AAV CSp-8.6 (See SEQ ID NO: 92 in |
| WO2016065001) | WO2016065001) |
| AAV CSp-8.7 (See SEQ ID NO: 43 in | AAV CSp-8.7 (See SEQ ID NO: 93 in |
| WO2016065001) | WO2016065001) |
| AAV CSp-8.8 (See SEQ ID NO: 44 in | AAV CSp-8.8 (See SEQ ID NO: 94 in |
| WO2016065001) | WO2016065001) |
| AAV CSp-8.9 (See SEQ ID NO: 45 in | AAV CSp-8.9 (See SEQ ID NO: 95 in |
| WO2016065001) | WO2016065001) |
| AAV CSp-9 842 (See SEQ ID NO: 54 in | AAV CSp-9 (See SEQ ID NO: 128 in |
| US8734809) | US8734809) |
| AAV.hu.48R3 (See SEQ ID NO: 183 in | AAV.VR-355 (See SEQ ID NO: 181 in |
| US8734809) | US8734809) |
| AAV3B (See SEQ ID NO: 48 in WO2016065001) | AAV3B (See SEQ ID NO: 98 in WO2016065001) |
| AAV4 (See SEQ ID NO: 49 in WO2016065001) | AAV4 (See SEQ ID NO: 99 in WO2016065001) |
| AAV5 (See SEQ ID NO: 50 in WO2016065001) | AAV5 (See SEQ ID NO: 100 in WO2016065001) |
| AAVF1/HSC1 (See SEQ ID NO: 20 in | AAVF1/HSC1 (See SEQ ID NO: 2 in |
| WO2016049230) | WO2016049230) |
| AAVF11/HSC11 (See SEQ ID NO: 26 in | AAVF11/HSC11 (See SEQ ID NO: 4 in |
| WO2016049230) | WO2016049230) |
| AAVF12/HSC12 (See SEQ ID NO: 30 in | AAVF12/HSC12 (See SEQ ID NO: 12 in |
| WO2016049230) | WO2016049230) |
| AAVF13/HSC13 (See SEQ ID NO: 31 in | AAVF13/HSC13 (See SEQ ID NO: 14 in |
| WO2016049230) | WO2016049230) |
| AAVF14/HSC14 (See SEQ ID NO: 32 in | AAVF14/HSC14 (See SEQ ID NO: 15 in |
| WO2016049230) | WO2016049230) |
| AAVF15/HSC15 (See SEQ ID NO: 33 in | AAVF15/HSC15 (See SEQ ID NO: 16 in |
| WO2016049230) | WO2016049230) |
| AAVF16/HSC16 (See SEQ ID NO: 34 in | AAVF16/HSC16 (See SEQ ID NO: 17 in |
| WO2016049230) | WO2016049230) |
| AAVF17/HSC17 (See SEQ ID NO: 35 in | AAVF17/HSC17 (See SEQ ID NO: 13 in |
| WO2016049230) | WO2016049230) |
| AAVF2/HSC2 (See SEQ ID NO: 21 in | AAVF2/HSC2 (See SEQ ID NO: 3 in |
| WO2016049230) | WO2016049230) |
| AAVF3/HSC3 (See SEQ ID NO: 22 in | AAVF3/HSC3 (See SEQ ID NO: 5 in |
| WO2016049230) | WO2016049230) |
| AAVF4/HSC4 (See SEQ ID NO: 23 in | AAVF4/HSC4 (See SEQ ID NO: 6 in |
| WO2016049230) | WO2016049230) |
| AAVF5/HSC5 (See SEQ ID NO: 25 in | AAVF5/HSC5 (See SEQ ID NO: 11 in |
| WO2016049230) | WO2016049230) |
| AAVF6/HSC6 (See SEQ ID NO: 24 in | AAVF6/HSC6 (See SEQ ID NO: 7 in |
| WO2016049230) | WO2016049230) |
| AAVF7/HSC7 (See SEQ ID NO: 27 in | AAVF7/HSC7 (See SEQ ID NO: 8 in |
| WO2016049230) | WO2016049230) |
| AAVF8/HSC8 (See SEQ ID NO: 28 in | AAVF8/HSC8 (See SEQ ID NO: 9 in |
| WO2016049230) | WO2016049230) |
| AAVF9/HSC9 (See SEQ ID NO: 10 in | AAVF9/HSC9 882 (see SEQ ID NO: 29 in |
| WO2016049230) | WO2016049230) |

Table 2 describe exemplary chimeric or variant capsid proteins that can be used as the AAV capsid in the rAAV vector described herein, or with any combination with wild type capsid proteins and/or other chimeric or variant capsid proteins now known or later identified and each is incorporated herein. In some embodiments, the rAAV vector encompassed for use is a chimeric vector, e.g., as disclosed in 9,012,224 and U.S. Pat. No. 7,892,809, which are incorporated herein in their entirety by reference.

In some embodiments, the rAAV vector is a haploid rAAV vector, as disclosed in PCT/US18/22725, or polyploid rAAV vector, e.g., as disclosed in PCT/US2018/044632 filed on Jul. 31, 2018 and in U.S. application Ser. No. 16/151,110, each of which are incorporated herein in their entirety by reference. In some embodiments, the rAAV vector is a rAAV3 vector, as disclosed in 9,012,224 and WO 2017/106236 which are incorporated herein in their entirety by reference.

TABLE 2
Exemplary chimeric and rAAV variant capsids

| Chimeric or | | Chimeric or | |
|---|---|---|---|
| variant capsid | reference | variant capsid | reference |
| LK03 and others | Lisowski et al. [REF 1] | AAV-leukemia | Michelfelder S [REF |
| LK0-19 | | targeting | 30] |
| AAV-DJ | Grimm et al., [REF 2] | AAV-tumor targeting | Muller O J, et al., |
| | | | [REF 31] |
| Olig001 | Powell S K et al., [REF 3] | AAV-tumor targeting | Grifman M et al., |
| | | | [REF 32] |
| rAAV2-retro | Tervo D et al., [REF 4] | AAV2 efficient | Girod et al., [REF 33] |
| | | targeting | |
| AAV-LiC | Marsic D et al., [REF 5] | AAVpo2.1, -po4, -poS, | Bello A, et al., [REF |
| | | and -po6). | 34] |
| (AAV-Keral, AAV- | Sallach et al., [REF 6] | AAV rh and AAV Hu | Gao G, et al., [REF |
| Kera2, and AAV- | | | 35] |
| Kera3) | | | |
| AAV 7m8 | Dalkara et al., [REF 7] | AAV-Go.1 | Arbetman A E et al., |
| | | | [REF 36] |
| (AAV1.9 | Asuri P et al., [REF 8] | AAV-mo.1 | Lochrie M A et al., |
| | | | [REF 37] |
| AAV r3.45 | Jang J H et al., [REF 9] | BAAV | Schmidt M, et al., |
| | | | [REF 38] |
| AAV clone 32 and | Gray S J, et al., [REF 10] | AAAV | Bossis I et al., [REF |
| 83) | | | 39] |
| AAV-U87R7-C5 | Maguire et al., [REF 11] | AAV variants | Chen C L et al., [REF |
| | | | 40] |
| AAV ShH13, AAV | Koerberetal., [REF 12] | AAV8 K137R | Sen D et al., [REF 41] |
| ShH19, AAV Ll-12 | | | |
| AAV HAE-1, AAV | Li W et al., [REF 13] | AAV2 Y | Li B, et al., [REF 42] |
| HAE-2 | | | |
| AAV variant ShH10 | Klimczak et al., [REF 14] | AAV2 | Gabriel N et al., [REF |
| | | | 43] |
| AAV2.5T | Excoffon et al., [REF 15] | AAV Anc80L65 | Zinn E, et al., [REF |
| | | | 44] |
| AAV LS1-4, AAV | Sellner L et al., [REF 16] | AAV2G9 | Shen S et al., [REF |
| Lsm | | | 45] |
| AAV1289 | Li W, et al., [REF 17] | AAV2 265 insertion- | Li C, et al., [REF 46] |
| | | AAV2/265D | |
| AAVHSC 1-17 | Charbel Issa P et al., | AAV2.5 | Bowles D E, et al., |
| | [REF 18] | | [REF 47] |
| AAV2 Rec 1-4 | Huang W. et al., [REF | AAV3 SASTG | Messina E L et al., |
| | 19] | | [REF 48] and [REF |
| | | | 55], (Piacentio et al., |
| | | | (Hum Gen Ther, |
| | | | 2012, 23: 635-646)) |
| AAV8BP2 | Cronin T, et al., [REF 20] | AAV2i8 | Asokan A et al., [REF |
| | | | 49] |
| AAV-B1 | Choudhury S R, et al., | AAV8G9 | Vance M, et al., [REF |
| | [REF 21] | | 50] |
| AAV-PHP.B | Deverman B E, et al., | AAV2 tyrosine | Zhong L et al., [REF |
| | [REF 22] | mutants AAV2 Y-F | 51] |
| AAV9.45, | Pulicherla N [REF 23], et | AAV8 Y-F and AAV9 | Petrs-Silva H et al., |
| AAV9.61, | al., | Y-F | [REF 52] |
| AAV9.47 | | | |
| AAVM41 | Yang L et al., [REF 24] | AAV6 Y-F | Qiao C et al., [REF |
| | | | 53] |
| AAV2 displayed | Korbelin J et al. [REF | (AAV6.2) PCT | Carlon M, et al., [REF |
| peptides) | 25], | Publication No. | 54] |
| | | WO2013158879Al | |
| | | (lysine mutants) | |
| AAV2-GMN | Geoghegan J C [REF 26] | | |
| AAV9-peptide | Varadi K, et al., [REF 27] | | |
| displayed | | | |
| AAV8 and AAV9 | Michelfelder et al., [REF | | |
| peptide displayed | 28] | | |
| AAV2-muscle | YuCY et al., [REF 29] | | |
| targeting peptide | | | |

In one embodiment, the rAAV vector as disclosed herein comprises a capsid protein, associated with any of the following biological sequence files listed in the file wrappers of USPTO issued patents and published applications, which describe chimeric or variant capsid proteins that can be incorporated into the AAV capsid of this invention in any combination with wild type capsid proteins and/or other chimeric or variant capsid proteins now known or later identified (for demonstrative purposes, 11486254 corresponds to U.S. patent application Ser. No. 11/486,254 and the other biological sequence files are to be read in a similar manner): 11486254.raw, 11932017.raw, 12172121.raw, 12302206.raw, 12308959.raw, 12679144.raw, 13036343.raw, 13121532.raw, 13172915.raw, 13583920.raw, 13668120.raw, 13673351.raw, 13679684.raw, 14006954.raw, 14149953.raw, 14192101.raw, 14194538.raw, 14225821.raw, 14468108.raw, 14516544.raw, 14603469.raw, 14680836.raw, 14695644.raw, 14878703.raw, 14956934.raw, 15191357.raw, 15284164.raw, 15368570.raw, 15371188.raw, 15493744.raw, 15503120.raw, 15660906.raw, and 15675677.raw.

In an embodiment, the AAV capsid proteins and virus capsids of this invention can be chimeric in that they can comprise all or a portion of a capsid subunit from another virus, optionally another parvovirus or AAV, e.g., as described in international patent publication WO 00/28004, which is incorporated by reference.

In some embodiments, an rAAV vector genome is single stranded or a monomeric duplex as described in U.S. Pat. No. 8,784,799, which is incorporated herein.

As a further embodiment, the AAV capsid proteins and virus capsids of this invention can be polyploid (also referred to as haploid) in that they can comprise different combinations of VP1, VP2 and VP3 AAV serotypes in a single AAV capsid as described in PCT/US18/22725, which is incorporated by reference.

In an embodiment, an rAAV vector useful in the treatment of Pompe Disease as disclosed herein is an AAV3b capsid. AAV3b capsids encompassed for use are described in 2017/106236, and 9,012,224 and 7,892,809, which are incorporated herein in its entirety by reference.

In some embodiments, the AAV3b capsid comprises SEQ ID NO: 44. In an embodiment, the AAV capsid used in the treatment of Pompe Disease can be a modified AAV capsid that is derived in whole or in part from the AAV capsid set forth in SEQ ID NO: 44. In some embodiments, the amino acids from an AAV3b capsid as set forth in SEQ ID NO: 44 can be, or are substituted with amino acids from another capsid of a different AAV serotype, wherein the substituted and/or inserted amino acids can be from any AAV serotype, and can include either naturally occurring or partially or completely synthetic amino acids.

In another embodiment, an AAV capsid used in the treatment of Pompe Disease is an AAV3b265D capsid. In this particular embodiment, an AAV3b265D capsid comprises a modification in the amino acid sequence of the two-fold axis loop of an AAV3b capsid via replacement of amino acid G265 of the AAV3b capsid with D265. In some embodiments, an AAV3b265D capsid comprises SEQ ID NO: 46. However, the modified virus capsids of the invention are not limited to AAV capsids set forth in SEQ ID NO: 46. In some embodiments, the amino acids from AAV3b265D as set forth in SEQ ID NO. 46 can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment an rAAV vector useful in the treatment of Pompe Disease as disclosed herein is an AAV3b265D549A capsid. In this particular embodiment, an AAV3b265D549A capsid comprises a modification in the amino acid sequence of the two-fold axis loop of an AAV3b capsid via replacement of amino acid G265 of the AAV3b capsid with D265 and replacement of amino acid T549 of the AAV3b capsid with A549. In some embodiments, an AAV3b265D549A capsid comprises SEQ ID NO: 50. However, the modified virus capsids of the invention are not limited to AAV capsids set forth in SEQ ID NO: 50. In some embodiments, the amino acids from AAV3b265D549A as set forth in SEQ ID NO: 50 can be, or are substituted with amino acids from a capsid from an AAV of a different serotype, wherein the substituted and/or inserted amino acids can be from any AAV serotype, and can include either naturally occurring or partially or completely synthetic amino acids. In some embodiments, the amino acids from AAV3bSASTG (i.e., a AAV3b capsid comprising Q263A/T265 mutations) can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment, an rAAV vector useful in the treatment of Pompe Disease as disclosed herein is an AAV3b549A capsid. In this particular embodiment, an AAV3b549A capsid comprises a modification in the amino acid sequence of the two-fold axis loop of an AAV3b capsid via replacement of amino acid T549 of the AAV3b capsid with A549. In some embodiments, an AAV3b549A capsid comprises SEQ ID NO: 52. However, the modified virus capsids of the invention are not limited to AAV capsids set forth in SEQ ID NO: 52. In some embodiments, the amino acids from AAV3b549A as set forth in SEQ ID NO: 52 can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment, an rAAV vector useful in the treatment of Pompe Disease as disclosed herein is an AAV3bQ263Y capsid. In this particular embodiment, an AAV3bQ263Y capsid comprises a modification in the amino acid sequence of the two-fold axis loop of an AAV3b capsid via replacement of amino acid Q263 of the AAV3b capsid with Y263. In some embodiments, an AAV3b549A capsid comprises SEQ ID NO: 54. However, the modified virus capsids of the invention are not limited to AAV capsids set forth in SEQ ID NO: 54. In some embodiments, the amino acids from AAV3bQ263Y as set forth in SEQ ID NO: 54 can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment, an rAAV vector useful in the treatment of Pompe Disease as disclosed herein is AAV3bSASTG serotype or comprises a AAV3bSASTG capsid. In this particular embodiment, an AAV3bSASTG capsid comprises a modification in the amino acid sequence to comprise a SASTG mutation, in particular, the AAV3b capsid was modified to resemble AAV2 Q263A/T265 subvariant by introducing these modifications at similar positions in the AAV3b capsid (as disclosed in Messina E L, et al., Adeno-associated viral vectors based on serotype 3b use components of the fibroblast growth factor receptor signaling complex for efficient transduction. Hum. Gene Ther. 2012 October: 23(10):1031-4, Piacentino III, Valentino, et al. “X-linked inhibitor of apoptosis protein-mediated attenuation of apoptosis, using a novel cardiac-enhanced adeno-associated viral vector.” Human gene therapy 23.6 (2012): 635-646. which are both incorporated herein in their entirety by reference). Accordingly, in some embodiments, an rAAV vector useful in the treatment of Pompe Disease as disclosed herein is AAV3bSASTG serotype or comprises a AAV3bSASTG capsid comprising a AAV3b Q263A/T265 capsid. In some embodiments, the amino acids from AAV3bSASTG can be, or are substituted with amino acids from a capsid from an AAV of a different serotype, wherein the substituted and/or inserted amino acids can be from any AAV serotype, and can include either naturally occurring or partially or completely synthetic amino acids.

In order to facilitate their introduction into a cell, an rAAV vector genome useful in the invention are recombinant nucleic acid constructs that include (1) a heterologous sequence to be expressed (in one embodiment, a polynucleotide encoding a GAA polypeptide) and (2) viral sequence elements that facilitate integration and expression of the heterologous genes. The viral sequence elements may include those sequences of an AAV vector genome that are required in cis for replication and packaging (e.g., functional ITRs) of the DNA into an AAV capsid. In an embodiment, the heterologous gene encodes GAA, which is useful for correcting a GAA-deficiency in a patient suffering from Pompe Disease. In an embodiment, such an rAAV vector genome may also contain marker or reporter genes. In an embodiment, an rAAV vector genome can have one or more of the AAV3b wild-type (WT) cis genes replaced or deleted in whole or in part, but retain functional flanking ITR sequences.

In one embodiment, an rAAV vector as disclosed herein useful in the treatment of Pompe Disease comprises a rAAV genome as disclosed herein, encapsulated by an AAV3b capsid. In some embodiments, an rAAV vector as disclosed herein useful in the treatment of Pompe Disease comprises a rAAV genome as disclosed herein, encapsulated by any AAV3b capsid selected from: AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54), or a AAV3bSASTG (i.e., Q263A/T265) capsid.

In some embodiments of the methods and compositions as disclosed herein, the rAAV vector as disclosed herein comprises the nucleic acid sequences of any of: SEQ ID NO: 57 (AAT-V43M-wtGAA (delta1-69aa)); SEQ ID NO: 58 (ratFN1-IGF2V43M-wtGAA (delta1-69aa)); SEQ ID NO: 59 (hFN1-IGF2V43M-wtGAA (delta1-69aa)); SEQ ID NO: 60 (ATT-IGF2Δ2-7-wtGAA (delta 1-69)); SEQ ID NO: 61 (FN1rat-IGFΔ2-7-wtGAA (delta 1-69)); SEQ ID NO: 62 (hFN1-IGFΔ2-7-wtGAA (delta 1-69)), or a nucleic acid sequence having at least 80%, 85%, 90%, 95% or 98% identity thereto. In some embodiments of the methods and compositions as disclosed herein, the rAAV vector comprises a nucleic acid sequence of any of: AAT_hIGF2-V43M_wtGAA_del1-69_Stuffer.V02 (SEQ ID NO: 79); FIBrat_hIGF2-V43M_wtGAA_del1-69_Stuffer.V02 (SEQ ID NO: 80); FIBhum_hIGF2-V43M_wtGAA_del1-69_Stuffer.V02 (SEQ ID NO: 81); AAT_GILT_wtGAA_del1-69_Stuffer.V02 (SEQ ID NO: 82); FIBrat_GILT_wtGAA_del1-69_Stuffer.V02 (SEQ ID NO: 83); FIBhum_GILT_wtGAA_del1-69_Stuffer.V02 (SEQ ID NO: 84) or a nucleic acid sequence having at least 80%, 85%, 90%, 95% or 98% identity thereto.

### IV. Optimized rAAV Vector Genome

In some embodiments of the methods and compositions as disclosed herein, an optimized rAAV vector genome is created from any of the elements disclosed herein and in any combination, including nucleic acid sequences encoding a promoter, an ITR, a poly-A tail, elements capable of increasing or decreasing expression of a heterologous gene, and in one embodiment, a nucleic acid sequence that is codon optimized for expression of GAA protein in vivo (i.e., coGAA) and optionally, one or more element to reduce immunogenicity. Such an optimized rAAV vector genome can be used with any AAV capsid that has tropism for the tissue and cells in which the rAAV vector genome is to be transduced and expressed.

### AAV3b Capsid Modifications

In some embodiments of the methods and compositions as disclosed herein, an AAV3b capsid for use in a rAAV vector as disclosed herein, has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to any of AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54) or a AAV3bSASTG capsid (i.e., a AAV3b capsid comprising Q263A/T265 mutations) disclosed in Nienaber et al., Hum. Gen Ther, 2012, 23(10); 1031-42 and Piacentino III, Valentino, et al. “X-linked inhibitor of apoptosis protein-mediated attenuation of apoptosis, using a novel cardiac-enhanced adeno-associated viral vector.” Human gene therapy 23.6 (2012): 635-646, both of which are incorporated herein in their entirety by reference. In yet other aspects of this embodiment, an AAV derived from AAV3b has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to any of the amino acid sequence for AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54) a AAV3bSASTG capsid (i.e., a AAV3b capsid comprising Q263A/T265 mutations) as disclosed in Nienaber et al., Hum. Gen Ther, 2012, 23(10); 1031-42, and Piacentino III, Valentino, et al. “X-linked inhibitor of apoptosis protein-mediated attenuation of apoptosis, using a novel cardiac-enhanced adeno-associated viral vector.” Human gene therapy 23.6 (2012): 635-646. but the capsid still is a functionally active AAV protein.

In some embodiments of the methods and compositions as disclosed herein, the AAV serotype (e.g. AAV3b) comprises an SASTG mutation as described in Messina E L, et al., Adeno-associated viral vectors based on serotype 3b use components of the fibroblast growth factor receptor signaling complex for efficient transduction. Hum. Gene Ther. 2012 October: 23(10):1031-42, which is incorporated herein in its entirety by reference.

In some embodiments of the methods and compositions as disclosed herein, an AAV3b capsid for use in a rAAV vector as disclosed herein, has, e.g., at least 1, 2, 3, 4, 5, 6, 7, 8, 9, 10, 15, 20, 25, 30, 35, 40, 45, or 50 contiguous amino acid deletions, additions, and/or substitutions relative to any of the amino acid sequence for AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54), or a AAV3bSASTG capsid (i.e., a AAV3b capsid comprising Q263A/T265 mutations) (as disclosed in Nienaber et al., Hum. Gen Ther, 2012, 23(10); 1031-42); or at most 1, 2, 3, 4, 5, 6, 7, 8, 9, 10, 15, 20, 25, 30, 35, 40, 45, or 50 contiguous amino acid deletions, additions, and/or substitutions relative to any of the amino acid sequence for AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54) a AAV3bSASTG capsid (i.e., a AAV3b capsid comprising Q263A/T265 mutations) (as disclosed in Nienaber et al., Hum. Gen Ther, 2012, 23(10); 1031-42). In yet another embodiment, an AAV3b capsid for use in a rAAV vector as disclosed herein, has, e.g., at least 1, 2, 3, 4, 5, 6, 7, 8, 9, 10, 15, 20, 25, 30, 35, 40, 45, or 50 contiguous amino acid deletions, additions, and/or substitutions relative to any of the amino acid sequence for AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54), or a AAV3bSASTG capsid (i.e., a AAV3b capsid comprising Q263A/T265 mutations) (as disclosed in Nienaber et al., Hum. Gen Ther, 2012, 23(10); 1031-42); or at most 1, 2, 3, 4, 5, 6, 7, 8, 9, 10, 15, 20, 25, 30, 35, 40, 45, or 50 contiguous amino acid deletions, additions, and/or substitutions relative to any of the amino acid sequence for AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54), or a AAV3bSASTG capsid (i.e., a AAV3b capsid comprising Q263A/T265 mutations) (as disclosed in Nienaber et al., Hum. Gen Ther, 2012, 23(10); 1031-42), but is still a functionally active AAV.

In some embodiments of the methods and compositions as disclosed herein, an AAV3b capsid for use in a rAAV vector as disclosed herein, has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to any of the amino acid sequence for AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54), a AAV3bSASTG capsid (i.e., a AAV3b capsid comprising Q263A/T265 mutations) (as disclosed in Nienaber et al., Hum. Gen Ther, 2012, 23(10); 1031-42). In yet a further embodiment, an AAV3b capsid for use in a rAAV vector as disclosed herein has an amino acid identity in the range of, e.g., about 75% to about 100%, about 80% to about 100%, about 85% to about 100%, about 90% to about 100%, about 95% to about 100%, about 75% to about 99%, about 80% to about 99%, about 85% to about 99%, about 90% to about 99%, about 95% to about 99%, about 75% to about 97%, about 80% to about 97%, about 85% to about 97%, about 90% to about 97%, or about 95% to about 97%, to any of the amino acid sequence for AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54), a AAV3bSASTG capsid (i.e., a AAV3b capsid comprising Q263A/T265 mutations) (as disclosed in Nienaber et al., Hum. Gen Ther, 2012, 23(10); 1031-42), but is still a functionally active AAV.

### V. Methods of Treatment

### A. Pompe Disease

Pompe disease is a rare genetic disorder caused by a deficiency in the enzyme acid alpha-glucosidase (GAA), which is needed to break down glycogen, a stored form of sugar used for energy. Pompe disease is also known as glycogen storage disease type II, GSD II, type II glycogen storage disease, glycogenosis type II, acid maltase deficiency, alpha-1,4-glucosidase deficiency, cardiomegalia glycogenic diffusa, and cardiac form of generalized glycogenosis. The build-up of glycogen causes progressive muscle weakness (myopathy) throughout the body and affects various body tissues, particularly in the heart, skeletal muscles, liver, respiratory and nervous system.

The presenting clinical manifestations of Pompe disease can vary widely depending on the age of disease onset and residual GAA activity. Residual GAA activity correlates with both the amount and tissue distribution of glycogen accumulation as well as the severity of the disease. Infantile-onset Pompe disease (less than 1% of normal GAA activity) is the most severe form and is characterized by hypotonia, generalized muscle weakness, and hypertrophic cardiomyopathy, and massive glycogen accumulation in cardiac and other muscle tissues. Death usually occurs within one year of birth due to cardiorespiratory failure. Hirschhorn et al. (2001) “Glycogen Storage Disease Type II: Acid Alpha-glucosidase (Acid Maltase) Deficiency,” in Scriver et al., eds., The Metabolic and Molecular Basis of Inherited Disease, 8th Ed., New York: McGraw-Hill, 3389-3420. Juvenile-onset (1-10% of normal GAA activity) and adult-onset (10-40% of normal GAA activity) Pompe disease are more clinically heterogeneous, with greater variation in age of onset, clinical presentation, and disease progression. Juvenile- and adult-onset Pompe disease are generally characterized by lack of severe cardiac involvement, later age of onset, and slower disease progression, but eventual respiratory or limb muscle involvement results in significant morbidity and mortality. While life expectancy can vary, death generally occurs due to respiratory failure. Hirschhorn et al. (2001) “Glycogen Storage Disease Type II: Acid Alpha-glucosidase (Acid Maltase) Deficiency,” in Scriver et al., eds., The Metabolic and Molecular Basis of Inherited Disease, 8th Ed., New York: McGraw-Hill, 3389-3420.

In any embodiment of the methods and compositions as disclosed herein, a GAA enzyme suitable for treating Pompe disease includes a wild-type human GAA, or a fragment or sequence variant thereof which retains the ability to cleave al-4 linkages in linear oligosaccharides. In some embodiments of the methods and compositions as disclosed herein, the GAA protein encoded by a wild type GAA nucleic acid sequence, e.g., SEQ ID NO: 11 or SEQ ID NO: 72. In some embodiments of the methods and compositions as disclosed herein, the GAA protein is encoded by a codon optimized GAA nucleic acid sequence, for example, for any one or more of: (1) enhanced expression in vivo, (2) to reduce CpG islands or (3) reduce the innate immune response. In some embodiments of the methods and compositions as disclosed herein, the GAA protein is encoded by a codon optimized GAA nucleic sequence, for example, any nucleic acid sequence selected from any of: SEQ ID NO: 73, SEQ ID NO: 74, SEQ ID NO: 75 and SEQ ID NO: 76, or a nucleic acid sequence having at least 60%, or 70%, or 80%, 85% or 90% or 95%, or 98%, or 99% sequence identity to SEQ ID NO: 73, SEQ ID NO: 74, SEQ ID NO: 75 and SEQ ID NO: 76.

In some embodiments of the methods and compositions as disclosed herein, a rAAV vector as described herein transduces the liver of a subject and secretes the hGAA polypeptide into the blood, which perfuses patient tissues where the hGAA polypeptide, with the assistance of the fused IGF2-sequence, is taken up by cells and transported to the lysosome, where the enzyme acts to eliminate material that has accumulated in the lysosomes due to the enzyme deficiency. For lysosomal enzyme replacement therapy to be effective, the therapeutic enzyme must be delivered to lysosomes in the appropriate cells in tissues where the storage defect is manifest.

The terms “cation-independent mannose-6-phosphate receptor (CI-MPR),” “M6P/IGF-II receptor,” “CI-MPR/IGF-II receptor,” “IGF-II receptor” or “IGF2 Receptor,” or abbreviations thereof, are used interchangeably herein, referring to the cellular receptor which binds both M6P and IGF-II.

### B. Modulating GAA Levels in a Cell Ex Vivo

The nucleic acids, vector, and virions as described herein can be used to modulate levels of GAA in a cell. The method includes the step of administering to the cell a composition including a nucleic acid that includes a polynucleotide encoding GAA interposed between two AAV ITRs. The cell can be from any animal into which a nucleic acid of the invention can be administered.

Mammalian cells (e.g., humans, dogs, cats, pigs, sheep, mice, rats, rabbits, cattle, goats, etc.) from a subject with GAA deficiency are typical target cells for use in the invention. In some embodiments, the cell is a liver cell or a myocardial cell e.g., a myocardiocyte.

In an embodiment ex vivo delivery of cells transduced with rAAV vector is disclosed herein. In a further embodiment, ex vivo gene delivery may be used to transplant cells transduced with a rAAV vector as disclosed herein back into the host. In a further embodiment, ex vivo stem cell (e.g., mesenchymal stem cell) therapy may be used to transplant cells transduced with a rAAV vector as disclosed herein cells back into the host. In another embodiment, a suitable ex vivo protocol may include several steps.

In some embodiments, a segment of target tissue (e.g., muscle, liver tissue) may be harvested from the subject, and the rAAV vector described herein used to transduce a GAA-encoding nucleic acid into a host's cells. These genetically modified cells may then be transplanted back into the host. Several approaches may be used for the reintroduction of cells into the host, including intravenous injection, intraperitoneal injection, subcutaneous injection, or in situ injection into target tissue. Microencapsulation of modified ex vivo cells transduced or infected with an rAAV vector as described herein is another technique that may be used within the invention. Autologous and allogeneic cell transplantation may be used according to the invention.

In yet another embodiment, disclosed herein is a method of treating a deficiency of GAA in a subject, comprising administering to the subject a cell expressing GAA as disclosed herein, in a pharmaceutically acceptable carrier and in a therapeutically effective amount. In some embodiments, the subject is a human.

### C. Increasing GAA Activity in a Subject

The nucleic acids, vectors, and virions as described herein can be used to modulate levels of functional GAA polypeptide in a subject, e.g., a human subject, or subject with Pompe disease or at risk of having Pompe disease. The method includes administering to the subject a composition comprising the rAAV vector, comprising the rAAV genome as described herein, comprising a heterologous nucleic acid encoding GAA interposed between two AAV ITRs, where the hGAA is linked to a signal peptide as described herein, and optionally a IGF-2 sequence as disclosed herein. The subject can be any animal, e.g., mammals (e.g., human beings, dogs, cats, pigs, sheep, mice, rats, rabbits, cattle, goats, etc.) are suitable subjects. The methods and compositions of the invention are particularly applicable to GAA-deficient human subjects.

Furthermore, the nucleic acids, vectors, and virions described herein may be administered to animals including human beings in any suitable formulation by any suitable method. For example, in any embodiment of the methods and compositions as disclosed herein, an rAAV vector, or rAAV genome as disclosed herein can be directly introduced into an animal, including through administration by oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain) or other parenteral route depending on the desired route of administration and the tissue that is being targeted.

In any embodiment of the methods and compositions as disclosed herein, administration to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles include but are not limited to abductor digiti minimi (in the hand), abductor digiti minimi (in the foot), abductor hallucis, abductor ossis metatarsi quinti, abductor pollicis brevis, abductor pollicis longus, adductor brevis, adductor hallucis, adductor longus, adductor magnus, adductor pollicis, anconeus, anterior scalene, articularis genus, biceps brachii, biceps femoris, brachialis, brachioradialis, buccinator, coracobrachialis, corrugator supercilii, deltoid, depressor anguli oris, depressor labii inferioris, digastric, dorsal interossei (in the hand), dorsal interossei (in the foot), extensor carpi radialis brevis, extensor carpi radialis longus, extensor carpi ulnaris, extensor digiti minimi, extensor digitorum, extensor digitorum brevis, extensor digitorum longus, extensor hallucis brevis, extensor hallucis longus, extensor indicis, extensor pollicis brevis, extensor pollicis longus, flexor carpi radialis, flexor carpi ulnaris, flexor digiti minimi brevis (in the hand), flexor digiti minimi brevis (in the foot), flexor digitorum brevis, flexor digitorum longus, flexor digitorum profundus, flexor digitorum superficialis, flexor hallucis brevis, flexor hallucis longus, flexor pollicis brevis, flexor pollicis longus, frontalis, gastrocnemius, geniohyoid, gluteus maximus, gluteus medius, gluteus minimus, gracilis, iliocostalis cervicis, iliocostalis lumborum, iliocostalis thoracis, illiacus, inferior gemellus, inferior oblique, inferior rectus, infraspinatus, interspinalis, intertransversi, lateral pterygoid, lateral rectus, latissimus dorsi, levator anguli oris, levator labii superioris, levator labii superioris alaeque nasi, levator palpebrae superioris, levator scapulae, long rotators, longissimus capitis, longissimus cervicis, longissimus thoracis, longus capitis, longus colli, lumbricals (in the hand), lumbricals (in the foot), masseter, medial pterygoid, medial rectus, middle scalene, multifidus, mylohyoid, obliquus capitis inferior, obliquus capitis superior, obturator externus, obturator internus, occipitalis, omohyoid, opponens digiti minimi, opponens pollicis, orbicularis oculi, orbicularis oris, palmar interossei, palmaris brevis, palmaris longus, pectineus, pectoral's major, pectoral's minor, peroneus brevis, peroneus longus, peroneus tertius, piriformis, plantar interossei, plantaris, platysma, popliteus, posterior scalene, pronator quadratus, pronator teres, psoas major, quadratus femoris, quadratus plantae, rectus capitis anterior, rectus capitis lateralis, rectus capitis posterior major, rectus capitis posterior minor, rectus femoris, rhomboid major, rhomboid minor, risorius, sartorius, scalenus minimus, semimembranosus, semispinalis capitis, semispinalis cervicis, semispinalis thoracis, semitendinosus, serratus anterior, short rotators, soleus, spinalis capitis, spinalis cervicis, spinalis thoracis, splenius capitis, splenius cervicis, sternocleidomastoid, sternohyoid, sternothyroid, stylohyoid, subclavius, subscapularis, superior gemellus, superior oblique, superior rectus, supinator, supraspinatus, temporalis, tensor fascia lata, teres major, teres minor, thoracis, thyrohyoid, tibialis anterior, tibialis posterior, trapezius, triceps brachii, vastus intermedius, vastus lateralis, vastus medialis, zygomaticus major, and zygomaticus minor, and any other suitable skeletal muscle as known in the art.

In any embodiment of the methods and compositions as disclosed herein, administration to cardiac muscle includes administration to the left atrium, right atrium, left ventricle, right ventricle and/or septum. The virus vector and/or capsid can be delivered to cardiac muscle by intravenous administration, intra-arterial administration such as intra-aortic administration, direct cardiac injection (e.g., into left atrium, right atrium, left ventricle, right ventricle), and/or coronary artery perfusion.

In any embodiment of the methods and compositions as disclosed herein, administration to a diaphragm muscle can be by any suitable method including intravenous administration, intra-arterial administration, and/or intra-peritoneal administration.

In any embodiment of the methods and compositions as disclosed herein, the rAAV vectors and/or rAAV genome as disclosed herein are administered to the skeletal muscle, liver, diaphragm, costal, and/or cardiac muscle cells of a subject. For example, a conventional syringe and needle can be used to inject a rAAV virion suspension into an animal. Parenteral administration of a the rAAV vectors and/or rAAV genome, by injection can be performed, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain agents for a pharmaceutical formulation, such as suspending, stabilizing and/or dispersing agents. Alternatively, the rAAV vectors and/or rAAV genome as disclosed herein can be in powder form (e.g., lyophilized) for constitution with a suitable vehicle, for example, sterile pyrogen-free water, before use.

In particular embodiments, more than one administration (e.g., two, three, four, five, six, seven, eight, nine, 10, etc., or more administrations) may be employed to achieve the desired level of gene expression over a period of various intervals, e.g., hourly, daily, weekly, monthly, yearly, etc. Dosing can be single dosage or cumulative (serial dosing), and can be readily determined by one skilled in the art. For instance, treatment of a disease or disorder may comprise a one-time administration of an effective dose of a pharmaceutical composition virus vector disclosed herein. Alternatively, treatment of a disease or disorder may comprise multiple administrations of an effective dose of a virus vector carried out over a range of time periods, such as, e.g., once daily, twice daily, trice daily, once every few days, or once weekly.

The timing of administration can vary from individual to individual, depending upon such factors as the severity of an individual's symptoms. For example, an effective dose of a virus vector disclosed herein can be administered to an individual once every six months for an indefinite period of time, or until the individual no longer requires therapy. A person of ordinary skill in the art will recognize that the condition of the individual can be monitored throughout the course of treatment and that the effective amount of a virus vector disclosed herein that is administered can be adjusted accordingly.

Injectables can be prepared in conventional forms, either as liquid solutions or suspensions, solid forms suitable for solution or suspension in liquid prior to injection, or as emulsions. Alternatively, one may administer the virus vector and/or virus capsids of the invention in a local rather than systemic manner, for example, in a depot or sustained-release formulation. Further, the virus vector and/or virus capsid can be delivered adhered to a surgically implantable matrix (e.g., as described in U.S. Patent Publication No. US-2004-0013645-A1). The virus vectors and/or virus capsids disclosed herein can be administered to the lungs of a subject by any suitable means, optionally by administering an aerosol suspension of respirable particles comprised of the virus vectors and/or virus capsids, which the subject inhales. The respirable particles can be liquid or solid. Aerosols of liquid particles comprising the virus vectors and/or virus capsids may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the virus vectors and/or capsids may likewise be produced with any solid particulate medicament aerosol generator, by techniques known in the pharmaceutical art.

In some embodiments, the rAAV vectors and/or rAAV genome as disclosed herein can be formulated in a solvent, emulsion or other diluent in an amount sufficient to dissolve an rAAV vector disclosed herein. In other aspects of this embodiment, the rAAV vectors and/or rAAV genome as disclosed herein can herein may be formulated in a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects, the rAAV vectors and/or rAAV genome as disclosed herein can disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

In any embodiment of the methods and compositions as disclosed herein, the rAAV vectors and/or rAAV genome as disclosed herein, of any serotype, including but not limited to encapsulated by any AAV3b capsid selected from: AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54) or AAV3bSASTG capsid (i.e., a AAV3b capsid comprising Q263A/T265 mutations). can comprise a therapeutic compound in a therapeutically effective amount. In an embodiment, as used herein, without limitation, the term “effective amount” is synonymous with “therapeutically effective amount”, “effective dose”, or “therapeutically effective dose.” In an embodiment, the effectiveness of a therapeutic compound disclosed herein to treat Pompe Disease can be determined, without limitation, by observing an improvement in an individual based upon one or more clinical symptoms, and/or physiological indicators associated with Pompe Disease. In an embodiment, an improvement in the symptoms associated with Pompe Disease can be indicated by a reduced need for a concurrent therapy.

Exemplary modes of administration include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). Administration can also be to a tumor (e.g., in or near a tumor or a lymph node). The most suitable route in any given case will depend on the nature and severity of the condition being treated and/or prevented and on the nature of the particular vector that is being used.

To facilitate delivery of a rAAV vector and/or rAAV genome as disclosed herein, it can be mixed with a carrier or excipient. Carriers and excipients that might be used include saline (especially sterilized, pyrogen-free saline) saline buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of virions to human subjects.

In addition to the formulations described previously, a rAAV vector and/or rAAV genome as disclosed herein can also be formulated as a depot preparation. Such long acting formulations may be administered by implantation (for example subcutaneously or intramuscularly) or by IM injection. Thus, for example, a rAAV vector and/or rAAV genome as disclosed herein may be formulated with suitable polymeric or hydrophobic materials (for example as an emulsion in an acceptable oil) or ion exchange resins, or as sparingly soluble derivatives.

In any embodiment of the methods and compositions as disclosed herein, the method is directed to treating Pompe Disease that results from a deficiency of GAA in a subject, wherein a rAAV vector and/or rAAV genome as disclosed herein is administered to a patient suffering from Pompe Disease, and following administration, GAA is secreted from cells in the liver and there is uptake of the secreted GAA by cells in skeletal muscle tissue, cardiac muscle tissue, diaphragm muscle tissue or a combination thereof, wherein uptake of the secreted GAA results in a reduction in lysosomal glycogen stores in the tissue(s). In some embodiments, the rAAV vector and/or rAAV genome as disclosed herein is encapsulated in a capsid, e.g., encapsulated by any AAV3b capsid selected from: AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54).

In a particular embodiment, at least about 10² to about 10⁸ cells or at least about 10³ to about 10⁶ cells will be administered per dose in a pharmaceutically acceptable carrier. In a further embodiment, dosages of the virus vector and/or capsid to be administered to a subject depend upon the mode of administration, the disease or condition to be treated and/or prevented, the individual subject's condition, the particular virus vector or capsid, the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10³, 10¹⁴, 10¹⁵ transducing units, optionally about 10⁸-10¹³ transducing units.

In another aspect, disclosed herein is a method of administering a nucleic acid encoding a GAA to a cell, comprising contacting the cell with a rAAV vector and/or rAAV genome as disclosed herein, under conditions for the nucleic acid to be introduced into the cell and expressed to produce GAA. In some embodiments, the cell is a cultured cell. In some embodiments, the cell is a cell in vivo. In some embodiments, the cell is a mammalian cell. In some embodiments, method of administering a nucleic acid encoding a GAA to a cell further comprises collecting the GAA secreted into a cell culture medium.

### D. Increasing Motoneuron Function in a Mammal

In any embodiment of the methods and compositions as disclosed herein, a rAAV vector and/or rAAV genome as disclosed herein is useful in compositions and methods to increase phrenic nerve activity in a mammal having Pompe disease and/or insufficient GAA levels. For example, a rAAV vector and/or rAAV genome as disclosed herein, e.g., a rAAV vector and/or rAAV genome encapsulated in a capsid, e.g., encapsulated by any AAV3b capsid selected from: AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54), can be administered to the central nervous system (e.g., neurons). In another embodiment, retrograde transport of rAAV vector and/or rAAV genome as disclosed herein encoding GAA from the diaphragm (or other muscle) to the phrenic nerve or other motor neurons can result in biochemical and physiological correction of Pompe disease. These same principles could be applied to other neurodegenerative disease.

In an embodiment, a rAAV GAA construct of any serotype as described in Table 1, including AAV8 or AAV3, or AAV3b (including but not limited to AAV3b serotypes AAV3b265D, AAV3b265D549A, AAV3b549A, AAV3bQ263Y, AAV3bSASTG (i.e., a AAV3b capsid comprising Q263A/T265 mutations) serotypes) is capable of reducing the feeling of weakness in a patient's lower extremities, including, the legs, trunk and/or arms in a patient suffering from Pompe Disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, an AAV GAA of any serotype is capable of reducing the feeling of weakness in a patient's lower extremities, including, the legs, trunk and/or arms in a patient suffering from Pompe Disease by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In any embodiment of the methods and compositions as disclosed herein, a rAAV GAA construct of any serotype as described in Table 1, including AAV8 or AAV3b (including but not limited to AAV3b serotypes AAV3b265D, AAV3b265D549A, AAV3b549A, AAV3bQ263Y and AAV3bSASTG (i.e., a AAV3b capsid comprising Q263A/T265 mutations)) as disclosed herein is capable of reducing one or more of the following in a patient suffering from Pompe Disease: a shortness of breath, a hard time exercising, lung infections, a big curve in the spine, trouble breathing while sleeping, an enlarged liver, an enlarged tongue and/or a stiff joint by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of the methods and compositions of this embodiment, an AAV3bQ263Y GAA disclosed herein is capable of reducing one or more of the following in a patient suffering from Pompe Disease: a shortness of breath, a hard time exercising, lung infections, a big curve in the spine, trouble breathing while sleeping, an enlarged liver, an enlarged tongue and/or a stiff joint by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In any embodiment of the methods and compositions as disclosed herein, a rAAV vector and/or rAAV genome as disclosed herein of any serotype disclosed herein is capable of reducing one or more of the following in a patient suffering from Pompe Disease: a shortness of breath, a hard time exercising, lung infections, a big curve in the spine, trouble breathing while sleeping, an enlarged liver, an enlarged tongue and/or a stiff joint by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, rAAV vector and/or rAAV genome as disclosed herein of any serotype is capable of reducing one or more of the following in a patient suffering from Pompe Disease: a shortness of breath, a hard time exercising, lung infections, a big curve in the spine, trouble breathing while sleeping, an enlarged liver, an enlarged tongue and/or a stiff joint by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In any embodiment of the methods and compositions as disclosed herein, the symptoms associated with Pompe Disease are reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the severity of the symptoms associated with Pompe Disease are reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In another embodiment, the symptoms associated with Pompe Disease are reduced by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In an embodiment, the adverse effects associated with Pompe Disease are reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the severity of the adverse effects associated with Pompe Disease are reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In another embodiment, the adverse effects associated with Pompe Disease are reduced by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

### D. Mouse Models

### E. Immunosuppression

In any embodiment of the methods and compositions as disclosed herein, a subject being administered a rAAV vector or rAAV genome as disclosed herein is administered an immunosuppressive agent. Various methods are known to result in the immunosuppression of an immune response of a patient being administered AAV. Methods known in the art include administering to the patient an immunosuppressive agent, such as a proteasome inhibitor. One such proteasome inhibitor known in the art, for instance as disclosed in U.S. Pat. No. 9,169,492 and U.S. patent application Ser. No. 15/796,137, both of which are incorporated herein by reference, is bortezomib. In another embodiment, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors β1 and β2, TNF and others that are publicly known).

### V. Administration

Dosages of the a rAAV vector or rAAV genome as disclosed herein to be administered to a subject depend upon the mode of administration, the disease or condition to be treated and/or prevented, the individual subject's condition, the particular virus vector or capsid, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10¹³, 10¹⁴, 10¹⁵ transducing units, optionally about 10⁸ to about 10¹³ transducing units.

In a further embodiment, administration of rAAV vector or rAAV genome as disclosed herein to a subject results in production of a GAA protein with a circulatory half-life of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a rAAV vector or rAAV genome as disclosed herein to a subject is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In another embodiment, administration of a rAAV vector or rAAV genome as disclosed herein for the treatment of Pompe Disease results in an increase in weight by, e.g., at least 0.5 pounds, at least 1 pound, at least 1.5 pounds, at least 2 pounds, at least 2.5 pounds, at least 3 pounds, at least 3.5 pounds, at least 4 pounds, at least 4.5 pounds, at least 5 pounds, at least 5.5 pounds, at least 6 pounds, at least 6.5 pounds, at least 7 pounds, at least 7.5 pounds, at least 8 pounds, at least 8.5 pounds, at least 9 pounds, at least 9.5 pounds, at least 10 pounds, at least 10.5 pounds, at least 11 pounds, at least 11.5 pounds, at least 12 pounds, at least 12.5 pounds, at least 13 pounds, at least 13.5 pounds, at least 14 pounds, at least 14.5 pounds, at least 15 pounds, at least 20 pounds, at least 25 pounds, at least 30 pounds, at least 50 pounds. In another embodiment, an AAV GAA of any serotype, as disclosed herein for the treatment of Pompe Disease results in an increase in weight by, e.g., from 0.5 pounds to 50 pounds, from 0.5 pounds to 30 pounds, from 0.5 pounds to 25 pounds, from 0.5 pounds to 20 pounds, from 0.5 pounds to 15 pounds, from 0.5 pounds to ten pounds, from 0.5 pounds to 7.5 pounds, from 0.5 pounds to 5 pounds, from 1 pound to 15 pounds, from 1 pound to 10 pounds, from 1 pound to 7.5 pounds, form 1 pound to 5 pounds, from 2 pounds to ten pounds, from 2 pounds to 7.5 pounds.

All aspects of the compositions and methods of the technology disclosed herein can be defined in any one or more of the following numbered paragraphs:
1. A recombinant adenovirus associated (AAV) vector comprising in its genome:
   a. 5′ and 3′ AAV inverted terminal repeats (ITR) sequences, and
   b. located between the 5′ and 3′ ITRs, a heterologous nucleic acid sequence encoding a fusion polypeptide comprising a secretory signal peptide and an alpha-glucosidase (GAA) polypeptide, wherein the heterologous nucleic acid is operatively linked to a promoter.
2. The recombinant AAV vector of paragraph 1, wherein the heterologous nucleic acid sequence encoding a fusion polypeptide further comprises a IGF-2 sequence located between the secretory signal peptide and the alpha-glucosidase (GAA) polypeptide.
3. The recombinant AAV vector of paragraph 1 or 2, wherein the AAV genome comprises, in the 5′ to 3′ direction:
   a. a 5′ ITR,
   b. a promoter sequence,
   c. an intron sequence,
   d. a nucleic acid encoding a secretory signal peptide,
   e. a nucleic acid encoding an IGF-2 sequence,
   f. a nucleic acid encoding an alpha-glucosidase (GAA) polypeptide,
   g. a poly A sequence, and
   h. a 3′ ITR.
4. The recombinant AAV vector of any of paragraphs 1-3, wherein the secretory signal peptide is selected from an AAT signal peptide, a fibronectin signal peptide (FN), a GAA signal peptide, or an active fragment thereof having secretory signal activity.
5. The recombinant AAV vector of any of paragraphs 1-3, wherein the IGF-2 leader sequence binds human cation-independent mannose-6-phosphate receptor (CI-MPR) or the IGF-2 receptor.
6. The recombinant AAV vector of any of paragraphs 1-5, wherein the IGF-2 sequence comprises SEQ ID NO: 5 or comprises at least one amino modification in SEQ ID NO: 5 that binds to the IGF-2 receptor.
7. The recombinant AAV vector of any of paragraphs 1-6, wherein the at least one amino modification in SEQ ID NO: 5 is a V43M amino acid modification (SEQ ID NO: 8 or SEQ ID NO: 9) or 42-7 (SEQ ID NO: 6) or 41-7 (SEQ ID NO: 7).
8. The recombinant AAV vector of any of paragraphs 1-7, wherein the promoter is constitutive, cell specific or inducible.
9. The recombinant AAV vector of any of paragraphs 1-8, wherein the promoter is a liver-specific promoter.
10. The recombinant AAV vector of any of paragraphs 1-9, wherein the liver specific promoter is selected from any of: transthyretin promoter (TTR), LSP promoter (LSP), a synthetic liver specific promoter.
11. The recombinant AAV vector of any of paragraphs 1-10, wherein the nucleic acid sequence encodes a wild-type GAA polypeptide or a modified GAA polypeptide.
12. The recombinant AAV vector of any of paragraphs 1-11, wherein the nucleic acid sequence encoding the GAA polypeptide is the human GAA gene or a human codon optimized GAA gene (coGAA) or a modified GAA nucleic acid sequence.
13. The recombinant AAV vector of any of paragraphs 1-12, wherein the nucleic acid sequence encoding the GAA polypeptide is codon optimized for enhanced expression in vivo.
14. The recombinant AAV vector of any of paragraphs 1-13, wherein the nucleic acid sequence encoding the GAA polypeptide is codon optimized to reduce CpG islands.
15. The recombinant AAV vector of any of paragraphs 1-14, wherein the nucleic acid sequence encoding the GAA polypeptide is codon optimized to reduce the innate immune response.
16. The recombinant AAV vector of any of paragraphs 1-15, wherein the nucleic acid sequence encoding the GAA polypeptide is codon optimized to reduce CpG islands and reduce the innate immune response.
17. The recombinant AAV vector of any of paragraphs 1-16, wherein the encoded fusion polypeptide further comprising a spacer comprising a nucleotide sequence for at least 1 amino acids located amino-terminal to the GAA polypeptide, and C-terminal to the IGF-2 sequence.
18. The recombinant AAV vector of any of paragraphs 1-7, further comprising a nucleic acid encoding a spacer of at least 1 amino acids located between the nucleic acid encoding the IGF-2 sequence and the nucleic acid encoding the GAA polypeptide.
19. The recombinant AAV vector of any of paragraphs 1-8, further comprising at least one polyA sequence located 3′ of the nucleic acid encoding the GAA gene and 5′ of the 3′ ITR sequence.
20. The recombinant AAV vector of any of paragraphs 1-19, wherein the heterologous nucleic acid sequence further comprises at collagen stability (CS) sequence located 3′ of the nucleic acid encoding the GAA polypeptide and 5′ of the 3′ ITR sequence.
21. The recombinant AAV vector of any of paragraphs 1-20, further comprising a nucleic acid encoding a collagen stability (CS) sequence located between the nucleic acid encoding the GAA polypeptide and the poly A sequence
22. The recombinant AAV vector of any of paragraphs 1-21, further comprising an intron sequence located 5′ of the sequence encoding the secretory signal peptide, and 3′ of the promoter.
23. The recombinant AAV vector of any of paragraphs 1-22, wherein the intron sequence comprises a MVM sequence or a HBB2 sequence, wherein the MVN sequence comprises the nucleic acid sequence of SEQ ID NO: 13, or a nucleic acid sequence at least about 75%, or 80%, or 85%, or 90%, or 95%, or 98%, or 99% sequence identity to SEQ ID NO: 13, and the HBB2 sequence comprises the nucleic acid sequence of SEQ ID NO: 14, or a nucleic acid sequence at least about 75%, or 80%, or 85%, or 90%, or 95%, or 98%, or 99% sequence identity to SEQ ID NO: 14.
24. The recombinant AAV vector of any of paragraphs 1-23, wherein the ITR comprises an insertion, deletion or substitution.
25. The recombinant AAV vector of any of paragraphs 1-24, wherein one or more CpG islands in the ITR are removed.
26. The recombinant AAV vector of any of paragraphs 1-25, wherein the secretory signal peptide is a fibronectin signal peptide (FN1) or an active fragment thereof having secretory signal activity (e.g., a FN1 signal peptide has the sequence of any of SEQ ID NO: 18-21, or an amino acid sequence at having at least about 75%, or 80%, or 85%, or 90%, or 95%, or 98%, or 99% sequence identity to any of SEQ ID NOs: 18-21), and the heterologous nucleic acid sequence encodes a IGF-2 sequence selected from any of: SEQ ID NO: 5, SEQ ID NO: 6, SEQ ID NO: 7, SEQ ID NO: 8 or SEQ ID NO: 9, or a IGF2 peptide having at least about 75%, or 80%, or 85%, or 90%, or 95%, or 98%, or 99% sequence identity to SEQ ID NOs: 5-9.
27. The recombinant AAV vector of any of paragraphs 1-3, wherein the encoded secretory signal peptide is AAT signal peptide or an active fragment thereof having secretory signal activity, (e.g., a AAT signal peptide has the sequence of SEQ ID NO: 17, or an amino acid sequence at having at least about 75%, or 80%, or 85%, or 90%, or 95%, or 98%, or 99% sequence identity to SEQ ID NO: 17), and the heterologous nucleic acid sequence encodes a IGF-2 sequence selected from any of: SEQ ID NO: 5, SEQ ID NO: 6, SEQ ID NO: 7, SEQ ID NO: 8 or SEQ ID NO: 9, or a IGF2 peptide having at least about 75%, or 80%, or 85%, or 90%, or 95%, or 98%, or 99% sequence identity to SEQ ID NOs: 5-9.
28. The recombinant AAV vector of any of paragraphs 1-27, wherein the IGF-2 sequence is SEQ ID NO: 8 or SEQ ID NO: 9, or a IGF2 peptide having at least about 75%, or 80%, or 85%, or 90%, or 95%, or 98%, or 99% sequence identity to SEQ ID NO: 8 or 9.
29. The recombinant AAV vector of any of paragraphs 1-28, wherein the recombinant AAV vector is a chimeric AAV vector, haploid AAV vector, a hybrid AAV vector or polyploid AAV vector.
30. The recombinant AAV vector of any of paragraphs 1-29, wherein the recombinant AAV vector comprises a capsid protein selected from any AAV serotype in the group consisting of those listed in Table 1 and any combination thereof
31. The recombinant AAV vector of any of paragraphs 1-30, wherein the serotype is AAV3b.
32. The recombinant AAV vector of any of paragraphs 1-31, wherein the AAV3b serotype comprises one or mutations in a capsid protein selected from any of: 265D, 549A, Q263Y
33. The recombinant AAV vector of any of paragraphs 1-32, wherein the AAV3b serotype is selected from any of: AAV3b265D, AAV3b265D549A, AAV3b549A or AAV3bQ263Y, or AAV3bSASTG.
34. A recombinant adenovirus associated (AAV) vector comprising in its genome:
   a. 5′ and 3′ AAV inverted terminal repeats (ITR) sequences, and
   b. located between the 5′ and 3′ ITRs, a heterologous nucleic acid sequence encoding a fusion polypeptide comprising an alpha-glucosidase (GAA) polypeptide, wherein the heterologous nucleic acid is operatively linked to a liver specific promoter. wherein the recombinant AAV vector comprises a capsid protein of the AAV3b serotype.
35. The recombinant AAV vector of paragraph 34, wherein the fusion polypeptide further comprises a secretory signal peptide located at the N-terminal of the GAA polypeptide.
36. The recombinant AAV vector of paragraph 34 or 35, wherein the heterologous nucleic acid sequence encoding a fusion polypeptide further comprises a IGF-2 sequence located between the secretory signal peptide and the an alpha-glucosidase (GAA) polypeptide.
37. The recombinant AAV vector of paragraph 34, wherein the AAV genome comprises, in the 5′ to 3′ direction:
   a. a 5′ ITR,
   b. a liver specific promoter sequence,
   c. an intron sequence,
   d. a nucleic acid encoding a secretory signal peptide,
   e. a nucleic acid encoding an IGF-2 sequence,
   f. a nucleic acid encoding an alpha-glucosidase (GAA) polypeptide,
   g. a poly A sequence, and
   h. a 3′ ITR.
38. The recombinant AAV vector of any of paragraphs 34-37, wherein the secretory signal peptide is selected from an AAT signal peptide, a fibronectin signal peptide (FN), a GAA signal peptide, or an active fragment thereof having secretory signal activity.
39. The recombinant AAV vector of any of paragraphs 34-38, wherein the IGF-2 leader sequence binds human cation-independent mannose-6-phosphate receptor (CI-MPR) or the IGF-2 receptor.
40. The recombinant AAV vector of any of paragraphs 34-39, wherein the IGF-2 sequence comprises SEQ ID NO: 5 or comprises at least one amino modification in SEQ ID NO: 5 that affects binding to the IGF-2 receptor.
41. The recombinant AAV vector of paragraph 40, wherein the at least one amino modification in SEQ ID NO: 5 is a V43M amino acid modification (SEQ ID NO: 8 or SEQ ID NO: 9) or 42-7 (SEQ ID NO: 6) or 41-7 (SEQ ID NO: 7).
42. The recombinant AAV vector of any of paragraphs 34-41, wherein the liver specific promoter is selected from any of: transthyretin promoter (TTR), LSP promoter (LSP), a synthetic liver specific promoter.
43. The recombinant AAV vector of paragraphs 34-42, wherein the nucleic acid sequence encodes a wild-type GAA polypeptide or a modified GAA polypeptide.
44. The recombinant AAV vector of any of paragraphs 34-43, wherein the nucleic acid sequence encoding the GAA polypeptide is the human GAA gene or a human codon optimized GAA gene (coGAA) or a modified GAA nucleic acid sequence.
45. The recombinant AAV vector of any of paragraphs 34-44, wherein the nucleic acid sequence encoding the GAA polypeptide is codon optimized for enhanced expression in vivo.
46. The recombinant AAV vector of any of paragraphs 34-44, wherein the nucleic acid sequence encoding the GAA polypeptide is codon optimized to reduce CpG islands.
47. The recombinant AAV vector of any of paragraphs 34-44, wherein the nucleic acid sequence encoding the GAA polypeptide is codon optimized to reduce the innate immune response.
48. The recombinant AAV vector of any of paragraphs 34-44, wherein the nucleic acid sequence encoding the GAA polypeptide is codon optimized to reduce CpG islands and reduce the innate immune response.
49. The recombinant AAV vector any of paragraphs 34-49, wherein the intron sequence comprises a MVM sequence or a HBB2 sequence, wherein the MVN sequence comprises the nucleic acid sequence of SEQ ID NO: 13, or a nucleic acid sequence at least about 75%, or 80%, or 85%, or 90%, or 95%, or 98%, or 99% sequence identity to SEQ ID NO: 13, and the HBB2 sequence comprises the nucleic acid sequence of SEQ ID NO: 14, or a nucleic acid sequence at least about 75%, or 80%, or 85%, or 90%, or 95%, or 98%, or 99% sequence identity to SEQ ID NO: 14.
50. The recombinant AAV vector any of paragraphs 34-49, wherein the ITR comprises an insertion, deletion or substitution.
51. The recombinant AAV vector of paragraph 40, wherein one or more CpG islands in the ITR are removed.
52. The recombinant AAV vector of any of paragraphs 34-49, wherein the secretory signal peptide is a fibronectin signal peptide (FN1) or an active fragment thereof having secretory signal activity, (e.g., a FN1 signal peptide has the sequence of any of SEQ ID NO: 18-21, or an amino acid sequence at having at least about 75%, or 80%, or 85%, or 90%, or 95%, or 98%, or 99% sequence identity to any of SEQ ID NOs: 18-21), and the heterologous nucleic acid sequence encodes a IGF-2 sequence selected from any of: SEQ ID NO: 5, SEQ ID NO: 6, SEQ ID NO: 7, SEQ ID NO: 8 or SEQ ID NO: 9, or a IGF2 peptide having at least about 75%, or 80%, or 85%, or 90%, or 95%, or 98%, or 99% sequence identity to SEQ ID NOs: 5-9.
53. The recombinant AAV vector of any of paragraphs 34-49, wherein the encoded secretory signal peptide is AAT signal peptide or an active fragment thereof having secretory signal activity, (e.g., a AAT signal peptide has the sequence of SEQ ID NO: 17, or an amino acid sequence at having at least about 75%, or 80%, or 85%, or 90%, or 95%, or 98%, or 99% sequence identity to SEQ ID NO: 17), and the heterologous nucleic acid sequence encodes a IGF-2 sequence selected from any of: SEQ ID NO: 5, SEQ ID NO: 6, SEQ ID NO: 7, SEQ ID NO: 8 or SEQ ID NO: 9, or a IGF2 peptide having at least about 75%, or 80%, or 85%, or 90%, or 95%, or 98%, or 99% sequence identity to SEQ ID NOs: 5-9.
54. The recombinant AAV vector of any of paragraphs 34-49, wherein the IGF-2 sequence is SEQ ID NO: 8 or SEQ ID NO: 9, or a IGF2 peptide having at least about 75%, or 80%, or 85%, or 90%, or 95%, or 98%, or 99% sequence identity to SEQ ID NO: 8 or 9.
55. A pharmaceutical composition comprising the recombinant AAV vector of any one of the previous paragraphs in a pharmaceutically acceptable carrier.
56. A nucleic acid sequence comprising: a liver specific promoter operatively linked to a nucleic acid sequence comprising, in the following order: a nucleic acid encoding a secretory signal peptide, a nucleic acid encoding a IGF-2 sequence, a nucleic acid encoding a GAA polypeptide.
57. A nucleic acid sequence for a recombinant adenovirus associated (rAAV) vector genome comprising:
   a. 5′ and 3′ AAV inverted terminal repeats (ITR) nucleic acid sequences, and
   b. located between the 5′ and 3′ ITR sequence, a heterologous nucleic acid sequence encoding a fusion polypeptide comprising a secretory signal peptide and an alpha-glucosidase (GAA) polypeptide, wherein the heterologous nucleic acid is operatively linked to a promoter.
58. The nucleic acid sequence of paragraph 56 or 57, wherein the heterologous nucleic acid sequence encoding a fusion polypeptide further comprises a IGF-2 sequence located between the secretory signal peptide and the an alpha-glucosidase (GAA) polypeptide.
59. The nucleic acid sequence of paragraph 56 or 58 wherein the nucleic acid encoding the secretory signal is selected from any of SEQ ID NO: 17, 22-26, or a nucleic acid sequence at least about 75%, or 80%, or 85%, or 90%, or 95%, or 98%, or 99% sequence identity to any of SEQ ID NOs: 17 or 22-26.
60. The nucleic acid sequence of any of paragraphs 56-59, wherein the nucleic acid encoding the IGF-2 sequence is selected from any of SEQ ID NO: 2 (IGF2-Δ2-7), SEQ ID NO: 3 (IGF2-41-7), or SEQ ID NO: 4 (IGF2 V43M), or a nucleic acid sequence at least about 75%, or 80%, or 85%, or 90%, or 95%, or 98%, or 99% sequence identity to any of SEQ ID NOs: 2, 3 or 4.
61. The nucleic acid sequence of any of paragraphs 56-60, wherein the nucleic acid sequence encoding the GAA polypeptide is the human GAA gene or a human codon optimized GAA gene (coGAA) or a modified GAA nucleic acid sequence.
62. The nucleic acid sequence of any of paragraphs 56-61, wherein the nucleic acid sequence encoding the GAA polypeptide is codon optimized for enhanced expression in vivo.
63. The nucleic acid sequence of any of paragraphs 56-62, wherein the nucleic acid sequence encoding the GAA polypeptide is codon optimized to reduce CpG islands.
64. The nucleic acid sequence of any of paragraphs 56-63, wherein the nucleic acid sequence encoding the GAA polypeptide is codon optimized to reduce the innate immune response.
65. The nucleic acid sequence of any of paragraphs 56-64, wherein the nucleic acid sequence encoding the GAA polypeptide is codon optimized to reduce CpG islands and reduce the innate immune response.
66. The nucleic acid sequence of any of paragraphs 56-65, wherein the nucleic acid encoding the GAA polypeptide is selected from any of SEQ ID NO: 11 (full length hGAA), SEQ ID NO: 55 (Dwight cDNA), SEQ ID NO: 56 (hGAA Δ1-66) or a nucleic acid sequence at least about 75%, or 80%, or 85%, or 90%, or 95%, or 98%, or 99% sequence identity to any of SEQ ID NOs: 11, 55 or 56.
67. The nucleic acid sequence of paragraph 56 or 57, wherein the nucleic acid encoding the GAA polypeptide is selected from any of SEQ ID NO: 74 (codon optimized 1), SEQ ID NO: 75 (codon optimized 2), and SEQ ID NO: 76 (codon optimized 3), or a nucleic acid sequence at least about 75%, or 80%, or 85%, or 90%, or 95%, or 98%, or 99% sequence identity to any of SEQ ID NOs: 74, 75 or 76.
68. The nucleic acid sequence of paragraph 56 or 57, wherein the nucleic acid is selected from any of: SEQ ID NO: 57 (AAT-V43M-wtGAA (delta1-69aa)); SEQ ID NO: 58 (ratFN1-IGF2V43M-wtGAA (delta1-69aa)); SEQ ID NO: 59 (hFN1-IGF2V43M-wtGAA (delta1-69aa)); SEQ ID NO: 60 (ATT-IGF2Δ2-7-wtGAA (delta 1-69)); SEQ ID NO: 61 (FN1rat-IGFΔ2-7-wtGAA (delta 1-69)); SEQ ID NO: 62 (hFN1-IGFΔ2-7-wtGAA (delta 1-69)), SEQ ID NO: 79 (AAT_hIGF2-V43M_wtGAA_del1-69_Stuffer.V02); SEQ ID NO: 80 (FIBrat_hIGF2-V43M_wtGAA_del1-69_Stuffer.V02); SEQ ID NO: 81 (FIBhum_hIGF2-V43M_wtGAA_del1-69_Stuffer.V02); SEQ ID NO: 82 (AAT_GILT_wtGAA_del1-Stuffer.V02); SEQ ID NO: 83 (FIBrat_GILT_wtGAA_del1-69_Stuffer.V02); SEQ ID NO: 84 (FIBhum_GILT_wtGAA_del1-69_Stuffer.V02) or a nucleic acid sequence having at least 80%, 85%, 90%, 95% or 98% identity to SEQ ID Nos: 57, 58, 59, 60, 61, 62, 79, 80, 81, 82, 83 or 84.
69. A method to treat a subject with a glycogen storage disease type II (GSD II, Pompe Disease, Acid Maltase Deficiency) or having a deficiency in alpha-glucosidase (GAA) polypeptide, comprising administering any of the recombinant AAV vector, or the rAAV genome or the nucleic acid sequence of any one of the previous paragraphs 1-58 to the subject.
70. The method of paragraph 69, wherein GAA polypeptide is secreted from the subject's liver and there is uptake of the secreted GAA by skeletal muscle tissue, cardiac muscle tissue, diaphragm muscle tissue or a combination thereof, wherein uptake of the secreted GAA results in a reduction in lysosomal glycogen stores in the tissue(s).
71. The method of any of paragraphs 69-70, wherein the administering to the subject is selected from any of: intramuscular, sub-cutaneous, intraspinal, intracisternal, intrathecal, intravenous administration.
72. A cell comprising the nucleic acid sequence of any of paragraphs 56-68.
73. The cell of any of paragraphs 72-73, wherein the cell is a human cell.
74. The cell of any of paragraphs 72-73, wherein the cell is a non-human cell mammalian cell.
75. The cell of any of paragraphs 72-73, wherein the cell is an insect cell.
76. A cell comprising the recombinant AAV vector of any of paragraphs 1-54.
77. A host animal comprising the recombinant AAV vector of any of paragraphs 1-54.
78. The host animal of paragraph 78, wherein the host animal is a mammal.
79. The host animal of paragraph 78 or 79, wherein the host animal is a non-human mammal.
80. The host animal of paragraph 78, wherein the host animal is a human.
81. The pharmaceutical composition of paragraph 55, for use in the method of any of paragraphs 69-71.
82. A host animal comprising a cell of any of paragraphs 72-75.
83. A host animal comprising the recombinant AAV vector of any of paragraphs 1-54.
84. The host animal of paragraph 78, wherein the host animal is a mammal.
85. The host animal of paragraph 78 or 79, wherein the host animal is a non-human mammal.
86. The host animal of paragraph 78, wherein the host animal is a human.

### EXAMPLES

The following non-limiting examples are provided for illustrative purposes only in order to facilitate a more complete understanding of representative embodiments now contemplated. These examples are intended to be a mere subset of all possible contexts in which the AAV virions and rAAV vectors may be utilized. Thus, these examples should not be construed to limit any of the embodiments described in the present specification, including those pertaining to AAV virions and rAAV vectors and/or methods and uses thereof. Ultimately, the AAV virions and vectors may be utilized in virtually any context where gene delivery is desired.

### Example 1: Construction of the rAAV Genome

Numerous rAAV genomes were constructed using Gibson cloning methodology. The following rAAV genomes were generated: SEQ ID NO: 57 (AAT-V43M-wtGAA (delta1-69aa)); SEQ ID NO: 58 (ratFN1-IGF2V43M-wtGAA (delta1-69aa)); SEQ ID NO: 59 (hFN1-IGF2V43M-wtGAA (delta1-69aa)); SEQ ID NO: 60 (ATT-IGF2Δ2-7-wtGAA (delta 1-69)); SEQ ID NO: 61 (FN1rat-IGFΔ2-7-wtGAA (delta 1-69)); SEQ ID NO: 62 (hFN1-IGFΔ2-7-wtGAA (delta 1-69))

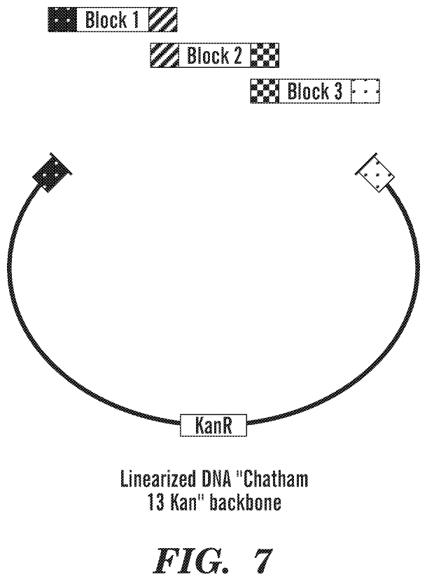

Gibson cloning involves cloning blocks (e.g., 3 blocks) of nucleic acid sequences together. The general protocol is as follows: the following reagents are combined into a single-tube reaction (i) Gibson Assembly Master Mix (Exonuclease, DNA polymerase, DNA Ligase, buffer) (ii) DNA inserts (Blocks 1-3) with 15-25 bp of homologous ends (see, FIG. 7) (iii) Linearized DNA backbone with 15-25 bp of homologous ends to the outermost DNA inserts (see, FIG. 7). The reaction is incubated at 50° C. for 15-60 minutes. The reaction mix is transformed into competent cells and plated on Kanamycin agar plates. Minipreps of fully-assembled plasmid DNA are screened via restriction digestion and/or colony PCR analysis and verified by DNA sequencing analysis. Verified clone is expanded for maxiprep production and transiently transfected in suspension HEK293 cells alongside the Adenovirus helper, XX680 Kan, and the appropriate Rep/Cap helper to produce rAAV.

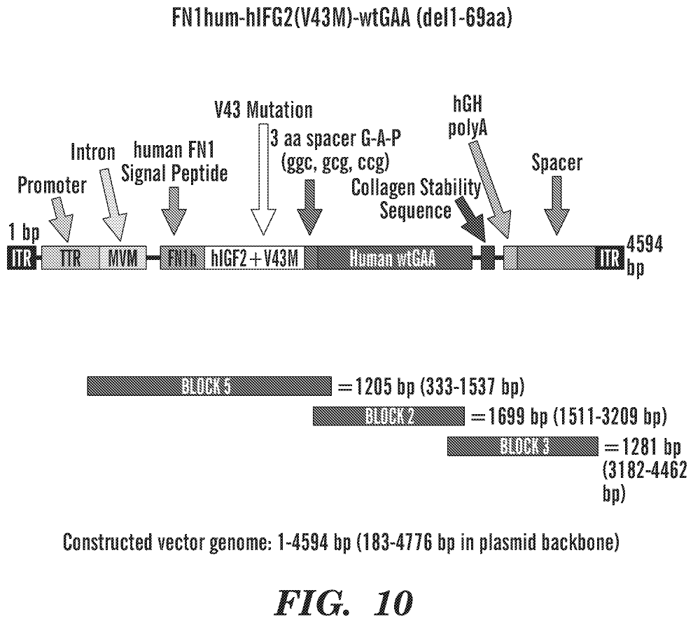

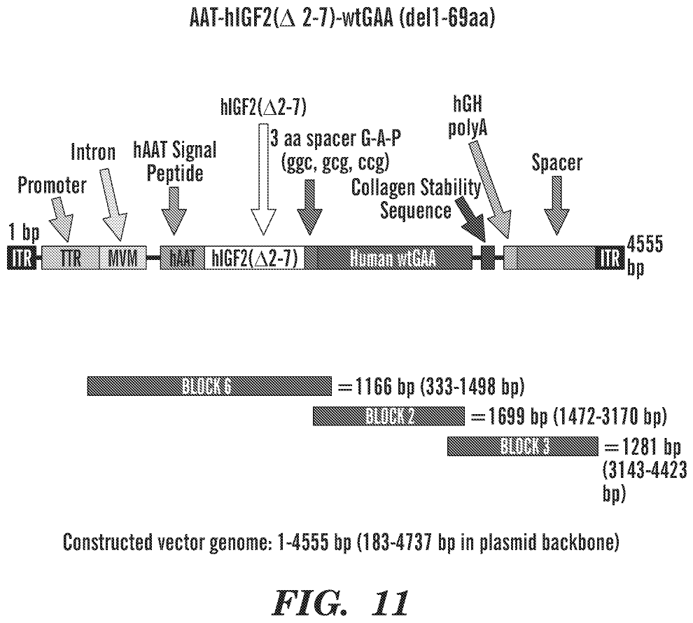

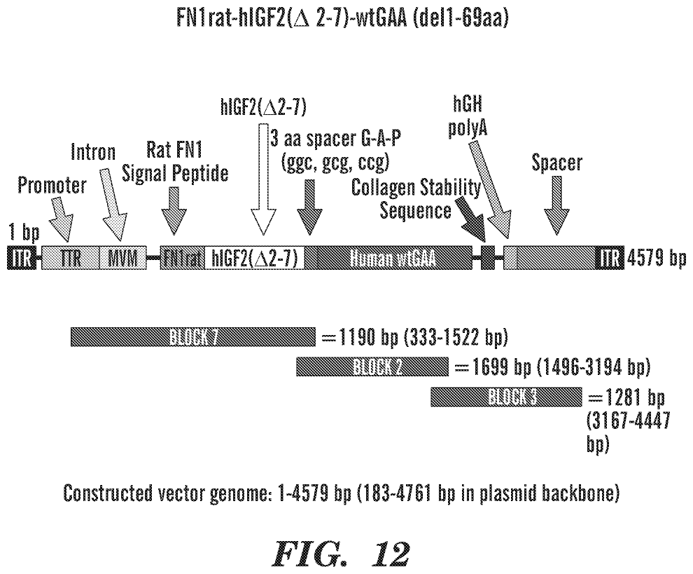

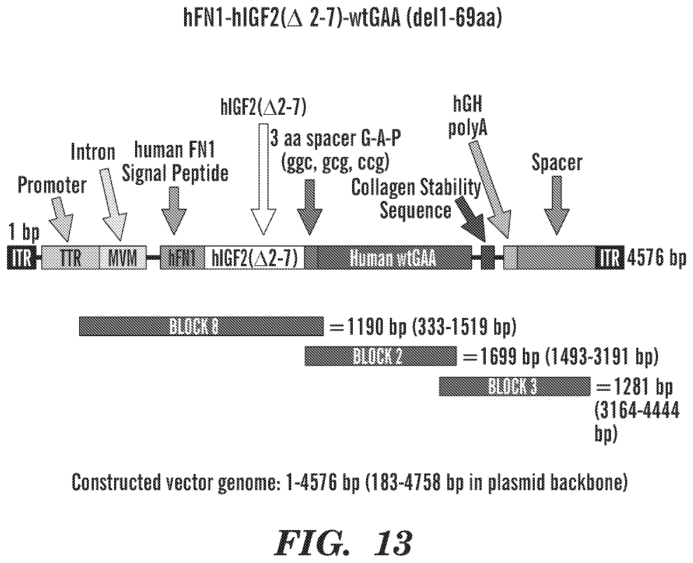

FIGS. 8-13 show the cloning nucleic blocks to generate exemplary rAAV genomes. For instance, FIG. 8 shows the generation of a rAAV genome comprising AAT-V43M-wtGAA (delta1-69aa)); FIG. 9 shows the generation of a rAAV genome comprising ratFN1-IGF2V43M-wtGAA (delta1-69aa)); FIG. 10 shows the generation of a rAAV genome comprising hFN1-IGF2V43M-wtGAA (delta1-69aa); FIG. 11 shows the generation of a rAAV genome comprising ATT-IGF2Δ2-7-wtGAA (delta 1-69); FIG. 12 shows the generation of a rAAV genome comprising FN1rat-IGFΔ2-7-wtGAA (delta 1-69), and FIG. 13 shows the generation of a rAAV genome comprising hFN1-IGFΔ2-7-wtGAA (delta 1-69).

While FIGS. 8-13 show wtGAA(41-69) is an exemplary GAA enzyme, this nucleic acid sequence can easily be replaced by one of ordinary skill with a nucleic acid sequence that has been codon optimized for enhanced expression in vivo, and/or to reduce immune response, and/or to reduce CpG islands. Also shown in the cloning blocks exemplified in FIGS. 8-13 is a generation of a rAAV genome a 3 amino acid (3aa) spacer nucleic acid sequence located 3′ of the nucleic acid sequence encoding the IGF(V42M) or IGFΔ2-7 targeting peptide and 5′ of the nucleic acid encoding a GAA enzyme, and a stuffer nucleic acid sequence a stuffer sequence (referred to in FIGS. 8-10 as a “spacer” sequence) which is located 3′ of the polyA sequence and 5′ of the 3′ITR sequence.

### Example 2: Generating rAAV Vectors

The rAAV genomes were packed into capsids to generate rAAV vectors using a rAAV Pro 10 cell line. Solely for proof of principal of rAAV vector construction, the capsids used were AAV3b capsids.

Making rAAV Pro 10 cell line: triple transfection technique was used to make rAAV in suspension HEK293 cells, which can be scaled up for making clinical grade vector. Alternatively, different plasmids can be used, e.g., 1) pXX680-ad helper and 2) pXR3 the Rep and Cap 3) and the Transgene plasmid (ITR—transgene-ITR).

The rAAV genomes generated in Example 1 are used to generate rAVV vectors using the Pro10 cell line as described in U.S. Pat. No. 9,441,206, which is incorporated herein in its entirety by reference. In particular, rAAV vectors or rAAV virions are produced using a method comprising: (a) providing to the HEK293 cells (e.g., ATTC No. PTA 13274) an AAV expression system; (b) culturing the cells under conditions in which AAV particles are produced; and (c) optionally isolating the AAV particles. Ratios of triple transfection of the plasmid and transfection cocktail volumes can be optimized, with varying plasmid ratios of XX680, AAV rep/cap helper and TR plasmid to determine the optimal plasmid ratio for rAAV vector production.

In some instances, the cells are cultured in suspension under conditions in which AAV particles are produced. In another embodiment, the cells are cultured in animal component-free conditions. The animal component-free medium can be any animal component-free medium (e.g., serum-free medium) compatible with HEK293 cells. Examples include, without limitation, SFM4Transfx-293 (Hyclone), Ex-Cell 293 (JRH Biosciences), LC-SFM (Invitrogen), and Pro293-S (Lonza). Conditions sufficient for the replication and packaging of the AAV particles can be, e.g., the presence of AAV sequences sufficient for replication of an rAAV genome described herein and encapsidation into AAV capsids (e.g., AAV rep sequences and AAV cap sequences) and helper sequences from adenovirus and/or herpesvirus.

### Example 3: Assessing rAAV Vectors

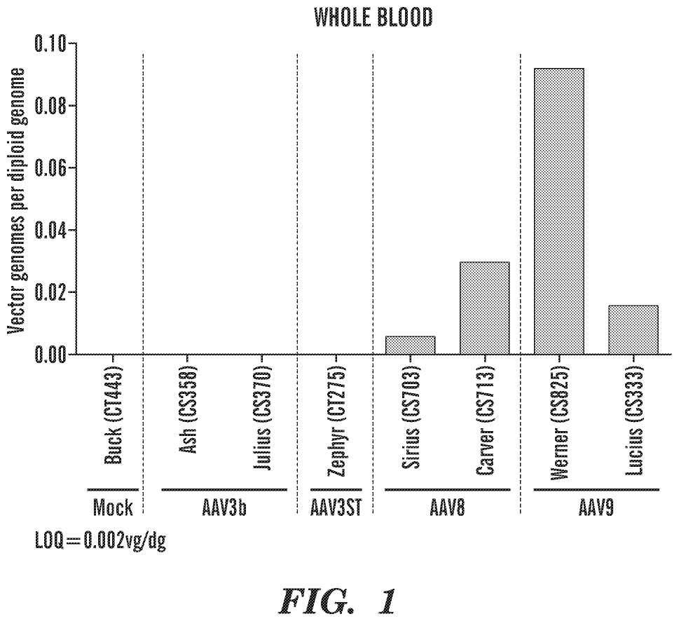

Whole Blood Clearance. FIG. 1 shows the results derived from an experiment where 3×10¹² vg/kg of different AAV serotypes (AAV3b, AAV3ST, AAV8, AAV9) were injected intravenously into 3 kg seronegative male macaques. The macaques were euthananized 60 days post administration of the different AAV serotypes. Vector genomes were searched in whole blood and results indicated that AAV3b was cleared within a week and was undetectable at sacrifice, whereas AAV8 and AAV9 were still detectable in whole blood when the macaques were sacrificed.

Liver Specific Vector Potency: FIG. 2 shows the results derived from an experiment where 3×10¹² vg/kg of different AAV serotypes (AAV3b, AAV3ST, AAV8, AAV9) were injected intravenously into 3 kg seronegative male macaques. The macaques were euthananized 60 days post administration of the different AAV serotypes. Vector genomes were quantified in each of the three lobes of the liver from each of the macaques. The limit of quantitation was 0.002 vg/dg. Based on the results presented in FIG. 2, AAV3b was found to be a potent liver vector. AAV3b is more liver specific than AAV8 and cleared from the blood more rapidly than AAV9. The AAV3ST mutant did not provide any significant beneficial affect.

### Example 4: Measuring Secretion of GAA into the Supernatant and GAA Uptake Assays

Measuring GAA in Supernatant.

Accordingly, the rAAV genomes generated in Example 1 are tested for secretion of GAA polypeptide into the supernatant. Measurement of GAA in the supernatant can be assessed using a 4-methyl-umbelliferyl-alpha-D-glucoside (4-MU) substrate (4-MU assay), as described in Kikuchi et al. (Kikuchi, Tateki, et al. “Clinical and metabolic correction of Pompe disease by enzyme therapy in acid maltase-deficient quail.” The Journal of clinical investigation 101.4 (1998): 827-833.).

In brief, HEK293 cells can be transfected with rAAV genomes SEQ ID NO: 57 (AAT-V43M-wtGAA (delta1-69aa)); SEQ ID NO: 58 (ratFN1-IGF2V43M-wtGAA (delta1-69aa)); SEQ ID NO: 59 (hFN1-IGF2V43M-wtGAA (delta1-69aa)); SEQ ID NO: 60 (ATT-IGF2Δ2-7-wtGAA (delta 1-69)); SEQ ID NO: 61 (FN1rat-IGFΔ2-7-wtGAA (delta 1-69)); SEQ ID NO: 62 (hFN1-IGFΔ2-7-wtGAA (delta 1-69)). GAA activity is measured based on the % of initial activity (t=0) over 24 hours. Samples were assayed for GAA enzyme activity based on the hydrolysis of the fluorogenic substrate 4-MU-α-glucose at 0, 3, 6 and 24 hours. The GAA activity was expressed as % of initial activity, i.e. residual activity.

Alternatively, after harvest, culture supernatants were partially purified by HIC chromatography. All samples were treated with PNGase prior to electrophoresis. The expression of GAA polypeptides by the cells can be assessed using SDS-PAGE and immunoblotting.

GAA Uptake Assays & Measuring Uptake of GAA in Tissues.

Next, the rAAV genomes generated in Examples 1 and 2 are tested for retention of uptake activity into cells. For example, HEK293 cells can be transfected with rAAV genomes SEQ ID NO: 57 (AAT-V43M-wtGAA (delta1-69aa)); SEQ ID NO: 58 (ratFN1-IGF2V43M-wtGAA (delta1-69aa)); SEQ ID NO: 59 (hFN1-IGF2V43M-wtGAA (delta1-69aa)); SEQ ID NO: 60 (ATT-IGF2Δ2-7-wtGAA (delta 1-69)); SEQ ID NO: 61 (FN1rat-IGFΔ2-7-wtGAA (delta 1-69)); SEQ ID NO: 62 (hFN1-IGFΔ2-7-wtGAA (delta 1-69)).

A 4-MU assay (as described above) can be to assess uptake of rhGAA into mammalian cells is described in US patent Application US2009/0117091A1, which is incorporated herein in its entirety by reference. rAAV vectors or rAAV genomes generated in Examples 1 and 2 are incubated in 20 μl reaction mixtures containing 123 mM sodium acetate pH 4.0 with 10 mM 4-methylumbelliferyl α-D-glucosidase substrate (Sigma, catalog #M-9766). Reactions were incubated at 37° C. for 1 hour and stopped with 200 μl of buffer containing 267 mM sodium carbonate, 427 mM glycine, pH 10.7. Fluorescence was measured with 355 nm excitation and 460 nm filters in 96-well microtiter plates and compared to standard curves derived from 4-methylumbelliferone (Sigma, catalog #M1381). 1 GAA 4 MU unit is defined as 1 nmole 4-methylumbelliferone hydrolyzed/hour. Specific activities of exemplary rAAV genomes in fibroblast cells are assessed, e.g., SEQ ID NO: 57 (AAT-V43M-wtGAA (delta1-69aa)); SEQ ID NO: 58 (ratFN1-IGF2V43M-wtGAA (delta1-69aa)); SEQ ID NO: 59 (hFN1-IGF2V43M-wtGAA (delta1-69aa)); SEQ ID NO: 60 (ATT-IGF2Δ2-7-wtGAA (delta 1-69)); SEQ ID NO: 61 (FN1rat-IGFΔ2-7-wtGAA (delta 1-69)); SEQ ID NO: 62 (hFN1-IGFΔ2-7-wtGAA (delta 1-69)). The enzymatic activity of IGF2-GAA fusion polypeptides and/or SS-IGF2-GAA double fusion polypeptide are assessed and compared to an untagged GAA (wtGAA).

Cell-based uptake assays can also be performed to demonstrate the ability of IGF2-tagged or untagged GAA to enter the target cell. Rat L6 myoblasts are plated at a density of 1×105 cells per well in 24-well plates 24 hours prior to uptake. At the start of the experiment, media is removed from the cells and replaced with 0.5 ml of uptake media which contains the rAAV vectors generated in Examples 1 and 2. In order to demonstrate specificity of uptake, some wells additionally contained the competitors M6P (5 mM final concentration) and/or IGF-2 (18 μg/ml final concentration). After 18 hours, media is aspirated off of cells, and cells are washed 4 times with PBS. Then, cells are lysed with 200 μl CelLytic MTM lysis buffer. The lysate is assayed for GAA activity as described above using the 4 MU substrate. Protein is determined using the Pierce BCATM Protein Assay Kit.

A typical uptake experiment is performed in CHO cells, although other cell lines and myoblast cell lines can be used. It is expected that uptake of the GAA polypeptides into Rat L6 myoblasts will be virtually unaffected by the addition of a large molar excess of M6P, whereas uptake is expected to be significantly abolished by excess IGF-2. In contrast, it is expected that uptake of wtGAA to be significantly abolished by addition of excess M6P but virtually unaffected by competition with IGF2. In addition, it is expected that uptake of IGF2V43M-wtGAA and IGFdelta2-7wtGAA will be unaffected significantly by excess IGF-2.

### Example 5 Half-Life of GAA in Rat L6 Myoblasts

An uptake experiment was performed as described above (see Example 3 & 4) with the rAAV vectors produced in Example 1 and 2 in rat L6 myoblasts. After 18 hours, media from cells transfected with the rAAV vectors was aspirated off and the cells were washed 4 times with PBS. At this time, duplicate wells were lysed (Time 0) and lysates were frozen at −80. Each day thereafter, duplicate wells were lysed and stored for analysis. After 14 days, all of the lysates were assayed for GAA activity, to assess the half-lives, and assess if, once inside cells, the IGF-2-tagged GAA enzyme persists with similar kinetics to untagged GAA.

### Example 6: Processing of GAA after Uptake

Mammalian GAA typically undergoes sequential proteolytic processing in the lysosome as described by Moreland et al. (2005) J. Biol. Chem., 280:6780-6791 and references contained therein. The processed protein gives rise to a pattern of peptides of 70 kDa, 20 kDa, 10 kDa and some smaller peptides. To determine whether IGF2-GAA fusion polypeptide and/or SS-IGF2-GAA double fusion polypeptide is processed similarly to the untagged GAA, aliquots of lysates from the above uptake experiment were analyzed by Western blot using a monoclonal antibody that recognizes the 70 kDa IGF-2 peptide and larger intermediates with the IGF-2 tag. A similar profile of polypeptides identified in this experiment indicates that once entering the cell, the IGF-2 sequence is lost and the IGF-2-GAA polypeptide is processed similarly to untagged GAA, which demonstrates that the IGF-2 sequence has little or no impact on the behavior of GAA once it is inside the cell.

### Example 7: Pharmacokinetics

Pharmacokinetics of IGF2-GAA fusion polypeptide and/or SS-IGF2-GAA double fusion polypeptide produced by the rAAV vectors can be measured in wild-type 129 mice. 129 mice are injected with the rAAV vectors generated in Example 1 and 2. Serum samples are taken preinjection and at 15 min, 30 min, 45 min, 60 min, 90 min, 120 min, 4 hours, and 8 hours post injection. The animals are then sacrificed. Serum samples are assayed by quantitative western blot. The half-lives for the GAA from rAAV vectors expressing IGF2-GAA fusion polypeptide or SS-IGF2-GAA double fusion polypeptide are assessed to determine if the IGF-2 fused GAA polypeptide is cleared from the circulation excessively rapidly.

### Example 8: Tissue Half-Life of GAA

The objective of this experiment was to determine the rate at which GAA activity is lost once the IGF2-GAA fusion polypeptide or SS-IGF2-GAA double fusion polypeptide expressed from the rAAV vector reaches its target tissue. In the Pompe mouse model, MYOZYME® appears to have a tissue half-life of about 6-7 days in various muscle tissues (Application Number 125141/0 to the Center for Drug Evaluation and Research and Center for Biologics Evaluation and Research, Pharmacology Reviews).

Pompe mice (Pompe mouse model 6neo/6neo as described in Raben (1998) JBC, 273:19086-19092, the disclosure of which is hereby incorporated by reference) are injected in the jugular vein with the rAAV vectors generated in Examples 1 and 2. Mice are then sacrificed at 1, 5, 10, and 15 days post injection. Tissue samples were homogenized and GAA activity measured according to standard procedures. The tissue half-life of GAA activity from IGF2-GAA fusion polypeptide and/or SS-IGF2-GAA double fusion polypeptide and the untagged GAA are calculated from the decay curves in different tissues (e.g., quadriceps tissue; heart tissue; diaphragm tissue; and liver tissue), and the half-life in each tissue calculated. This can be compared to the half-life in rat L6 myoblasts to determine if, once inside cells in Pompe mice, IGF2-GAA fusion polypeptide and/or SS-IGF2-GAA double fusion polypeptide expressed from the rAAV vectors described herein appears to persist with kinetics similar to the untagged GAA. Furthermore, the knowledge of the decay kinetics of the IGF2-GAA fusion polypeptide and/or SS-IGF2-GAA double fusion polypeptide can help in the design of appropriate dosing intervals.

### Example 9: Uptake of IGF2-GAA Fusion Polypeptide and/or SS-IGF2-GAA Double Fusion Polypeptide into Lysosomes of C2C12 Mouse Myoblasts

C2C12 mouse myoblasts grown on poly-lysine coated slides (BD Biosciences) are transduced with the rAAV vectors produced in Examples 1 and 2. After washing the cells, the cells are then incubated in growth media for 1 hour, then washed four times with D-PBS before fixing with methanol at room temperature for 15 minutes. The following incubations were all at room temperature, each separated by three washes in D-PBS. Slides are permeabilized with 0.1% triton X-100 for 15 minutes, then blocked with blocking buffer (10% heat-inactivated horse serum (Invitrogen) in D-PBS). Slides are incubated with primary mouse monoclonal anti-GAA antibody 3A6-1F2 (1:5,000 in blocking buffer), then with secondary rabbit anti-mouse IgG AF594 conjugated antibody (Invitrogen A11032, 1:200 in blocking buffer). A FITC-conjugated rat anti-mouse LAMP-1 (BD Pharmingen 553793, 1:50 in blocking buffer) is the incubated. Slides are mounted with DAPI-containing mounting solution (Invitrogen) and viewed with a Nikon Eclipse 80i microscope equipped with fluorescein isothiocyanate, texas red and DAPI filters (Chroma Technology). Images can be captured with a photometric Cascade camera controlled by MetaMorph software (Universal Imaging), and merged using Photoshop software (Adobe). Co-localization of signal detected by anti-GAA antibody with signal detected by antibody directed against a lysosomal marker, LAMP1 can be assessed to demonstrates that IGF2-tagged GAA is delivered to lysosomes.

### Example 10: Assessing the Treatment of the rAAV Vectors in a Pompe Mouse Model and Reversing Pompe Pathology

The rAAV vectors generated in Example 1 can be assessed in Pompe mouse mode, e.g., according to the methods described in Peng et al., “Reveglucosidase alfa (BMN 701), an IGF2-Tagged rhAcid α-Glucosidase, Improves Respiratory Functional Parameters in a Murine Model of Pompe Disease.” Journal of Pharmacology and Experimental Therapeutics 360.2 (2017): 313-323), which is incorporated herein in its entirety by reference.

Any Pompe mouse model can be used to assess the effect of the rAAV vectors at treating Pomoe disease. One mouse model of Pompe is described in Raben et al., JBC, 1998; 273(30); 19086-19092, which describes a disrupted GAA mouse model, and recapitulates critical features of both the infantile and the adult forms of the disease. In other instances, a Pompe mouse model (Sidman et al., 2008) can be used, as well as a strain of mice with a disrupted acid α-glucosidase gene (B6;129-GAAtm1Rabn/J; Pompe) (Jackson Laboratory, Bar Harbor, Me.). The Pompe mice develop the same cellular and clinical characteristics as in human adult Pompe disease (Raben et al., 1998). Animals are maintained in a 12-hour light/dark cycle, provided with fresh water and standard rodent chow ad libitum.

4.5-5 month old Pompe mice can be administered the rAAV vectors described herein, and evaluated for glycogen clearance after administration for 4 or more weeks. Following a macroscopic assessment, the heart (left ventricle), quadriceps, diaphragm, psoas, and soleus muscles were collected, weighed, snap-frozen in liquid nitrogen, and stored at −60 to −90° C. prior to a quantitative analysis of glycogen-derived glucose. Muscles were homogenized in buffer (0.2 M NaOAc/0.5% NP40) on ice using ceramic spheres. Amyloglucosidase was added to clarified lysates at 37° C. to digest glycogen into glucose for subsequent colorimetric detection (430 nm, SpectraMax; Molecular Devices, Sunnyvale, Calif.) using a peroxidase-glucose oxidase enzyme reaction system (Sigma-Aldrich, St. Louis, Mo.). Paired samples are also measured without amyloglucosidase to correct for endogenous tissue glucose that was not in glycogen form at harvest. Glucose values were extrapolated from a six-point standard curve. The measured glucose concentration (mg/ml) is proportional to the glycogen concentration of the sample and is converted to mg glycogen/g tissue by adjusting for the homogenization step (5 μl buffer added per gram of tissue).

The effect of rAAV vectors described herein on individual mouse muscle glycogen levels can be evaluated using Phoenix-WinNonlin classic PD modeling (Phoenix build version 6.4; Certara, L.P., Princeton, N.J.). Results can be obtained for hGAA in heart, diaphragm, quadriceps, psoas, and soleus muscles. For pharmacokinetic analysis, WT mice can be administered the rAAV vectors generated in Example 1 and blood samples collected as terminal cardiac punctures at predose, 0.083, 0.5, 1, 2, and 4 hours postdose. Plasma hGAA concentrations can be quantified using a bridging electrochemiluminescent method with an LOQ of 100 ng/ml. Briefly, 0.5 μg/ml ruthenium-labeled anti-rhGAA (affinity purified goat polyclonal) and 0.5 μg/ml biotin-labeled anti-IGF2 (MAB792; R&D Systems, Minneapolis, Minn.) can be combined with K2EDTA plasma samples diluted 1:10 in buffer [Starting Block T20 (PBS); ThermoFisher Scientific, Sunnyvale, Calif.] and incubated for 1 hour before transfer to a blocked streptavidin assay plate (Meso Scale Diagnostics, Rockville, Md.). After a 30-minute incubation, the plate is washed, lx Read Buffer T (Meso Scale Diagnostics) was added, and the electrochemiluminescent signal read on an SECTOR Imager 2400 (Meso Scale Diagnostics). hGAA concentrations can be extrapolated from a standard curve.

Alternatively, Heart and Diaphragm tissue homogenates can be harvested and rhGAA activity measured using the fluorogenic substrate (4-MUG).

The therapeutic effect of the GAA polypeptide produced using rAAV vectors generated in Examples 1 and 2 herein can be compared wt GAA in vivo. A study can be performed to compare the ability of a rAAV vector disclosed in Example 1 to that expressing a non-tagged wt GAA to clear glycogen from skeletal muscle tissue in Pompe mice (e.g., Pompe mouse model 6neo/6neo animals were used (Raben (1998) JBC 273:19086-19092)). Groups of Pompe mice (5/group) received IV injections of one of two doses of wt GAA or a rAAV vector generated in Example 1 or vehicle. Five untreated animals can be used as control, and receive four weekly injection of saline solution. Animals receive oral diphenhydromine, 5 mg/kg one hour prior to injections 2, 3, and 4. Mice were sacrificed one week after the injection, and tissues (diaphragm, heart, lung, liver, soleus, quadriceps, gastrocnemius, TA, EDL, tongue) are harvested for histological and biochemical analysis. Glycogen content in the tissue homogenates can be measured using A. niger amyloglucosidase and the Amplex Red Glucose assay kit, and GAA enzyme levels assessed in different tissue homogenates using standard procedures.

Glycogen content in tissue homogenates can be measured using A. niger amyloglucosidase and the Amplex® Red Glucose assay kit (Invitrogen) essentially as described by Zhu et al. (2005) Biochem J., 389:619-628.

It is expected that the rAAV vector ss-IGF2-GAA rAAV as described herein and produced by the methods of Examples 1 and 2 will have more uptake into muscle and greater therapeutic effect in the Pompe mouse model as compared to a IGF-2-GAA rAAV (i.e., without the secretory signal sequence), which is expected to be greater than wtGAA rAAV vector (i.e., without either of the secretory signal and the IGF2 sequence), and/or MYOZYME®. Given the established Pompe model, these results are expected to translate into the clinic and correlate with therapeutic effect for the treatment of Pompe disease.

### Example 11: Clearance of Glycogen In Vivo

The objective of this experiment is to determine the rate at which glycogen is cleared from heart tissue of Pompe mice after a single injection of rAAV vector expressing IGF2-GAA fusion polypeptide and/or SS-IGF2-GAA double fusion polypeptide produced in Examples 1 and 2.

Pompe mice (Pompe mouse model 6neo/6neo as described in Raben (1998) JBC, 273:19086-19092, the disclosure of which is hereby incorporated by reference) are injected in the jugular vein with a rAAV vector expressing produced in Examples 1 and 2. Mice were are sacrificed at 1, 5, 10, and 15 days post injection. Heart tissue samples are homogenized according to standard procedures and analyzed for glycogen content. Glycogen content in these tissue homogenates is measured using A. niger amyloglucosidase and the Amplex® Red Glucose assay kit (Invitrogen) essentially as described by Zhu et al. (2005) Biochem J., 389:619-628. Assessment of the heart tissue from mice can determine if there is almost complete clearance of glycogen in the mice administered rAAV vector expressing IGF2-GAA fusion polypeptide and/or SS-IGF2-GAA double fusion polypeptide produced in Examples 1 and 2 as compared to mice administered a rAAV where GAA was not fused to a IGF2 sequence and/or SS as described herein, where only a small change in glycogen content would indicate minimal clearance.

In closing, regarding the exemplary embodiments of the present invention as shown and described herein, it will be appreciated that a genomic construct, comprising an AAV (adeno-associated virus) viral virion is disclosed and configured for delivery of AAV vectors. Because the principles of the invention may be practiced in a number of configurations beyond those shown and described, it is to be understood that the invention is not in any way limited by the exemplary embodiments, but is generally directed to a genomic construct, comprising an AAV (adeno-associated virus) viral virion apparatus and is able to take numerous forms to do so without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of” As such, embodiments described herein or so claimed with the phrase “comprising” are expressly or inherently unambiguously described, enabled and supported herein for the phrases “consisting essentially of” and “consisting of.”

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

### REFERENCES

The references disclosed in the specification and Examples, including but not limited to patents and patent applications, and international patent applications are all incorporated herein in their entirety by reference.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### REFERENCES FOR TABLE 2

1. L Lisowski, A P Dane, K Chu, Y Zhang, S C Cunninghamm, E M Wilson, et al. Selection and evaluation of clinically relevant AAV variants in a xenograft liver model Nature, 506 (2014), pp. 382-386 (LKO3 and others LKO-19)
2. Grimm D, Lee J S, Wang L, Desai T, Akache B Storm T A, Kay M A. In vitro and in vivo gene therapy vector evolution via multispecies interbreeding and retargeting of adeno-associated viruses. J Virol. 2008 June: 82(12):5887-911. (AAV-DJ)
3. Powell S K, Khan N, Parker C L, Samulski R J, Matsushima G, Gray S J, McCown T J. Characterization of a novel adeno-associated viral vector with preferential oligodendrocyte tropism. Gene Ther. 2016 November: 23(11):807-814. (Olig001)
4. Tervo D G, Hwang B Y, Viswanathan S, Gaj T, Lavzin M, Ritola K D, Lindo S, Michael S, Kuleshova E, Ojala D, Huang C C, Gerfen C R, Schiller J, Dudman J T, Hantman A W, Looger L L, Schaffer D V, Karpova A Y. A Designer AAV Variant Permits Efficient Retrograde Access to Projection Neurons. Neuron. 2016 Oct. 19: 92(2):372-382. (rAAV2-retro)
5. Marsic D, Govindasamy L, Currlin S, Markusic D M, Tseng Y S, Herzog R W, Agbandje-McKenna M, Zolotukhin S. Vector design Tour de Force: integrating combinatorial and rational approaches to derive novel adeno-associated virus variants. Mol Ther. 2014 November: 22(11):1900-9. (AAV-LiC)
6. Sallach J, Di Pasquale G, Larcher F, Niehoff N, Rubsam M, Huber A, Chiarini J, Almarza D, Eming S A, Ulus H, Nishimura S, Hacker U T, Ballek M, Niessen C M, Buning H. Tropism-modified AAV vectors overcome barriers to successful cutaneous therapy. Mol Ther. 2014 May: 22(5):929-39. (AAV-Kera1, AAV-Kera2, and AAV-Kera3)
7. Dalkara D, Byrne L C, Klimczak R R, Visel M, Yin L, Merigan W H, Flannery J G, Schaffer D V. In vivo-directed evolution of a new adeno-associated virus for therapeutic outer retinal gene delivery from the vitreous. Sci Transl Med. 2013 Jun. 12:5(189):189ra76. (AAV 7m8)
8. Asuri P, Bartel M A, Vazin T, Jang J H, Wong T B, Schaffer D V. Directed evolution of adeno-associated virus for enhanced gene delivery and gene targeting in human pluripotent stem cells. Mol Ther. 2012 February: 20(2):329-38. (AAV1.9)
9. Jang J H, Koerber J T, Kim J S, Asuri P, Vazin T, Bartel M, Keung A, Kwon I, Park K I, Schaffer D V. An evolved adeno-associated viral variant enhances gene delivery and gene targeting in neural stem cells. Mol Ther. 2011 April: 19(4):667-75. doi: 10.1038/mt.2010.287. (AAV r3.45)
10. Gray S J, Blake B L, Criswell H E, Nicolson S C, Samulski R I, McCown T J, Li W. Directed evolution of a novel adeno-associated virus (AAV) vector that crosses the seizure-compromised blood-brain barrier (BBB). Mol Ther. 2010 March: 18(3):570-8 (AAV clone 32 and 83)
11. Maguire C A, Gianni D, Meijer D H, Shaket L A, Wakimoto H, Rabkin S D, Gao G, Sena-Esteves M. Directed evolution of adeno-associated virus for glioma cell transduction. J Neurooncol. 2010 February: 96(3):337-47. (AAV-U87R7-05)
12. Koerber J T, Klimczak R, Jang J H, Dalkara D, Flannery J G, Schaffer D V. Molecular evolution of adeno-associated virus for enhanced glial gene delivery. Mol Ther. 2009 December: 17(12):2088-95. (AAV ShH13, AAV ShH19, AAV L1-12)
13. Li W, Zhang L, Johnson J S, Zhijian W, Grieger J C, Ping-Jie X, Drouin L M, Agbandje-McKenna M, Pickles R J, Samulski R J. Generation of novel AAV variants by directed evolution for improved CFTR delivery to human ciliated airway epithelium. Mol Ther. 2009 December: 17(12):2067-77. (AAV HAE-1, AAV HAE-2)
14. Klimczak R R, Koerber J T, Dalkara D, Flannery J G, Schaffer D V. A novel adeno-associated viral variant for efficient and selective intravitreal transduction of rat Muller cells. PLoS One. 2009 Oct. 14: 4(10):e7467. (AAV variant ShH10)
15. Excoffon K J, Koerber J T, Dickey D D, Murtha M, Keshavjee S, Kaspar B K, Zabner J, Schaffer D V. Directed evolution of adeno-associated virus to an infectious respiratory virus. Proc Natl Acad Sci USA. 2009 Mar. 10:106(10):3865-70. (AAV2.5T)
16. Sellner L, Stiefelhagen M, Kleinschmidt J A, Laufs S, Wenz F, Fruehauf S, Zeller W J, Veldwijk M R. Generation of efficient human blood progenitor-targeted recombinant adeno-associated viral vectors (AAV) by applying an AAV random peptide library on primary human hematopoietic progenitor cells. Exp Hematol. 2008 August: 36(8):957-64. (AAV LS1-4, AAV Lsm)
17. Li W, Asokan A, Wu Z, Van Dyke T, DiPrimio N, Johnson J S, Govindaswamy L, Agbandje-McKenna M, Leichtle S, Redmond D E Jr, McCown T J, Petermann K B, Sharpless N E, Samulski R T. Engineering and selection of shuffled AAV genomes: a new strategy for producing targeted biological nanoparticles. Mol Ther. 2008 July: 16(7):1252-60. (AAV1289)
18. Charbel Issa P, De Silva S R, Lipinski D M, Singh M S, Mouravlev A, You Q. Assessment of tropism and effectiveness of new primate-derived hybrid recombinant AAV serotypes in the mouse and primate retina. PLoS ONE. 2013:8:e60361. (AAVHSC 1-17)
19. Huang W, McMurphy T, Liu X, Wang C, Cao L. Genetic Manipulation of Brown Fat Via Oral Administration of an Engineered Recombinant Adeno-associated Viral Serotype Vector. Mol Ther. 2016 June: 24(6):1062-9. (AAV2 Rec 1-4)
20. Cronin T, Vandenberghe L H, Hantz P, et al. Efficient transduction and optogenetic stimulation of retinal bipolar cells by a synthetic adeno-associated virus capsid and promoter. EMBO Mol Med 2014:6:1175-1190 (AAV8BP2)
21. Choudhury S R, Fitzpatrick Z, Harris A F, Maitland S A, Ferreira J S, Zhang Y, Ma S, Sharma R B, Gray-Edwards H L, Johnson J A, Johnson A K, Alonso L C, Punzo C, Wagner K R, Maguire C A, Katin R M, Martin D R, Sena-Esteves M. In Vivo Selection Yields AAV-B1 Capsid for Central Nervous System and Muscle Gene Therapy. Mol Ther. 2016 August: 24(7):1247-57. (AAV-B1)
22. Deverman B E, Pravda P L, Simpson B P, Kumar S R, Chan K Y, Banerjee A, Wu W L, Yang B, Huber N, Pasca S P, Gradinaru V. Cre-dependent selection yields AAV variants for widespread gene transfer to the adult brain. Nat Biotechnol. 2016 February: 34(2):204-9. doi: 10.1038/nbt.3440. (AAV-PHP.B)
23. Pulicherla N, Shen S, Yadav S, Debbink K, Govindasamy L, Agbandje-McKenna M, Asokan A. Engineering liver-detargeted AAV9 vectors for cardiac and musculoskeletal gene transfer. Mol Ther. 2011 June: 19(6):1070-8. (AAV9 derived mutants-AAV9.45, AAV9.61, AAV9.47)
24. Yang L, Jiang J, Drouin L M, Agbandje-McKenna M, Chen C, Qiao C, Pu D, Hu X, Wang D Z, Li J, Xiao X. A myocardium tropic adeno-associated virus (AAV) evolved by DNA shuffling and in vivo selection. Proc Natl Acad Sci USA. 2009 Mar. 10:106(10):3946-51. (AAVM41)
25. Korbelin J, Sieber T, Michelfelder S, Lunding L, Spies E, Hunger A, Alawi M, Rapti K, Indenbirken D, Muller O J, Pasqualini R, Arap W, Kleinschmidt J A, Trepel M. Pulmonary Targeting of Adena-associated Viral Vectors by Next-generation Sequencing-guided Screening of Random Capsid Displayed Peptide Libraries. Mol Ther. 2016 June: 24(6):1050-61. (AAV2 displayed peptides)
26. Geoghegan J C, Keiser N W, Okulist A, Martins I, Wilson M S, Davidson B L. Chondroitin Sulfate is the Primary Receptor for a Peptide-Modified AAV That Targets Brain Vascular Endothelium In Vivo. Mol Ther Nucleic Acids. 2014 Oct. 14:3:e202. (AAV2-GMN)
27. Varadi K, Michelfelder S, Korff T, Hecker M, Trepel M, Katus H A, Kleinschmidt J A, Muller O J. Novel random peptide libraries displayed on AAV serotype 9 for selection of endothelial cell-directed gene transfer vectors. Gene Ther. 2012 August: 19(8):800-9. (AAV9-peptide displayed)
28. Michelfelder S, Varadi K, Raupp C, Hunger A, Korbelin J, Pahrmann C, Schrepfer S, Muller O J, Kleinschmidt J A, Trepel M. Peptide ligands incorporated into the threefold spike capsid domain to re-direct gene transduction of AAV8 and AAV9. in vivo. PLoS One. 2011:6(8):e23101. (AAV8 and AAV9 peptide displayed)
29. Yu C Y, Yuan Z, Cao Z, Wang B, Qiao C, Li J, Xiao X. A muscle-targeting peptide displayed on AAV2 improves muscle tropism on systemic delivery. Gene Ther. 2009 August: 16(8):953-62.
30. Michelfelder S, Lee M K, deLima-Hahn E, Wilmes T, Kaul F, Muller O, Kleinschmidt J A, Trepel M. Vectors selected from adeno-associated viral display peptide libraries for leukemia cell-targeted cytotoxic gene therapy. Exp Hematol. 2007 December: 35(12): 1766-76.
31. Muller O J, Kaul F, Weitzman M D, Pasqualini R, Arap W, Kleinschmidt J A, Trepel M. Random peptide libraries displayed on adeno-associated virus to select for targeted gene therapy vectors. Nat Biotechnol. 2003 September: 21(9):1040-6.
32. Grifman M, Trepel M, Speece P, Gilbert L B, Arap W, Pasqualini R, Weitzman M D. Incorporation of tumor-targeting peptides into recombinant adeno-associated virus capsids. Mol Ther. 2001 June: 3(6):964-75.
33. Anne Girod, Martin Ried, Christiane Wobus, Harald Lahm, Kristin Leike, Jurgen Kleinschmidt, Gilbert Deleage & Michael Ballek. Genetic capsid modifications allow efficient re-targeting of adeno-associated virus type 2. Nature Medicine, 1052-1056 (1999)
34. Bello A, Chand A, Aviles J, Soule G, Auricchio A, Kobinger G P. Novel adeno-associated viruses derived from pig tissues transduce most major organs in mice. Sci Rep. 2014 Oct. 22:4:6644. (AAVpo2.1, -po4, -poS, and -po6).
35. Gao G, Vandenberghe L H, Alvira M R, Lu Y, Calcedo R, Zhou X, Wilson J M. Clades of Adena-associated viruses are widely disseminated in human tissues. J Virol. 2004 June: 78(12):6381-8. (AAV rh and AAV Hu)
36. Arbetman A E, Lochrie M, Zhou S, Wellman J, Scallan C, Doroudchi M M, et al. Novel caprine adeno-associated virus (AAV) capsid (AAV-Go.1) is closely related to the primate AAV-5 and has unique tropism and neutralization properties. J Virol. 2005:79:15238-15245. (AAV-Go.1)
37. Lochrie M A, Tatsuno G P, Arbetman A E, Jones K, Pater C, Smith P H, et al. Adena-associated virus (AAV) capsid genes isolated from rat and mouse liver genomic DNA define two new AAV species distantly related to AAV-5. Virology. 2006:353:68-82. (AAV-mo.1)
38. Schmidt M, Katano H, Bossis I, Chiarini J A. Cloning and characterization of a bovine adeno-associated virus. J Virol. 2004:78:6509-6516. (BAAV)
39. Bossis I, Chiarini J A. Cloning of an avian adeno-associated virus (AAAV) and generation of recombinant AAAV particles. J Virol. 2003:77:6799-6810. (AAAV)
40. Chen C L, Jensen R L, Schnepp B C, Connell M J, Shell R, Sferra T J, Bartlett J S, Clark K R, Johnson P R. Molecular characterization of adeno-associated viruses infecting children. J Virol. 2005 December: 79(23):14781-92. (AAV variants)
41. Sen D, Gadkari R A, Sudha G, Gabriel N, Kumar Y S, Selot R, Samuel R, Rajalingam S, Ramya V, Nair S C, Srinivasan N, Srivastava A, Jayandharan G R. Targeted modifications in adeno-associated virus serotype 8 capsid improves its hepatic gene transfer efficiency in vivo. Hum Gene Ther Methods. 2013 April: 24(2):104-16. (AAV8 K137R)
42. Li B, Ma W, Ling C, Van Vliet K, Huang L Y, Agbandje-McKenna M, Srivastava A, Aslanidi G V. Site-Directed Mutagenesis of Surface-Exposed Lysine Residues Leads to Improved Transduction by AAV2, But Not AAV8, Vectors in Murine Hepatocytes In Vivo. Hum Gene Ther Methods. 2015 December: 26(6):211-20.
43. Gabriel N, Hareendran S, Sen D, Gadkari R A, Sudha G, Selot R, Hussain M, Dhaksnamoorthy R, Samuel R, Srinivasan N, et al. Bioengineering of AAV2 capsid at specific serine, threonine, or lysine residues improves its transduction efficiency in vitro and in vivo. Hum Gene Ther Methods. 2013 April: 24(2):80-93.
44. Zinn E, Pacouret S, Khaychuk V, Turunen H T, Carvalho L S, Andres-Mateos E, Shah S, Shelke R, Maurer A C, Plovie E, Xiao R, Vandenberghe L H. In Silico Reconstruction of the Viral Evolutionary Lineage Yields a Potent Gene Therapy Vector. Cell Rep. 2015 Aug. 11:12(6):1056-68. (AAV Anc80L65)
45. Shen S, Horowitz E D, Troupes A N, Brown S M, Pulicherla N, Sarnulski R J, Agbandje-McKenna M, Asokan A. Engraftment of a galactose receptor footprint onto adeno-associated viral capsids improves transduction efficiency. J Biol Chem. 2013 Oct. 4:288(40):28814-23. (AAV2G9)
46. Li C, Diprirnio N, Bowles D E, Hirsch M L, Monahan P E, Asokan A, Rabinowitz J, Agbandje-McKenna M, Sarnulski R J. Single amino acid modification of adeno-associated virus capsid changes transduction and humoral immune profiles. J Virol. 2012 August: 86(15):7752-9. (AAV2 265 insertion-AAV2/265D)
47. Bowles D E, McPhee S W, Li C, Gray S J, Sarnulski J J, Camp A S, Li J, Wang B, Monahan P E, Rabinowitz J E, et al. Phase 1 gene therapy for Duchenne muscular dystrophy using a translational optimized AAV vector. Mol Ther. 2012 February: 20(2):443-55 (AAV2.5)
48. Messina E L, Nienaber J, Daneshrnand M, Villarnizar N, Sarnulski J, Milano C, Bowles D E. Adena-associated viral vectors based on serotype 3b use components of the fibroblast growth factor receptor signaling complex for efficient transduction. Hum. Gene Ther. 2012 October: 23(10):1031-42. (AAV3 SASTG)
49. Asokan A, Conway J C, Phillips J L, Li C, Hegge J, Sinnott R, Yadav S, DiPrirnio N, Nam H J, Agbandje-McKenna M, McPhee S, Wolff J, Sarnulski R J. Reengineering a receptor footprint of adeno-associated virus enables selective and systemic gene transfer to muscle. Nat Biotechnol. 2010 January: 28(1):79-82. (AAV2i8)
50. Vance M, Llanga T, Bennett W, Woodard K, Murlidharan G, Chungfat N, Asokan A, Gilger B, Kurtzberg J, Sarnulski R J, Hirsch M L. AAV Gene Therapy for MPS1-associated Corneal Blindness. Sci Rep. 2016 Feb. 22:6:22131. (AAV8G9)
51. Zhong L, Li B, Mah C S, Govindasarny L, Agbandje-McKenna M, Cooper M, Herzog R W, Zolotukhin I, Warrington K H Jr, Weigel-Van Aken K A, Hobbs J A, Zolotukhin S, Muzyczka N, Srivastava A. Next generation of adeno-associated virus 2 vectors: point mutations in tyrosines lead to high-efficiency transduction at lower doses. Proc Natl Acad Sci USA. 2008 Jun. 3:105(22):7827-32. (AAV2 tyrosine mutants AAV2 Y-F)
52. Petrs-Silva H, Dinculescu A, Li Q, Min S H, Chiodo V, Pang J J, Zhong L, Zolotukhin S, Srivastava A, Lewin A S, Hauswirth W W. High-efficiency transduction of the mouse retina by tyrosine-mutant AAV serotype vectors. Mol Ther. 2009 March: 17(3):463-71. (AAV8 Y-F and AAV9 Y-F)
53. Qiao C, Zhang W, Yuan Z, Shin J H, Li J, Jayandharan G R, Zhong L, Srivastava A, Xiao X, Duan D. Adena-associated virus serotype 6 capsid tyrosine-to-phenylalanine mutations improve gene transfer to skeletal muscle. Hum Gene Ther. 2010 October: 21(10):1343-8 (AAV6 Y-F)
54. Carlon M, Toelen J, Van der Perren A, Vandenberghe L H, Reumers V, Sbragia L, Gijsbers R, Baekelandt V, Himmelreich U, Wilson J M, Deprest J, Debyser Z. Efficient gene transfer into the mouse lung by fetal intratracheal injection of rAAV2/6.2. Mol Ther. 2010 December: 18(12):2130-8. (AAV6.2) PCT Publication No. WO2013158879A1 (lysine mutants)
55. Piacentino III, Valentino, et al. “X-linked inhibitor of apoptosis protein-mediated attenuation of apoptosis, using a novel cardiac-enhanced adeno-associated viral vector.” Human gene therapy 23.6 (2012): 635-646.

1.-7. (canceled)

8. The recombinant AAV vector of claim 34, wherein the promoter is constitutive, cell specific or inducible.

9.-31. (canceled)

32. The recombinant AAV vector of claim 34, wherein the AAV3b serotype comprises one or mutations in a capsid protein selected from any of: 265D, 549A, Q263Y

33. The recombinant AAV vector of claim 34, wherein the AAV3b serotype is selected from any of: AAV3b265D, AAV3b265D549A, AAV3b549A or AAV3bQ263Y, or AAV3bSASTG.

34. A recombinant adenovirus associated (AAV) vector comprising in its genome:
a. 5′ and 3′ AAV inverted terminal repeats (ITR) sequences, and
b. located between the 5′ and 3′ ITRs, a heterologous nucleic acid sequence encoding a polypeptide comprising an alpha-glucosidase (GAA) polypeptide, wherein the heterologous nucleic acid is operatively linked to a liver specific promoter.
wherein the recombinant AAV vector comprises a capsid protein of the AAV3b or AAV8 serotype.

35. The recombinant AAV vector of claim 34, wherein the polypeptide further comprises a secretory signal peptide located at the N-terminal of the GAA polypeptide.

36. The recombinant AAV vector of claim 34, wherein the heterologous nucleic acid sequence encoding a polypeptide further comprises a IGF-2 sequence located between the secretory signal peptide and the an alpha-glucosidase (GAA) polypeptide.

37. The recombinant AAV vector of claim 34, wherein the AAV genome comprises, in the 5′ to 3′ direction:
a. a 5′ ITR,
b. a liver specific promoter sequence,
c. an intron sequence,
d. a nucleic acid encoding a secretory signal peptide,
e. a nucleic acid encoding an IGF-2 sequence,
f. a nucleic acid encoding an alpha-glucosidase (GAA) polypeptide,
g. a poly A sequence, and
h. a 3′ ITR.

38. The recombinant AAV vector of claim 35, wherein the secretory signal peptide is selected from an AAT signal peptide, a fibronectin signal peptide (FN), a GAA signal peptide, or an active fragment thereof having secretory signal activity.

39. The recombinant AAV vector of claim 34, wherein the IGF-2 leader sequence binds human cation-independent mannose-6-phosphate receptor (CI-MPR) or the IGF-2 receptor, or
comprises SEQ ID NO: 5 or comprises at least one amino modification in SEQ ID NO: 5 that affects binding to the IGF21 receptor.

40. (canceled)

41. The recombinant AAV vector of claim 39, wherein the at least one amino modification in SEQ ID NO: 5 is a V43M amino acid modification (SEQ ID NO: 8 or SEQ ID NO: 9) or 42-7 (SEQ ID NO: 6) or 41-7 (SEQ ID NO: 7).

42. The recombinant AAV vector of claim 34, wherein the liver specific promoter is selected from any of: transthyretin promoter (TTR), LSP promoter (LSP), a synthetic liver specific promoter.

43. The recombinant AAV vector of claim 34, wherein the heterologous nucleic acid sequence encodes a wild-type GAA polypeptide or a modified GAA polypeptide.

44. The recombinant AAV vector of claim 34, wherein the heterologous nucleic acid sequence encoding the GAA polypeptide is the human GAA gene or a human codon optimized GAA gene (coGAA) or a modified GAA nucleic acid sequence.

45. The recombinant AAV vector of claim 34, wherein the nucleic acid sequence encoding the GAA polypeptide is codon optimized for enhanced expression in vivo, or
wherein the nucleic acid sequence encoding the GAA polypeptide is codon optimized to reduce CpG islands, or
wherein the nucleic acid sequence encoding the GAA polypeptide is codon optimized to reduce the innate immune response.

46.-48. (canceled)

49. The recombinant AAV vector of claim 34, wherein the intron sequence comprises a MVM sequence or a HBB2 sequence.

50. The recombinant AAV vector of claim 34, wherein the ITR comprises an insertion, deletion or substitution.

51. The recombinant AAV vector of claim 45, wherein one or more CpG islands in the ITR are removed.

52. The recombinant AAV vector of claim 34, wherein the secretory signal peptide is a fibronectin signal peptide (FN1) or an active fragment thereof having secretory signal activity, and the IGF-2 sequence is selected from any of: SEQ ID NO: 5, SEQ ID NO: 6, SEQ ID NO: 7, SEQ ID NO: 8 or SEQ ID NO: 9.

53. (canceled)

54. The recombinant AAV vector of claim 34, wherein the IGF-2 sequence is SEQ ID NO: 8 or SEQ ID NO: 9.

55. A pharmaceutical composition comprising the recombinant AAV vector of claim 34 in a pharmaceutically acceptable carrier.

56. (canceled)

57. A nucleic acid sequence for a recombinant adenovirus associated viral (rAAV) vector genome comprising:
a. 5′ and 3′ AAV inverted terminal repeats (ITR) nucleic acid sequences, and
b. located between the 5′ and 3′ ITR sequence, a heterologous nucleic acid sequence encoding a fusion polypeptide comprising a secretory signal peptide and an alpha-glucosidase (GAA) polypeptide, wherein the heterologous nucleic acid is operatively linked to a promoter.

58.-68. (canceled)

69. A method to treat a subject with a glycogen storage disease type II (GSD II, Pompe Disease, Acid Maltase Deficiency) or having a deficiency in alpha-glucosidase (GAA) polypeptide, comprising administering the recombinant AAV vector of claim 34 to the subject.

70.-71. (canceled)


SEQUENCE LISTING (85 sequences)

SEQ ID NO: 1 - DNA, 201 bp, Homo sapiens
gcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgag

SEQ ID NO: 2 - DNA, 183 bp, Artificial Sequence
  Description of Artificial Sequence: Synthetic polynucleotide
gctctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgag

SEQ ID NO: 3 - DNA, 180 bp, Artificial Sequence
  Description of Artificial Sequence: Synthetic polynucleotide
ctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgag

SEQ ID NO: 4 - DNA, 201 bp, Artificial Sequence
  Description of Artificial Sequence: Synthetic polynucleotide
gcttaccgccccagtgagaccctgtgcggcggggagctggtggacaccctccagttcgtctgtggggaccgcggcttctacttcagcaggcccgcaagccgtgtgagccgtcgcagccgtggcatcatggaggagtgctgtttccgcagctgtgacctggccctcctggagacgtactgtgctacccccgccaagtccgag

SEQ ID NO: 5 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 6 - protein, 61 aa, Artificial Sequence
  Description of Artificial Sequence: Synthetic polypeptide
ALCGGELVDTLQFVCGDRGFYFSRPASRVSRRSRGIVEECCFRSCDLALLETYCATPAKSE

SEQ ID NO: 7 - protein, 60 aa, Artificial Sequence
  Description of Artificial Sequence: Synthetic polypeptide
LCGGELVDTLQFVCGDRGFYFSRPASRVSRRSRGIVEECCFRSCDLALLETYCATPAKSE

SEQ ID NO: 8 - protein, 24 aa, Artificial Sequence
  Description of Artificial Sequence: Synthetic peptide
EECCFRSCDLALLETYCATPAKSE

SEQ ID NO: 9 - protein, 67 aa, Artificial Sequence
  Description of Artificial Sequence: Synthetic polypeptide
AYRPSETLCGGELVDTLQFVCGDRGFYFSRPASRVSRRSRGIMEECCFRSCDLALLETYCATPAKSE

SEQ ID NO: 10 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 11 - DNA, 3837 bp, Homo sapiens
gccccgcgacgagctcccgccggtcacgtgacccgcctctgcgcgcccccgggcacgaccccggagtctccgcgggcggccagggcgcgcgtgcgcggaggtgagccgggccggggctgcggggcttccctgagcgcgggccgggtcggtggggcggtcggctgcccgcgcggcctctcagttgggaaagctgaggttgtcgccggggccgcgggtggaggtcggggatgaggcagcaggtaggacagtgacctcggtgacgcgaaggaccccggccacctctaggttctcctcgtccgcccgttgttcagcgagggaggctctgcgcgtgccgcagctgacggggaaactgaggcacggagcgggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggccctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgctgcccggctgcccagagggctggatgcctgccggtccccgagcaagcctgggaactcaggaaaattcacaggacttgggagattctaaatcttaagtgcaattatttttaataaaaggggcatttggaatcagcaaaaaaaaaaaaaaaaaaaaaaaaaaaaaa

SEQ ID NO: 12 - DNA, 349 bp, Unknown
  Description of Unknown: TTR Promoter (trunc) sequence
tcgagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacaga

SEQ ID NO: 13 - DNA, 85 bp, Mouse parvovirus
ctagccctaaggtaagttggcgccgtttaagggatggttggttggtggggtattaatgtttaattaccttttttacaggcctgaa

SEQ ID NO: 14 - DNA, 441 bp, Artificial Sequence
  Description of Artificial Sequence: Synthetic polynucleotide
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaag

SEQ ID NO: 15 - DNA, 196 bp, Homo sapiens
gatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttggg

SEQ ID NO: 16 - DNA, 251 bp, Artificial Sequence
  Description of Artificial Sequence: Synthetic polynucleotide
aaggccgacccgcgaatagtagatcccgcgagggcttgaatctatcacctagagtacaccctagagaatagctagctctcaatgactaaggactaaacttggtatttcgactgaagcctgtcccctcactgttggcgctaggaggagagttcgtagaaaggatagtacgatttaagtatctctaagccttgtgaagcactaaggttgcgtacagacgtgcttgaattacggataattcgggaaccttggga

SEQ ID NO: 17 - DNA, 72 bp, Homo sapiens
atgccgtcttctgtctcgtggggcatcctcctgctggcaggcctgtgctgcctggtccctgtctccctggct

SEQ ID NO: 18 - protein, 32 aa, Rattus norvegicus
MLRGPGPGRLLLLAVLCLGTSVRCTETGKSKR

SEQ ID NO: 19 - protein, 38 aa, Rattus norvegicus
MLRGPGPGRLLLLAVLCLGTSVRCTETGKSKRLALQIV

SEQ ID NO: 20 - protein, 26 aa, Homo sapiens
MLRGPGPGLLLLAVQCLGTAVPSTGA

SEQ ID NO: 21 - protein, 31 aa, Xenopus laevis
MRRGALTGLLLVLCLSVVLRAAPSATSKKRR

SEQ ID NO: 22 - DNA, 72 bp, Homo sapiens
atgggaatcccaatggggaagtcgatgctggtgcttctcaccttcttggccttcgcctcgtgctgcattgct

SEQ ID NO: 23 - DNA, 96 bp, Rattus norvegicus
atgctcaggggtccgggacccgggcggctgctgctgctagcagtcctgtgcctggggacatcggtgcgctgcaccgaaaccgggaagagcaagagg

SEQ ID NO: 24 - DNA, 114 bp, Rattus norvegicus
atgctcaggggtccgggacccgggcggctgctgctgctagcagtcctgtgcctggggacatcggtgcgctgcaccgaaaccgggaagagcaagaggcaggctcagcaaatcgtg

SEQ ID NO: 25 - DNA, 78 bp, Homo sapiens
atgcttaggggtccggggcccgggctgctgctgctggccgtccagtgcctggggacagcggtgccctccacgggagcc

SEQ ID NO: 26 - DNA, 93 bp, Xenopus laevis
atgcgccggggggccctgaccgggctgctcctggtcctgtgcctgagtgttgtgctacgtgcagccccctctgcaacaagcaagaagcgcagg

SEQ ID NO: 27 - protein, 17 aa, Rattus norvegicus
MTPLLLLAVLCLGTALA

SEQ ID NO: 28 - protein, 22 aa, Homo sapiens
MLSFVDTRTLLLLAVTLCLATC

SEQ ID NO: 29 - protein, 21 aa, Artificial Sequence
  Description of Artificial Sequence: Synthetic peptide
MWWRLWWLLLLLLLLWPMVWA

SEQ ID NO: 30 - DNA, 9 bp, Artificial Sequence
  Description of Artificial Sequence: Synthetic oligonucleotide
ggcgcgccg

SEQ ID NO: 31 - protein, 3 aa, Artificial Sequence
  Description of Artificial Sequence: Synthetic peptide
GAP

SEQ ID NO: 32 - protein, 6 aa, Artificial Sequence
  Description of Artificial Sequence: Synthetic peptide
GGGGGP

SEQ ID NO: 33 - protein, 5 aa, Artificial Sequence
  Description of Artificial Sequence: Synthetic peptide
GGGGS

SEQ ID NO: 34 - protein, 5 aa, Artificial Sequence
  Description of Artificial Sequence: Synthetic peptide
EAAAK

SEQ ID NO: 35 - protein, 11 aa, Artificial Sequence
  Description of Artificial Sequence: Synthetic peptide
GGGTVGDDDDK

SEQ ID NO: 36 - DNA, 167 bp, Artificial Sequence
  Description of Artificial Sequence: Synthetic polynucleotide
aggaacccctagtgatggagttggccactccctctctgcgcgctcgctcgctcactgaggcaatttgataaaaatcgtcaaattataaacaggctttgcctgtttagcctcagtgagcgagcgagcgcgcagagagggagtggccaactccatcactaggggttcct

SEQ ID NO: 37 - DNA, 143 bp, Artificial Sequence
  Description of Artificial Sequence: Synthetic polynucleotide
aggaacccctagtgatggagttggccactccctctctgcgcgctcgctcgctcactgagggataaaaatccaggctttgcctgcctcagtgagcgagcgagcgcgcagagagggagtggccaactccatcactaggggttcct

SEQ ID NO: 38 - DNA, 143 bp, Artificial Sequence; identical to SEQ ID NO: 37
  Description of Artificial Sequence: Synthetic polynucleotide

SEQ ID NO: 39 - DNA, 208 bp, Artificial Sequence
  Description of Artificial Sequence: Synthetic polynucleotide
aggaacccctagtgatggagttggccactccctctctgggattgggattgcgcgctcgctcgcgggattgggattgggattgggattgggattgggattgataaaaatcaatcccaatcccaatcccaatcccaatcccaatcccgcgagcgagcgcgcaatcccaatcccagagagggagtggccaactccatcactaggggttcct

SEQ ID NO: 40 - DNA, 199 bp, Artificial Sequence
  Description of Artificial Sequence: Synthetic polynucleotide
aggaacccctagtgatggagttggccactccctctctgcgcgctcgctcgctcgggattgggattgggattgggattgggattgggattgataaaaatcaatcccaatcccaatcccaatcccaatcccaatcccgcgagcgagcgcgcaggagagggagtggccaactccatcactaggggttcctaagcttattata

SEQ ID NO: 41 - DNA, 154 bp, Artificial Sequence
  Description of Artificial Sequence: Synthetic polynucleotide
aggaacccctagtgatggagttggccactccctctctgcgcgctcgctcgctcactgagggcgcctataaagataaaaatccaggctttgcctgcctcagttagcgagcgagcgcgcagagagggagtggccaactccatcactaggggttcct

SEQ ID NO: 42 - DNA, 127 bp, Artificial Sequence
  Description of Artificial Sequence: Synthetic polynucleotide
ctagtgatggagttggccactccctctctgcgcgctcgctcgctcactgagggataaaaatccaggctttgcctgcctcagtgagcgagcgagcgcgcagagagggagtggccaactccatcactag

SEQ ID NO: 43 - DNA, 2208 bp, Adeno-associated virus 3B
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttg

SEQ ID NO: 44 - protein, 736 aa, Adeno-associated virus 3B
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 45 - DNA, 2208 bp, Artificial Sequence
  Description of Artificial Sequence: Synthetic polynucleotide
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcagatgcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttg

SEQ ID NO: 46 - protein, 736 aa, Artificial Sequence
  Description of Artificial Sequence: Synthetic polypeptide
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSDASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 47 - DNA, 2208 bp, Artificial Sequence
  Description of Artificial Sequence: Synthetic polynucleotide
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcagatgcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaaggtagctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcgtaccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttg

SEQ ID NO: 48 - protein, 736 aa, Artificial Sequence
  Description of Artificial Sequence: Synthetic polypeptide
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKVANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFVPAKFASFITQYSTGQVSVEIEWELQKENSKRWNPEIQYTSNYNKSVNVDFTVDTNGVYSEPRPIGTRYLTRNL

SEQ ID NO: 49 - DNA, 2134 bp, Artificial Sequence
  Description of Artificial Sequence: Synthetic polynucleotide
gaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttg

SEQ ID NO: 50 - protein, 736 aa, Artificial Sequence
  Description of Artificial Sequence: Synthetic polypeptide
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSDASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTAASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 51 - DNA, 2208 bp, Artificial Sequence; identical to SEQ ID NO: 43
  Description of Artificial Sequence: Synthetic polynucleotide

SEQ ID NO: 52 - protein, 736 aa, Artificial Sequence
  Description of Artificial Sequence: Synthetic polypeptide
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTAASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 53 - DNA, 2208 bp, Artificial Sequence; identical to SEQ ID NO: 43
  Description of Artificial Sequence: Synthetic polynucleotide

SEQ ID NO: 54 - protein, 736 aa, Artificial Sequence
  Description of Artificial Sequence: Synthetic polypeptide
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSYSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 55 - DNA, 2859 bp, Homo sapiens
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 56 - DNA, 2652 bp, Homo sapiens
gcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 57 - DNA, 4573 bp, Artificial Sequence
  Description of Artificial Sequence: Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgagtttaaacttcgtcgacgattcgagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagactagccctaaggtaagttggcgccgtttaagggatggttggttggtggggtattaatgtttaattaccttttttacaggcctgaactaggcgcgccaccgccaccatgccgtcttctgtctcgtggggcatcctcctgctggcaggcctgtgctgcctggtccctgtctccctggctgcttaccgccccagtgagaccctgtgcggcggggagctggtggacaccctccagttcgtctgtggggaccgcggcttctacttcagcaggcccgcaagccgtgtgagccgtcgcagccgtggcatcatggaggagtgctgtttccgcagctgtgacctggccctcctggagacgtactgtgctacccccgccaagtccgagggcgcgccggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagcgagcggccgctcttagtagcagtatcgatcccagcccacttttccccaatacgactacgagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaaggccgacccgcgaatagtagatcccgcgagggcttgaatctatcacctagagtacaccctagagaatagctagctctcaatgactaaggactaaacttggtatttcgactgaagcctgtcccctcactgttggcgctaggaggagagttcgtagaaaggatagtacgatttaagtatctctaagccttgtgaagcactaaggttgcgtacagacgtgcttgaattacggataattcgggaaccttgggacacacaaaaaaccaacacacagatctaatgaaaataaagatcttttatttaggcgcctctgacttcctggggattgacctgagttctactctagcgtttgctggttcggtgaactaatctgtgagatccccaactctccgtttgggatctccactctctggtgtcctaaccttggtgccccactgtctactgctagtgagaccttacgcgctgagaaacgtggcgttactctaactaagcgacgcgcacttgcactctgaatacttctaccgtaactaaccccggacctcagaactcagacggatctacgctgtccatcaacaccagacttagattacctctgttaagtttaattaagctcgcgaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 58 - DNA, 4597 bp, Artificial Sequence
  Description of Artificial Sequence: Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgagtttaaacttcgtcgacgattcgagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagactagccctaaggtaagttggcgccgtttaagggatggttggttggtggggtattaatgtttaattaccttttttacaggcctgaactaggcgcgccaccgccaccatgctcaggggtccgggacccgggcggctgctgctgctagcagtcctgtgcctggggacatcggtgcgctgcaccgaaaccgggaagagcaagagggcttaccgccccagtgagaccctgtgcggcggggagctggtggacaccctccagttcgtctgtggggaccgcggcttctacttcagcaggcccgcaagccgtgtgagccgtcgcagccgtggcatcatggaggagtgctgtttccgcagctgtgacctggccctcctggagacgtactgtgctacccccgccaagtccgagggcgcgccggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagcgagcggccgctcttagtagcagtatcgatcccagcccacttttccccaatacgactacgagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaaggccgacccgcgaatagtagatcccgcgagggcttgaatctatcacctagagtacaccctagagaatagctagctctcaatgactaaggactaaacttggtatttcgactgaagcctgtcccctcactgttggcgctaggaggagagttcgtagaaaggatagtacgatttaagtatctctaagccttgtgaagcactaaggttgcgtacagacgtgcttgaattacggataattcgggaaccttgggacacacaaaaaaccaacacacagatctaatgaaaataaagatcttttatttaggcgcctctgacttcctggggattgacctgagttctactctagcgtttgctggttcggtgaactaatctgtgagatccccaactctccgtttgggatctccactctctggtgtcctaaccttggtgccccactgtctactgctagtgagaccttacgcgctgagaaacgtggcgttactctaactaagcgacgcgcacttgcactctgaatacttctaccgtaactaaccccggacctcagaactcagacggatctacgctgtccatcaacaccagacttagattacctctgttaagtttaattaagctcgcgaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 59 - DNA, 4594 bp, Artificial Sequence
  Description of Artificial Sequence: Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgagtttaaacttcgtcgacgattcgagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagactagccctaaggtaagttggcgccgtttaagggatggttggttggtggggtattaatgtttaattaccttttttacaggcctgaactaggcgcgccaccgccaccatgcttaggggtccggggcccgggctgctgctgctggccgtccagtgcctggggacagcggtgccctccacgggagcctcgaagagcaagagggcttaccgccccagtgagaccctgtgcggcggggagctggtggacaccctccagttcgtctgtggggaccgcggcttctacttcagcaggcccgcaagccgtgtgagccgtcgcagccgtggcatcatggaggagtgctgtttccgcagctgtgacctggccctcctggagacgtactgtgctacccccgccaagtccgagggcgcgccggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagcgagcggccgctcttagtagcagtatcgatcccagcccacttttccccaatacgactacgagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaaggccgacccgcgaatagtagatcccgcgagggcttgaatctatcacctagagtacaccctagagaatagctagctctcaatgactaaggactaaacttggtatttcgactgaagcctgtcccctcactgttggcgctaggaggagagttcgtagaaaggatagtacgatttaagtatctctaagccttgtgaagcactaaggttgcgtacagacgtgcttgaattacggataattcgggaaccttgggacacacaaaaaaccaacacacagatctaatgaaaataaagatcttttatttaggcgcctctgacttcctggggattgacctgagttctactctagcgtttgctggttcggtgaactaatctgtgagatccccaactctccgtttgggatctccactctctggtgtcctaaccttggtgccccactgtctactgctagtgagaccttacgcgctgagaaacgtggcgttactctaactaagcgacgcgcacttgcactctgaatacttctaccgtaactaaccccggacctcagaactcagacggatctacgctgtccatcaacaccagacttagattacctctgttaagtttaattaagctcgcgaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 60 - DNA, 4555 bp, Artificial Sequence
  Description of Artificial Sequence: Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgagtttaaacttcgtcgacgattcgagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagactagccctaaggtaagttggcgccgtttaagggatggttggttggtggggtattaatgtttaattaccttttttacaggcctgaactaggcgcgccaccgccaccatgccgtcttctgtctcgtggggcatcctcctgctggcaggcctgtgctgcctggtccctgtctccctggctgctctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagggcgcgccggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagcgagcggccgctcttagtagcagtatcgatcccagcccacttttccccaatacgactacgagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaaggccgacccgcgaatagtagatcccgcgagggcttgaatctatcacctagagtacaccctagagaatagctagctctcaatgactaaggactaaacttggtatttcgactgaagcctgtcccctcactgttggcgctaggaggagagttcgtagaaaggatagtacgatttaagtatctctaagccttgtgaagcactaaggttgcgtacagacgtgcttgaattacggataattcgggaaccttgggacacacaaaaaaccaacacacagatctaatgaaaataaagatcttttatttaggcgcctctgacttcctggggattgacctgagttctactctagcgtttgctggttcggtgaactaatctgtgagatccccaactctccgtttgggatctccactctctggtgtcctaaccttggtgccccactgtctactgctagtgagaccttacgcgctgagaaacgtggcgttactctaactaagcgacgcgcacttgcactctgaatacttctaccgtaactaaccccggacctcagaactcagacggatctacgctgtccatcaacaccagacttagattacctctgttaagtttaattaagctcgcgaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 61 - DNA, 4579 bp, Artificial Sequence
  Description of Artificial Sequence: Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgagtttaaacttcgtcgacgattcgagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagactagccctaaggtaagttggcgccgtttaagggatggttggttggtggggtattaatgtttaattaccttttttacaggcctgaactaggcgcgccaccgccaccatgctcaggggtccgggacccgggcggctgctgctgctagcagtcctgtgcctggggacatcggtgcgctgcaccgaaaccgggaagagcaagagggctctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagggcgcgccggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagcgagcggccgctcttagtagcagtatcgatcccagcccacttttccccaatacgactacgagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaaggccgacccgcgaatagtagatcccgcgagggcttgaatctatcacctagagtacaccctagagaatagctagctctcaatgactaaggactaaacttggtatttcgactgaagcctgtcccctcactgttggcgctaggaggagagttcgtagaaaggatagtacgatttaagtatctctaagccttgtgaagcactaaggttgcgtacagacgtgcttgaattacggataattcgggaaccttgggacacacaaaaaaccaacacacagatctaatgaaaataaagatcttttatttaggcgcctctgacttcctggggattgacctgagttctactctagcgtttgctggttcggtgaactaatctgtgagatccccaactctccgtttgggatctccactctctggtgtcctaaccttggtgccccactgtctactgctagtgagaccttacgcgctgagaaacgtggcgttactctaactaagcgacgcgcacttgcactctgaatacttctaccgtaactaaccccggacctcagaactcagacggatctacgctgtccatcaacaccagacttagattacctctgttaagtttaattaagctcgcgaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 62 - DNA, 4576 bp, Artificial Sequence
  Description of Artificial Sequence: Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgagtttaaacttcgtcgacgattcgagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagactagccctaaggtaagttggcgccgtttaagggatggttggttggtggggtattaatgtttaattaccttttttacaggcctgaactaggcgcgccaccgccaccatgcttaggggtccggggcccgggctgctgctgctggccgtccagtgcctggggacagcggtgccctccacgggagcctcgaagagcaagagggctctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagggcgcgccggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagcgagcggccgctcttagtagcagtatcgatcccagcccacttttccccaatacgactacgagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaaggccgacccgcgaatagtagatcccgcgagggcttgaatctatcacctagagtacaccctagagaatagctagctctcaatgactaaggactaaacttggtatttcgactgaagcctgtcccctcactgttggcgctaggaggagagttcgtagaaaggatagtacgatttaagtatctctaagccttgtgaagcactaaggttgcgtacagacgtgcttgaattacggataattcgggaaccttgggacacacaaaaaaccaacacacagatctaatgaaaataaagatcttttatttaggcgcctctgacttcctggggattgacctgagttctactctagcgtttgctggttcggtgaactaatctgtgagatccccaactctccgtttgggatctccactctctggtgtcctaaccttggtgccccactgtctactgctagtgagaccttacgcgctgagaaacgtggcgttactctaactaagcgacgcgcacttgcactctgaatacttctaccgtaactaaccccggacctcagaactcagacggatctacgctgtccatcaacaccagacttagattacctctgttaagtttaattaagctcgcgaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 63 - protein, 6 aa, Homo sapiens
YRPSET

SEQ ID NO: 64 - protein, 11 aa, Rattus norvegicus
LLLLAVLCLGT

SEQ ID NO: 65 - DNA, 20 bp, Unknown
  Description of Unknown: Collagen Stability (CS) sequence
cccagcccacttttccccaa

SEQ ID NO: 66 - protein, 6 aa, Unknown
  Description of Unknown: Collagen Stability (CS) sequence
PSPLFP

SEQ ID NO: 67 - DNA, 192 bp, Artificial Sequence
  Description of Artificial Sequence: Synthetic polynucleotide
gctctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagggcgcgccg

SEQ ID NO: 68 - DNA, 189 bp, Artificial Sequence
  Description of Artificial Sequence: Synthetic polynucleotide
ctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagggcgcgccg

SEQ ID NO: 69 - DNA, 210 bp, Artificial Sequence
  Description of Artificial Sequence: Synthetic polynucleotide
gcttaccgccccagtgagaccctgtgcggcggggagctggtggacaccctccagttcgtctgtggggaccgcggcttctacttcagcaggcccgcaagccgtgtgagccgtcgcagccgtggcatcatggaggagtgctgtttccgcagctgtgacctggccctcctggagacgtactgtgctacccccgccaagtccgagggcgcgccg

SEQ ID NO: 70 - DNA, 264 bp, Artificial Sequence
  Description of Artificial Sequence: Synthetic polynucleotide
atgggaatcccaatggggaagtcgatgctggtgcttctcaccttcttggccttcgcctcgtgctgcattgctgctctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagggcgcgccg

SEQ ID NO: 71 - DNA, 300 bp, Artificial Sequence
  Description of Artificial Sequence: Synthetic polynucleotide
taggcgcctctgacttcctggggattgacctgagttctactctagcgtttgctggttcggtgaactaatctgtgagatccccaactctccgtttgggatctccactctctggtgtcctaaccttggtgccccactgtctactgctagtgagaccttacgcgctgagaaacgtggcgttactctaactaagcgacgcgcacttgcactctgaatacttctaccgtaactaaccccggacctcagaactcagacggatctacgctgtccatcaacaccagacttagattacctctgttaagt

SEQ ID NO: 72 - DNA, 2859 bp, Homo sapiens
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 73 - DNA, 2859 bp, Artificial Sequence; identical to SEQ ID NO: 55
  Description of Artificial Sequence: Synthetic polynucleotide

SEQ ID NO: 74 - DNA, 2859 bp, Artificial Sequence
  Description of Artificial Sequence: Synthetic polynucleotide
atgggagtaaggcatccaccatgctctcataggctcctcgccgtatgcgcgttggtcagccttgcgaccgcagcccttctgggccacattctgctgcacgattttctcctcgttccgcgagagttgtctggaagctctccagtactcgaggaaacgcatccagcgcaccagcagggcgcatctcggcccggtccaagagacgcacaagcacaccccgggcggccaagggctgttccaactcagtgcgatgttccgcccaattcaagattcgattgcgcacccgataaagctattacgcaagagcagtgcgaagctcgcggctgctgttatatcccggcaaagcagggccttcagggcgctcaaatgggacagccgtggtgtttttttccaccgtcatatccatcctacaagcttgagaacctgtcctcatctgagatgggatacacggctaccctgacacggacaacgccaaccttcttccccaaagacattcttacactgcgactggacgtgatgatggaaacagagaatcgactgcattttactataaaggacccggctaacaggcggtacgaggttcccctggagacccctcgcgtacattctcgagctcctagcccgctctactccgtggaattttccgaagagccgtttggtgtaatagtgcatcgccaacttgacggtcgcgtactgctcaacaccacggtggcacctctgttctttgcggaccaattcttgcagctctctacttctctcccttcacaatatataaccgggctcgcagagcatctgtccccgttgatgttgtctacgtcatggacaagaatcacgctttggaaccgagacctggcccctacgccgggagctaatctgtacgggtcacatccattctatttggctctggaggatggcggtagtgcacacggggtatttctgcttaactctaatgcgatggatgtcgtgctgcagccatcccctgcgctgtcttggcgaagcaccggcggcattctggacgtgtatatctttcttggacccgagccaaagagcgtcgtacaacagtacctcgacgtagtggggtatcccttcatgcccccctattgggggctcggtttccatttgtgcagatggggctactcatccactgcaataacacgccaggtagtagaaaatatgacccgggctcactttcctcttgacgtgcagtggaatgaccttgactacatggatagtcgccgcgatttcaccttcaacaaggacggtttcagagatttccctgctatggtgcaagagctgcaccaagggggccgccggtatatgatgatcgtagatccggccatatctagctctggccctgccgggtcttatagaccgtatgatgaaggtctgaggcgcggtgtcttcatcacaaacgaaactggtcaaccactcatcggcaaggtctggcccgggtcaacggcgtttcctgatttcactaatccgacggcgctggcttggtgggaagatatggttgccgagtttcacgatcaagttccgtttgacggaatgtggattgatatgaatgagccatctaactttatacgcggtagtgaagatggttgccctaataatgagttggaaaacccaccctatgtacccggtgtcgtcggcggcacgcttcaggccgctaccatatgcgctagcagtcaccaattccttagtacgcactataacctccataatttgtacggacttactgaggcgattgctagtcatcgagcgctcgtaaaagctcgcggtacccggcccttcgtgattagtaggagtacattcgccgggcatggtcgatatgcgggccactggactggggatgtctggtccagctgggagcagttggcttcctcagtccctgaaattttgcaatttaacctgcttggtgtgccgcttgttggcgcggacgtttgcgggtttttgggtaataccagtgaagaactgtgtgttaggtggacacagcttggggcattctatcctttcatgcggaatcacaacagtcttctgtcattgccgcaggaaccgtactctttcagtgagcctgcccagcaggcgatgcggaaggccctcacattgcgatacgcacttctcccgcatctttatacgcttttccaccaggcccatgttgcgggcgagacagtcgccaggccactcttccttgagtttccgaaagacagcagcacctggacggttgaccatcagctcctctggggtgaagctttgttgattacccccgtgctgcaggcgggcaaggcggaagtaacaggctacttcccgctgggaacctggtatgaccttcagacagtgccgatcgaagctcttggctcactgccaccacctccggcagccccgcgggaaccagcgattcactccgagggccaatgggttacgcttccggctccacttgacaccatcaatgtccatcttcgcgcgggatacattatcccgcttcagggacctggcttgactactacagagtctcgccaacagccgatggcactcgcagtagctctgactaaaggaggtgaggcgcggggtgaactcttttgggatgatggggaatccctggaggttttggagaggggtgcgtacacgcaagtaatctttctcgctcgaaacaatacgatcgtcaatgagctcgtcagggtaacgtctgagggggccggtttgcaacttcagaaagtcactgtacttggcgtagccactgctcctcaacaagtcctttcaaacggggttcctgtgtcaaactttacctattcacccgataccaaggttttggacatatgcgtgtctttgttgatgggcgaacaatttctggtttcttggtgctag

SEQ ID NO: 75 - DNA, 2859 bp, Artificial Sequence
  Description of Artificial Sequence: Synthetic polynucleotide
atgggcgttagacaccctccatgctctcatagactgctggccgtgtgtgctctggtgtctcttgctacagctgccctgctgggacatatcctgctgcacgattttctgctggtgcccagagagctgtctggcagctctccagtgctggaagaaacacaccctgcacatcagcagggcgcctctagacctggacctagagatgctcaagcccatcctggcagacctagagccgtgcctacacagtgtgacgtgccacctaacagcagattcgactgcgcccctgacaaggccatcacacaagagcagtgtgaagccagaggctgctgctacattcctgccaaacaaggactgcagggcgctcagatgggacagccttggtgcttcttcccaccatcttaccccagctacaagctggaaaacctgagcagcagcgagatgggctacaccgccacactgaccagaaccacacctacattcttcccaaaggacatcctgacactgcggctggacgtgatgatggaaaccgagaaccggctgcacttcaccatcaaggaccccgccaatagaagatacgaggtgcccctggaaacccctagagtgcattctagagccccatctccactgtacagcgtggaattcagcgaggaacccttcggcgtgatcgtgcacagacagctggatggcagagtgctgctgaataccacagtggcccctctgttcttcgccgaccagtttctgcagctgagcacaagcctgcctagccagtatatcacaggcctggccgaacacctgtctccactgatgctgagcaccagctggaccagaatcaccctgtggaacagagatctggcccctacacctggcgccaatctgtacggctctcaccctttttatctggccctggaagatggcggaagcgcccacggtgtctttctgctgaacagcaacgccatggacgtggtgctgcaaccatctcctgctctgtcttggagaagcaccggcggcatcctggacgtgtacatctttctgggccctgagcctaagagcgtggtgcagcagtatctggatgtcgtgggctaccccttcatgcctccttattggggcctgggcttccacctgtgtagatggggatacagctccaccgccatcaccagacaggtggtggaaaacatgacccgggctcacttcccactggatgtgcagtggaacgacctggactacatggacagcagacgggacttcaccttcaacaaggacggcttcagagacttccccgccatggtgcaagaactgcatcaaggcggcagacggtacatgatgatcgtggatcctgccatctcttctagcggccctgccggctcttacagaccttatgatgagggcctgagaagaggcgtgttcatcaccaatgagacaggccagcctctgatcggcaaagtgtggcctggatccaccgcctttccagacttcaccaatcctaccgctctggcttggtgggaagatatggtggccgagtttcacgatcaggtgcccttcgacggcatgtggatcgacatgaacgagcccagcaacttcatcagaggcagcgaggacggctgccccaacaacgaactggaaaatcctccttacgtgccaggcgttgtcggaggaacactgcaggccgccacaatttgtgccagcagccatcagtttctgagcacccactacaacctgcacaacctgtacggcctgaccgaggccattgcttctcacagagccctggttaaggccagaggcaccagacctttcgtgatcagcagaagcacattcgccggccacggcagatatgctggacattggacaggcgacgtgtggtctagttgggagcagctggctagctctgtgcccgagatcctgcagtttaatctgctgggagtgcctctcgtgggcgccgatgtttgtggctttctgggaaacacctccgaggaactgtgtgtgcgttggacacagctgggcgccttctatcccttcatgagaaaccacaacagcctgctgagcctgcctcaagagccttacagctttagcgaacccgcacagcaggccatgagaaaggccctgactctgagatacgctctgctgccccacctgtacaccctgtttcatcaggctcatgtggccggggaaacagtggctagacccctgttcctggaattccccaaggatagcagcacctggaccgtggatcatcagctgctttggggcgaagctctgctgattacacctgtgctgcaggctggcaaggccgaagtgacaggctattttcccctcggcacttggtacgacctgcagacagtgcctattgaggccctgggatctcttcctccacctcctgctgctcctagagagcctgccattcactctgaaggccagtgggttacactgcccgctcctctggacaccatcaatgtgcacctgagagccggctacatcatccctctgcaaggccctggactgaccacaaccgaaagcagacagcagccaatggctctggccgtggcactgacaaaaggcggagaagctagaggcgagctgttctgggatgatggcgagtctctggaagtgctcgagagaggcgcctacacacaagtgatctttctcgcccggaacaacaccatcgtgaacgaactcgtcagagtgaccagtgaaggtgccggactgcagctccagaaagtgacagtgcttggagtggccacagctcctcagcaggttctgtctaatggcgtgcccgtgtccaacttcacatacagccctgacaccaaggtgctggatatctgcgtgtcactgctgatgggcgagcagttcctggtgtcctggtgttga

SEQ ID NO: 76 - DNA, 2859 bp, Artificial Sequence
  Description of Artificial Sequence: Synthetic polynucleotide
atgggcgtgaggcacccaccttgctctcacaggctgctggccgtgtgcgcactggtgagcctggcaaccgccgccctgctgggccacatcctgctgcacgacttcctgctggtgccaagggagctgtccggaagctccccagtgctggaggagacccacccagcacaccagcagggagcatctaggccaggccccagagatgcacaggcacacccaggcagacccagagcagtgccaacccagtgcgacgtgccaccaaacagccggtttgactgtgcccccgataaggccatcacacaggagcagtgcgaggccagaggctgctgttatatccctgcaaagcagggactgcagggagcacagatgggacagccatggtgtttctttcctccatcttaccccagctataagctggagaatctgtctagctccgagatgggctacacagccaccctgacaagaaccacaccaacattctttcccaaggatatcctgaccctgcggctggacgtgatgatggagacagagaacagactgcacttcaccatcaaggatcccgccaatcggagatatgaggtgcctctggagaccccaagggtgcactctagggcacctagcccactgtactccgtggagttctctgaggagccatttggcgtgatcgtgcaccggcagctggatggcagagtgctgctgaacaccacagtggcccccctgttctttgccgaccagttcctgcagctgagcacatccctgccctcccagtatatcaccggcctggccgagcacctgtctcctctgatgctgtctaccagctggacaaggatcaccctgtggaacagggacctggcaccaacccctggagcaaatctgtacggcagccaccccttctatctggccctggaggatggaggatccgcccacggcgtgtttctgctgaactctaatgccatggacgtggtgctgcagccaagccccgccctgtcctggaggtctaccggaggcatcctggacgtgtacatcttcctgggccctgagccaaagtccgtggtgcagcagtacctggacgtggtgggctatcctttcatgcccccttactggggactgggatttcacctgtgcagatggggctattctagcacagccatcacccggcaggtggtggagaacatgaccagagcccactttccactggatgtgcagtggaatgacctggattacatggactccaggcgcgacttcaccttcaacaaggacggcttccgggattttcccgccatggtgcaggagctgcaccagggaggccggagatacatgatgatcgtggatcctgcaatctcctctagcggacctgcaggaagctacagaccatatgacgagggcctgaggcgcggcgtgttcatcacaaacgagaccggccagcctctgatcggcaaggtctggccaggctccaccgccttcccagacttcaccaatccaaccgccctggcatggtgggaggacatggtggccgagttccacgaccaggtgccttttgatggcatgtggatcgacatgaacgagccatctaatttcatcaggggaagcgaggacggatgcccaaacaatgagctggagaatccaccatatgtgcctggagtggtgggaggcacactgcaggcagcaaccatctgtgcctcctctcaccagtttctgtctacacactataacctgcacaatctgtacggactgaccgaggcaatcgcaagccacagagccctggtgaaggcaaggggaacaaggcctttcgtgatctccaggtctacctttgccggacacggccgctacgcaggacactggaccggcgacgtgtggagctcctgggagcagctggcctctagcgtgccagagatcctgcagttcaacctgctgggagtgccactggtgggagcagacgtgtgcggctttctgggcaatacaagcgaggagctgtgcgtgcggtggacccagctgggagccttctatccctttatgagaaaccacaatagcctgctgtccctgcctcaggagccatacagcttctccgagcctgcacagcaggcaatgaggaaggccctgacactgagatatgccctgctgccacacctgtacaccctgtttcaccaggcacacgtggcaggagagacagtggccaggcccctgttcctggagtttcctaaggattcctctacctggacagtggaccaccagctgctgtggggagaggccctgctgatcacccccgtgctgcaggcaggcaaggcagaggtgacaggctatttccctctgggcacatggtacgatctgcagaccgtgccaatcgaggccctgggaagcctgcctccaccacctgcagcaccaagggagcctgccatccactccgagggacagtgggtgacactgccagcacctctggacaccatcaacgtgcacctgagggccggctatatcatcccactgcagggacctggactgaccacaaccgagagcaggcagcagccaatggcactggcagtggccctgaccaagggaggcgaggccaggggcgagctgttctgggacgatggagagtccctggaggtgctggagaggggagcctacacacaggtcatcttcctggccaggaacaatacaatcgtgaatgagctggtgcgcgtgacctctgagggagcaggactgcagctgcagaaggtgacagtgctgggagtggcaaccgcaccacagcaggtgctgtccaacggcgtgcccgtgagcaatttcacatactcccctgataccaaggtgctggacatctgcgtgagcctgctgatgggcgagcagtttctggtgtcctggtgttga

SEQ ID NO: 77 - skipped

SEQ ID NO: 78 - DNA, 2032 bp, Artificial Sequence
  Description of Artificial Sequence: Synthetic polynucleotide
aagtgccgacccgtctgaatagtagatcccgtcgagggcttgaatctatcaccatagagtacacccatagagaatagctagctctcaattgactaaggactaaacttggtatttcgactgaagcctgtcactcctcactgttggcgctagtgaggagagttcgtagaaaggatagtacgatttaagtatctctaagccttgtgaagcactaaggttgcgtacagacgtgcttgaattacggataattcgtggaaccttgggatagtgcgcctctgacttctctgtgggtattgacctgagttctactctagcgtttgctggttcgtgtgaactaatctgtgagatcctcacaactctccgtttgggatctccactctctggtgtcctaacacttggtgccacacactgtctactgctagtgagaccttatctgtctgctgagaaactgtgtgctgttactctaactaagcgatctgtctgcacttgcactctgaatacttctaccgtaactaactctcacggactctcagaactcagacggatctactgctgtctcaaatcaacaccagacttagattactctctgttaagttgaaagactctctacaaatccacactgtggtctagttgaacaagcttgagagagtatctgtctgcatatcaagagtgagagtgtctctggatttggagtagttgagaagtggaaccatagcgagtctactgcctgtgtcaacttcaattagagtgttgaatagaggactgactctcgaattgaagtatcctgccaactgtctcaacttaattcaaacgctattttcgttaactgtctcaactctcgtcaagttgagaggaagagtagagccatagtttgaagcctgataagagttcgtctgaggacgtattgtgtgtcaagacgaactgaaggatttcaacaaatagagaagcttgacactgtccttcaactgtctgaactgtagcactctggatagctagctagcgtttgtagatagtttcaaggatagcaaccaacaacacgatagtttagaaattgttctagatcttctgctactactcgagagatagttgacttctagaagactctggatagagagcttcctgtggtttctatttcaacacttctgagcgatttagatagaggcgtcaattgagaacaaaaactggacacctgtttctagaacactagtgctagtcttctcaaggcttgatactgtcctgccactcaagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattagagagcgagtggatagtttagagtcgcttgagacttatcctgtgtccaactagccaagagtagagagagagtgactaggtggatcgagagatcgtcgtctatttgactgctagcctgtgttctcgtcgtagtccaaatcttcaatactctggcgactagtaacttgaatctgtgtatctaacacgacaaggctactcctttgagtttctagtgataagtgcgactggatctagctagttgagtttctagaaactactgtccaaagctactagcactaaggtctctctgtgaaagagtggtctacttttgaggagaagaaagttctagcgtagtagcaattcctttggtggaagaggctatcgagagtcaactctgccttcaagtagcttccaaaagtaaacaactgtggatcgtttctaatctcaagacgcttgaaagagcaagagcttgtattgatcgaggtggtattgagatagttaagactgtccaagaattacgactaccaaagatcttcgttagcaacgaaacttgagaacgtttctaagcctgtgacttaaactctcctgcttggtgcctgcctttgaatctggactcgtagagcacgaggaagagaagtcgatttctagagcctgct

SEQ ID NO: 79 - DNA, 4256 bp, Artificial Sequence
  Description of Artificial Sequence: Synthetic polynucleotide
tcgagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagactagccctaaggtaagttggcgccgtttaagggatggttggttggtggggtattaatgtttaattaccttttttacaggcctgaactaggcgcgccaccgccaccatgccgtcttctgtctcgtggggcatcctcctgctggcaggcctgtgctgcctggtccctgtctccctggctgcttaccgccccagtgagaccctgtgcggcggggagctggtggacaccctccagttcgtctgtggggaccgcggcttctacttcagcaggcccgcaagccgtgtgagccgtcgcagccgtggcatcatggaggagtgctgtttccgcagctgtgacctggccctcctggagacgtactgtgctacccccgccaagtccgagggcgcgccggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagcgagcggccgctcttagtagcagtatcgatcccagcccacttttccccaatacgactacgagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttggggtccttcaacgtcgaactgtagcactctggatagctagctagcgtttgtagatagtttcaaggatagcaaccaacaacacgatagtttagaaattgttctagatcttctgctactactcgagagatagttgacttctagaagactctggatagagagcttcctgtggtttctatttcaacacttcgagcgatttagatagaggcgtcaattgagaacaaaaactggacacctgtttctagaacactagtgctagtcttctcaaggcttgatacgtcctgccactcacacacaaaaaaccaacacacagattaatgaaaataaagatcttttattagagagcgagtggatagtttagagtcgcttgagacttatcctgtgtccaactagccaagagtagagagagagtgactaggtggatcgagagatcgtcgtctatttgactgctagcctgtgttctcgtcgtagtccaaatcttcaatactctggcgactagtaacttgaatctgtgtatctaacacgacaaggctactcctttgagtttctagtgataagtgcgactggatctagctagttgagtttctagaaactacgtccaaagctactagcacta

SEQ ID NO: 80 - DNA, 4280 bp, Artificial Sequence
  Description of Artificial Sequence: Synthetic polynucleotide
tcgagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagactagccctaaggtaagttggcgccgtttaagggatggttggttggtggggtattaatgtttaattaccttttttacaggcctgaactaggcgcgccaccgccaccatgctcaggggtccgggacccgggcggctgctgctgctagcagtcctgtgcctggggacatcggtgcgctgcaccgaaaccgggaagagcaagagggcttaccgccccagtgagaccctgtgcggcggggagctggtggacaccctccagttcgtctgtggggaccgcggcttctacttcagcaggcccgcaagccgtgtgagccgtcgcagccgtggcatcatggaggagtgctgtttccgcagctgtgacctggccctcctggagacgtactgtgctacccccgccaagtccgagggcgcgccggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagcgagcggccgctcttagtagcagtatcgatcccagcccacttttccccaatacgactacgagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttggggtccttcaacgtcgaactgtagcactctggatagctagctagcgtttgtagatagtttcaaggatagcaaccaacaacacgatagtttagaaattgttctagatcttctgctactactcgagagatagttgacttctagaagactctggatagagagcttcctgtggtttctatttcaacacttcgagcgatttagatagaggcgtcaattgagaacaaaaactggacacctgtttctagaacactagtgctagtcttctcaaggcttgatacgtcctgccactcacacacaaaaaaccaacacacagattaatgaaaataaagatcttttattagagagcgagtggatagtttagagtcgcttgagacttatcctgtgtccaactagccaagagtagagagagagtgactaggtggatcgagagatcgtcgtctatttgactgctagcctgtgttctcgtcgtagtccaaatcttcaatactctggcgactagtaacttgaatctgtgtatctaacacgacaaggctactcctttgagtttctagtgataagtgcgactggatctagctagttgagtttctagaaactacgtccaaagctactagcacta

SEQ ID NO: 81 - DNA, 4277 bp, Artificial Sequence
  Description of Artificial Sequence: Synthetic polynucleotide
tcgagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagactagccctaaggtaagttggcgccgtttaagggatggttggttggtggggtattaatgtttaattaccttttttacaggcctgaactaggcgcgccaccgccaccatgcttaggggtccggggcccgggctgctgctgctggccgtccagtgcctggggacagcggtgccctccacgggagcctcgaagagcaagagggcttaccgccccagtgagaccctgtgcggcggggagctggtggacaccctccagttcgtctgtggggaccgcggcttctacttcagcaggcccgcaagccgtgtgagccgtcgcagccgtggcatcatggaggagtgctgtttccgcagctgtgacctggccctcctggagacgtactgtgctacccccgccaagtccgagggcgcgccggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagcgagcggccgctcttagtagcagtatcgatcccagcccacttttccccaatacgactacgagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttggggtccttcaacgtcgaactgtagcactctggatagctagctagcgtttgtagatagtttcaaggatagcaaccaacaacacgatagtttagaaattgttctagatcttctgctactactcgagagatagttgacttctagaagactctggatagagagcttcctgtggtttctatttcaacacttcgagcgatttagatagaggcgtcaattgagaacaaaaactggacacctgtttctagaacactagtgctagtcttctcaaggcttgatacgtcctgccactcacacacaaaaaaccaacacacagattaatgaaaataaagatcttttattagagagcgagtggatagtttagagtcgcttgagacttatcctgtgtccaactagccaagagtagagagagagtgactaggtggatcgagagatcgtcgtctatttgactgctagcctgtgttctcgtcgtagtccaaatcttcaatactctggcgactagtaacttgaatctgtgtatctaacacgacaaggctactcctttgagtttctagtgataagtgcgactggatctagctagttgagtttctagaaactacgtccaaagctactagcacta

SEQ ID NO: 82 - DNA, 4238 bp, Artificial Sequence
  Description of Artificial Sequence: Synthetic polynucleotide
tcgagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagactagccctaaggtaagttggcgccgtttaagggatggttggttggtggggtattaatgtttaattaccttttttacaggcctgaactaggcgcgccaccgccaccatgccgtcttctgtctcgtggggcatcctcctgctggcaggcctgtgctgcctggtccctgtctccctggctgctctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagggcgcgccggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagcgagcggccgctcttagtagcagtatcgatcccagcccacttttccccaatacgactacgagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttggggtccttcaacgtcgaactgtagcactctggatagctagctagcgtttgtagatagtttcaaggatagcaaccaacaacacgatagtttagaaattgttctagatcttctgctactactcgagagatagttgacttctagaagactctggatagagagcttcctgtggtttctatttcaacacttcgagcgatttagatagaggcgtcaattgagaacaaaaactggacacctgtttctagaacactagtgctagtcttctcaaggcttgatacgtcctgccactcacacacaaaaaaccaacacacagattaatgaaaataaagatcttttattagagagcgagtggatagtttagagtcgcttgagacttatcctgtgtccaactagccaagagtagagagagagtgactaggtggatcgagagatcgtcgtctatttgactgctagcctgtgttctcgtcgtagtccaaatcttcaatactctggcgactagtaacttgaatctgtgtatctaacacgacaaggctactcctttgagtttctagtgataagtgcgactggatctagctagttgagtttctagaaactacgtccaaagctactagcacta

SEQ ID NO: 83 - DNA, 4262 bp, Artificial Sequence
  Description of Artificial Sequence: Synthetic polynucleotide
tcgagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagactagccctaaggtaagttggcgccgtttaagggatggttggttggtggggtattaatgtttaattaccttttttacaggcctgaactaggcgcgccaccgccaccatgctcaggggtccgggacccgggcggctgctgctgctagcagtcctgtgcctggggacatcggtgcgctgcaccgaaaccgggaagagcaagagggctctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagggcgcgccggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagcgagcggccgctcttagtagcagtatcgatcccagcccacttttccccaatacgactacgagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttggggtccttcaacgtcgaactgtagcactctggatagctagctagcgtttgtagatagtttcaaggatagcaaccaacaacacgatagtttagaaattgttctagatcttctgctactactcgagagatagttgacttctagaagactctggatagagagcttcctgtggtttctatttcaacacttcgagcgatttagatagaggcgtcaattgagaacaaaaactggacacctgtttctagaacactagtgctagtcttctcaaggcttgatacgtcctgccactcacacacaaaaaaccaacacacagattaatgaaaataaagatcttttattagagagcgagtggatagtttagagtcgcttgagacttatcctgtgtccaactagccaagagtagagagagagtgactaggtggatcgagagatcgtcgtctatttgactgctagcctgtgttctcgtcgtagtccaaatcttcaatactctggcgactagtaacttgaatctgtgtatctaacacgacaaggctactcctttgagtttctagtgataagtgcgactggatctagctagttgagtttctagaaactacgtccaaagctactagcacta

SEQ ID NO: 84 - DNA, 4259 bp, Artificial Sequence
  Description of Artificial Sequence: Synthetic polynucleotide
tcgagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagactagccctaaggtaagttggcgccgtttaagggatggttggttggtggggtattaatgtttaattaccttttttacaggcctgaactaggcgcgccaccgccaccatgcttaggggtccggggcccgggctgctgctgctggccgtccagtgcctggggacagcggtgccctccacgggagcctcgaagagcaagagggctctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagggcgcgccggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagcgagcggccgctcttagtagcagtatcgatcccagcccacttttccccaatacgactacgagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttggggtccttcaacgtcgaactgtagcactctggatagctagctagcgtttgtagatagtttcaaggatagcaaccaacaacacgatagtttagaaattgttctagatcttctgctactactcgagagatagttgacttctagaagactctggatagagagcttcctgtggtttctatttcaacacttcgagcgatttagatagaggcgtcaattgagaacaaaaactggacacctgtttctagaacactagtgctagtcttctcaaggcttgatacgtcctgccactcacacacaaaaaaccaacacacagattaatgaaaataaagatcttttattagagagcgagtggatagtttagagtcgcttgagacttatcctgtgtccaactagccaagagtagagagagagtgactaggtggatcgagagatcgtcgtctatttgactgctagcctgtgttctcgtcgtagtccaaatcttcaatactctggcgactagtaacttgaatctgtgtatctaacacgacaaggctactcctttgagtttctagtgataagtgcgactggatctagctagttgagtttctagaaactacgtccaaagctactagcacta

SEQ ID NO: 85 - DNA, 165 bp, Homo sapiens
ccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatga